(19) United States
(12) Patent Application Publication
(10) Pub. No.: US 2014/0342932 A1
(43) Pub. Date: Nov. 20, 2014

(54) FUNCTIONAL CELL SURFACE DISPLAY OF LIGANDS FOR THE INSULIN AND/OR INSULIN GROWTH FACTOR 1 RECEPTOR AND APPLICATIONS THEREOF

(71) Applicant: Merck Sharp & Dohme Corp., Rahway, NJ (US)

(72) Inventors: Ming-Tang Chen, Lebanon, NH (US); Byung-Kwon Choi, Norwich, VT (US); Song Lin, Hanover, NH (US); Natarajan Sethuraman, Hanover, NH (US); Hussam Shaheen, Lebanon, NH (US); Terrance Stadheim, Lyme, NH (US); Dongxing Zha, Etha, NH (US)

(21) Appl. No.: 14/345,257

(22) PCT Filed: Sep. 18, 2012
(86) PCT No.: PCT/US2012/055889

§ 371 Date: Mar. 17, 2014

Related U.S. Application Data
(60) Provisional application No. 61/538,378, filed on Sep. 23, 2011.

(51) Int. Cl. G01N 33/74

(52) U.S. Cl. CPC G01N 33/74; G01N 2333/62
USPC 506/9; 435/7.2; 435/7.32; 435/7.31; 435/7.21

(57) ABSTRACT

Systems for making, identifying, and selecting recombinant cells that express a ligand for the insulin receptor (IR) or insulin growth factor I (IGF-1) receptor are described. In general, libraries of recombinant cells are constructed that are capable of displaying a plurality of ligand molecules on the cell surface. Recombinant cells that display a ligand in a form accessible for binding to the IR and/or IGF-1 receptor can be detected and the recombinant cells displaying said ligands can be selected and isolated using cell sorting technologies. In particular aspects, the system is useful for constructing and screening libraries of recombinant cells that express and displaying insulin analogue precursors molecules to identify and select recombinant cells in the library that bind the IR and/or IGF-1 receptor with a desired affinity and/or avidity.

### CROSS REFERENCE TO RELATED APPLICATIONS

This application claims benefit of U.S. Provisional Application No. 61/538,378, which was filed Sep. 23, 2011, and which is incorporated herein in its entirety.

### BACKGROUND OF THE INVENTION

(1) Field of the Invention

The present invention relates to systems and methods for making, identifying, and selecting recombinant cells that express a ligand for the insulin (IR) or insulin growth factor 1 (IGF-1). In general, libraries of recombinant cells are constructed that are capable of displaying a plurality of ligand molecules on the cell surface. Recombinant cells that display a ligand in a form accessible for binding to the IR and/or IGF-1 receptor can be detected and the recombinant cells displaying said ligands can be selected and isolated using cell sorting technologies. In particular aspects, the system is useful for constructing and screening libraries of recombinant cells that express and displaying insulin analogue precursors molecules to identify and select recombinant cells in the library that bind the IR and/or IGF-1 receptor with a desired affinity and/or avidity.

(2) Description of Related Art

Insulin is a peptide hormone that is essential for maintaining proper glucose levels in most higher eukaryotes, including humans. Diabetes is a disease in which the individual cannot make insulin or develops insulin resistance. Type I diabetes is a form of diabetes mellitus that results from autoimmune destruction of insulin-producing beta cells of the pancreas. Type II diabetes is a metabolic disorder that is characterized by high blood glucose in the context of insulin resistance and relative insulin deficiency. Left untreated, an individual with Type I or Type II diabetes will die. While not a cure, insulin is effective for lowering glucose in virtually all forms of diabetes. Unfortunately, its pharmacology is not glucose sensitive and as such it is capable of excessive action that can lead to life-threatening hypoglycemia. Inconsistent pharmacology is a hallmark of insulin therapy such that it is extremely difficult to normalize blood glucose without occurrence of hypoglycemia. Furthermore, native insulin is of short duration of action and requires modification to render it suitable for use in control of basal glucose.

A central goal in insulin therapy has been designing recombinant insulin molecules that have modified pharmacokinetics and/or pharmacodynamics. For example, insulin glargine, which is marketed under the trade name LANTUS, is a recombinant insulin that has an amino acid sequence that has been modified to increase the pI of the molecule. The increased pI decreases the solubility of the molecule at physiological pH; therefore, when the patient injects insulin glargine into the muscle, the insulin glargine precipitates and then slowly dissolves and enters the blood stream over the following 24 hours post-administration. This property of insulin glargine enables the patient to maintain a basal level of insulin thereby reducing but not eliminating the risk of hypoglycemicia. Insulin lispro, which is marketed under the tradename HUMALOG, is an example of a recombinant insulin in which the order of the amino acids at position 28 and 29 have been reversed. The reversed amino acid sequence destabilizes hexamer formation which in turn enables the molecule to more rapidly enter the bloodstream of the patient than native insulin. This property of insulin lispro enables it to be used prandially thereby reducing but not eliminating the risk of hyperglycemia. In addition to modifying the amino acid sequence of the insulin molecule, insulin molecules have also been modified by linking various moieties to the molecule in an effort to modify the pharmacokinetic or pharmacodynamic properties of the molecule. For example, acylated insulin analogs have been disclosed in a number of publications, which include for example U.S. Pat. Nos. 5,693,609 and 6,011,007. PEGylated insulin analogs have been disclosed in a number of publications including, for example, U.S. Pat. Nos. 5,681,811, 6,309,633; 6,323,311; 6,890,518; 6,890,518; and, 7,585,837. Glycoconjugated insulin analogs have been disclosed in a number of publications including, for example, Internal Publication Nos. WO06082184, WO09089396, WO9010645, U.S. Pat. Nos. 3,847,890; 4,348,387; 7,531,191; and, 7,687,608. Remodeling of peptides, including insulin to include glycan structures for PEGylation and the like have been disclosed in publications including, for example, U.S. Pat. No. 7,138,371 and U.S. Published Application No. 20090053167.

Currently, the discovery of recombinant insulin molecules that display particular pharmacokinetic or pharmacodynamic properties is a time-consuming and laborious process. The discovery of recombinant insulin molecules with particular pharmacokinetic and/or pharmacodynamic properties would be facilitated by the development of a selection system that enabled a large number of recombinant insulin molecules to be constructed and screened to identify insulin molecules with particular physiochemical, pharmacokinetic and/or pharmacodynamic properties. Combinatorial library screening and selection methods have become a common tool for altering the recognition properties of proteins (Ellman et al., Proc. Natl. Acad. Sci. USA 94: 2779-2782 (1997): Phizicky & Fields, Microbiol. Rev. 59: 94-123 (1995)). The ability to construct and screen antibody libraries in vitro promises improved control over the strength and specificity of antibody-antigen interactions.

The most widespread technique for constructing and screening antibody libraries is phage display, whereby the protein of interest is expressed as a polypeptide fusion to a bacteriophage coat protein and subsequently screened by binding to immobilized or soluble biotinylated ligand. (See for example, Choo & Klug, Curr. Opin. Biotechnol. 6: 431-436 (1995); Hoogenboom, Trends Biotechnol. 15: 62-70 (1997); Ladner, Trends Biotechnol. 13: 426-430 (1995); Lowman et al., Biochemistry 30: 10832-10838 (1991); Markland et al., Methods Enzymol. 267: 28-51 (1996); Matthews & Wells, Science 260: 1113-1117 (1993); Wang et al., Methods Enzymol. 267: 52-68 (1996)).

Additional bacterial cell surface display methods have been developed (Francisco, et al., Proc. Natl. Acad. Sci. USA 90: 10444-10448 (1993); Georgiou et al., Nat. Biotechnol. 15: 29-34 (1997)). However, use of a prokaryotic expression system occasionally introduces unpredictable expression biases (Knappik & Pluckthun, Prot. Eng. 8: 81-89 (1995); Ulrich et al., Proc. Natl. Acad. Sci. USA 92: 11907-11911 (1995); Walker & Gilbert, J. Biol. Chem. 269: 28487-28493 (1994)) and bacterial capsular polysaccharide layers present a diffusion barrier that restricts such systems to small molecule ligands (Roberts, Annu. Rev. Microbiol. 50: 285-315 (1996)). E. coli possesses a lipopolysaccharide layer or capsule that may interfere sterically with macromolecular binding reactions. In fact, a presumed physiological function of the bacterial capsule is restriction of macromolecular diffusion to the cell membrane, in order to shield the cell from the immune system (DiRienzo et al., Ann. Rev. Biochem. 47: 481-532, (1978)). Since the periplasm of E. coli has not evolved as a compartment for the folding and assembly of antibody fragments, expression of antibodies in E. coli has typically been very clone dependent, with some clones expressing well and others not at all. Such variability introduces concerns about equivalent representation of all possible sequences in an antibody library expressed on the surface of E. coli. Moreover, phage display does not allow some important posttranslational modifications such as glycosylation that can affect specificity or affinity of the antibody. About a third of circulating monoclonal antibodies contain one or more N-linked glycans in the variable regions. In some cases it is believed that these N-glycans in the variable region may play a significant role in antibody function. Finally, prokaryotes do not express insulin molecules in a conformation that is functional.

To avoid some of the shortcoming of prokaryote-based display systems, lower eukaryote surface display systems have been developed. The ease of growth culture and facility of genetic manipulation available with yeast has enabled large populations of mutagenized proteins to be synthesized and screened rapidly.

U.S. Pat. Nos. 6,300,065 and 6,699,658 describe the development of a yeast surface display system for screening combinatorial antibody libraries and a screen based on antibody-antigen dissociation kinetics. The system relies on transforming yeast with vectors that express an antibody or antibody fragment fused to a yeast cell surface anchoring protein, using mutagenesis to produce a variegated population of mutants of the antibody or antibody fragment and then screening and selecting those cells that produce the antibody or antibody fragment with the desired enhanced phenotypic properties. U.S. Pat. No. 7,132,273 discloses various yeast cell wall anchor proteins and a surface expression system that uses them to immobilize foreign enzymes or polypeptides on the cell wall.

U.S. Published Application No. 2005/0142562 discloses compositions, kits and methods are provided for generating highly diverse libraries of proteins such as antibodies via homologous recombination in vivo, and screening these libraries against protein, peptide and nucleic acid targets using a two-hybrid method in yeast. The method for screening a library of tester proteins against a target protein or peptide comprises expressing a library of tester proteins in yeast cells, each tester protein being a fusion protein comprised of a first polypeptide subunit whose sequence varies within the library, a second polypeptide subunit whose sequence varies within the library independently of the first polypeptide, and a linker peptide which links the first and second polypeptide subunits; expressing one or more target fusion proteins in the yeast cells expressing the tester proteins, each of the target fusion proteins comprising a target peptide or protein; and selecting those yeast cells in which a reporter gene is expressed, the expression of the reporter gene being activated by binding of the tester fusion protein to the target fusion protein.

Of interest are Tanino et al, Biotechnol. Prog. 22: 989-993 (2006), which discloses construction of a Pichia pastoris cell surface display system using Flo1p anchor system; Ren et al., Molec. Biotechnol. 35:103-108 (2007), which discloses the display of adenoregulin in a Pichia pastoris cell surface display system using the Flo1p anchor system; Mergler et al., Appl. Microbiol. Biotechnol. 63:418-421 (2004), which discloses display of K. lactis yellow enzyme fused to the C-terminal half of S. cerevisiae α-agglutinin; Jacobs et al., Abstract T23, Pichia Protein expression Conference, San Diego, Calif. (Oct. 8-11, 2006), which discloses display of proteins on the surface of Pichia pastoris using α-agglutinin; Ryckaert et al., Abstracts BVBMB Meeting, Vrije Universiteit Brussel, Belgium (Dec. 2, 2005), which discloses using a yeast display system to identify proteins that bind particular lectins; U.S. Pat. No. 7,166,423, which discloses a method for identifying cells based on the product secreted by the cells by coupling to the cell surface a capture moiety that binds the secreted product, which can then be identified using a detection means; U.S. Published Application No. 2004/0219611, which discloses a biotin-avidin system for attaching protein A or G to the surface of a cell for identifying cells that express particular antibodies; U.S. Pat. No. 6,919,183, which discloses a method for identifying cells that express a particular protein by expressing in the cell a surface capture moiety and the protein wherein the capture moiety and the protein form a complex which is displayed on the surface of the cell; U.S. Pat. No. 6,114,147, which discloses a method for immobilizing proteins on the surface of a yeast or fungal using a fusion protein consisting of a binding protein fused to a cell surface anchoring protein which is expressed in the cell; and U.S. Published Application No. 20090005264 which discloses methods for surface display of protein in host cells including yeast.

Recombinant production of insulin or insulin analogues are expressed in a host cell as a proinsulin precursor molecule. In general, proinsulin precursor molecules are secreted and processed in vitro to produce molecules that have a native insulin structure. The processed molecule is then evaluated for binding to the insulin receptor. Because the molecules are processed in vitro to have the native insulin structure prior to evaluation, combinatorial library screening has not been used to identify new recombinant insulin analogues.

### BRIEF SUMMARY OF THE INVENTION

The present invention provides a system or method for making, identifying, and selecting recombinant cells that express a ligand for the insulin receptor (IR) or insulin growth factor 1 (IGF-1) receptor based upon combinatorial library screening. In general, libraries of recombinant cells are constructed that are capable of displaying a plurality of ligand molecules on the cell surface. Recombinant cells that display a ligand in a form accessible for binding to the IR and/or IGF-1 receptor can be detected. Combining this method with a cell separation technology such as fluorescence-activated cell sorting (FACS) provides a system for selecting or isolating recombinant cells that express and display ligands with increased or decreased affinity for the IR or IR subtype and/or the IGF-1 receptor.

In particular aspects, the ligand is an IR agonist, for example, an insulin precursor molecule or insulin analogue precursor molecule. Insulin is a heterodimer molecule having an A-chain held in close proximity to a B-chain by disulfide linkages and each peptide chain having a free N-terminus and a free C-terminus. The tertiary conformation of the insulin molecule is important for its biological activity. The inventors have discovered that fusion proteins comprising a recombinant insulin precursor molecule fused to a cell surface anchoring moiety may be expressed in cells competent for protein folding (e.g., yeast or filamentous fungal cells) as a single-chain or linear fusion protein having the structure

X—(B-chain peptide or analogue thereof)-(connecting peptide)-(A-chain peptide or analogue thereof)-(cell surface anchoring moiety)

and that the single-chain or linear fusion protein is folded in vivo into a structure that renders the molecule capable of interacting with the IR when the single-chain or linear fusion protein is displayed on the surface of a cell by the cell surface anchoring moiety. X— is an amine group or N-terminal propeptide or spacer peptide having an N-terminal amine group.

The inventors have also discovered that fusion proteins comprising the IGF-1 C-peptide when expressed in cells competent for protein folding are folded in vivo into a structure which is capable of binding the IGF-1 receptor.

The inventors have further discovered that fusion proteins comprising the format

X—(B-chain peptide or analogue thereof)-(connecting peptide)-(A-chain peptide or analogue thereof)-(cell surface anchoring moiety)

in which the junction (or peptide bond) between the A-chain peptide or analogue thereof and the connecting peptide may be cleaved in vivo by an endogenous protease to produce a split proinsulin heterodimer molecule in which the N-terminus of the A-chain peptide or analogue thereof is an amine group and the C-terminus of the A-chain peptide or analogue thereof is covalently linked to the N-terminus of the cell surface targeting moiety and the N-terminus of the B-chain or analogue thereof is an amine group or an N-terminal propeptide or spacer peptide having an N-terminal amine group (X) and the C-terminus of the B-chain peptide or analogue thereof is covalently linked to the N-terminus of the connecting peptide are also capable of interacting with the IR when displayed on the surface of a cell by the cell surface anchoring moiety. For example, the connecting peptide may be any polypeptide having at least four amino acids and the junction (or peptide bond) between the connecting peptide and the A-chain peptide or analogue thereof is cleaved by a kex2 protease. The kex2 protease recognizes the amino acid sequence Leu-Xaa-Lys-Arg (SEQ ID NO:68) wherein Xaa is any amino acid and cleaves peptide bonds on the C-terminal side of the Arg residue. The connecting peptide of human insulin is the C-peptide, which has the amino acid sequence shown in SEQ ID NO:65. The C-terminus of the C-peptide forms a kex2 cleavage site having the amino acid sequence of Leu-Gln-Lys-Arg (SEQ ID NO:67) of which the peptide bond between the Arg at the C-terminus of the C-peptide and the N-terminal Gly of the A-chain peptide is cleaved by the kex2 protease. Therefore, in particular embodiments, the connecting peptide may be the C-peptide of human insulin, an analogue thereof, or any other peptide of polypeptide of at least four amino acids provided the analogue or peptide or polypeptide includes a kex2 cleavage site at the C-terminal end of the analogue or peptide or polypeptide such that cleavage is the peptide bond between the C-terminal end of the analogue, peptide, or polypeptide and the N-terminal end of the A-chain peptide or analogue thereof.

Therefore, provided is a system or method for detecting and isolating recombinant cells that express a ligand for the insulin receptor (IR) or insulin growth factor 1 (IGF-1) receptor, comprising (a) constructing recombinant cells wherein each recombinant cell transiently or stably expresses a fusion protein comprising a polypeptide fused at the C-terminus to a cell surface anchoring moiety or protein, wherein the fusion protein is secreted and capable of being displayed on the surface of the recombinant cell, by transforming host cells with nucleic acid molecules encoding the fusion protein; (b) detecting recombinant cells that display on the cell surface thereof a fusion protein comprising a polypeptide capable of binding the IR or IGF-1 receptor by contacting the recombinant cells produced in (a) with the IR or IGF-1 receptor; and (c) isolating the recombinant cells that display the fusion protein detected in step (b) from recombinant cells that display fusion proteins that have little or no detectable binding to the IR or IGF-1 receptor to provide the recombinant cells that express the ligand for the IR or IGF-1 receptor.

Further provided is a system or method for detecting recombinant cells that express a ligand for the insulin receptor (IR) or insulin growth factor 1 (IGF-1) receptor; comprising (a) constructing a library of recombinant cells wherein each cell transiently or stably expresses a secreted fusion protein comprising a polypeptide fused at the C-terminus to a cell surface anchoring moiety or protein by transfecting host cells with a plurality nucleic acid molecules encoding the fusion protein, wherein each recombinant cell in the library expresses a different fusion protein that is secreted and displayed on the surface of the recombinant cell; and (b) contacting the library of recombinant cells produced in (a) with the IR or IGF-1 receptor to detect the recombinant cells in the library that express the ligand for the insulin receptor (IR) or insulin growth factor 1 (IGF-1) receptor. The recombinant cells expressing a fusion protein capable of binding the IR or IGF-1 receptor may be separated from recombinant cells that display fusion proteins that have little or no detectable binding to the IR or IGF-1 receptor to provide the recombinant cells that express a ligand for the IR or IGF-1 receptor.

Further provided is a system or method for detecting and isolating recombinant cells that express a ligand for the insulin receptor (IR) or insulin growth factor 1 (IGF-1) receptor, comprising (a) constructing recombinant cells wherein each recombinant cell transiently or stably expresses a fusion protein comprising a polypeptide fused to a cell surface anchoring moiety (protein or cell surface binding portion thereof), wherein the fusion protein is secreted and capable of being displayed on the surface of the recombinant cell, by transfecting cells with nucleic acid molecules encoding the fusion protein; (b) detecting recombinant cells that display on the cell surface thereof a fusion protein that comprises a polypeptide capable of binding the IR or IGF-1 receptor by contacting the recombinant cells produced in (a) with the IR or IGF-1 receptor; and (c) separating the recombinant cells that display the fusion protein detected in step (b) from recombinant cells that display fusion proteins that have little or no detectable binding to the IR or IGF-1 receptor to provide the recombinant cells that express the ligand for the insulin IR or IGF-1 receptor.

In further aspects of the above systems or methods, the IR or IGF-1 receptor is labeled with or covalently linked to a detectable moiety, which may be a fluorescent moiety. In particular aspects, the IR or IGF-1 receptor is detected using an antibody specific for the IR or IGF-1 receptor or an antibody that is specific for a complex formed between the IR or IGF-1 receptor and the polypeptide. The antibody or an antibody specific for the antibody is labeled with or covalently linked to a detectable moiety.

In further aspects of the above systems or methods, the cell surface anchoring moiety or protein may be selected from the group consisting of α-agglutinin, Cwp1p, Cwp2p, Gas1p, Yap3p, Flo1p, Crh2p, Pir1p, Pir4p, Sed1p, Tip1p, Hpwp1p, Als3p, and Rbt5p. In a particular embodiment, the cell surface anchoring protein is Sed1p, for example, the Saccharomyces cerevisiae Sed1p. The cell surface anchoring moiety or protein may be a full-sized protein or a truncated protein that lacks a signal peptide or propeptide but which includes at least the cell surface anchoring portions thereof.

In further aspects of the above systems or methods, the recombinant cells in (a) are constructed by transforming or transfecting cells with first nucleic acid molecules encoding a cell surface anchoring moiety (protein or cell surface binding portion thereof) fused to a first binding moiety and second nucleic acid molecules encoding fusion proteins comprising a polypeptide fused to a second binding moiety that is specific for the first binding moiety. For example, in one embodiment, the second nucleic acid molecule encodes a recombinant insulin precursor molecule in which the recombinant insulin expressed is in a linear format of

X—(B-chain peptide or analogue thereof)-(connecting peptide)-(A-chain peptide or analogue thereof)-(second binding moiety)

in cells competent for protein folding (e.g., yeast or filamentous fungal cells) and the expressed molecule is capable of interacting with the IR when the expressed molecule is displayed on the surface of the cell by interaction of the second binding moiety covalently linked to the C-terminus of the A-chain peptide or analogue thereof with the first binding moiety attached to the cell surface by the cell surface anchoring moiety and wherein X is an amine group or an N-terminal propeptide of spacer peptide. In a further aspect, the junction between the A-chain peptide or analogue thereof and the connecting peptide may be cleaved in vivo by an endogenous protease to produce a split proinsulin heterodimer molecule in which the C-terminus of the A-chain peptide or analogue thereof is covalently linked to the N-terminus of the second binding moiety and the C-terminus of the B-chain peptide or analogue thereof is covalently linked to the N-terminus of the connecting peptide.

In particular aspects, the first binding moiety is a first peptide and the second binding moiety is a second peptide wherein the first and second peptides are capable of a specific pairwise interaction. In further aspects, the first and second peptides are coiled-coil peptides that capable of the specific pairwise interaction. In a further aspect, the coiled-coil peptides are GABAB-R1 and GABAB-R2 subunits that are capable of the specific pairwise interaction.

In particular embodiments, the cell surface anchoring moiety or protein may be selected from the group consisting of α-agglutinin, Cwp1p, Cwp2p, Gas1p, Yap3p, Flo1p, Crh2p, Pir1p, Pir4p, Sed1p, Tip1p, Hpwp1p, Als3p, and Rbt5p. In a particular embodiment, the cell surface anchoring moiety or protein is Sed1p, for example, the Saccharomyces cerevisiae Sed1p. The cell surface anchoring moiety or protein may be a full-sized protein or a truncated protein that lacks a signal peptide or propeptide but which includes at least the cell surface anchoring portions thereof.

In further aspects of the above systems or methods, the polypeptide is fused to a modification motif that is coupled to a first binding partner when the fusion proteins are expressed and which binds to a second binding partner displayed on the surface of the recombinant cells. In particular aspects, the first binding partner is biotin and the second binding partner is an avidin or an avidin-like protein such as streptavidin or neutravidin.

In further aspects of the above systems or methods, the recombinant cells are mutagenized to produce a library of recombinant cells expressing a variegated population of polypeptides.

In further aspects of the above systems or methods, the recombinant cells in (a) are produced by transforming or transfecting cells with a plurality of nucleic acid molecules in which the majority of the nucleic acid molecules comprise at least one mutation in the nucleotide sequence encoding the recombinant insulin analogue precursor to produce a library of recombinant cells wherein each recombinant cell in the library produces a single species of polypeptide.

In further aspects of the above systems or methods, the recombinant cells display on the cell surface thereof a plurality of different fusion proteins, wherein each fusion protein is encoded on a different nucleic acid molecule in a different recombinant cell. In further aspects, the different fusion proteins are sequence variants of each other.

Further provided is a system or method for detecting and isolating recombinant cells that express a ligand for the insulin receptor (IR) or insulin growth factor 1 (IGF-1) receptor, comprising (a) constructing recombinant cells wherein each recombinant cell transiently or stably expresses a fusion protein comprising a polypeptide fused to a cell surface anchoring moiety or protein or cell surface binding portion thereof, wherein the fusion protein is secreted and capable of being displayed on the surface of the recombinant cell, by transfecting cells with nucleic acid molecules encoding the fusion protein; (b) detecting recombinant cells that display on the cell surface thereof a fusion protein that comprises a polypeptide capable of binding the IR or IGF-1 receptor by contacting the recombinant cells produced in (a) with the IR or IGF-1 receptor; and (c) isolating the recombinant cells that display the fusion protein detected in step (b) from recombinant cells that display fusion proteins that have little or no detectable binding to the IR or IGF-1 receptor to provide the recombinant cells that express the ligand for the insulin IR or IGF-1 receptor.

Further provided is a system or method for detecting recombinant cells that express a ligand for the insulin receptor (IR) or insulin growth factor 1 (IGF-1) receptor; comprising (a) constructing a library of recombinant cells wherein each cell transiently or stably expresses a secreted fusion protein comprising a polypeptide fused to a cell surface anchoring moiety or protein or portion thereof by transforming or transfecting cells with a plurality nucleic acid molecules encoding the fusion protein, wherein each recombinant cell in the library expresses a different fusion protein; and (b) contacting the library of recombinant cells produced in (a) with the IR or IGF-1 receptor to detect the recombinant cells in the library that express the ligand for the IR or IGF-1 receptor. The recombinant cells expressing a fusion protein capable of binding the IR or IGF-1 receptor may be separated from recombinant cells that display fusion proteins that have little or no detectable binding to the IR or IGF-1 receptor to provide the recombinant cells that express a ligand for the IR or IGF-1 receptor.

Further provided is a system or method for detecting and isolating recombinant cells that express a ligand for the insulin receptor (IR) or insulin growth factor 1 (IGF-1) receptor, comprising (a) providing recombinant cells comprising a first nucleic acid molecule encoding a cell surface anchoring protein or cell surface binding portion thereof fused to a first binding moiety and a second nucleic acid molecule encoding a fusion protein comprising a polypeptide fused to a second binding moiety that is specific for the first binding moiety; (b) detecting recombinant cells that display on the cell surface thereof a fusion protein that comprises a polypeptide capable of binding the IR or IGF-1 receptor by contacting the recombinant cells produced in (a) with the IR or IGF-1 receptor; and (c) isolating the recombinant cells that display the fusion protein detected in step (b) from recombinant cells that express fusion proteins that have little or no detectable binding to the IR or IGF-1 receptor to provide the host cells that express the ligand for the insulin IR or IGF-1 receptor.

In further aspects of the above systems or methods, the IR or IGF-1 receptor is labeled with a detectable moiety, which may be a fluorescent moiety. In particular aspects, the IR or IGF-1 receptor is detected using an antibody specific for the IR or IGF-1 receptor or an antibody that is specific for a complex formed between the IR or IGF-1 receptor and the polypeptide.

In further aspects of the above systems or methods, the recombinant cells in (a) are constructed by transforming or transfecting cells with first nucleic acid molecules encoding a cell surface anchoring protein or cell surface binding portion thereof fused to a first binding moiety and second nucleic acid molecules encoding fusion proteins comprising a polypeptide fused to a second binding moiety that is specific for the first binding moiety. In particular aspects, the first binding moiety is a first peptide and the second binding moiety is a second peptide wherein the first and second peptides are capable of a specific pairwise interaction. In further aspects, the first and second peptides are coiled-coil peptides that capable of the specific pairwise interaction. In a further aspect, the coiled-coil peptides are GABAB-R1 and GABAB-R2 subunits that are capable of the specific pairwise interaction.

Further provided is a system or method for detecting and isolating recombinant cells that express a ligand for the insulin receptor (IR) or insulin growth factor 1 (IGF-1) receptor, comprising (a) constructing a cell line transiently or stably expressing a first nucleic acid molecule encoding a capture moiety comprising a cell surface anchoring protein fused to a first binding moiety; (b) transforming or transfecting the cell line constructed in (a) with a second nucleic acid molecule that encodes a fusion protein comprising an insulin analogue precursor fused to a second binding moiety that is capable of specifically interacting with the first binding moiety to produce recombinant cells wherein the fusion protein is secreted; (c) detecting the fusion protein displayed on the surface of a recombinant cell of the recombinant cells produced in (b) by contacting the recombinant cells produced in (b) with the IR or IGF-1 receptor; and (d) isolating the recombinant cells bearing the surface displayed fusion protein detected in step (c) from recombinant cells that display fusion proteins that have little or no detectable binding to the IR or IGF-1 receptor to provide the recombinant cells that express the ligand for the IR or IGF-1 receptor.

In further aspects of the above methods, the cell surface anchoring moiety or protein may be selected from the group consisting of α-agglutinin, Cwp1p, Cwp2p, Gas1p, Yap3p, Flo1p, Crh2p, Pir1p, Pir4p, Sed1p, Tip1p, Hpwp1p, Als3p, and Rbt5p. In a particular embodiment, the cell surface anchoring moiety or protein is Sed1p. The cell surface anchoring moiety or protein may be a full-sized protein or a truncated protein that lacks a signal peptide or propeptide but which includes at least the cell surface anchoring portions thereof.

Further provided is a system or method for detecting and isolating recombinant cells that express a recombinant insulin analogue precursor molecule of interest, comprising (a) constructing recombinant cells wherein each recombinant cell transiently or stably expresses a fusion protein comprising an insulin analogue precursor, wherein the fusion protein is secreted and capable of being displayed on the surface of the recombinant cell, by transforming or transfecting cells with nucleic acid molecules encoding the fusion protein; (b) detecting the recombinant cells that display on the cell surface thereof the fusion protein comprising the recombinant insulin analogue precursor molecule of interest by contacting the recombinant cells produced in (a) with an insulin receptor; and (c) isolating the recombinant cells that display the fusion protein detected in step (b) from recombinant cells that display fusion proteins that have little or no detectable binding to the IR or IGF-1 receptor to provide the recombinant cells that express the recombinant insulin analogue precursor molecule of interest.

Further provided is a system or method for detecting recombinant cells that express a recombinant insulin analogue precursor molecule of interest; comprising (a) constructing a library of recombinant cells wherein each cell transiently or stably expresses a secreted fusion protein comprising a recombinant insulin analogue precursor molecule fused to a cell surface anchoring protein or portion thereof by transforming or transfecting cells with a plurality nucleic acid molecules encoding the fusion protein, wherein each recombinant cell in the library expresses a different fusion protein; and (b) contacting the library of recombinant cells produced in (a) with the insulin receptor to detect the recombinant cells in the library that express the insulin analogue precursor molecule of interest.

Further provided is a system or method for detecting and isolating recombinant cells that express a recombinant insulin analogue precursor molecule, comprising (a) constructing a cell line transiently or stably expressing a first nucleic acid molecule encoding a capture moiety comprising a cell surface anchoring protein fused to a first binding moiety; (b) transforming or transfecting the cell line constructed in (a) with a second nucleic acid molecule that encodes a fusion protein comprising an insulin analogue precursor fused to a second binding moiety that is capable of specifically interacting with the first binding moiety to produce recombinant cells wherein the fusion protein is secreted; (c) detecting the fusion protein displayed on the surface of a recombinant cell of the recombinant cells produced in (b) by contacting the recombinant cells produced in (b) with an insulin receptor; and (d) isolating the recombinant cells bearing the surface displayed fusion protein detected in step (c) from recombinant cells that display fusion proteins that have little or no detectable binding to the IR or IGF-1 receptor to provide the recombinant cells that express the recombinant insulin analogue precursor molecule.

Further provided is a system or method for producing a recombinant cell that expresses a recombinant insulin analogue precursor molecule of interest, comprising (a) constructing recombinant cells that transiently or stably express fusion proteins comprising an insulin analogue precursor, wherein the fusion proteins are secreted and capable of being displayed on the surface of the recombinant cells, by transforming or transfecting cells with nucleic acid molecules encoding the fusion protein; (b) detecting the recombinant cells that display on the cell surface thereof the fusion protein comprising the recombinant insulin analogue precursor molecule of interest by contacting the recombinant cells produced in (a) with an insulin receptor; (c) isolating the recombinant cells that display the fusion protein detected in step (b) to provide host cells that display the recombinant insulin analogue precursor molecule of interest; (d) isolating the nucleic acid molecule encoding the recombinant insulin analogue precursor molecule of interest from recombinant cells that display fusion proteins that have little or no detectable binding to the IR or IGF-1 receptor and determining the sequence of the nucleic acid molecule encoding the recombinant insulin analogue precursor molecule of interest; (e) constructing an expression vector that encodes the recombinant insulin analogue precursor molecule of interest wherein the recombinant insulin analogue precursor molecule of interest is not capable of display on the cell surface; and (0 transforming or transfecting a cell with the expression vector to produce the recombinant cell that expresses the recombinant insulin analogue precursor molecule of interest.

In further aspects of the above systems or methods, the insulin receptor is labeled with a detectable moiety, which may be a fluorescent moiety. In particular aspects, the insulin receptor is detected using an antibody specific for the insulin receptor or an antibody that is specific for a complex formed between the insulin receptor and the recombinant insulin analogue precursor.

In further aspects of the above systems or methods, the insulin analogue precursor is fused to a cell surface anchoring protein or cell surface binding portion thereof. In particular embodiments, the cell surface anchoring moiety or protein may be selected from the group consisting of α-agglutinin, Cwp1p, Cwp2p, Gas1p, Yap3p, Flo1p, Crh2p, Pir1p, Pir4p, Sed1p, Tip1p, Hpwp1p, Als3p, and Rbt5p. In a particular embodiment, the cell surface anchoring moiety or protein is Sed1p. The cell surface anchoring moiety or protein may be a full-sized protein or a truncated protein that lacks a signal peptide or propeptide but which includes at least the cell surface anchoring portions thereof.

In a further aspects of the above systems or methods, the recombinant cells in (a) are constructed by transforming or transfecting cells with first nucleic acid molecules encoding a cell surface anchoring protein or cell surface binding portion thereof fused to a first binding moiety and second nucleic acid molecules encoding fusion proteins comprising an insulin analogue precursor fused to a second binding moiety that is specific for the first binding moiety. In particular aspects, the first binding moiety is a first peptide and the second binding moiety is a second peptide wherein the first and second peptides are capable of a specific pairwise interaction. In further aspects, the first and second peptides are coiled-coil peptides that capable of the specific pairwise interaction. In a further aspect, the coiled-coil peptides are GABAB-R1 and GABAB-R2 subunits that are capable of the specific pairwise interaction.

In a further embodiment of the above systems or methods, the insulin analogue precursor is fused to a modification motif that is coupled to a second binding partner when the fusion proteins are expressed and which binds to a first binding partner displayed on the surface of the recombinant cells. In particular aspects, the second binding partner is biotin and the first binding partner is an avidin or an avidin-like protein such as streptavidin or neutravidin.

In a further aspects of the above systems or methods, the recombinant cells are mutagenized to produce a library of recombinant cells expressing a variegated population of mutant recombinant insulin analogue precursors.

In further aspects of the above systems or methods, the recombinant cells in (a) are produced by transfecting cells with a plurality of nucleic acid molecules in which the majority of the nucleic acid molecules comprise at least one mutation in the nucleotide sequence encoding the recombinant insulin analogue precursor to produce a library of recombinant cells wherein each recombinant cell in the library produces a single species of recombinant insulin analogue precursor.

In further aspects of the above systems or methods, the recombinant cells in (a) are produced by transfecting cells with a plurality of nucleic acid molecules in which the majority of the nucleic acid molecules comprise at least one N-glycan attachment site in the nucleotide sequence encoding the recombinant insulin analogue precursor to produce a library of recombinant cells wherein each recombinant cell in the library produces a single species of recombinant insulin analogue precursor.

In a further aspects of the above systems or methods, the recombinant cells display on the cell surface thereof a plurality of different fusion proteins, wherein each fusion protein is encoded on a different nucleic acid molecule in a different recombinant cell. In further aspects, the different fusion proteins are sequence variants of each other.

In a further aspects of the above systems or methods, the recombinant cells in step (c) are contacted with the insulin growth factor 1 (IGF-1) receptor and the recombinant cells that display a fusion protein that lacks detectable binding to the IGF-1 are isolated to provide the recombinant cells that express the recombinant insulin analogue precursor molecule of interest.

In particular aspects of any one of the above systems or methods, the cell or recombinant cell is a bacteria cell, engineered bacteria cell, mammalian cell, insect cell, or plant cell, e.g., suspension culture of any one of the foregoing cells. In a further aspects, the cell or recombinant cell is a yeast or filamentous fungi cell which may be selected from the group consisting of Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Yarrowia lypolytica, and Neurospora crassa. In a further aspect, the above cell is Pichia pastoris.

In a particular aspect of any one of the above recombinant cells, the recombinant cell is Pichia pastoris. In a further aspect, the recombinant cell is an och1 mutant of Pichia pastoris. In a further aspect, the recombinant cell is an och1 alg3 double mutant of Pichia pastoris.

In further embodiments of any one of the above systems or methods, the host cell is genetically engineered to minimize or lack detectable O-glycosylation by deleting or disrupting one or more of the genes encoding protein mannosyltransferases (PMT).

In further embodiments of any one of the above systems or methods, the cell is genetically engineered to produce glycoproteins comprising one or more mammalian- or human-like complex N-glycans.

In particular aspects, the cell includes one or more nucleic acid molecules encoding one or more catalytic domains of a glycosidase, mannosidase, or glycosyltransferase activity derived from a member of the group consisting of UDP-GlcNAc transferase (GnT) I, GnT II, GnT III, GnT IV, GnT V, GnT VI, UDP-galactosyltransferase (GalT), fucosyltransferase, and sialyltransferase. In particular embodiments, the mannosidase is selected from the group consisting of C. elegans mannosidase IA, C. elegans mannosidase IB, D. melanogaster mannosidase IA, H. sapiens mannosidase IB, P. citrinum mannosidase I, mouse mannosidase IA, mouse mannosidase IB, A. nidulans mannosidase IA, A. nidulans mannosidase IB, A. nidulans mannosidase IC, mouse mannosidase II, C. elegans mannosidase II, H. sapiens mannosidase II, and mannosidase III.

In particular aspects, at least one catalytic domain is localized by forming a fusion protein comprising the catalytic domain and a cellular targeting signal peptide. The fusion protein can be encoded by at least one genetic construct formed by the in-frame ligation of a DNA fragment encoding a cellular targeting signal peptide with a DNA fragment encoding a catalytic domain having enzymatic activity. Examples of targeting signal peptides include, but are not limited to, those to membrane-bound proteins of the ER or Golgi, retrieval signals such as HDEL or KDEL, Type II membrane proteins, Type I membrane proteins, membrane spanning nucleotide sugar transporters, mannosidases, sialyltransferases, glucosidases, mannosyltransferases, and phosphomannosyltransferases.

In particular aspects of any one of the above cells, the cell further includes one or more nucleic acid molecules encoding one or more enzymes selected from the group consisting of UDP-GlcNAc transporter, UDP-galactose transporter, GDP-fucose transporter, CMP-sialic acid transporter, and nucleotide diphosphatases.

In further aspects of any one of the above cells, the cell includes one or more nucleic acid molecules encoding an α1,2-mannosidase activity, a UDP-GlcNAc transferase (GnT) I activity, a mannosidase II activity, and a GnT II activity.

In further still aspects of any one of the above cells, the cell includes one or more nucleic acid molecules encoding an α1,2-mannosidase activity, a UDP-GlcNAc transferase (GnT) I activity, a mannosidase II activity, a GnT II activity, and a UDP-galactosyltransferase (GalT) activity.

In further still aspects of any one of the above cells, the cell is deficient in the activity of one or more enzymes selected from the group consisting of mannosyltransferases and phosphomannosyltransferases. In further still aspects, the host cell does not express an enzyme selected from the group consisting of 1,6 mannosyltransferase, 1,3 mannosyltransferase, and 1,2 mannosyltransferase.

Further provided is a recombinant cell comprising a nucleic acid molecule encoding a fusion protein comprising an insulin analogue precursor fused to a cell surface anchoring protein. In particular embodiments, the cell surface anchoring moiety or protein may be selected from the group consisting of α-agglutinin, Cwp1p, Cwp2p, Gas1p, Yap3p, Flo1p, Crh2p, Pir1p, Pir4p, Sed1p, Tip 1p, Hpwp1p, Als3p, and Rbt5p. In a particular embodiment, the cell surface anchoring moiety or protein is Sed1p. The cell surface anchoring moiety or protein may be a full-sized protein or a truncated protein that lacks a signal peptide or propeptide but which includes at least the cell surface anchoring portions thereof.

Further provided is a recombinant cell comprising a nucleic acid molecule encoding a fusion protein comprising an insulin analogue precursor fused to a binding moiety. In particular aspects, the binding moiety is capable of a specific pairwise interaction with a second binding moiety. In further aspects, the binding moiety is a coiled coil peptide that is capable of the specific pairwise interaction. In a further aspect, the coiled coil peptide is GABAB-R1 or GABAB-R2 subunit capable of the specific pairwise interaction.

In particular aspects, the recombinant cell is a bacterial, mammalian, insect, or plant cell. In a further aspects, the recombinant cell is a yeast or filamentous fungi cell which may be selected from the group consisting of Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa.

In a particular aspect of any one of the above recombinant cells, the recombinant cell is Pichia pastoris. In a further aspect, the recombinant cell is an och1 mutant of Pichia pastoris. In a further aspect, the recombinant cell is an och1alg3 double mutant of Pichia pastoris.

Further provided is a plasmid comprising a nucleic acid molecule encoding a fusion protein comprising an insulin analogue precursor fused to a cell surface anchoring protein. In particular embodiments, the cell surface anchoring moiety or protein may be selected from the group consisting of α-agglutinin, Cwp1p, Cwp2p, Gas1p, Yap3p, Flo1p, Crh2p, Pir1p, Pir4p, Sed1p, Tip1p, Hpwp1p, Als3p, and Rbt5p. In a particular embodiment, the cell surface anchoring moiety or protein is Sed1p. The cell surface anchoring moiety or protein may be a full-sized protein or a truncated protein that lacks a signal peptide or propeptide but which includes at least the cell surface anchoring portions thereof.

Further provided is a plasmid comprising a nucleic acid molecule encoding a fusion protein comprising an insulin analogue precursor fused to a binding moiety. In particular aspects, the binding moiety is capable of a specific pairwise interaction with a second binding moiety. In further aspects, the binding moiety is a coiled-coil peptide that is capable of the specific pairwise interaction. In a further aspect, the coiled-coil peptide is GABAB-R1 or GABAB-R2 subunit capable of the specific pairwise interaction.

Further provided is an insulin analogue comprising an amino acid sequence determined using the methods disclosed herein.

Further provided is the use of the method herein in the manufacture of a medicament for treating diabetes.

### DEFINITIONS

As used herein, the term “insulin” means the active principle of the pancreas that affects the metabolism of carbohydrates in the animal body and which is of value in the treatment of diabetes mellitus. The term includes synthetic and biotechnologically-derived products that are the same as, or similar to, naturally occurring insulins in structure, use, and intended effect and are of value in the treatment of diabetes mellitus.

The term “insulin” or “insulin molecule” is a generic term that designates the 51 amino acid heterodimer comprising the A-chain peptide having the amino acid sequence shown in SEQ ID NO: 38 and the B-chain peptide having the amino acid sequence shown in SEQ ID NO: 39.

The term “insulin analogue” as used herein includes any heterodimer analogue or single-chain analogue that comprises one or more modification(s) of the native A-chain peptide and/or B-chain peptide. Modifications include but are not limited to any amino acid substitution or deletion at any position in the A-chain peptide, B-chain peptide, and/or C-peptide or conjugating directly or by a polymeric or non-polymeric linker one or more acyl, polyethylglycine (PEG), or saccharide moiety (moieties); or any combination thereof. The term further includes any insulin heterodimer and single-chain analogue that has been modified to have at least one N-linked glycosylation site and in particular, embodiments in which the N-linked glycosylation site is linked to or occupied by an N-glycan. Examples of insulin analogues include but are not limited to the heterodimer and single-chain analogues disclosed in published international application WO20100080606, WO2009/099763, and WO2010080609, the disclosures of which are incorporated herein by reference. Examples of single-chain insulin analogues also include but are not limited to those disclosed in published International Applications WO9634882, WO95516708, WO2005054291, WO2006097521, WO2007104734, WO2007104736, WO2007104737, WO2007104738, WO2007096332, WO2009132129; U.S. Pat. Nos. 5,304,473 and 6,630,348; and Kristensen et al., Biochem. J. 305: 981-986 (1995), the disclosures of which are each incorporated herein by reference.

The term “insulin analogues” further includes single-chain and heterodimer polypeptide molecules that have little or no detectable activity at the insulin receptor but which have been modified to include one or more amino acid modifications or substitutions to have an activity at the insulin receptor that has at least 1%, 10%, 50%, 75%, or 90% of the activity at the insulin receptor as compared to native insulin and which further includes at least one N-linked glycosylation site. In particular aspects, the insulin analogue is a partial agonist that has from 2× to 100× less activity at the insulin receptor as does native insulin. In other aspects, the insulin analogue has enhanced activity at the insulin receptor, for example, the IGF^{B16B17} derivative peptides disclosed in published international application WO2010080607 (which is incorporated herein by reference). These insulin analogues, which have reduced activity at the insulin-like growth factor receptor and enhanced activity at the insulin receptor, include both heterodimers and single-chain analogues.

As used herein, the term “single-chain insulin analogue” encompasses a group of structurally-related proteins wherein the insulin A-chain peptide and B-chain peptide are covalently linked by a polypeptide or non-peptide polymeric or non-polymeric linker and the analogue has at least 1%, 10%, 50%, 75%, or 90% of the activity of insulin at the insulin receptor as compared to native insulin.

As used herein, the term “connecting peptide” or “C-peptide” refers to the connection moiety “C” of the B-C-A polypeptide sequence of a single chain preproinsulin-like molecule. Specifically, in the natural insulin chain, the C-peptide connects the amino acid at position 30 of the B-chain and the amino acid at position 1 of the A-chain peptide. The term can refer to both the native insulin C-peptide, the monkey C-peptide, and any other peptide from 3 to 35 amino acids that connects the B-chain peptide to the A-chain peptide thus is meant to encompass any peptide linking the B-chain peptide to the A-chain peptide in a single-chain insulin analogue (See for example, U.S. Published application Nos. 20090170750 and 20080057004 and WO9634882) and in insulin precursor molecules such as disclosed in WO9516708 and U.S. Pat. No. 7,105,314.

As used herein, the term “pre-proinsulin analogue precursor” refers to a fusion protein comprising a leader peptide, which targets the prepro-insulin analogue precursor to the secretory pathway of the host cell, fused to the N-terminus of a B-chain peptide or B-chain peptide analogue, which is fused to the N-terminus of a C-peptide, which in turn is fused at its C-terminus to the N-terminus of an A-chain peptide or A-chain peptide analogue. The fusion protein may optionally include one or more extension or spacer peptides between the C-terminus of the leader peptide and the N-terminus of the B-chain peptide or B-chain peptide analogue. The extension or spacer peptide when present may protect the N-terminus of the B-chain or B-chain analogue from protease digestion during fermentation.

As used herein, the term “proinsulin analogue precursor” refers to a molecule in which the signal or pre-peptide of the pre-proinsulin analogue precursor has been removed.

As used herein, the term “insulin analogue precursor” refers to a molecule in which the propeptide of the proinsulin analogue precursor has been removed. The insulin analogue precursor may optionally include the extension or spacer peptide at the N-terminus of the B-chain peptide or B-chain peptide analogue. The insulin analogue precursor is a single-chain molecule since it includes a C-peptide; however, the insulin analogue precursor will contain correctly formed disulphide bridges (three) as in human insulin and may by one or more subsequent chemical and/or enzymatic processes be converted into a heterodimer or single-chain insulin analogue.

The term “split proinsulin” or “split proinsulin analogue” refers to a molecule in which the propeptide of the molecule has been removed and the junction between the C-peptide and the A-chain peptide has been cleaved. The “split proinsulin is a heterodimer molecule that has three disulphide bridges as in native human insulin and which may by one or more subsequent chemical and/or enzymatic processes be converted into a heterodimer insulin or insulin analogue.

As used herein, the term “leader peptide” refers to a polypeptide comprising a pre-peptide (the signal peptide) and a pro-peptide.

As used herein, the term “signal peptide” refers to a pre-peptide which is present as an N-terminal peptide on a precursor form of a protein. The function of the signal peptide is to enable or facilitate translocation of the expressed polypeptide to which it is attached into the endoplasmic reticulum. The signal peptide is normally cleaved off in the course of this process. The signal peptide may be heterologous or homologous to the organism used to produce the polypeptide. A number of signal peptides which may be used include the yeast aspartic protease 3 (YAP3) signal peptide or any functional analog (Egel-Mitani et al. YEAST 6:127 137 (1990) and U.S. Pat. No. 5,726,038) and the signal peptide of the Saccharomyces cerevisiae alpha-mating factor α1 gene (ScMF α1) gene (Thorner (1981) in The Molecular Biology of the Yeast Saccharomyces cerevisiae, Strathern et al., eds., pp 143 180, Cold Spring Harbor Laboratory, NY and U.S. Pat. No. 4,870,008.

As used herein, the term “propeptide” refers to a peptide whose function is to allow the expressed polypeptide to which it is attached to be directed from the endoplasmic reticulum to the Golgi apparatus and further to a secretory vesicle for secretion into the culture medium (i.e., exportation of the polypeptide across the cell wall or at least through the cellular membrane into the periplasmic space of the yeast cell). The propeptide may be the ScMF α1 (See U.S. Pat. Nos. 4,546,082 and 4,870,008). Alternatively, the pro-peptide may be a synthetic propeptide, which is to say a propeptide not found in nature, including but not limited to those disclosed in U.S. Pat. Nos. 5,395,922; 5,795,746; and 5,162,498 and in WO 9832867. The propeptide will preferably contain an endopeptidase processing site at the C-terminal end, such as a Lys-Arg sequence or any functional analog thereof.

As used herein with the term “insulin”, the term “desB30” or “B(1-29)” is meant to refer to an insulin B-chain peptide lacking the B30 amino acid residue and “A(1-21)” means the insulin A chain.

As used herein, the term “immediately N-terminal to” is meant to illustrate the situation where an amino acid residue or a peptide sequence is directly linked at its C-terminal end to the N-terminal end of another amino acid residue or amino acid sequence by means of a peptide bond.

As used herein an amino acid “modification” refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids.

As used herein an amino acid “substitution” refers to the replacement of one amino acid residue by a different amino acid residue. Throughout, the application, all references to a particular amino acid position by letter and number (e.g. position A5) refer to the amino acid at that position of either the A-chain (e.g. position A5) or the B-chain (e.g. position B5) in the respective native human insulin A-chain (SEQ ID NO: 38) or B-chain (SEQ ID NO: 39), or the corresponding amino acid position in any analogues thereof.

The term “glycoprotein” is meant to include any glycosylated insulin analogue, including single-chain insulin analogue, comprising one or more attachment groups to which one or more oligosaccharides is covalently linked thereto.

As used herein, an “N-linked glycosylation site” refers to the tri-peptide amino acid sequence NX(S/T) or AsnXaa(Ser/Thr) wherein “N” represents an asparagine (Asn) residue, “X” represents any amino acid (Xaa) except proline (Pro), “S” represents a serine (Ser) residue, and “T” represents a threonine (Thr) residue.

As used herein, the term “N-glycan” and “glycoform” are used interchangeably and refer to the oligosaccharide group per se that is attached by an asparagine-N-acetylglucosamine linkage to an attachment group comprising an N-linked glycosylation site. The N-glycan oligosaccharide group may be attached in vitro to any amino acid residue other than asparagine or in vivo to an asparagine residue comprising an N-linked glycosylation site.

The term “N-linked glycan” refers to an N-glycan in which the N-acetylglucosamine residue at the reducing end is linked in a β1 linkage to the amide nitrogen of an asparagine residue of an attachment group in the protein.

As used herein, the terms “N-linked glycosylated” and “N-glycosylated” are used interchangeably and refer to an N-glycan attached to an attachment group comprising an asparagine residue or an N-linked glycosylation site or motif.

As used herein, the term “N-glycan conjugate” refers to an N-glycan that is conjugated to an attachment group in vitro. The attachment group may or may not include an asparagine residue.

As used herein, the term “glycosylated insulin or insulin analogue” refers to an insulin or insulin analogue to which an N-glycan is attached thereto either in vivo or in vitro.

As used herein, the term “in vivo glycosylation” or “in vivo N-glycosylation” or “in vivo N-linked glycosylation” refers to the attachment of an oligosaccharide or glycan moiety to an asparagine residue of an N-linked glycosylation site occurring in vivo, i.e., during posttranslational processing in a glycosylating cell expressing the polypeptide by way of N-linked glycosylation. The exact oligosaccharide structure depends, to a large extent, on the host cell used to produce the glycosylated protein or polypeptide.

As used herein, the term “in vitro glycosylation” refers to a synthetic glycosylation performed in vitro, normally involving covalently linking an N-glycan having a functional group capable of being conjugated or linked to an attachment group of a polypeptide, optionally using a cross-linking agent to provide an N-glycan conjugate. In vitro glycosylation further includes chemically synthesizing the protein or polypeptide wherein an amino acid covalently linked to an N-glycan is incorporated into the protein or polypeptide during synthesis. In vivo and in vitro glycosylation are discussed in detail further below.

The term “attachment group” is intended to indicate a functional group of the polypeptide, in particular of an amino acid residue thereof, capable of being covalently linked to a macromolecular substance such as an oligosaccharide or glycan, a polymer molecule, a lipophilic molecule, or an organic derivatizing agent.

For in vivo N-glycosylation, the term “attachment group” is used in an unconventional way to indicate the amino acid residues constituting an “N-linked glycosylation site” or “N-glycosylation site” comprising N—X—S/T, wherein X is any amino acid except proline. Although the asparagine (N) residue of the N-glycosylation site is where the oligosaccharide or glycan moiety is attached during glycosylation, such attachment cannot be achieved unless the other amino acid residues of the N-glycosylation site are present. While the N-linked glycosylated insulin analogue precursor will include all three amino acids comprising the “attachment group” to enable in vivo N-glycosylation, the N-linked glycosylated insulin analogue may be processed subsequently to lack X and/or S/T. Accordingly, when the conjugation is to be achieved by N-glycosylation, the term “amino acid residue comprising an attachment group for the oligosaccharide or glycan” as used in connection with alterations of the amino acid sequence of the polypeptide is to be understood as meaning that one or more amino acid residues constituting an N-glycosylation site are to be altered in such a manner that a functional N-glycosylation site is introduced into the amino acid sequence. The attachment group may be present in the insulin analogue precursor but in the heterodimer insulin analogue one or two of the amino acid residues comprising the attachment site but not the asparagine (N) residue linked to the oligosaccharide or glycan may be removed. For example, an insulin analogue precursor may comprise an attachment group consisting of NKT at positions B28, 29, and 30, respectively, but the mature heterodimer of the analogue may be a desB30 insulin analogue wherein the T at position 30 has been removed.

In general, for the conjugate disclosed herein comprising an introduced amino acid residue with an attachment group for the macromolecular substance, it is preferred that the macromolecular substance is attached to the introduced amino acid residue. More specifically, it is generally understood for the positions specifically indicated herein as attachment sites for the macromolecular substance, that the conjugate of the invention comprises at least the macromolecular substance attached to one of said positions.

As used herein, “N-glycans” have a common pentasaccharide core of Man₃GlcNAc₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid (“Sia”) or derivatives (e.g., “NANA” or “NeuAc” where “Neu” refers to neuraminic acid and “Ac” refers to acetyl, or the derivative NGNA, which refers to N-glycolylneuraminic acid). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. N-glycans consisting of a Man₃GlcNAc₂ structure are called paucimannose. The various N-glycans are also referred to as “glycoforms.”

With respect to complex N-glycans, the terms “G-2”, “G-1”, “G0”, “G1”, “G2”, “A1”, and “A2” mean the following. “G-2” refers to an N-glycan structure that can be characterized as Man₃GlcNAc₂; the term “G-1” refers to an N-glycan structure that can be characterized as GlcNAcMan₃GlcNAc₂; the term “G0” refers to an N-glycan structure that can be characterized as GlcNAc₂Man₃GlcNAc₂; the term “G1” refers to an N-glycan structure that can be characterized as GalGlcNAc₂Man₃GlcNAc₂; the term “G2” refers to an N-glycan structure that can be characterized as Gal₂GlcNAc₂Man₃GlcNAc₂; the term “A1” refers to an N-glycan structure that can be characterized as SiaGal₂GlcNAc₂Man₃GlcNAc₂; and, the term “A2” refers to an N-glycan structure that can be characterized as Sia₂Gal₂GlcNAc₂Man₃GlcNAc₂. Unless otherwise indicated, the terms G-2″, “G-1”, “G0”, “G1”, “G2”, “A1”, and “A2” refer to N-glycan species that lack fucose attached to the GlcNAc residue at the reducing end of the N-glycan. When the term includes an “F”, the “F” indicates that the N-glycan species contain a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

With respect to multiantennary N-glycans, the term “multiantennary N-glycan” refers to N-glycans that further comprise a GlcNAc residue on the mannose residue comprising the non-reducing end of the 1,6 arm or the 1,3 arm of the N-glycan or a GlcNAc residue on each of the mannose residues comprising the non-reducing end of the 1,6 arm and the 1,3 arm of the N-glycan. Thus, multiantennary N-glycans can be characterized by the formulas GlcNAc₍₂₋₄₎Man₃GlcNAc₂, Gal₍₁₋₄₎GlcNAc₍₂₋₄₎Man₃GlcNAc₂, or Sia₍₁₋₄₎Gal₍₁₋₄₎GlcNAc₍₂₋₄₎Man₃GlcNAc₂. The term “1-4” refers to 1, 2, 3, or 4 residues.

With respect to bisected N-glycans, the term “bisected N-glycan” refers to N-glycans in which a GlcNAc residue is linked to the mannose residue at the non-reducing end of the N-glycan. A bisected N-glycan can be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the formula indicates that two GlcNAc residues are linked to the mannose residue at the non-reducing end of one of the two arms of the N-glycans and one GlcNAc residue is linked to the mannose residue at the non-reducing end of the other arm of the N-glycan.

Abbreviations used herein are of common usage in the art, see, e.g., abbreviations of sugars, above. Other common abbreviations include “PNGase”, or “glycanase” which all refer to glycopeptide N-glycosidase; glycopeptidase; N-oligosaccharide glycopeptidase; N-glycanase; glycopeptidase; Jack-bean glycopeptidase; PNGase A; PNGase F; glycopeptide N-glycosidase (EC 3.5.1.52, formerly EC 3.2.2.18).

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein A recombinant host cell may be an isolated cell or cell line grown in culture or may be a cell which resides in a living tissue or organism. Host cells may be yeast, fungi, mammalian cells, plant cells, insect cells, and prokaryotes and archaea that have been genetically engineered to produce glycoproteins.

When referring to “mole percent” or “mole %” of a glycan present in a preparation of a glycoprotein, the term means the molar percent of a particular glycan present in the pool of N-linked oligosaccharides released when the protein preparation is treated with PNGase and then quantified by a method that is not affected by glycoform composition, (for instance, labeling a PNGase released glycan pool with a fluorescent tag such as 2-aminobenzamide and then separating by high performance liquid chromatography or capillary electrophoresis and then quantifying glycans by fluorescence intensity). For example, 50 mole percent GlcNAc₂Man₃GlcNAc₂Gal₂NANA₂ means that 50 percent of the released glycans are GlcNAc₂Man₃GlcNAc₂Gal₂NANA₂ and the remaining 50 percent are comprised of other N-linked oligosaccharides. In embodiments, the mole percent of a particular glycan in a preparation of glycoprotein will be between 20% and 100%, preferably above 25%, 30%, 35%, 40% or 45%, more preferably above 50%, 55%, 60%, 65% or 70% and most preferably above 75%, 80% 85%, 90% or 95%.

The term “operably linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and as used herein refer to polynucleotide sequences that are necessary to affect the expression of coding sequences to which they are operably linked. Expression control sequences are sequences that control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “transfect”, “transfection”, “transfecting” and the like refer to the introduction of a heterologous nucleic acid into eukaryote cells, both higher and lower eukaryote cells. Historically, the term “transformation” has been used to describe the introduction of a nucleic acid into a prokaryote, yeast, or fungal cell; however, the term “transfection” is also used to refer to the introduction of a nucleic acid into any prokaryotic or eukaryote cell, including yeast and fungal cells. Furthermore, introduction of a heterologous nucleic acid into prokaryotic or eukaryotic cells may also occur by viral or bacterial infection or ballistic DNA transfer, and the term “transfection” is also used to refer to these methods in appropriate host cells.

The term “eukaryotic” refers to a nucleated cell or organism, and includes insect cells, plant cells, mammalian cells, animal cells and lower eukaryotic cells.

The term “lower eukaryotic cells” includes yeast and filamentous fungi. Yeast and filamentous fungi include, but are not limited to Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa.

As used herein, the term “consisting essentially of” will be understood to imply the inclusion of a stated integer or group of integers; while excluding modifications or other integers that would materially affect or alter the stated integer. For example, with respect to a species of N-glycans attached to an insulin or insulin analogue, the term “consisting essentially of” a stated N-glycan will be understood to include the N-glycan whether or not that N-glycan is fucosylated at the N-acetylglucosamine (GlcNAc) which is directly linked to the asparagine residue of the glycoprotein provided that for the particular N-glycan species the fucose does not materially affect the glycosylated insulin or insulin analogue compared to the glycosylated insulin or insulin analogue in which the N-glycan lacks the fucose.

As used herein, the term “predominantly” or variations such as “the predominant” or “which is predominant” will be understood to mean the glycan species that has the highest mole percent (%) of total neutral N-glycans after the insulin analogue has been treated with PNGase and released glycans analyzed by mass spectroscopy, for example, MALDI-TOF MS or HPLC. In other words, the phrase “predominantly” is defined as an individual entity, such as a specific glycoform, is present in greater mole percent than any other individual entity. For example, if a composition consists of species A at 40 mole percent, species B at 35 mole percent and species C at 25 mole percent, the composition comprises predominantly species A, and species B would be the next most predominant species. Some host cells may produce compositions comprising neutral N-glycans and charged N-glycans such as mannosylphosphate. Therefore, a composition of glycoproteins can include a plurality of charged and uncharged or neutral N-glycans. In the present invention, it is within the context of the total plurality of neutral N-glycans in the composition in which the predominant N-glycan determined. Thus, as used herein, “predominant N-glycan” means that of the total plurality of neutral N-glycans in the composition, the predominant N-glycan is of a particular structure.

As used herein, the term “essentially free of” a particular sugar residue, such as fucose, or galactose and the like, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent. Thus, substantially all of the N-glycan structures in an insulin analogue composition disclosed herein are free of, for example, fucose, or galactose, or both.

As used herein, an insulin analogue composition “lacks” or “is lacking” a particular sugar residue, such as fucose or galactose, when no detectable amount of such sugar residue is present on the N-glycan structures at any time. For example, in preferred embodiments of the present invention, the insulin analogue compositions are produced by lower eukaryotic organisms, as defined above, including yeast (for example, Pichia sp.; Saccharomyces sp.; Kluyveromyces sp.; Aspergillus sp.), and will “lack fucose,” because the cells of these organisms do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the term “treating” includes prophylaxis of the specific disorder or condition, or alleviation of the symptoms associated with a specific disorder or condition and/or preventing or eliminating said symptoms. For example, as used herein the term “treating diabetes” will refer in general to maintaining glucose blood levels near normal levels and may include increasing or decreasing blood glucose levels depending on a given situation.

As used herein an “effective” amount or a “therapeutically effective amount” of an insulin analogue refers to a nontoxic but sufficient amount of an insulin analogue to provide the desired effect. For example one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as intranasal, inhalation, subcutaneous, intramuscular, intraspinal, or intravenous.

As used herein, the term “pharmacokinetic” refers to in vivo properties of an insulin or insulin analogue commonly used in the field that relate to the liberation, absorption, distribution, metabolism, and elimination of the protein. Such pharmacokinetic properties include, but are not limited to, dose, dosing interval, concentration, elimination rate, elimination rate constant, area under curve, volume of distribution, clearance in any tissue or cell, proteolytic degradation in blood, bioavailability, binding to plasma, half-life, first-pass elimination, extraction ratio, Cₘₐₓ, tₘₐₓ, Cₘᵢₙ, rate of absorption, and fluctuation.

As used herein, the term “pharmacodynamic” refers to in vivo properties of an insulin or insulin analogue commonly used in the field that relate to the physiological effects of the protein. Such pharmacokinetic properties include, but are not limited to, maximal glucose infusion rate, time to maximal glucose infusion rate, and area under the glucose infusion rate curve.

### BRIEF DESCRIPTION OF STRAIN CONSTRUCTION INFORMATION

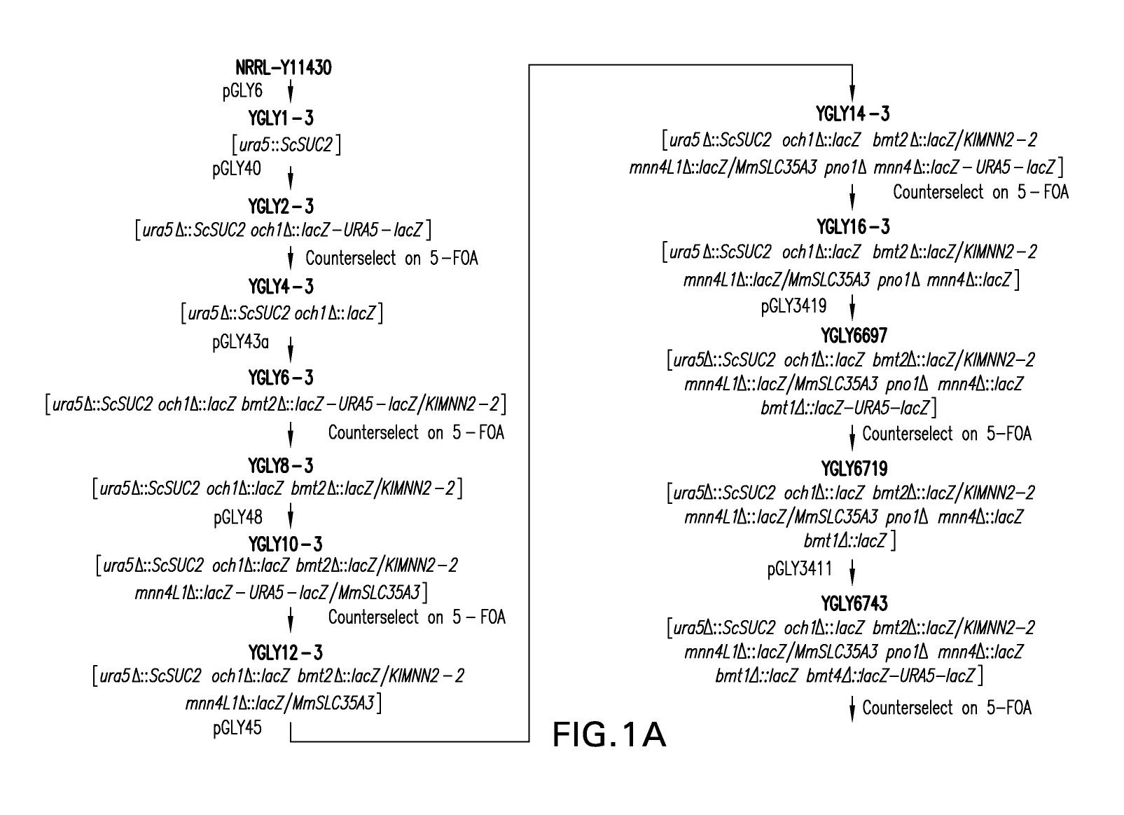

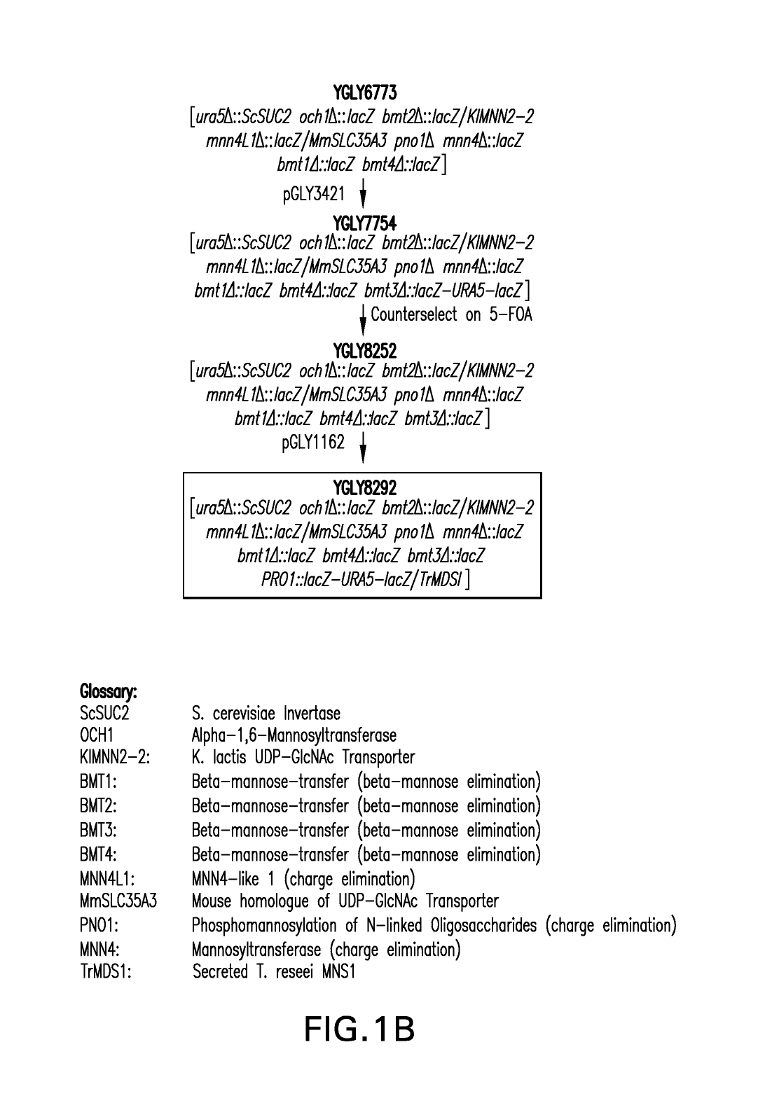

FIGS. 1A and 1B show the genealogy P. pastoris strain YGLY82925 beginning from wild-type strain NRRL-Y11430.

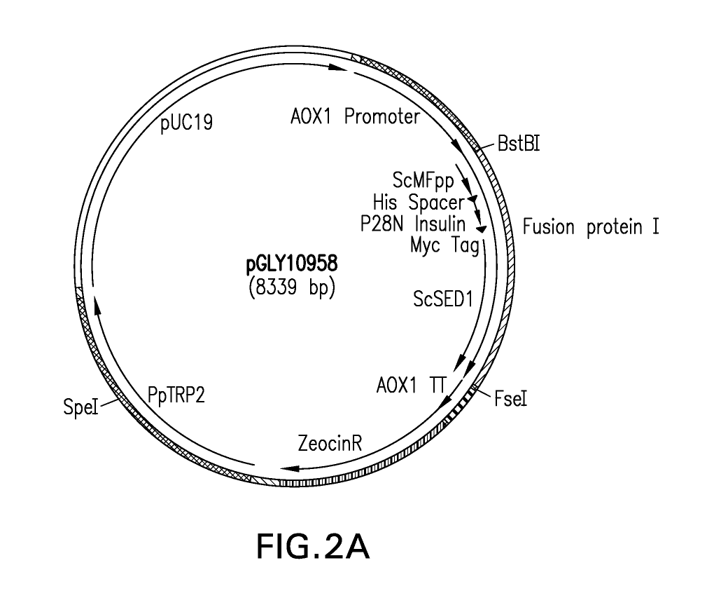

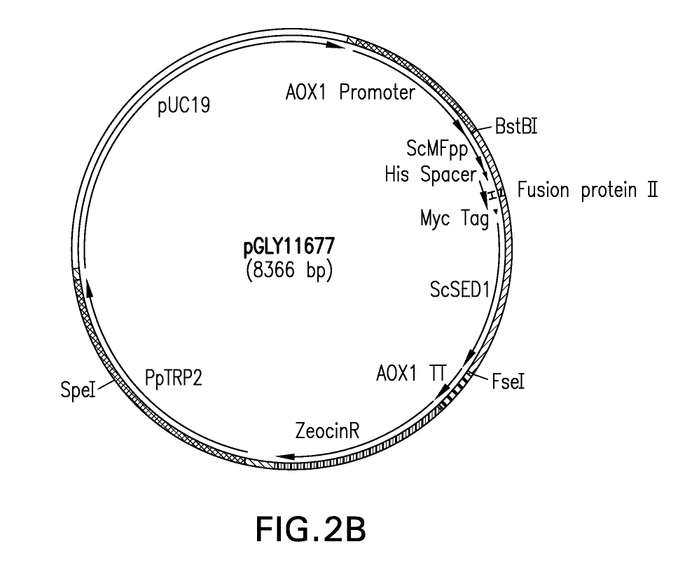

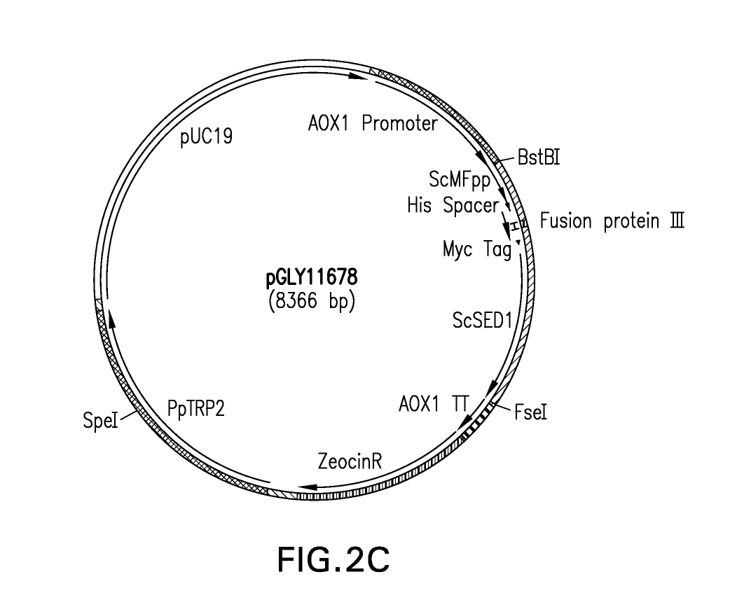

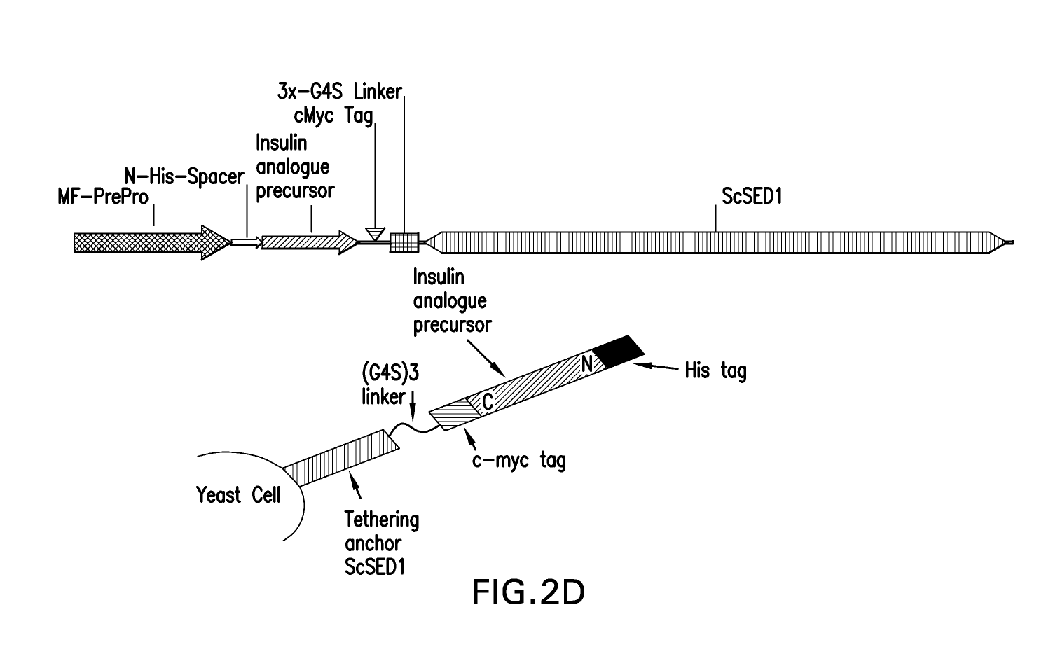

FIG. 2A shows a diagram of pGLY10958 encoding the surface display protein: fusion protein I comprising insulin analogue precursor IA. The plasmid is a roll-in vector that targets the TRP2 locus in P. pastoris. The ORF encoding the insulin analogue precursor is under the control of a P. pastoris AOX1 promoter and the P. pastoris AOX1 3UTR transcription termination sequence. Selection of transformants uses zeocin resistance encoded by the zeocin resistance protein (ZeocinR) ORF under the control of the S. cerevisiae TEF1 promoter and S. cerevisiae CYC termination sequence.

FIG. 2B shows a diagram of pGLY11677 encoding the surface display proteins: fusion protein II comprising insulin analogue precursor IIA. The plasmid is a roll-in vector that targets the TRP2 locus in P. pastoris. The ORF encoding the insulin analogue precursor is under the control of a P. pastoris AOX1 promoter and the P. pastoris AOX1 3UTR transcription termination sequence. Selection of transformants uses zeocin resistance encoded by the zeocin resistance protein (ZeocinR) ORF under the control of the S. cerevisiae TEF1 promoter and S. cerevisiae CYC termination sequence.

FIG. 2C shows a diagram of pGLY11678, encoding the surface display proteins: fusion protein III comprising insulin analogue precursor IIIA. The plasmid is a roll-in vector that targets the TRP2 locus in P. pastoris. The ORF encoding the insulin analogue precursor is under the control of a P. pastoris AOX1 promoter and the P. pastoris AOX1 3UTR transcription termination sequence. Selection of transformants uses zeocin resistance encoded by the zeocin resistance protein (ZeocinR) ORF under the control of the S. cerevisiae TEF1 promoter and S. cerevisiae CYC termination sequence.

FIG. 2D shows a diagram depicting the fusion protein encoded by the vectors in FIGS. 2A-C in the upper portion and the proinsulin precursor analogue obtained from the fusion protein tethered to the cell surface in the lower portion. The fusion protein comprises the Saccharomyces cerevisiae alpha-mating factor prepro polyptide (MF-Pro) fused to the N-terminus of a His spacer epitope peptide (N-His-Spacer) fused to the N-terminus of proinsulin (Insulin) that includes the B-chain peptide, C-peptide, and A-chain peptide fused to the N-terminus of a peptide encoding the cMyc epitope peptide (cMyc tag) fused to the N-terminus of the 3×-G4S linker (3×-G4S or (G4S)₃) fused to the N-terminus of a truncated Saccharomyces cerevisiae Sed1p (ScSED1). The lower portion of the figure shows the in vivo processed fusion protein attached or tethered to the yeast cell surface and displaying the pro insulin precursor analogue (disulfide bonds between the A and B chain peptides are not shown). The N-terminal His and C-terminal cMyc epitopes are optional but were included to simplify detection of the displayed insulin precursor analogue with anti-His or anti-cMyc antibodies.

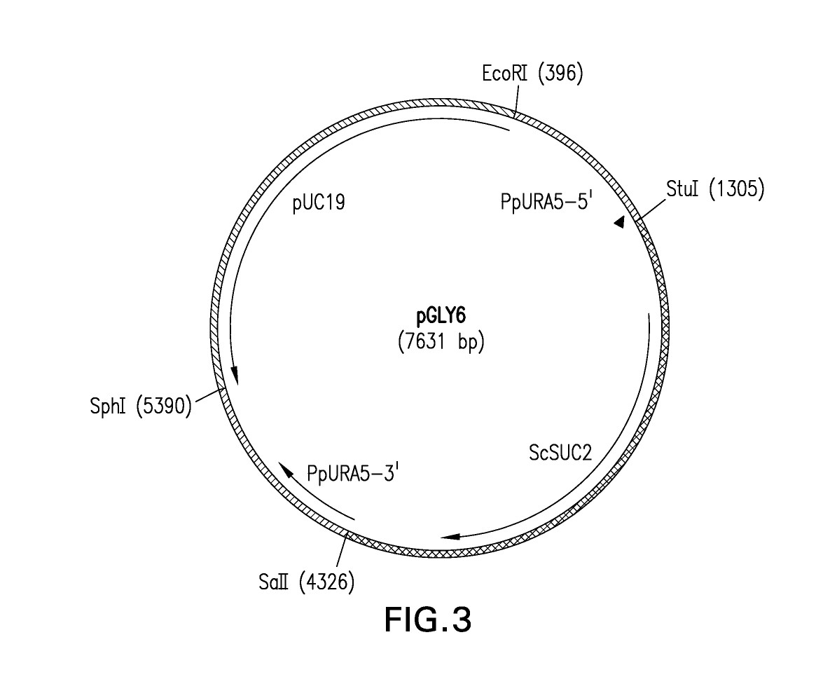

FIG. 3 shows a map of plasmid pGLY6. Plasmid pGLY6 is an integration vector that targets the URA5 locus and contains a nucleic acid molecule comprising the S. cerevisiae invertase gene or transcription unit (ScSUC2) flanked on one side by a nucleic acid molecule comprising a nucleotide sequence from the 5′ region of the P. pastoris URA5 gene (PpURA5-5′) and on the other side by a nucleic acid molecule comprising the a nucleotide sequence from the 3′ region of the P. pastoris URA5 gene (PpURA5-3′).

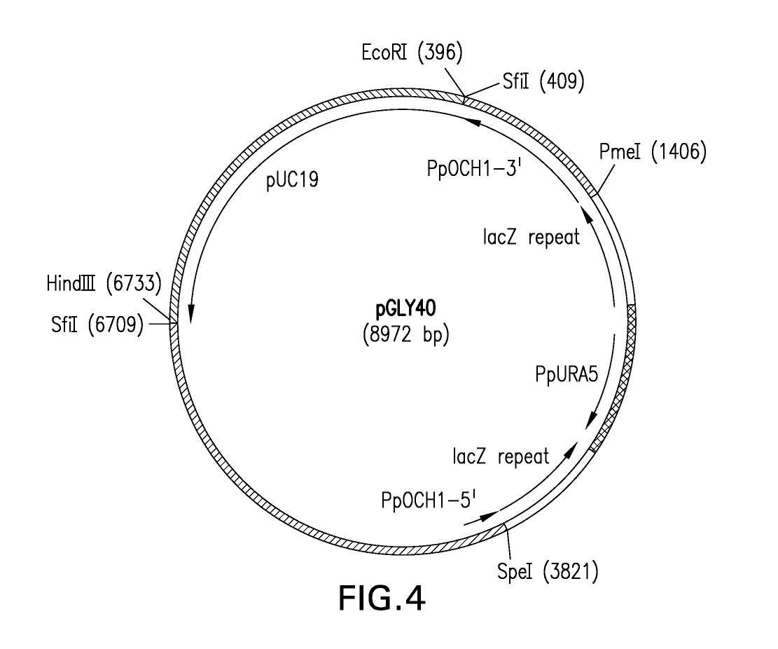

FIG. 4 shows a map of plasmid pGLY40. Plasmid pGLY40 is an integration vector that targets the OCH1 locus and contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the OCH1 gene (PpOCH1-5′) and on the other side by a nucleic acid molecule comprising a nucleotide sequence from the 3′ region of the OCH1 gene (PpOCH1-3′).

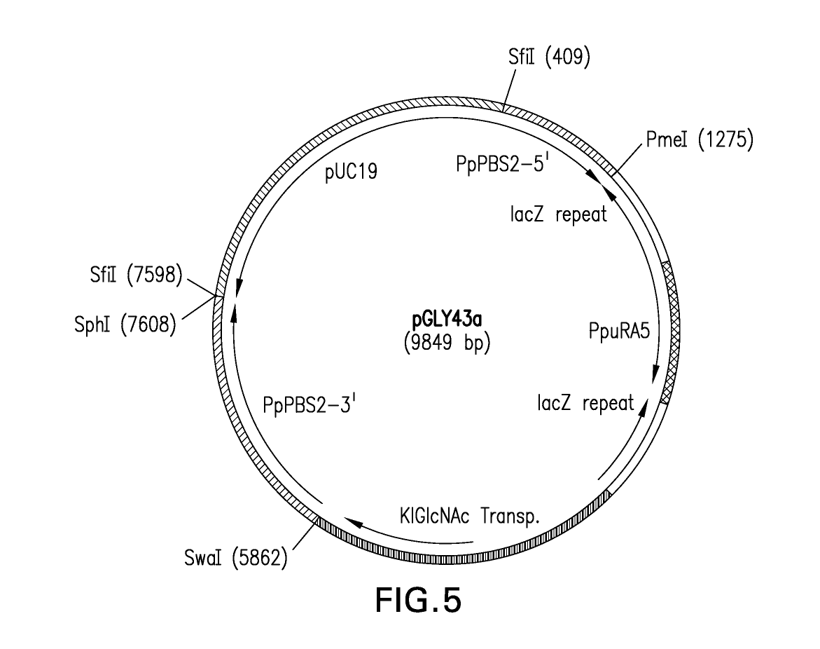

FIG. 5 shows a map of plasmid pGLY43a. Plasmid pGLY43a is an integration vector that targets the BMT2 locus and contains a nucleic acid molecule comprising the K. lactis UDP-N-acetylglucosamine (UDP-GlcNAc) transporter gene or transcription unit (KlGlcNAc Transp.) adjacent to a nucleic acid molecule comprising the P. pastoris URA5 gene or transcription unit (PpURA5) flanked by nucleic acid molecules comprising lacZ repeats (lacZ repeat). The adjacent genes are flanked on one side by a nucleic acid molecule comprising a nucleotide sequence from the 5′ region of the BMT2 gene (PpPBS2-5′) and on the other side by a nucleic acid molecule comprising a nucleotide sequence from the 3′ region of the BMT2 gene (PpPBS2-3′).

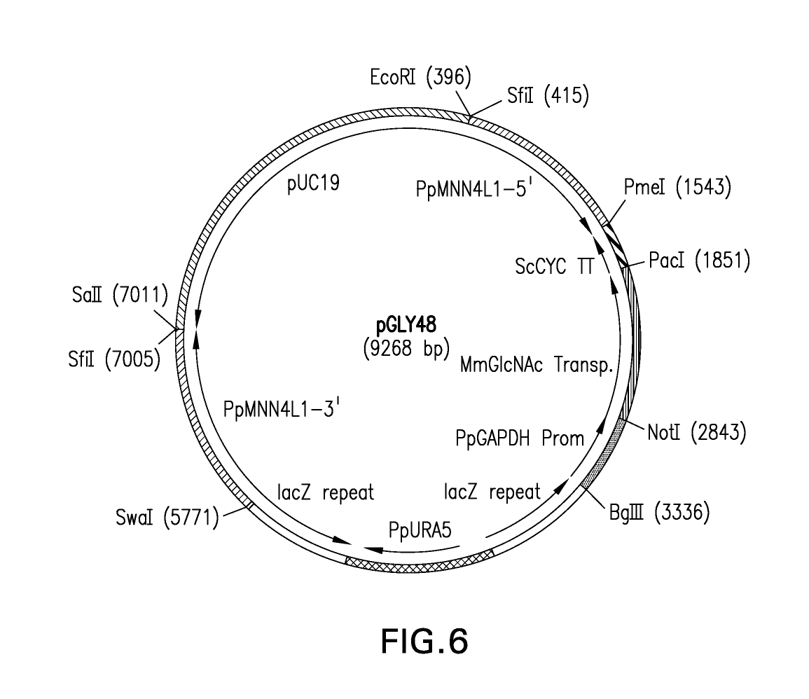

FIG. 6 shows a map of plasmid pGLY48. Plasmid pGLY48 is an integration vector that targets the MNN4L1 locus and contains an expression cassette comprising a nucleic acid molecule encoding the mouse homologue of the UDP-GlcNAc transporter (MmGlcNAc Transp.) open reading frame (ORF) operably linked at the 5′ end to a nucleic acid molecule comprising the P. pastoris GAPDH promoter (PpGAPDH Prom) and at the 3′ end to a nucleic acid molecule comprising the S. cerevisiae CYC termination sequence (ScCYC TT) adjacent to a nucleic acid molecule comprising the P. pastoris URA5 gene or transcription unit (PpURA5) flanked by lacZ repeats (lacZ repeat) and in which the expression cassettes together are flanked on one side by a nucleic acid molecule comprising a nucleotide sequence from the 5′ region of the P. pastoris MNN4L1 gene (PpMNN4L1-5′) and on the other side by a nucleic acid molecule comprising a nucleotide sequence from the 3′ region of the MNN4L1 gene (PpMNN4L1-3′).

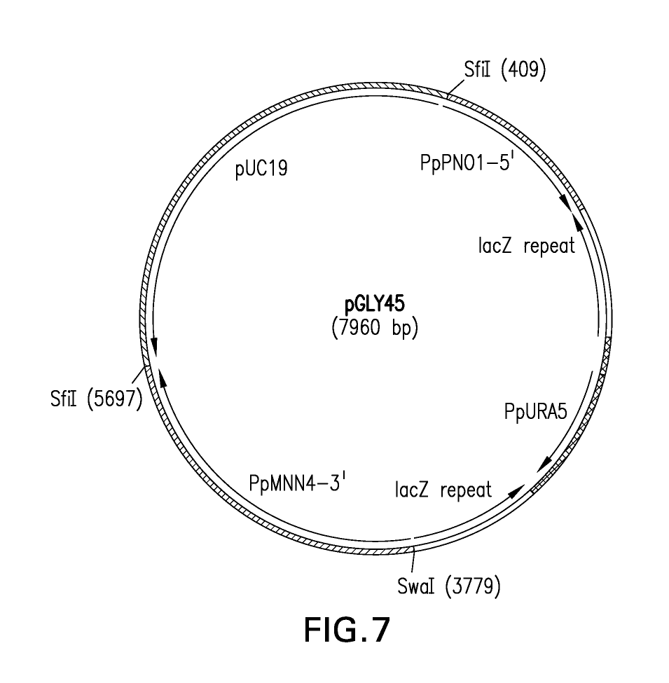

FIG. 7 shows as map of plasmid pGLY45. Plasmid pGLY45 is an integration vector that targets the PNO1/MNN4 loci contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the PNO1 gene (PpPNO1-5′) and on the other side by a nucleic acid molecule comprising a nucleotide sequence from the 3′ region of the MNN4 gene (PpMNN4-3′).

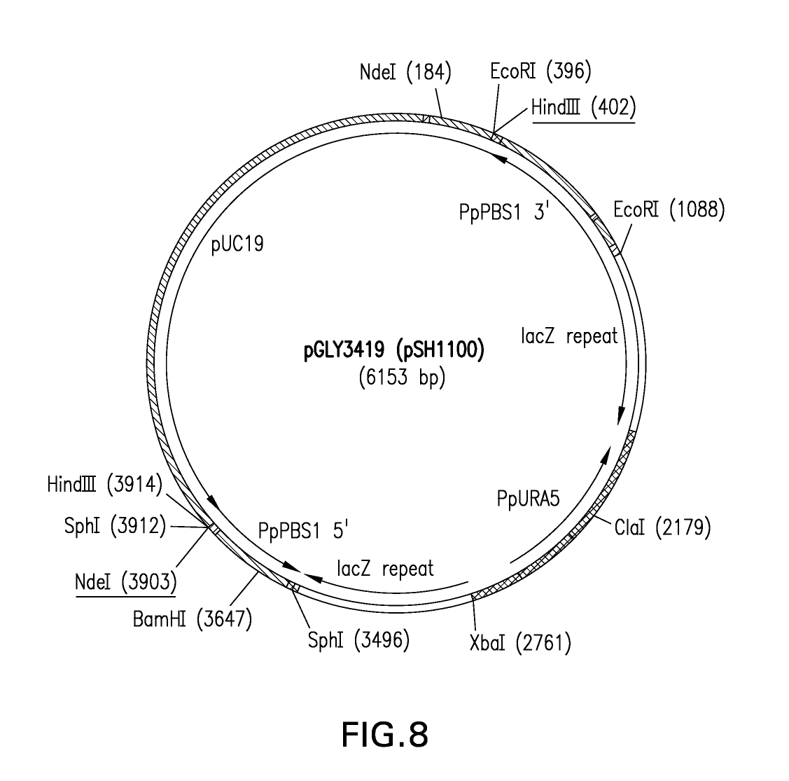

FIG. 8 shows a map of plasmid pGLY3419 (pSH1110). Plasmid pGLY3430 (pSH1115) is an integration vector that contains an expression cassette comprising the P. pastoris URA5 gene or transcription unit (PpURA5) flanked by lacZ repeats (lacZ repeat) flanked on one side with the 5′ nucleotide sequence of the P. pastoris BMT1 gene (PBS1 5′) and on the other side with the 3′ nucleotide sequence of the P. pastoris BMT1 gene (PBS1 3′).

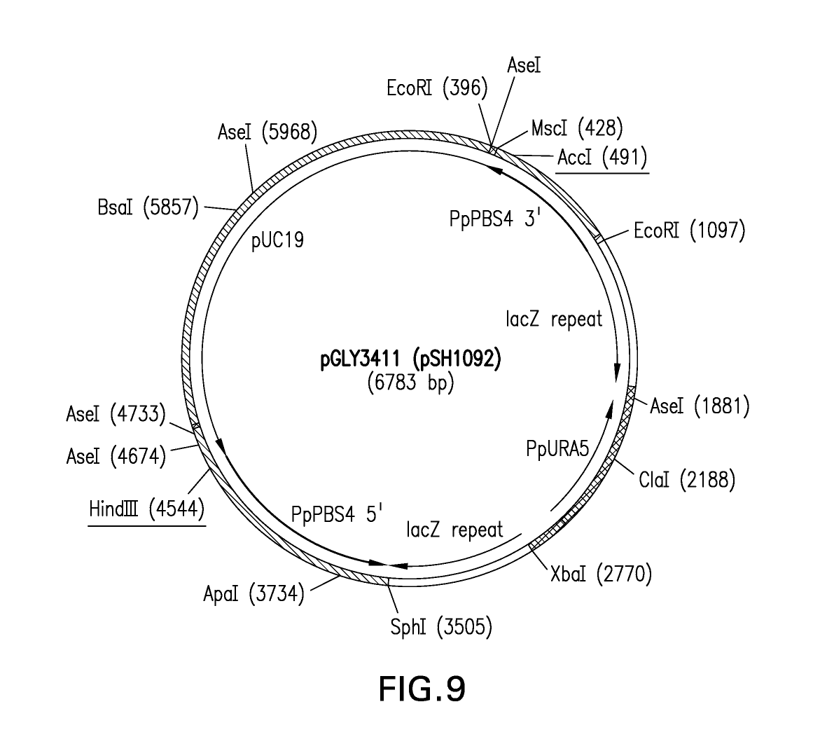

FIG. 9 shows a map of plasmid pGLY3411 (pSH1092). Plasmid pGLY3411 (pSH1092) is an integration vector that contains the expression cassette comprising the P. pastoris URA5 gene or transcription unit (PpURA5) flanked by lacZ repeats (lacZ repeat) flanked on one side with the 5′ nucleotide sequence of the P. pastoris BMT4 gene (PpPBS4 5′) and on the other side with the 3′ nucleotide sequence of the P. pastoris BMT4 gene (PpPBS4 3′).

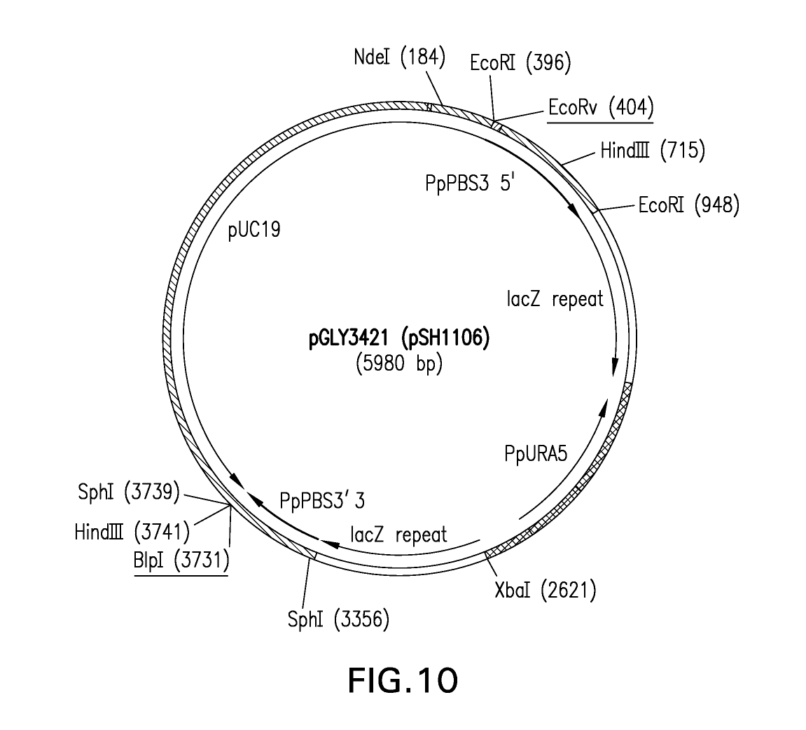

FIG. 10 shows a map of plasmid pGLY3421 (pSH1106). Plasmid pGLY4472 (pSH1186) contains an expression cassette comprising the P. pastoris URA5 gene or transcription unit (PpURA5) flanked by lacZ repeats (lacZ repeat) flanked on one side with the 5′ nucleotide sequence of the P. pastoris BMT3 gene (PpPBS3 5′) and on the other side with the 3′ nucleotide sequence of the P. pastoris BMT3 gene (PpPBS3 3′).

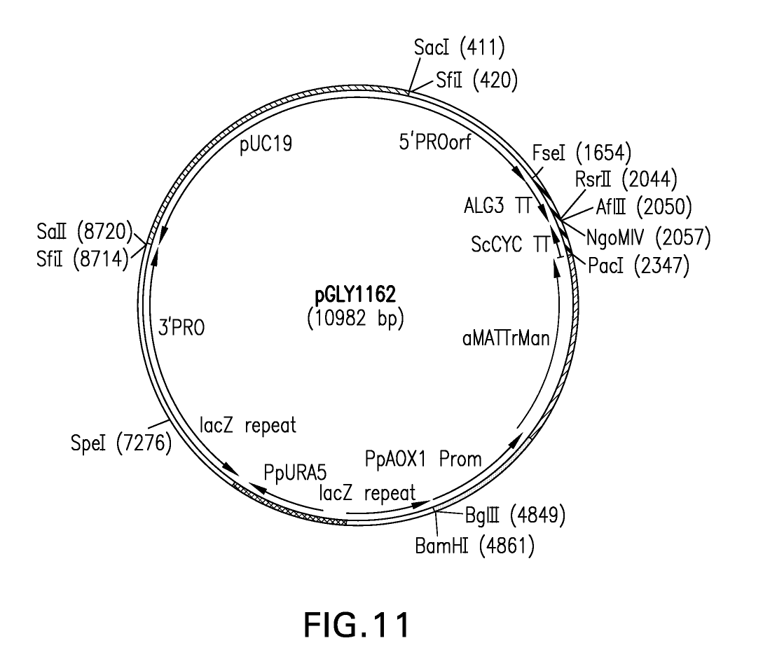

FIG. 11 shows a map of plasmid pGLY1162. Plasmid pGLY1162 is a KINKO integration vector that targets the PRO1 locus without disrupting expression of the locus and contains expression cassettes encoding the T. reesei α-1,2-mannosidase catalytic domain fused at the N-terminus to S. cerevisiae αMATpre signal peptide (aMATTrMan) to target the chimeric protein to the secretory pathway and secretion from the cell.

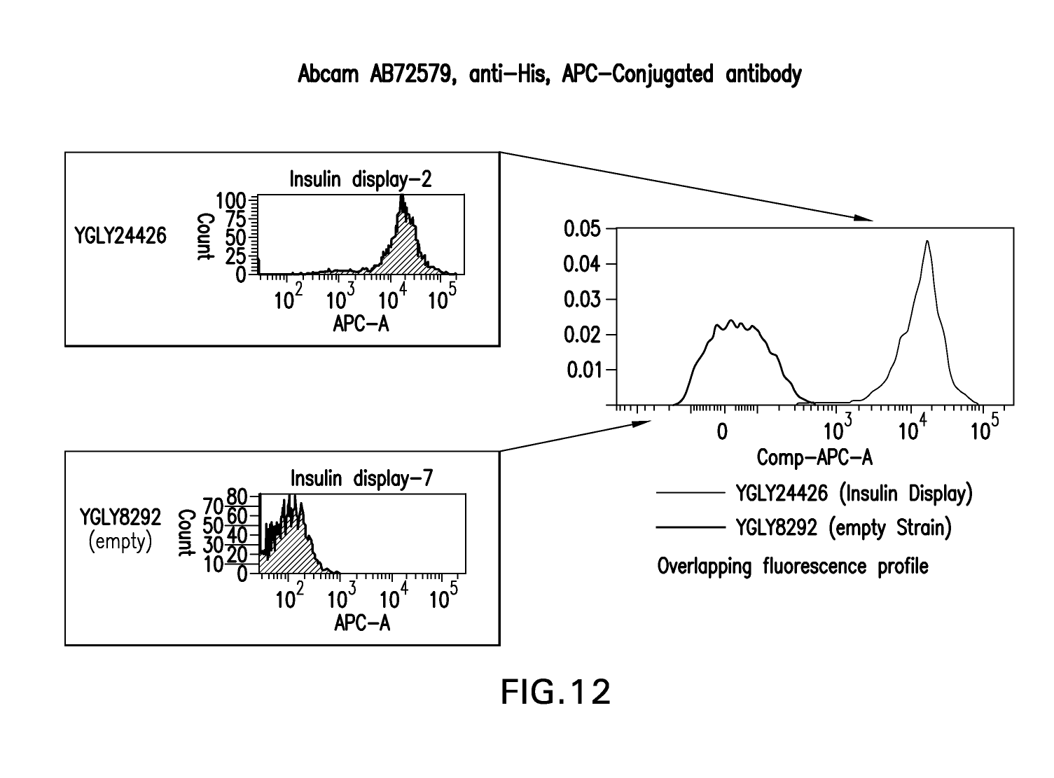

FIG. 12 depicts the flow cytometric analysis of display of recombinant insulin analogue precursor IA on yeast strain YGLY24426 detected using an anti-His antibody conjugated to APC. The green histogram represents the background auto-fluorescence of empty parental strain YGLY8292. The red histogram represents the cells that display the recombinant insulin analogue precursor. The entire cell population is bound to the anti-His antibodies, indicating that the insulin analogue precursor is well expressed and displayed on the yeast surface.

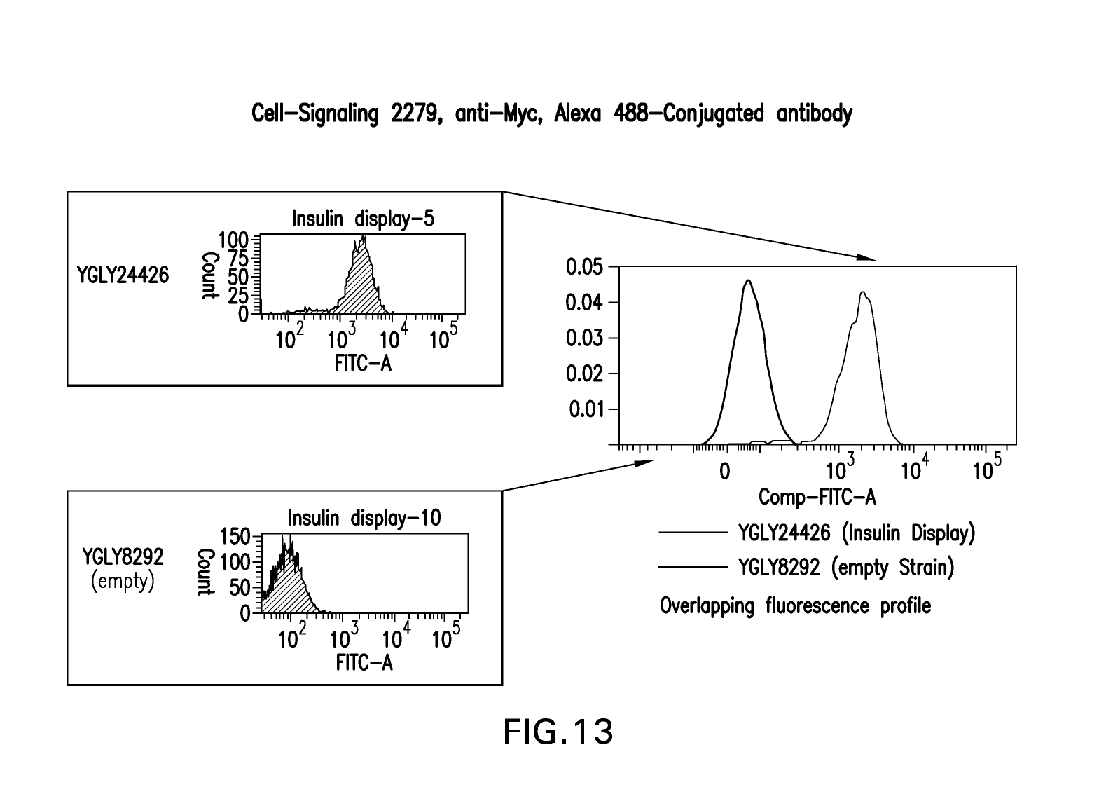

FIG. 13 depicts the flow cytometric analysis of display of insulin analogue precursor-truncated SED1 fusion protein IA on yeast strain YGLY24426 detected using an anti-cMyc antibody conjugated fluorephore ALEXA488. The green histogram represents the background auto-fluorescence of empty parental strain YGLY8292. The red histogram represents the cells that display the recombinant insulin analogue precursor. The entire cell population is bound to the anti-cMyc antibodies, indicating that recombinant insulin analogue is well expressed and displayed on the yeast surface.

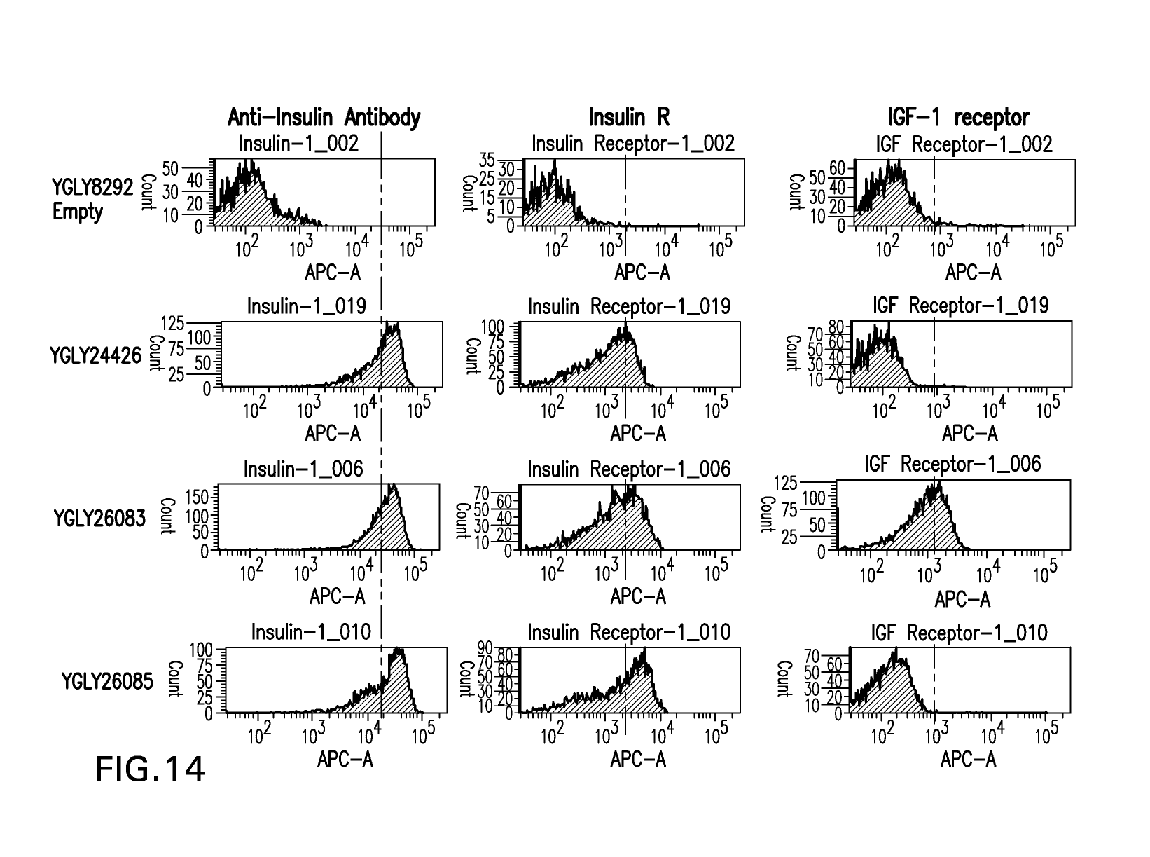

FIG. 14 depicts the flow cytometric analysis of insulin analogue expression on yeast detected using anti-insulin antibody; soluble IR and detection complex, and IGF-1 receptor and detection complex. Empty parental strain YGLY8292 is a negative control. All strains except strain YGLY8292 exhibited positive signals when incubated with anti-insulin antibody and soluble IR. Only strain YGLY26083, which displays a recombinant insulin analogue precursor with the native IGF-1 C-peptide, exhibited strong binding to IGF-1 receptor while strain YGLY26085, which displays a recombinant insulin analogue precursor having an IGF-1 C-peptide mutated to reduce binding to the IGF-1 receptor, exhibited low but above background binding to the IGF-1 receptor. Strains YGLY8292 and YGLY24426 did not appear to bind to soluble IGF-1 receptor.

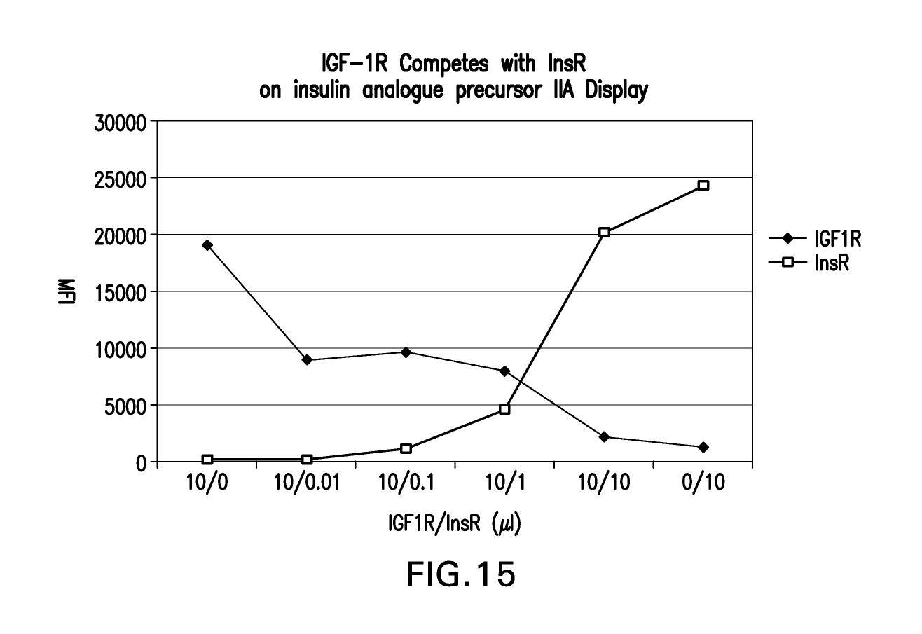

FIG. 15 depicts the flow cytometric analysis of strain YGLY26083, which displays a recombinant insulin analogue precursor with the native IGF-1 C-peptide, in a competition between binding the IR versus the IGF-1 receptor.

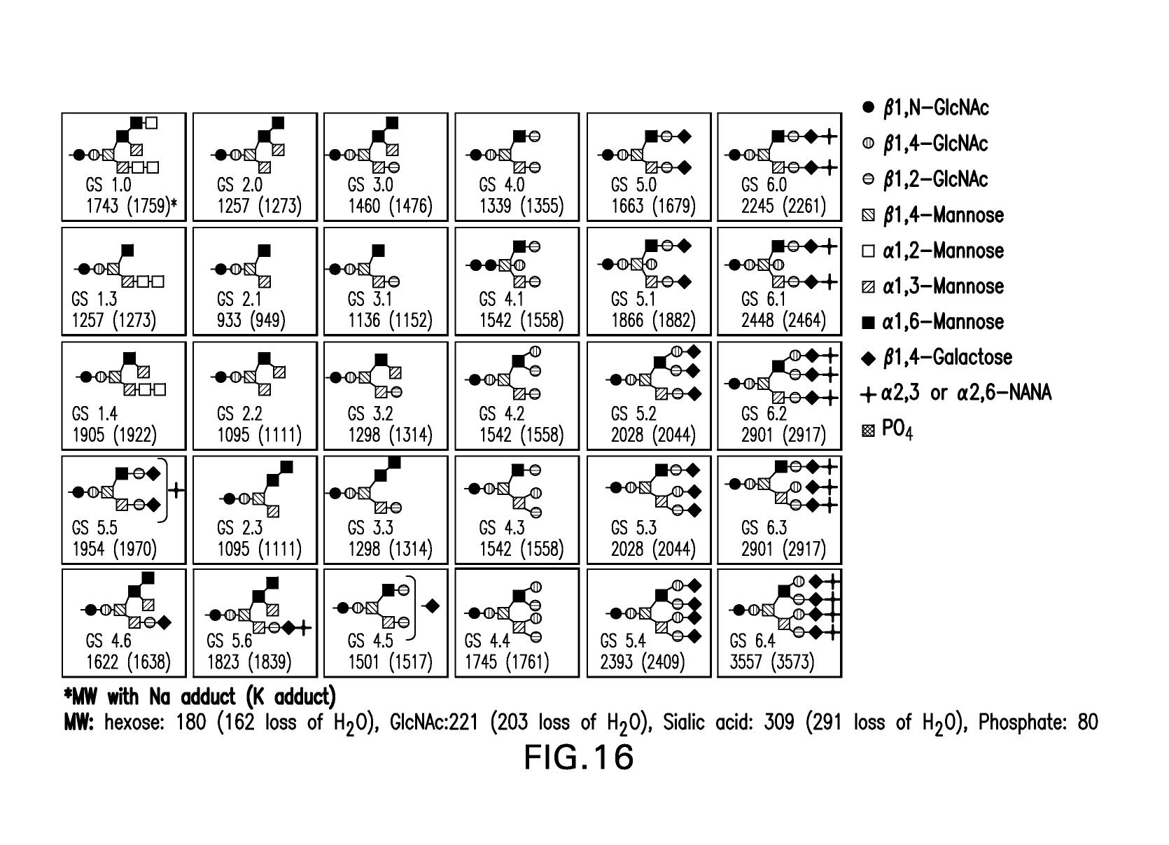

FIG. 16 shows examples of N-glycan structures that can be attached to the asparagine residue in the motif Asn-Xaa-Ser/Thr wherein Xaa is any amino acid other than proline of a glycoprotein.

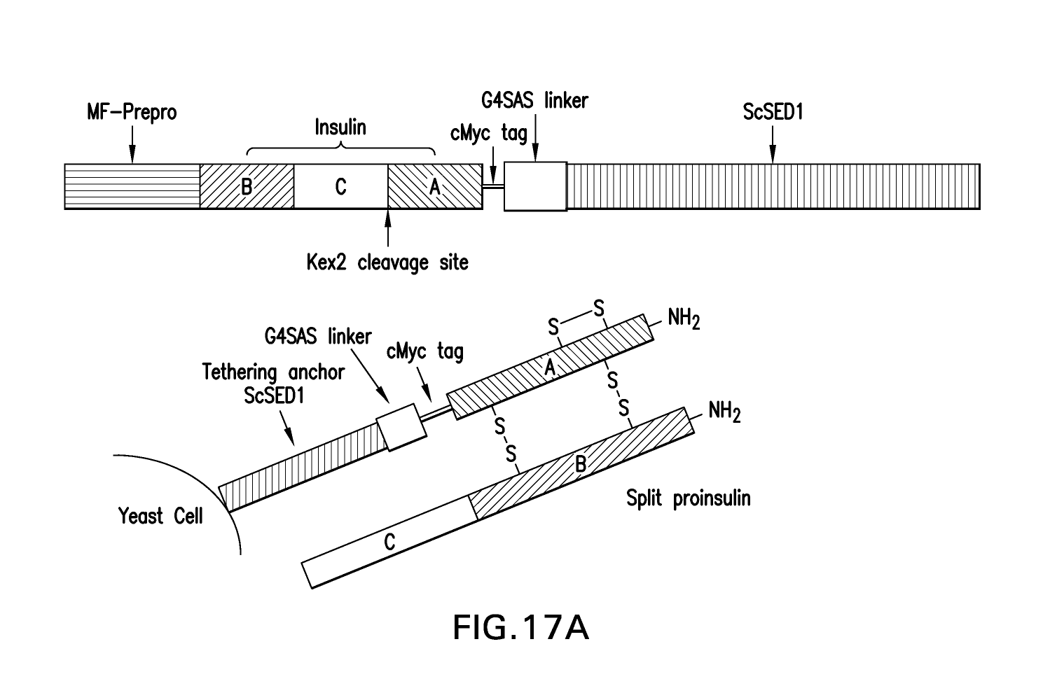

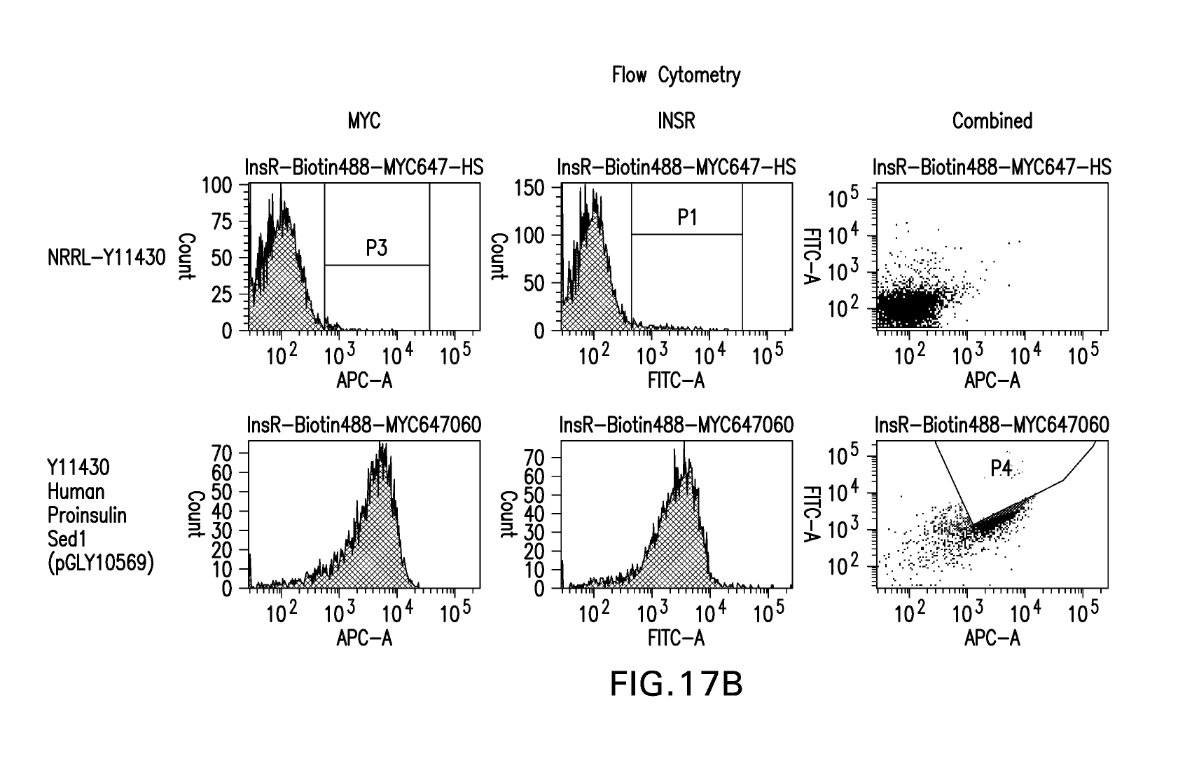

FIG. 17A shows a diagram depicting the fusion protein encoded by pGLY11680 in the upper portion and the split proinsulin obtained from the fusion protein tethered to the cell surface in the lower portion. The fusion protein comprises the Saccharomyces cerevisiae alpha-mating factor prepro polyptide (MF-Pro) fused to the N-terminus of the human native proinsulin (Insulin) that includes the B-chain peptide, C-peptide, and A-chain peptidefused to the N-terminus of a peptide encoding the cMyc epitope peptide (cMyc tag) fused to the N-terminus of the G4SAS linker fused to the N-terminus of a truncated Saccharomyces cerevisiae Sed1p (ScSED1). The location of the kex2 cleavage site is shown. The lower portion of the figure shows the in vivo processed fusion protein attached or tethered to the yeast cell surface and displaying the split proinsulin. The C-terminal cMyc epitope is optional but was included to simplify detection of the displayed split proinsulin with anti-cMyc antibodies

FIG. 17B shows flow cytometric analysis of the displayed split proinsulin molecule in wild-type Pichia pastoris detected with anti-cMyc antibodies (MYC), biotinylated insulin receptor (INSR), or both to detect the split proinsulin molecules on the cell surface.

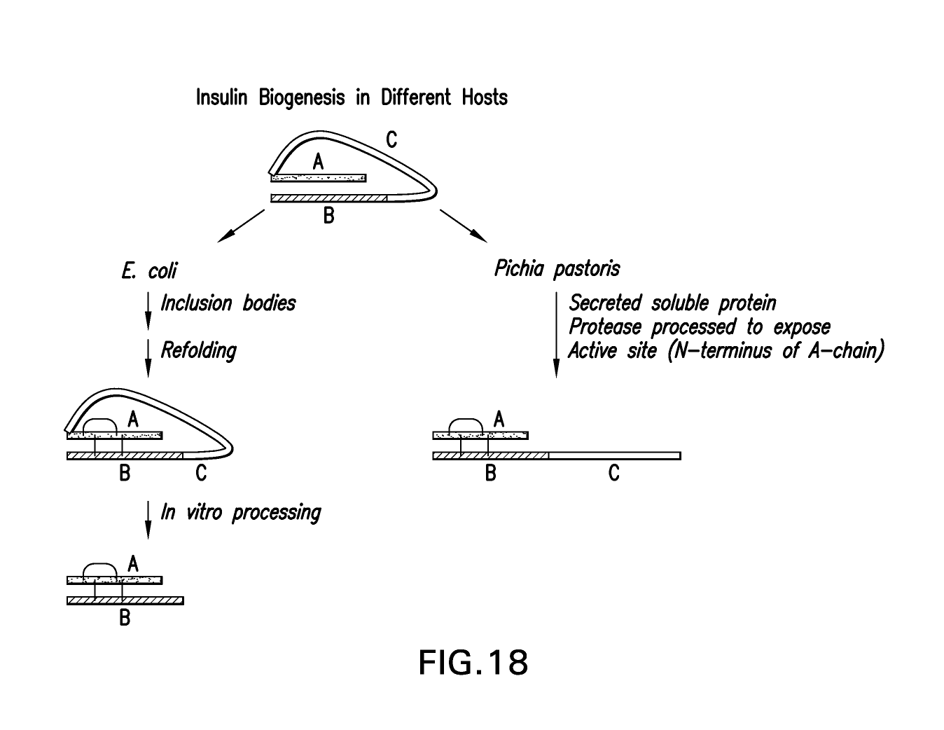

FIG. 18 shows a schematic diagram of the biogenesis steps of human proinsulin in Pichia pastoris. The C-terminus of the proinsulin C-peptide contains the LQKR (SEQ ID NO:67) motif, which is a substrate for Pichia pastoris Kex2 protease. The processing of this site by kex2 protease results in production of a two-chain biologically active split proinsulin molecule.

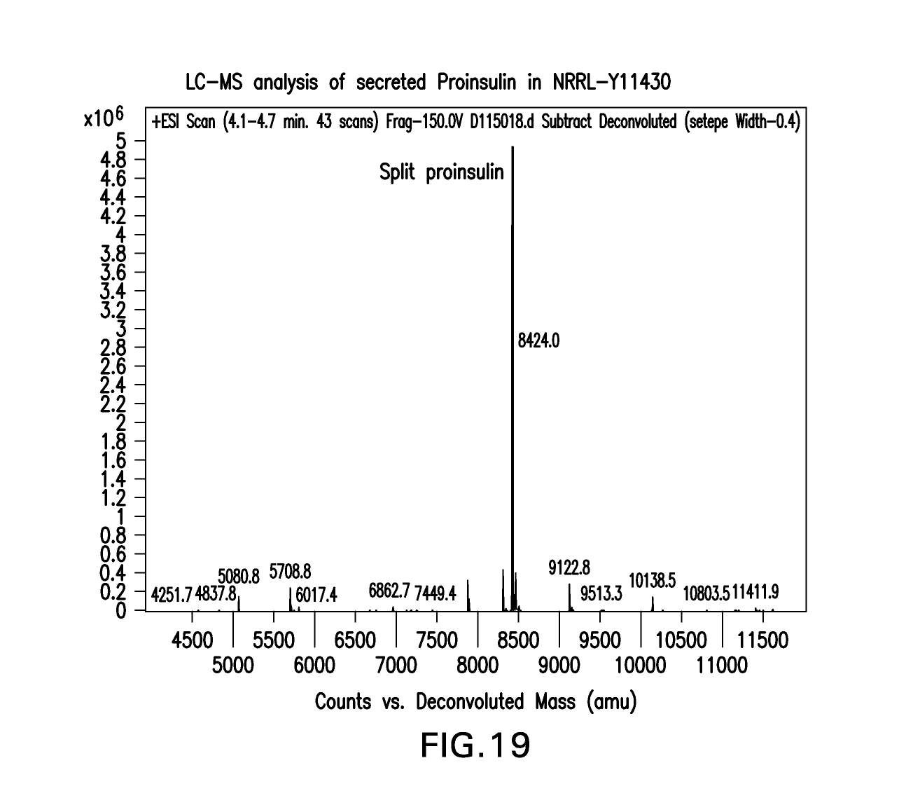

FIG. 19 shows LC-MS analysis of freely secreted, non-displayed, split proinsulin produced from wild-type Pichia pastoris. The peak shows a mass that corresponds to a fully processed two chain molecule.

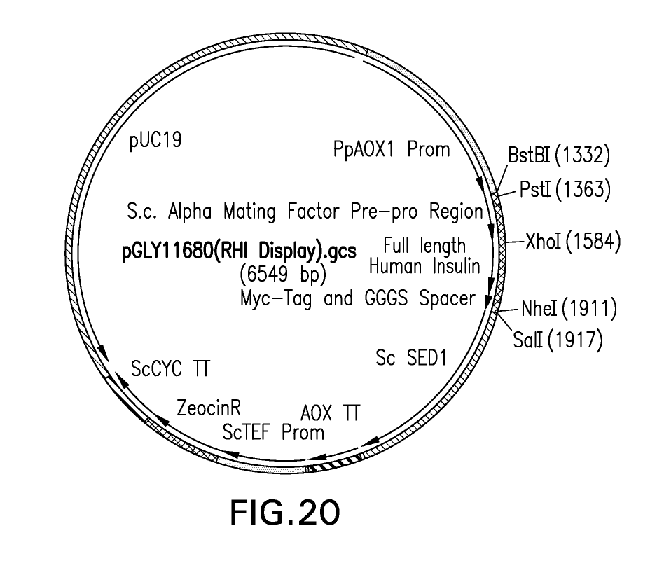

FIG. 20 shows a map of plasmid pGLY11680. Plasmid pGLY11680) is a roll-in vector that targets the AOX1 promoter and contains an expression cassette encoding recombinant human insulin fused to a truncated Saccharomyces cerevisiae Sed1p operably linked to the P. pastoris AOX1 promoter and an expression cassette encoding the zeocin resistance protein (ZeocinR) ORF under the control of the S. cerevisiae TEF1 promoter and S. cerevisiae CYC termination sequence.

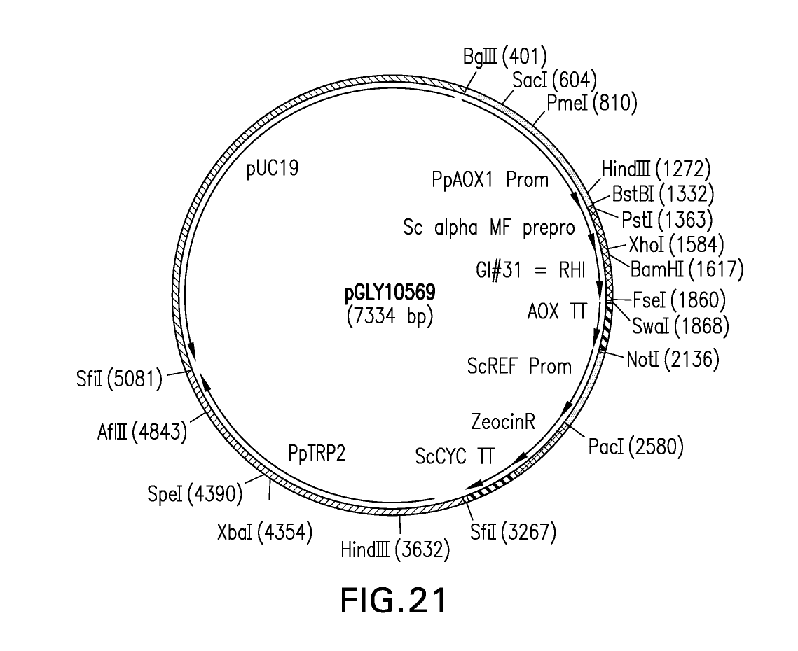

FIG. 21 shows a map of plasmid pGLY11680. Plasmid pGLY11680) is a roll-in vector that targets the TRP2 locus and contains an expression cassette encoding recombinant human insulin operably linked to the P. pastoris AOX1 promoter and an expression cassette encoding the zeocin resistance protein (ZeocinR) ORF under the control of the S. cerevisiae TEF1 promoter and S. cerevisiae CYC termination sequence.

### DETAILED DESCRIPTION OF THE INVENTION

The present invention provides a combinatorial library or protein display system or method for identifying ligands for the insulin receptor (IR) or insulin growth factor 1 (IGF-1) receptor (e.g., IR or IGF-1 receptor agonists) and which may used to identify ligands that have a particular or desired affinity and/or avidity for the IR or IGF-1 receptor. In general, the protein display system enables the display of diverse libraries of ligands for the IR or IGF-1 receptor on the surface of cells and the subsequent selection and isolation of those cells that express a ligand with an affinity or a particular or desired affinity and/or avidity for the IR or IGF-1 receptor. The nucleotide sequence of the nucleic acid molecule encoding the ligand or the amino acid sequence of the ligand can be determined and the sequence information used to construct a cell line that may be used to produce the ligand. The methods disclosed herein are particularly useful for identifying ligands for treating diabetes.

As used herein, the terms “ligand for the IR or IGF-1 receptor” and “ligand” both refer to any peptide, polypeptide, or protein, examples including but not limited to heterodimer insulin analogues, single-chain insulin analogues, fusion proteins comprising a polypeptide corresponding to an insulin analogue precursor molecule, IGF-1 analogues, IGF-1 analogues modified to preferentially bind the IR, and immunoglobulins, scFv molecules, or Fab molecules that may bind the IR or IGF-1 receptor. In a further embodiment, the terms “ligand for the IR or IGF-1 receptor” and “ligand” both refer to heterodimer insulin analogues, single-chain insulin analogues, fusion proteins comprising a polypeptide corresponding to an insulin analogue precursor molecule, IGF-1 analogues, or IGF-1 analogues modified to preferentially bind the IR. In a further embodiment, the terms “ligand for the IR or IGF-1 receptor” and “ligand” both refer heterodimer insulin analogues, single-chain insulin analogues, and fusion proteins comprising a polypeptide corresponding to an insulin analogue precursor molecule. In general, ligands for the IR are IR agonists. The IR ligands or agonists may be used in a therapy for treating diabetes that is insulin-dependent, e.g., Type I diabetes or Type II diabetes that is at a disease state where the therapy for the patient includes administering to the patient an exogenous insulin. In the methods herein the ligand is fused to a cell surface anchoring moiety or protein that displays the ligand on the surface of the cell. Nucleic acid molecules encoding ligands fused to a cell surface anchoring moiety protein that have been identified as being capable of binding to the IR or IGF-1 receptor may be sequenced. The sequence may be used to synthesize nucleic acid molecules that encode the ligand without the cell anchoring moiety or protein fused thereto.

The compositions and methods comprising the protein display system or method are particularly useful for the display of collections or libraries of ligands for the IR and/or IGF-1 receptor (e.g., recombinant insulin analogue precursor molecules) in the context of discovery (that is, screening) or molecular evolution protocols. A salient feature of the method is that it provides a display system in which a library of cells may be constructed wherein each cell in the library is capable of displaying on the surface thereof a particular ligand or recombinant insulin analogue precursor molecule (ligand or recombinant insulin analogue precursor molecule of interest) and that these cells may be screened using the IR and/or IGF-1 receptor to identify and select those cells in the library that express a ligand or recombinant insulin analogue precursor molecule with a particular or desired affinity and/or avidity to the IR and to the IGF-1 receptor from recombinant cells that express molecules that have little or no affinity and/or avidity for the IR or IGF-1 receptor.

In general, the methods disclosed herein enable recombinant host cells that express a ligand that preferentially binds the IR to be identified and separated from recombinant cells that express a molecule that has little or no detectable activity at the IGF-1 receptor. For example, in a first step, recombinant cells that express molecules that bind the IR are separated from molecules that express molecules that have little or no detectable binding to the IR. In a second step, the recombinant cells that express molecules that bind the IR are then contacted with the IGF-1 receptor and recombinant cells that express molecules that have little or no detectable binding to the IGF-1 receptor are separated from recombinant cells that express molecules that bind the IGF-1 receptor to provide the recombinant cells that preferentially bind the IR and have little or no detectable binding to the IGF-1 receptor. In another example, in a first step, recombinant cells that express molecules that bind the IGF-1 receptor are separated from molecules that express molecules that have little or no detectable binding to the IGF-1 receptor. In a second step, the recombinant cells that express molecules that have little or no detectable binding to the IGF-1 receptor are then contacted with the IR and recombinant cells that express molecules that bind the IR are separated from recombinant cells that have little or no detectable binding to the IR to provide the recombinant cells that preferentially bind the IR and which have little or no detectable binding to the IGF-1 receptor.

Libraries of recombinant cells that express a plurality of ligands (e.g., recombinant insulin analogue precursor molecules) may be constructed by transfecting cells with a library of nucleic acid molecules encoding a plurality of ligands fused to a cell surface anchoring moiety or protein wherein each particular or different ligand is encoded on a different nucleic acid molecule in a different cell in the library and wherein each ligand is fused to a cell surface anchoring moiety. In particular embodiments, each ligand will be fused to a cell surface anchoring moiety or protein of the same kind or type. The ligands that are expressed are sequence variants of each other and each recombinant cell in the library expresses one species of ligand or recombinant insulin analogue precursor molecule. The libraries of nucleic acids can be constructed for example by cassette mutagenesis, error-prone PCR, or DNA shuffling. Methods for error-prone PCR and DNA shuffling can be found for example, Otten & Quax,. “Directed evolution: selecting today's biocatalysts”, Biomolecular engineering 22 (1-3): 1-9 (2005); Besenmatteret al., “New Enzymes from Combinatorial Library Modules”, Methods in Enzymology 388: 91-102 (2004); Reetz & Carballeira, “Iterative saturation mutagenesis (ISM) for rapid directed evolution of functional enzymes”, Nature Prot. 2 (4): 891-903 (2007); Stemmer, “Rapid evolution of a protein in vitro by DNA shuffling”, Nature 370 (6488): 389-391 (1994); Voigt et al., “Rational evolutionary design: the theory of in vitro protein evolution” Advances in Protein Chemistry 55: 79-160 (2001); Arnold, “Design by directed evolution”, Accounts of Chemical Research 31 (3): 125-131 (1998).

In particular embodiments, a library of ligands may be constructed by amplifying a nucleic acid molecule encoding a ligand for the IR or IGF-1 receptor using error-prone PCR to produce a plurality of mutagenized nucleic acid molecules, each encoding a mutated ligand having one or more amino acid substitutions and/or deletions. The plurality of mutagenized nucleic acid molecules encoding the mutated ligands are cloned into an expression vector downstream of a promoter and adjacent to an open reading frame (ORF) encoding the cell surface anchoring moiety or protein to provide an expression cassette in which the ORF encoding the mutated ligand and the ORF encoding the cell surface anchoring moiety or protein are in frame. Expression of the expression cassette in the cell produces a fusion protein in which the mutated ligand is covalently linked by a peptide bond to the cell surface anchoring moiety or protein. The fusion protein is secreted from the cell and attaches to the cell surface by the cell surface anchoring moiety or protein to display the ligand. Identification of cells that express a ligand that is capable of binding the IR or IGF-1 receptor may be achieved by contacting the cells with the IR or IGF-1 receptor covalently linked to a detection moiety or contacting the cells with the IR or IGF-1 receptor and detecting the bound IR or IGF-1 receptor with an antibody covalently linked to a detection moiety. Cell sorting, e.g. FACS cell sorting, may be used to separate cells that express a ligand that is capable of binding the IR or IGF-1 receptor from cells that do not bind or poorly bind the IR or IGF-1 receptor.

In further embodiment, a library of ligands may be constructed by amplifying a nucleic acid molecule encoding native insulin or insulin analogue (e.g., native human insulin or human insulin analogue) using error-prone PCR to produce a plurality of mutagenized nucleic acid molecules, each encoding a mutated insulin analogue having one or more amino acid substitutions and/or deletions. The plurality of mutagenized nucleic acid molecules encoding the mutated insulin analogues are cloned into an expression vector downstream of a promoter and adjacent to an open reading frame (ORF) encoding the cell surface anchoring moiety or protein to provide an expression cassette in which the ORF encoding the mutated insulin analogue and the ORF encoding the cell surface anchoring moiety or protein are in frame. Expression of the expression cassette in the cell produces a fusion protein in which the mutated insulin analogue is covalently linked by a peptide bond to the cell surface anchoring moiety or protein. The fusion protein is secreted from the cell and attaches to the cell surface by the cell surface anchoring moiety or protein to display the ligand. Identification of cells that express a mutated insulin analogue that is capable of binding the IR may be achieved by contacting the cells with the IR covalently linked to a detection moiety or contacting the cells with the IR and detecting the bound IR with an antibody covalently linked to a detection moiety. Cell sorting, e.g. FACS cell sorting, may be used to separate cells that express a ligand that is capable of binding the IR from cells that do not bind or poorly bind the IR.

In a further embodiment, the cells that express a mutated insulin analogue that is capable of binding the IR but which does not bind or poorly bind the IGF-1 receptor may be identified by contacting the cells with the IGF-1 covalently linked to a detection moiety or contacting the cells with the IGF-1 receptor and detecting the bound IGF-1 receptor with an antibody covalently linked to a detection moiety. The cells that express a mutated insulin analogue that is capable of binding the IR but which does not bind or poorly bind the IGF-1 receptor may be separated by a cell sorting method such as FACS cell sorting.

Libraries of recombinant insulin analogue precursor molecules may also be constructed by transfecting cells with nucleic acid molecules encoding a single species of ligand fused to a cell surface anchoring moiety or protein and then contacting the recombinant cells with a mutagenizing agent for a time sufficient to mutagenize the nucleic acid molecules encoding the ligand to produce a library of recombinant cells wherein each particular or different ligand is encoded on a different nucleic acid molecule in a different recombinant cell in the library. The ligands expressed are sequence variants of each other and each recombinant cell in the library expresses one species of ligand or recombinant insulin analogue precursor molecule. Methods for mutagenizing cells and nucleic acids are well known in the art and include but not limited to UV irradiation, gamma irradiation, x-rays, a restriction enzyme, a mutagenic or teratogenic chemical, a DNA repair inhibitor, N-ethyl-N-nitrosourea (ENU), ethylmethanesulphonate (EMS) and ICR191. U.S. Pat. Nos. 7,972,853; 7,033,781; and 5,736,383 all disclose methods for mutagenizing cells and are all incorporated herein by reference.

The library of recombinant cells may be screened using the IR to identify those recombinant cells in the library that express a ligand (e.g., recombinant insulin analogue precursor molecule) fused to a cell surface anchoring moiety or protein that has a desired or particular affinity and/or avidity to the IR. Recombinant cells that express the desired or particular ligand may be separated from the other cells in the library using methods such as cell sorting. In general, the recombinant cells may be screened using the IR-A or IR-B receptor. Because it is desirable that the ligands have low or no detectable affinity for the insulin growth factor 1 (IGF-1) receptor, the protein display system enables the libraries of recombinant cells to be screened for affinity and/or avidity to the IGF-1 receptor to identify recombinant cells that express ligands with reduced or no detectable affinity and/or avidity to the IGF-1 receptor.

In a further embodiment, provided herein is a method for identifying N-glycosylated ligands (e.g., insulin analogue precursor molecule) that have a desired or particular affinity and/or avidity to the IR or IGF-1 receptor. In this embodiment a plurality of nucleic acid molecules are synthesized wherein each molecule encodes a ligand fused to a cell surface anchoring moiety or protein and wherein the ligand comprises one or more N-glycosylation sites. For example, the ligand may be an insulin analogue precursor molecule that comprises at least one N-glycosylation site in the A-chain peptide or analogue thereof, B-chain peptide or analogue thereof, or C-chain or connecting peptide or in a peptide adjacent to the N-terminus of the B-chain or analogue thereof or A chain or analogue thereof or a peptide adjacent to the C-terminus of the B-chain or analogue thereof or the A-chain or analogue thereof. The plurality of nucleic acid molecules are introduced into recombinant host cells that have been genetically engineered as disclosed herein to produce glycoprotein compositions that have predominantly a particular N-glycan species therein to produce a library of recombinant host cells. Recombinant cells in the library that express an N-glycosylated ligand that binds the IR may be separated from the other cells in the library using methods such as cell sorting. In general, the recombinant cells may be screened using the IR-A or IR-B receptor. Because it is desirable that the ligands have low or no detectable affinity for the insulin growth factor 1 (IGF-1) receptor, the recombinant host cells may be screened for affinity and/or avidity to the IGF-1 receptor to identify recombinant cells that express N-glycosylated ligands with reduced or no detectable affinity and/or avidity to the IGF-1 receptor.

The present invention is based on the discovery that ligands such as recombinant insulin analogue precursor molecules when fused to a cell surface anchoring moiety or protein and displayed on the surface of a cell competent for folding of the ligand or insulin analogue precursor molecule during expression, e.g., a yeast or fungal host cell, may have a structure or form that can bind to the IR or IGF-1 receptor and that the binding to the IR or IGF-1 receptor correlates with the binding of the ligand to the IR or IGF-1 receptor as measured in a conventional assay for measuring affinity and/or avidity of an insulin analogue. The discovery provides the basis for the display methods disclosed herein in which ligands (e.g., recombinant insulin analogue precursor molecules) fused to a cell surface anchoring protein and displayed on the surface of recombinant cells may be in a form that is accessible to binding to an IR, IGF-1 receptor, or other macromolecule or receptor, and cells expressing such ligands or recombinant insulin precursor molecules fused to a cell surface anchoring protein that are capable of binding the IR or IGF-1 receptor can be identified and separated from cells that express a form of the ligand or recombinant insulin analogue precursor that does not bind or poorly binds the IR or IGF-1 receptor. Further, the diplay methods herein enable the identification and selection of cells that express ligands that may preferentially bind one IR isoform over another IR isoform. For example, it is well known that the human IR exists in at least two isoforms, isoform A (IR-A) and isoform B (IR-B). The relative expression of the two isoforms varies in a tissue-specific manner. IR-A is expressed predominantly in central nervous system and hematopoietic cells while IR-B is expressed predominantly in adipose tissue, liver, and muscle, the major target tissues for the metabolic effects of insulin (Moller et al., Mol. Endocrinol. 3: 1263-1269 (19890). IR-A has a slightly higher binding affinity and IR-B has a more efficient signaling activity as evaluated by its tyrosine kinase activity and phosphorylation of insulin receptor substrate 1 (Kosaki & Webster, J. Biol. Chem. 268: 21990-21996 (1993)). The present invention enables identification of ligands with particular ratios of binding to the IR-A versus IR-B and selection of cells encoding the identified ligands.

In a general embodiment of the present invention, a host cell is transformed with a nucleic acid molecule comprising an expression cassette comprising a nucleic acid molecule encoding a fusion protein comprising a ligand that may bind the IR and/or IGF-1 receptor fused at its C-terminus to a protein or peptide that enables the fusion protein to be displayed on the surface of the transformed cell. Examples of proteins or peptides that may enable the fusion protein to be displayed on the surface of the host cell include but are not limited to (1) a cell anchoring protein or cell surface binding portion thereof, (2) a first peptide binding moiety that is capable of specifically binding to a second peptide binding moiety displayed or linked to the surface of the host cell (for example, a second peptide binding moiety fused to a cell anchoring moiety or protein or cell binding portion thereof), and (3) a peptide that comprises a modification motif that binds an acceptor molecule which may then bind a binding partner linked to the cell surface. U.S. Published Application No. 20090005264 discloses surface display methods in which fusion proteins comprising a modification motif are expressed and the modification motif is modified by a coupling enzyme to include a first binding partner which can bind a second binding partner immobilized on the cell surface. The expression of the encoded fusion protein may be regulated by a constitutive or inducible promoter. When the nucleic acid molecule encoding the fusion protein is expressed, i.e., transcribed into an mRNA molecule that is translated into the fusion protein comprising the ligand that may bind the IR and/or IGF-1 receptor therein, the fusion protein is targeted to secretory pathway. As the fusion protein traverses the secretory pathway, the ligand component of the fusion protein is folded into a tertiary structure and if it contains N- or O-linked glycosylation sites, may be glycosylated. The fusion protein is then transferred to secretory vesicles and transported to the cell surface where it is secreted and anchored to the cell surface. The cells with the fusion protein comprising the ligand that may bind the IR and/or IGF-1 receptor displayed on the surface thereof may be screened by contacting the cells with the IR to identify those cells displaying a fusion protein comprising a ligand with the desired binding to the IR (or to the IGF-1 receptor or other macromolecule or receptor).

In a specific embodiment, a host cell is transformed with a nucleic acid molecule comprising an expression cassette comprising a nucleic acid molecule encoding a fusion protein comprising a pre-proinsulin analogue precursor fused at its C-terminus to protein or peptide that enables the fusion protein to be displayed on the surface of the cell. Examples of proteins or peptides that may enable the fusion protein to be displayed on the surface of the cell include but are not limited to a cell anchoring protein or cell binding portion thereof, a peptide binding moiety that is capable of specifically binding to a second peptide binding moiety displayed or linked to the surface of the cell, and a peptide that comprises a modification motif that binds an acceptor molecule which may then bind a binding partner linked to the cell surface. The expression of the encoded fusion protein is regulated by a constitutive or inducible promoter. When the nucleic acid molecule encoding the fusion protein is expressed, i.e., transcribed into an mRNA molecule that is translated into the fusion protein comprising a pre-proinsulin analogue precursor therein, the fusion protein is targeted to secretory pathway where the pre-peptide is removed to produce a second fusion protein comprising a proinsulin analogue precursor. As the second fusion protein traverses the secretory pathway, the proinsulin analogue precursor component of the fusion protein while still linear is folded into a tertiary structure and may be glycosylated if the fusion protein comprises a glycosylation recognition motif. The second fusion protein comprising the folded proinsulin analogue precursor is then transferred to secretory vesicles where the propeptide is removed to produce a third fusion protein comprising an insulin analogue precursor molecule. The third fusion protein is transported to the cell surface where it is anchored to the cell surface. The cells with the third fusion protein comprising the insulin analogue precursor molecule displayed on the surface thereof may be screened by contacting the cells with the IR to identify those cells displaying a third fusion protein comprising an insulin analogue precursor molecule with the desired binding to the IR (or to the IGF-1 receptor or other macromolecule or receptor). In general, an insulin analogue precursor that is capable of binding the IR will have been folded into a tertiary structure that enables it to bind the IR and which may include the same disulfide linkages as those of native insulin.

When used herein in the context of displayed on the surface, the term “insulin analogue precursor” will be understood to refer to the third fusion protein. Thus, when it is stated that an insulin analogue precursor molecule is displayed on the cell surface, it will be understood that the statement refers to the third fusion protein as being displayed on the cell surface. The insulin analogue precursor fusion protein may be a single-chain molecule in which the C-terminus of the B-chain peptide is connected to the N-terminus of the connecting peptide and the C-terminus of the connecting peptide is connected to the N-terminus of the A-chain peptide but in which the connecting peptide enables or does not significantly interfere with the insulin analogue precursor molecule to maintain an active conformation or form capable of binding the IR. In general, the insulin precursor analogue will have the three disulfide bond linkages characteristic of native human insulin. The insulin precursor analogue fusion protein may be a heterodimer in which the A-chain peptide or analog thereof is covalently linked to the B-chain peptide or analogue thereof by two disulfide bonds as characteristic of native human insulin. In particular embodiments, the insulin precursor analogue fusion protein may be a split proinsulin heterodimer in which the A-chain peptide or analogue thereof is covalently linked to the B-chain peptide or analogue thereof by two disulfide bonds as native human insulin but wherein the B-chain peptide or analogue thereof is covalently linked to the N-terminus of the native insulin C-peptide or analogue thereof or other connecting peptide or polypeptide and the N-terminus of the A-chain peptide or analogue thereof an unbound NH₂ group. For example, insulin or insulin analogues comprising the native human or monkey C-peptide have a kex2 cleavage site at the junction between the C-peptide and the N-terminus of the A-chain peptide, which is cleaved by a kex2 protease in Pichia pastoris host cells to produce a split proinsulin heterodimer molecule. In each above embodiment, the C-terminus of the A-chain peptide or analogue thereof is covalently linked to the N-terminus of the cell surface anchoring moiety or protein or second binding moiety.

In a general embodiment of the present invention, a host cell is transformed with a nucleic acid molecule comprising an expression cassette comprising a nucleic acid molecule encoding a fusion protein comprising a ligand that may bind the IR and/or IGF-1 receptor fused at its C-terminus to protein or polypeptide comprising a cell surface anchoring moiety or protein. The expression of the encoded fusion protein is regulated by a constitutive or an inducible promoter. When the nucleic acid molecule encoding the fusion protein is expressed, the encoded fusion protein is transported to the cell surface via the cell secretory pathway where it is anchored to the cell surface such that the ligand portion of the fusion protein is exposed to the extracellular environment and available to bind the IR and/or IGF-1 receptor. The cells with the fusion protein displayed thereon may be screened to identify those cells displaying a fusion protein comprising a ligand with the desired binding to the IR (or to the IGF-1 receptor or other macromolecule or receptor) by contacting the host cells with the IR (or to the IGF-1 receptor or other macromolecule or receptor).

In the above embodiment, the cells may contacted with a mutagenic agent to generate a plurality of cells comprising nucleic acid molecules encoding a variegated population of mutants of the fusion protein or the cells are transformed with a plurality of nucleic acid molecules which differ in nucleotide sequence encoding the ligand portion of the fusion protein. In either case, a library of cells is produced wherein each cell in the library expresses and displays thereon a ligand having a particular amino acid sequence. The cells can then be screened for binding to the IR, IGF-1 receptor, or other macromolecule and cells displaying a particular ligand capable of binding the IR with a desired affinity and/or avidity may be separated from host cells displaying polypeptides or proteins not capable of binding the IR or which binds the IR with an undesired affinity and/or avidity. In addition, the cells displaying the particular ligand capable of binding the IR with the desired affinity and/or avidity may then be screened using the IGF-1 receptor to identify and isolate those cells that display a particular ligand capable of binding the IR with the desired affinity and/or avidity but which have reduced or no detectable binding affinity and/or avidity for the IGF-1 receptor.

In a specific embodiment, a host cell is transformed with a nucleic acid molecule comprising an expression cassette comprising a nucleic acid molecule encoding a fusion protein comprising a pre-proinsulin analogue precursor fused at its C-terminus to protein comprising a cell surface anchoring protein. The expression of the encoded fusion protein is regulated by a constitutive or inducible promoter. When the nucleic acid molecule encoding the fusion protein is expressed, i.e., transcribed into an mRNA molecule that is translated into the fusion protein comprising a pre-proinsulin analogue precursor therein, the fusion protein is targeted to secretory pathway where the pre-peptide is removed to produce a second fusion protein comprising a proinsulin analogue precursor. As the second fusion protein traverses the secretory pathway, the proinsulin analogue precursor component of the fusion protein is folded into a tertiary structure. The second fusion protein comprising the folded proinsulin analogue precursor is then transferred to secretory vesicles where the propeptide is removed to produce a third fusion protein comprising an insulin analogue precursor molecule. The third fusion protein is transported to the cell surface where it is anchored to the cell surface. The cells with the third fusion protein comprising the insulin analogue precursor molecule displayed on the surface thereof may be screened by contacting the cells with the IR to identify those cells displaying a third fusion protein comprising an insulin analogue precursor molecule with the desired binding to the IR (or to the IGF-1 receptor or other macromolecule or receptor).

In the above embodiment, mutagenesis of the cells may be used to generate a plurality of cells encoding a variegated population of mutants of the fusion proteins or the cells are transformed with a plurality of nucleic acid molecules which differ in nucleotide sequence. In either case, a library of cells is produced wherein each cell expresses and displays thereon a particular insulin analogue precursor molecule. The cells can then be screened for binding to the IR, IGF-1 receptor, or other macromolecule and cells displaying a particular insulin analogue molecule capable of binding the IR with a desired affinity and/or avidity may be separated from cells displaying insulin analogue precursors not capable of binding the IR or which binds the IR with an undesired affinity and/or avidity. In addition, the cells displaying the particular insulin analogue precursor molecule capable of binding the IR with the desired affinity and/or avidity may then be screened using the IGF-1 receptor to identify and isolate those cells that display a particular insulin analogue precursor molecule capable of binding the IR with the desired affinity and/or avidity but which have reduced or no detectable binding affinity and/or avidity for the IGF-1 receptor.

In a further general embodiment, a first host cell that comprises a first nucleic acid molecule encoding a first expression cassette encoding a capture moiety comprising a cell surface anchoring protein or portion thereof fused at its N-terminus to a protein or peptide comprising a first binding moiety is constructed. The first host cell or the cell line is transformed with a second nucleic acid molecule comprising a second expression cassette comprising a nucleic acid molecule encoding a fusion protein comprising a ligand that may bind the IR and/or IGF-1 receptor fused at its C-terminus to a protein or peptide comprising a second binding moiety that is capable of specifically interacting with the first binding moiety fused to the cell surface anchoring protein to produce a second host cell or second cell line. In particular aspects, the first and second binding moieties are capable of pairwise binding. The expression of the encoded capture moiety and fusion protein is regulated by a constitutive or inducible promoter. Expression of the capture moiety may coincide with expression of the fusion protein or expression of the capture moiety may be temporal to expression of the fusion protein. That is, expression of the capture moiety is induced while expression of the fusion protein is repressed. After a sufficient period of time, expression of the capture moiety is repressed and expression of the fusion protein is induced. In particular aspects, induction of expression of the fusion protein results in inhibition of expression of the capture moiety. When the nucleic acid molecule encoding the capture moiety is expressed, the encoded capture moiety is expressed and transported to the cell surface where it anchored to the cell surface via the cell surface anchoring protein. When the nucleic acid molecule encoding the fusion protein is expressed, as discussed previously, the fusion protein is transported to the cell surface via the secretory pathway where it is anchored to the cell surface via binding of the second binding moiety to the first binding moiety comprising the cell surface anchoring protein.

In the above embodiment, mutagenesis of the above second host cells or cell line may used to generate a plurality of cells encoding a variegated population of mutants of the fusion proteins or the first cell or cell line is transformed with a plurality of nucleic acid molecules which differ in nucleotide sequence. In either case, a library of cells is produced wherein each cell displays a particular ligand. The cells can then be screened for binding to the IR, IGF-1 receptor, or other macromolecule, and cells displaying a ligand capable of binding the IR with a desired affinity and/or avidity may be separated from cells displaying ligands not capable of binding the IR or which bind the IR with an undesired affinity and/or avidity. In addition, the cells displaying the particular ligand capable of binding the IR with the desired affinity and/or avidity may then be screened using the IGF-1 receptor to identify and isolate those cells that display a particular ligand capable of binding the IR with the desired affinity and/or avidity but which have reduced or no detectable binding affinity and/or avidity for the IGF-1 receptor.

In a specific embodiment, a host cell that comprises a first nucleic acid molecule encoding a first expression cassette encoding a capture moiety comprising a cell surface anchoring protein or portion thereof fused at its N-terminus to a protein or peptide comprising a first binding moiety is constructed. The first host cell or cell line is transformed with a second nucleic acid molecule comprising a second expression cassette comprising a nucleic acid molecule encoding a fusion protein comprising a pre-proinsulin analogue precursor fused at its C-terminus to a protein or peptide comprising a second binding moiety that is capable of specifically interacting with the first binding moiety fused to the cell surface anchoring protein to produce a second host cell or cell line. In particular aspects, the first and second binding moieties are capable of pairwise binding. The expression of the encoded capture moiety and fusion protein is regulated by a constitutive or inducible promoter. Expression of the capture moiety may coincide with expression of the fusion protein or expression of the capture moiety may be temporal to expression of the fusion protein. That is, expression of the capture moiety is induced while expression of the fusion protein is repressed. After a sufficient period of time, expression of the capture moiety is repressed and expression of the fusion protein is induced. In particular aspects, induction of expression of the fusion protein results in inhibition of expression of the capture moiety. When the nucleic acid molecule encoding the capture moiety is expressed, the encoded capture moiety is expressed and transported to the cell surface where it is anchored to the cell surface via the cell surface anchoring protein. When the nucleic acid molecule encoding the fusion protein is expressed, as discussed previously, the fusion protein is targeted to the secretory pathway where the pre-peptide is removed to provide a second fusion protein. As the second fusion protein traverses the secretory pathway, the proinsulin analogue precursor component of the fusion protein is folded into a tertiary structure. The propeptide is removed from the second fusion protein to provide a third fusion protein which is then secreted to the cell surface where it is anchored to the cell surface via binding of the second binding moiety to the first binding moiety comprising the cell surface anchoring protein.

In the above embodiment, mutagenesis of the cells may be used to generate a plurality of cells encoding a variegated population of mutants of the fusion proteins or the cells are transformed with a plurality of nucleic acid molecules which differ in nucleotide sequence. In either case, a library of cells is produced wherein each cell displays a particular recombinant insulin analogue precursor molecule. The cells can then be screened for binding to the IR, IGF-1 receptor, or other macromolecule, and cells displaying a particular insulin analogue precursor molecule capable of binding the IR with a desired affinity and/or avidity may be separated from cells displaying recombinant insulin analogue precursor molecules not capable of binding the IR or which binds the IR with an undesired affinity and/or avidity. In addition, the cells displaying the particular insulin analogue precursor molecule capable of binding the IR with the desired affinity and/or avidity may then be screened using the IGF-1 receptor to identify and isolate those cells that display a particular insulin analogue precursor molecule capable of binding the IR with the desired affinity and/or avidity but which have reduced or no detectable binding affinity and/or avidity for the IGF-1 receptor.

A consideration in the embodiments that use a capture moiety is to select a pair of binding moiety proteins or peptides capable of binding to each other or forming a pairwise interaction (See for example, U.S. Published Application No. 2010/0331192, which is incorporated herein by reference.). Whereas a nucleic acid molecule encoding one of the binding moiety peptides is inserted in-frame with the nucleic acid molecule encoding a ligand, a nucleic acid molecule encoding the other binding moiety is fused in-frame with a nucleic acid molecule encoding a cell surface anchoring protein capable of attaching to the outer wall or membrane of the cell. By “pairwise interaction” is meant that the two binding moieties can interact with and bind to each other to form a stable complex. The stable complex must be sufficiently long-lasting to permit detecting the protein of interest on the outer surface of the cell. The complex or dimer must be able to withstand whatever conditions exist or are introduced between the moment of formation and the moment of detecting the displayed ligand, these conditions being a function of the assay or reaction which is being performed. The stable complex or dimer may be irreversible or reversible as long as it meets the other requirements of this definition. Thus, a transient complex or dimer may form in a reaction mixture, but it does not constitute a stable complex if it dissociates spontaneously and yields no detectable polypeptide displayed on the outer surface of a genetic package.

The pairwise interaction between the first and second binding moieties may be covalent or non-covalent interactions. Non-covalent interactions encompass every exiting stable linkage that does not result in the formation of a covalent bond. Non-limiting examples of noncovalent interactions include electrostatic bonds, hydrogen bonding, Van der Waal's forces, steric interdigitation of amphiphilic peptides. By contrast, covalent interactions result in the formation of covalent bonds, including but not limited to disulfide bond between two cysteine residues, C—C bond between two carbon-containing molecules, C—O or C—H between a carbon and oxygen- or hydrogen-containing molecules respectively, and O—P bond between an oxygen- and phosphate-containing molecule.

Binding moiety peptides may be derived from a variety of sources. Generally, any protein sequences involved in the formation of stable multimers are candidate binding moiety peptides. As such, these peptides may be derived from any homomultimeric or heteromultimeric protein complexes. Representative homomultimeric proteins are homodimeric receptors (e.g., platelet-derived growth factor homodimer BB (PDGF), homodimeric transcription factors (e.g. Max homodimer, NF-kappaB p65 (RelA) homodimer), and growth factors (e.g., neurotrophin homodimers). Non-limiting examples of heteromultimeric proteins are complexes of protein kinases and SH2-domain-containing proteins (Cantley et al., Cell 72: 767-778 (1993); Cantley et al., J. Biol. Chem. 270: 26029-26032 (1995)), heterodimeric transcription factors, and heterodimeric receptors.

Currently used heterodimeric transcription factors are α-Pal/Max complexes and Hox/Pbx complexes. Hox represents a large family of transcription factors involved in patterning the anterior-posterior axis during embryogenesis. Hox proteins bind DNA with a conserved three alpha helix homeodomain. In order to bind to specific DNA sequences, Hox proteins require the presence of hetero-partners such as the Pbx homeodomain. Wolberger et al. solved the 2.35 Å crystal structure of a HoxB1-Pbx1-DNA ternary complex in order to understand how Hox-Pbx complex formation occurs and how this complex binds to DNA. The structure shows that the homeodomain of each protein binds to adjacent recognition sequences on opposite sides of the DNA. Heterodimerization occurs through contacts formed between a six amino acid hexapeptide N-terminal to the homeodomain of HoxB1 and a pocket in Pbx1 formed between helix 3 and helices 1 and 2. A C-terminal extension of the Pbx1 homeodomain forms an alpha helix that packs against helix 1 to form a larger four helix homeodomain (Wolberger et al., Cell 96: 587-597 (1999); Wolberger et al., J Mol. Biol. 291: 521-530).

A vast number of heterodimeric receptors have also been identified. They include but are not limited to those that bind to growth factors (e.g. heregulin), neurotransmitters (e.g. γ-Aminobutyric acid), and other organic or inorganic small molecules (e.g. mineralocorticoid, glucocorticoid). Currently used heterodimeric receptors are nuclear hormone receptors (Belshaw et al., Proc. Natl. Acad. Sci. U.S.A 93:4604-4607 (1996)), erbB3 and erbB2 receptor complex, and G-protein-coupled receptors including but not limited to opioid (Gomes et al., J. Neuroscience 20: RC110 (2000)); Jordan et al. Nature 399: 697-700 (1999)), muscarinic, dopamine, serotonin, adenosine/dopamine, and GABA_{B} families of receptors. For majority of the known heterodimeric receptors, their C-terminal sequences are found to mediate heterodimer formation.

Peptides derived from antibody chains that are involved in dimerizing the L and H chains can also be used as binding moiety peptides for constructing the subject display systems. These peptides include but are not limited to constant region sequences of an L or H chain. Additionally, binding moiety peptides can be derived from antigen-binding site sequences and its binding antigen.

Based on the wealth of genetic and biochemical data on vast families of genes, one of ordinary skill will be able to select and obtain suitable binding moiety peptides for constructing the subject display system without undue experimentation.

Where desired, sequences from novel hetermultimeric proteins may be used. In such situation, the identification of candidate peptides involved in formation of heteromultimers can be determined by any genetic or biochemical assays without undue experimentation. Additionally, computer modeling and searching technologies further facilitates detection of heteromultimeric peptide sequences based on sequence homologies of common domains appeared in related and unrelated genes. Non-limiting examples of programs that allow homology searches are Blast (http://www.ncbi.nlm.nih.gov/BLAST/), Fasta (Genetics Computing Group package, Madison, Wis.), DNA Star, Clustlaw, TOFFEE, COBLATH, Genthreader, and MegAlign. Any sequence databases that contains DNA sequences corresponding to a target receptor or a segment thereof can be used for sequence analysis. Commonly employed databases include but are not limited to GenBank, EMBL, DDBJ, PDB, SWISS-PROT, EST, STS, GSS, and HTGS.

The subject binding moieties that are derived from heterodimerization sequences can be further characterized based on their physical properties. Current heterodimerization sequences exhibit pairwise affinity resulting in predominant formation of heterodimers to a substantial exclusion of homodimers. Preferably, the predominant formation yields a heteromultimeric pool that contains at least 60% heterodimers, more preferably at least 80% heterodimers, more preferably between 85-90% heterodimers, and more preferably between 90-95% heterodimers, and even more preferably between 96-99% heterodimers that are allowed to form under physiological buffer conditions and/or physiological body temperatures. In certain embodiments of the present invention, at least one of the heterodimerization sequences of the binding moiety pair is essentially incapable of forming a homodimer in a physiological buffer and/or at physiological body temperature. By “essentially incapable” is meant that the selected heterodimerization sequences when tested alone do not yield detectable amounts of homodimers in an in vitro sedimentation experiment as detailed in Kammerer et al., Biochemistry 38: 13263-13269 (1999)), or in the in vivo two-hybrid yeast analysis (see e.g. White et al., Nature 396: 679-682 (1998)). In addition, individual heterodimerization sequences can be expressed in a host cell and the absence of homodimers in the host cell can be demonstrated by a variety of protein analyses including but not limited to SDS-PAGE, Western blot, and immunoprecipitation. The in vitro assays must be conducted under a physiological buffer conditions, and/or preferably at physiological body temperatures. Generally, a physiological buffer contains a physiological concentration of salt and at adjusted to a neutral pH ranging from about 6.5 to about 7.8, and preferably from about 7.0 to about 7.5.

An illustrative binding moiety pair exhibiting the above-mentioned physical properties is GABA_{B}-R1/GABA_{B}-R2 receptors. These two receptors are essentially incapable of forming homodimers under physiological conditions (e.g. in vivo) and at physiological body temperatures. Research by Kuner et al. and White et al. (Science 283: 74-77 (1999)); Nature 396: 679-682 (1998)) has demonstrated the heterodimerization specificity of GABA_{B}-R1 and GABA_{B}-R2 in vivo. In fact, White et al. were able to clone GABA_{B}-R2 from yeast cells based on the exclusive specificity of this heterodimeric receptor pair. In vitro studies by Kammerer et al. supra has shown that neither GABA_{B}-R1 nor GABA_{B}-R2 C-terminal sequence is capable of forming homodimers in physiological buffer conditions when assayed at physiological body temperatures. Specifically, Kammerer et al. have demonstrated by sedimentation experiments that the heterodimerization sequences of GABA_{B} receptor 1 and 2, when tested alone, sediment at the molecular mass of the monomer under physiological conditions and at physiological body temperatures (e.g., at 37° C.). When mixed in equimolar amounts, GABA_{B} receptor 1 and 2 heterodimerization sequences sediment at the molecular mass corresponding to the heterodimer of the two sequences (see Table 1 of Kammerer et al.). However, when the GABA_{B}-R1 and GABA_{B}-R2 C-terminal sequences are linked to a cysteine residue, homodimers may occur via formation of disulfide bond.

Binding moieties can be further characterized based on their secondary structures. Current binding moieties consist of amphiphilic peptides that adopt a coiled-coil helical structure. The helical coiled-coil is one of the principal subunit oligomerization sequences in proteins. Primary sequence analysis reveals that approximately 2-3% of all protein residues form coiled coils (Wolf et al., Protein Sci. 6: 1179-1189 (1997)). Well-characterized coiled coil-containing proteins include members of the cytoskeletal family (e.g., α-keratin, vimentin), cytoskeletal motor family (e.g., myosine, kinesins, and dyneins), viral membrane proteins (e.g. membrane proteins of Ebola or HIV), DNA binding proteins, and cell surface receptors (e.g. GABA_{B} receptors 1 and 2). Coiled-coil adapters of the present invention can be broadly classified into two groups, namely the left-handed and right-handed coiled-coils. The left-handed coiled coils are characterized by a heptad repeat denoted “abcdefg” with the occurrence of apolar residues preferentially located at the first (a) and fourth (d) position. The residues at these two positions typically constitute a zig-zag pattern of “knobs and holes” that interlock with those of the other stand to form a tight-fitting hydrophobic core. In contrast, the second (b), third (c) and sixth (f) positions that cover the periphery of the coiled-coil are preferably charged residues. Examples of charged amino acids include basic residues such as lysine, arginine, histidine, and acidic residues such as aspartate, glutamate, asparagine, and glutamine. Uncharged or apolar amino acids suitable for designing a heterodimeric coiled-coil include but are not limited to glycine, alanine, valine, leucine, isoleucine, serine and threonine. While the uncharged residues typically form the hydrophobic core, inter-helical and intra-helical salt-bridge including charged residues even at core positions may be employed to stabilize the overall helical coiled-coiled structure (Burkhard et al (2000) J. Biol. Chem. 275:11672-11677). Whereas varying lengths of coiled coil may be employed, the subject coiled-coil binding moieties preferably contain two to ten heptad repeats. More preferably, the binding moieties contain three to eight heptad repeats, even more preferably contain four to five heptad repeats.

In designing optimal coiled-coil binding moieties, a variety of existing computer software programs that predict the secondary structure of a peptide can be used. An illustrative computer analysis uses the COILS algorithm which compares an amino acid sequence with sequences in the database of known two-stranded coiled coils, and predicts the high probability coiled-coil stretches (Kammerer et al., Biochemistry 38:13263-13269 (1999)).

While a diverse variety of coiled-coil peptides involved in multimer formation can be employed as the adapters in the subject display system. Current coiled-coils are derived from heterodimeric receptors. Accordingly, the present invention encompasses coiled-coil binding moieties derived from GABA_{B} receptors 1 and 2. In one aspect, the subject coiled-coil peptide binding moieties comprise the C-terminal sequences of GABA_{B} receptor 1 and GABA_{B} receptor 2. In another aspect, the subject binding moieties are composed of two distinct polypeptides of at least 30 amino acid residues, one of which is essentially identical to a linear sequence of comparable length depicted in SEQ ID NO:57 (GR1), and the other is essentially identical to a linear peptide sequence of comparable length depicted in SEQ ID NO:58 (GR2).

Another class of current coiled-coil peptides are leucine zippers. The leucine zipper have been defined in the art as a stretch of about 35 amino acids containing four-five leucine residues separated from each other by six amino acids (Maniatis and Abel, Nature 341:24 (1989)). The leucine zipper has been found to occur in a variety of eukaryotic DNA-binding proteins, such as GCN4, C/EBP, c-fos gene product (Fos), c-jun gene product (Jun), and c-Myc gene product. In these proteins, the leucine zipper creates a dimerization interface wherein proteins containing leucine zippers may form stable homodimers and/or heterodimers. Molecular analysis of the protein products encoded by two proto-oncogenes, c-fos and c-jun, has revealed such a case of preferential heterodimer formation (Gentz et al., Science 243: 1695 (1989); Nakabeppu et al., Cell 55: 907 (1988); Cohen et al., Genes Dev. 3: 173 (1989)). Synthetic peptides comprising the leucine zipper regions of Fos and Jun have also been shown to mediate heterodimer formation, and, where the amino-termini of the synthetic peptides each include a cysteine residue to permit intermolecular disulfide bonding, heterodimer formation occurs to the substantial exclusion of homodimerization.

In a further aspect of the above embodiments, the ligand for the IR and/or IGF-1 receptor is fused to the Fc fragment of an antibody and the capture moiety comprises a protein capable of binding the Fc fragment fused to the cell surface anchoring protein or cell surface binding portion thereof. Examples of Fc binding proteins include but are not limited to but are not limited to those selected from the group consisting of protein A, protein A ZZ domain, protein G, and protein L and fragments thereof that retain the ability to bind to the immunoglobulin. Examples of other binding moieties, include but are not limited to, Fc receptor (FcR) proteins and immunoglobulin-binding fragments thereof. The FCR proteins include members of the Fc gamma receptor (FcγR) family, which bind gamma immunoglobulin (IgG), Fc epsilon receptor (FcεR) family, which bind epsilon immunoglobulin (IgE), and Fc alpha receptor (FcαR) family, which bind alpha immunoglobulin (IgA). Particular FcR proteins that bind IgG that can comprise the binding moiety herein include at least the IgG binding region of FcγRI, FcγRIIA, FcγRIIB1, FcγRIIB2, FcγRIIIA, FcγRIIIB, or FcγRn (neonatal).

In a further general embodiment of the present invention, a recombinant cell is constructed that comprises a first nucleic acid molecule encoding a first binding partner that recognizes and binds or couples to a modification motif or an enzyme that facilitates the synthesis of the first binding partner and a second nucleic acid molecule comprising an expression cassette comprising a nucleic acid molecule encoding a fusion protein comprising a ligand that may bind the IR and/or IGF-1 receptor fused at its C-terminus to a protein or peptide comprising the modification motif. The expression of the first nucleic acid molecules are independently regulated by a constitutive or inducible promoter. In general, expression of the first nucleic acid molecule results in the production of the first binding partner, which binds or couples to the modification motif to form a complex. The ligand comprising the complex is transported to the cell surface via the secretory pathway where it is then secreted. The recombinant cell further displays a second binding partner on the cell surface which specifically binds the first binding partner bound comprising the secreted complex. The second binding partner may be chemically coupled to the cell surface or it may be encoded by a third nucleic acid molecule comprising an expression cassette encoding a fusion protein in which the second binding partner is fused to a cell surface anchoring protein. The fusion protein is independently expressed from a constitutive or inducible promoter. The recombinant cells with the ligand displayed on the surface thereof may be screened by contacting the host cells with the IR to identify those host cells displaying a ligand with the desired binding to the IR (or to the IGF-1 receptor or other macromolecule or receptor).

In a specific example of the above embodiment, the first binding partner may be biotin and the second binding partner may be avidin or an avidin-like molecule and the modification motif is a biotin acceptor peptide. U.S. Published application No. 2009/0005264, which is specifically incorporated herein by reference, discloses examples of library screening methods that comprise the above first and second binding pairs.

In the above embodiment, mutagenesis of the cells may used to generate a plurality of cells encoding a variegated population of mutants of the fusion proteins or the cells are transformed with a plurality of nucleic acid molecules which differ in nucleotide sequence. In either case, a library of cells is produced wherein each cell in the library displays a particular recombinant insulin analogue precursor molecule. The library cells may then be screened for binding to the IR, IGF-1 receptor, or other macromolecule, and host cells displaying a particular ligand capable of binding the IR with a desired affinity and/or avidity may be separated from cells displaying ligands not capable of binding the IR or which binds the IR with an undesired affinity and/or avidity. In addition, the cells displaying an insulin analogue precursor molecule capable of binding the IR with the desired affinity and/or avidity may then be screened using the IGF-1 receptor to identify and isolate those cells that display a ligand capable of binding the IR with the desired affinity and/or avidity but which have reduced or no detectable binding affinity and/or avidity for the IGF-1 receptor.

In a specific embodiment, a recombinant cell is constructed that comprises a first nucleic acid molecule encoding a first binding partner that recognizes and binds or couples to a modification motif or an enzyme that facilitates the synthesis of the first binding partner and a second nucleic acid molecule comprising an expression cassette comprising a nucleic acid molecule encoding a fusion protein comprising a pre-proinsulin analogue precursor fused at its C-terminus to protein or peptide comprising the modification motif. The expression of the first nucleic acid molecules is independently regulated by a constitutive or inducible promoter. In general, expression of the first nucleic acid molecule results in the production of the first binding partner, which binds or couples to the modification motif to form a complex. The insulin analogue precursor comprising the complex is folded into a structure that is similar to the tertiary structure of native insulin and secreted. The recombinant cell further displays a second binding partner on the cell surface that specifically binds the first binding partner bound comprising the secreted complex. The second binding partner may be chemically coupled to the cell surface or it may be encoded by a third nucleic acid molecule comprising an expression cassette encoding a fusion protein in which the second binding partner is fused to a cell surface anchoring protein. The fusion protein is independently expressed from a constitutive or inducible promoter. The recombinant cells with the insulin analogue precursor molecule displayed on the surface thereof may be screened by contacting the cells with the IR to identify those cells displaying a proinsulin analogue precursor molecule with the desired binding to the IR (or to the IGF-1 receptor or other macromolecule or receptor).

In the above embodiment, mutagenesis of the cells may used to generate a plurality of cells encoding a variegated population of mutants of the fusion proteins or the cells are transformed with a plurality of nucleic acid molecules that differ in nucleotide sequence. In either case, a library of cells is produced wherein each cell displays a particular recombinant insulin analogue precursor molecule. The cells may then be screened for binding to the IR, IGF-1 receptor, or other macromolecule, and cells displaying a particular insulin analogue precursor molecule capable of binding the IR with a desired affinity and/or avidity may be separated from cells displaying recombinant insulin analogue precursor molecules not capable of binding the IR or which binds the IR with an undesired affinity and/or avidity. In addition, the cells displaying an insulin analogue precursor molecule capable of binding the IR with the desired affinity and/or avidity may then be screened using the IGF-1 receptor to identify and isolate those cells that display a particular insulin analogue precursor molecule capable of binding the IR with the desired affinity and/or avidity but which have reduced or no detectable binding affinity and/or avidity for the IGF-1 receptor.

In any of the general or specific embodiments disclosed herein, the cell surface anchoring protein or cell binding portion thereof may be a Glycosylphosphatidylinositol-anchored (GPI) protein or cell binding portion thereof, which provides a suitable means for tethering the proinsulin analogue precursor molecules to the surface of the host cell. GPI proteins have been identified and characterized in a wide range of species from humans to yeast and fungi. Thus, in particular aspects of the methods disclosed herein, the cell surface anchoring protein is a GPI protein or fragment thereof that can anchor to the cell surface. Lower eukaryotic cells have systems of GPI proteins that are involved in anchoring or tethering expressed proteins to the cell wall so that they are effectively displayed on the cell wall of the cell from which they were expressed. For example, 66 putative GPI proteins have been identified in Saccharomyces cerevisiae (See, de Groot et al., Yeast 20: 781-796 (2003)). GPI proteins which may be used in the methods herein include, but are not limited to those encoded by Saccharomyces cerevisiae CWP1, CWP2, SED1, and GAS1; Pichia pastoris SP1 and GAS1; and H. polymorpha TIP1. Additional GPI proteins may also be useful. Alpha-agglutinin consists of a core subunit encoded by AGA1 and is linked through disulfide bridges to a small binding subunit encoded by AGA2. The insulin analogue precursor may be fused to the N-terminal region of Aga1p or on the N-terminal region of Aga2p. The examples exemplify the method using the Sed1p encoded by the Saccharomyces cerevisiae SED1 gene. Additional suitable GPI proteins can be identified using the methods and materials of the invention described and exemplified herein.

In particular embodiments, the cell surface anchoring protein is not a GPI protein. The cell surface anchoring protein may instead be a cell surface protein that is partially exposed to the extracellular environment at one of its termini and may have a high copy number. The recombinant insulin analogue precursor may be fused to the exposed terminus. Examples of non-GPI cell surface anchoring proteins include but are not limited to Ccw14p, Cis3p, Cwp1p, Pir1p, Pir4p, Sag1, Step 2, and Step 3.

Thus, a suitable cell surface anchoring proteins may include α-agglutinin, Ccw14p, Cwp1p, Cwp2p, Gas1p, Yap3p, Flo1p, Crh2p, Pir1p, Pir4p, Sed1p, Tip1p, Hpwp1p, Als3p, or Rbt5p. In general, the GPI or non-GPI protein that comprises the fusion protein will be a truncated molecule in which the cell surface anchoring portion or domain is fused at its N-terminus to the C-terminus of the polypeptide comprising the proinsulin analogue precursor and which comprises the recombinant insulin analogue precursor anchored and displayed upon the cell surface.

Detection and analysis of cells that display the recombinant insulin analogue precursor molecule of interest may be achieved by contacting the host cell with an IR or IGF-1 receptor. In particular aspects, the IR is labeled with a detection moiety. In other aspects, the IR or IGF-1 receptor is unlabeled and detection is achieved by using a detection immunoglobulin that is labeled with a detection moiety and binds an epitope of the IR or IGF-1 receptor. In another aspect, the detection immunoglobulin is specific for the IR or IGF-1 receptor-recombinant insulin analogue precursor molecule of interest complex. Regardless of the detection means, a high occurrence of the label indicates the displayed recombinant insulin analogue precursor molecule of interest binds the IR or IGF-1 receptor and a low occurrence of the label indicates the recombinant insulin analogue precursor molecule has been mutated or modified to have little or capability of binding the IR or IGF-1 receptor compared to native insulin.

Detection moieties that are suitable for labeling are well known in the art. Examples of detection moieties, include but are not limited to, fluorescein (FITC), Alexa Fluors such as Alexa Fuor 488 (Invitrogen), green fluorescence protein (GFP), Carboxyfluorescein succinimidyl ester (CFSE), DyLight Fluors (Thermo Fisher Scientific), HyLite Fluors (AnaSpec), and phycoerythrin. Other detection moieties include but are not limited to, magnetic beads which are coated with the IR or IGF-1 receptor or an antibody that is specific for the IR or IGF-1 receptor or a complex comprising the IR or IGF-1 receptor and fusion protein comprising the recombinant proinsulin analogue precursor molecule of interest. In particular aspects, the magnetic beads are coated with anti-fluorochrome immunoglobulins specific for the fluorescent label on the labeled IR or IGF-1 receptor. Thus, the host cells are incubated with the labeled-IR or IGF-1 receptor or immunoglobulin specific for the IR or IGF-1 receptor and then incubated with the magnetic beads specific for the fluorescent label.

Analysis of the cell population and cell sorting of those cells that display the recombinant insulin analogue precursor molecule of interest which are based upon the presence of the detection moiety can be accomplished by a number of techniques known in the art. Cells that display the recombinant insulin analogue precursor molecule of interest may be analyzed or sorted by, for example, flow cytometry, magnetic beads, or fluorescence-activated cell sorting (FACS). These techniques allow the analysis and sorting according to one or more parameters of the cells. Usually one or multiple secretion parameters can be analyzed simultaneously in combination with other measurable parameters of the cell, including, but not limited to, cell type, cell surface antigens, DNA content, etc. The data can be analyzed and cells that the recombinant insulin analogue precursor molecule of interest can be sorted using any formula or combination of the measured parameters. Cell sorting and cell analysis methods are known in the art and are described in, for example, The Handbook of Experimental Immunology, Volumes 1 to 4, (D. N. Weir, editor) and Flow Cytometry and Cell Sorting (A. Radbruch, editor, Springer Verlag, 1992). Cells can also be analyzed using microscopy techniques including, for example, laser scanning microscopy, fluorescence microscopy; techniques such as these may also be used in combination with image analysis systems. Other methods for cell sorting include, for example, panning and separation using affinity techniques, including those techniques using solid supports such as plates, beads, and columns.

When the protein display system herein is combined with fluorescence-activated cell sorting (FACS), the system provides a method for rapidly selecting host cells that display a recombinant insulin analogue precursor molecule with desired (1) a modified affinity and/or avidity for the insulin receptor (IR) and reduced affinity and avidity for the insulin-like growth factor (IGF) receptors, (2) conditional binding properties, eg., IR binding influenced by serum glucose levels, (3) protein stability, and/or (4) optimal signal peptide and C-peptide sequences from rationally designed or mutagenic libraries.

Regulatory sequences which may be used in the practice of the methods disclosed herein include signal sequences, promoters, and transcription terminator sequences. It is generally preferred that the regulatory sequences used be from a species or genus that is the same as or closely related to that of the host cell or is operational in the host cell type chosen. Examples of signal sequences include those of Saccharomyces cerevisiae invertase; Saccharomyces cerevisiae alpha-mating factor, the Aspergillus niger amylase and glucoamylase; human serum albumin; Kluyveromyces maxianus inulinase; and Pichia pastoris mating factor and Kar2. Signal sequences shown herein to be useful in yeast and filamentous fungi include, but are not limited to, the alpha-mating factor presequence and pre-prosequence from Saccharomyces cerevisiae; and signal sequences from numerous other species. Examples of signal sequences that have been used to express recombinant insulin precursors in yeast include but are not limited to the Yps1ss peptide, a synthetic leader or signal peptide disclosed in U.S. Pat. Nos. 5,639,642 and 5,726,038, and which are hereby incorporated herein by reference; and the TA57 propeptide and N-terminal spacer described by Kjeldsen et al., Gene 170:107-112 (1996) and in U.S. Pat. Nos. 6,777,207, and 6,214,547, which are hereby incorporated herein by reference. Other synthetic propeptides are disclosed in U.S. Pat. Nos. 5,395,922; 5,795,746; and 5,162,498; and WO 9832867, and which are hereby incorporated herein by reference. However, it may also be advantageous to use the endogenous signal sequence and/or terminator from the native recombinant protein. For example, the native signal sequence and/or terminator from human insulin could be used to drive secretion of the insulin display construct.

Examples of promoters include promoters from numerous species, including but not limited to alcohol-regulated promoter, tetracycline-regulated promoters, steroid-regulated promoters (e.g., glucocorticoid, estrogen, ecdysone, retinoid, thyroid), metal-regulated promoters, pathogen-regulated promoters, temperature-regulated promoters, and light-regulated promoters. Specific examples of regulatable promoter systems well known in the art include but are not limited to metal-inducible promoter systems (e.g., the yeast copper-metallothionein promoter), plant herbicide safner-activated promoter systems, plant heat-inducible promoter systems, plant and mammalian steroid-inducible promoter systems, Cym repressor-promoter system (Krackeler Scientific, Inc. Albany, N.Y.), RheoSwitch System (New England Biolabs, Beverly Mass.), benzoate-inducible promoter systems (See WO2004/043885), and retroviral-inducible promoter systems. Other specific regulatable promoter systems well-known in the art include the tetracycline-regulatable systems (See for example, Berens & Hillen, Eur J Biochem 270: 3109-3121 (2003)), RU 486-inducible systems, ecdysone-inducible systems, and kanamycin-regulatable system. Lower eukaryote-specific promoters include but are not limited to the Saccharomyces cerevisiae TEF-1 promoter, Pichia pastoris GAPDH promoter, Pichia pastoris GUT1 promoter, PMA-1 promoter, Pichia pastoris PCK-1 promoter, and Pichia pastoris AOX-1 and AOX-2 promoters. For temporal expression of a capture moiety comprising a surface anchoring moiety or protein fused to a first binding partner and an insulin analogue precursor fused to a second binding partner capable of binding the first binding partner, the Pichia pastoris GUT1 promoter is operably linked to the nucleic acid molecule encoding the capture moiety and the Pichia pastoris GAPDH promoter is operably linked to the nucleic acid molecule encoding the insulin analogue precursor fused to the second binding partner (See U.S. Published Application No. 20100009866, which is incorporated herein by reference, for temporal display of antibody molecules and capture moieties). Romanos et al., Yeast 8: 423-488 (1992) provide a review of yeast promoters and expression vectors. Hartner et al., Nucl. Acid Res. 36: e76 (pub on-line 6 Jun. 2008) describes a library of promoters for fine-tuned expression of heterologous proteins in Pichia pastoris as does Cregg et al. in U.S. Published Application No. 20080108108, which is incorporated herein by reference.

The promoters that are operably linked to the nucleic acid molecules disclosed herein can be constitutive promoters or inducible promoters. An inducible promoter, for example the AOX1 promoter, is a promoter that directs transcription at an increased or decreased rate upon binding of a transcription factor in response to an inducer. Transcription factors as used herein include any factor that can bind to a regulatory or control region of a promoter and thereby affect transcription. The RNA synthesis or the promoter binding ability of a transcription factor within the host cell can be controlled by exposing the host to an inducer or removing an inducer from the host cell medium. Accordingly, to regulate expression of an inducible promoter, an inducer is added or removed from the growth medium of the host cell. Such inducers can include sugars, phosphate, alcohol, metal ions, hormones, heat, cold and the like. For example, commonly used inducers in yeast are glucose, galactose, alcohol, and the like.

Transcription termination sequences that are selected are those that are operable in the particular host cell selected. For example, yeast transcription termination sequences are used in expression vectors when a yeast host cell such as Saccharomyces cerevisiae, Kluyveromyces lactis, or Pichia pastoris is the host cell whereas fungal transcription termination sequences would be used in host cells such as Aspergillus niger, Neurospora crassa, or Tricoderma reesei. Transcription termination sequences include but are not limited to the Saccharomyces cerevisiae CYC transcription termination sequence (ScCYC TT), the Pichia pastoris ALG3 transcription termination sequence (ALG3 TT), the Pichia pastoris ALG6 transcription termination sequence (ALG6 TT), the Pichia pastoris ALG12 transcription termination sequence (ALG12 TT), the Pichia pastoris AOX1 transcription termination sequence (AOX1 TT), the Pichia pastoris OCH1 transcription termination sequence (OCH1 TT) and Pichia pastoris PMA1 transcription termination sequence (PMA1 TT). Other transcription termination sequences can be found in the examples and in the art.

The displayed recombinant insulin analogue precursor molecule of interest may optionally include an N-terminal extension or spacer peptide, as described in U.S. Pat. No. 5,395,922 and European Patent No. 765,395A, both of which are herein specifically incorporated by reference. The N-terminal extension or spacer is a peptide that is positioned between the signal peptide or propeptide and the N-terminus of the B-chain. Following removal of the signal peptide and propeptide during passage through the secretory pathway, the N-terminal extension peptide remains attached to the N-glycosylated insulin precursor. Thus, during fermentation, the N-terminal end of the B-chain is protected against the proteolytic activity of yeast proteases such as DPAP. The presence of an N-terminal extension or spacer peptide may also serve as a protection of the N-terminal amino group during chemical processing of the protein, i.e., it may serve as a substitute for a BOC (t-butyl-oxycarbonyl) or similar protecting group.

The N-terminal extension or spacer may be removed from the insulin analogue precursor by means of a proteolytic enzyme that is specific for a basic amino acid (e.g., Lys) so that the terminal extension is cleaved off at the Lys residue. Examples of such proteolytic enzymes are trypsin, Achromobacter lyticus protease, or Lysobacter enzymogenes endoprotease Lys-C. Digestion of the displayed recombinant insulin analogue precursor with the proteolytic enzyme will remove the N-terminal extension or spacer peptide and when cleavage sites are present at the ends of the C-peptide, remove the C-peptide. In such embodiments, the displayed insulin analogue will be in a heterodimer configuration in which the A-chain and B-chain N-termini, Gly and Phe, respectively, are uncoupled and free, i.e., not in peptide bond to an another amino acid. The displayed insulin analogue may also be converted into an acylated derivative using methods such as disclosed in U.S. Pat. No. 5,750,497 and U.S. Pat. No. 5,905,140, the disclosures of which are incorporated by reference hereinto. The displayed recombinant insulin analogue precursors exemplified in the examples comprise an N-terminal extension or spacer comprising ten His (10×His) residues flanked by two Glu residues at the N-terminal end and by the tripeptide sequence Glu-Pro-Lys at the C-terminal end. The 10×His sequence provides a convenient detection sequence for demonstrating the recombinant insulin analogue precursor is displayed on the cell surface using an antibody against the 10×His sequence.

The displayed insulin analogue precursor molecule may further include a peptide spacer or linker that joins the polypeptide encoding the C-terminus of the A-chain to the N-terminus of the polypeptide encoding the truncated SED1 protein, second binding moiety capable of specifically binding the first binding moiety, or modification motif. For example, the peptide spacer or linker may be any amino acid sequence of between one and 100 amino acids. In particular embodiments, the peptide spacer or linker may provide an unstructured peptide sequence. U.S. Pat. No. 7,855,272 and WO2009023270 disclose unstructured peptides that may provide suitable peptide spacer or linker in the recombinant insulin analogue precursor molecules disclosed herein. In particular embodiments, the peptide spacer or linker has the formula (Gly₄Ser)ₙ wherein n is a positive integer selected from 1, 2, 3, 4, 5, 6, 7, 8, 9, or 10. The displayed recombinant insulin analogue precursors exemplified in the examples comprise the 3×G4S peptide linker or spacer. The exemplified spacer further includes a cMyc epitope at the N-terminal end which provides a convenient detection sequence for demonstrating the recombinant insulin analogue precursor is displayed on the cell surface using an antibody against the cMyc epitope.

When the above non-insulin analogue sequences are fused to the insulin analogue sequences comprising the A-chain and B-chain by a terminal Lys residue, this creates a protease (e.g., trypsin or LysC) cleavage site. Therefore, an isolated host cell that produces the recombinant insulin analogue precursor of interest displayed on the cell surface can be used to produce a recombinant insulin analogue by contacting the culture medium used to grow the host cells with a protease that cleaves after Lys residues, e.g., trypsin or LysC, which removes the optional N-terminal extension and non-insulin polypeptides/proteins downstream from the C-terminus of the A-chain and optionally removes the C-peptide. The treatment with the protease effects the release of the insulin analogue into the medium as a recombinant insulin analogue heterodimer. In embodiments where the C-peptide is not removed, recombinant single-chain insulin analogues are produced.

The displayed insulin analogue precursor molecule may include a connecting peptide, which may vary from 4 amino acid residues and up to a length corresponding to the length of the natural or native C-peptide in human proinsulin. The connecting peptide may be the native human or monkey insulin C-peptide or a polypeptide having a length from 3 to about 35, from 3 to about 30, from 4 to about 35, from 4 to about 30, from 5 to about 35, from 5 to about 30, from 6 to about 35 or from 6 to about 30, from 3 to about 25, from 3 to about 20, from 4 to about 25, from 4 to about 20, from 5 to about 25, from 5 to about 20, from 6 to about 25 or from 6 to about 20, from 3 to about 15, from 3 to about 10, from 4 to about 15, from 4 to about 10, from 5 to about 15, from 5 to about 10, from 6 to about 15 or from 6 to about 10, or from 6-9, 6-8, 6-7, 7-8, 7-9, or 7-10 amino acid residues in the peptide chain. In particular embodiments, the connecting peptide comprises a kex2 recognition sequence at the C-terminal end so that when the connecting peptide is covalently linked to the A-chain peptide by a peptide bond, the peptide bond is cleaved by the kex2 protease.

Single-chain peptides have been disclosed in U.S. Published Application No. 20080057004, U.S. Pat. No. 6,630,348, International Application Nos. WO2005054291, WO2007104734, WO2010080609, WO20100099601, and WO2011159895, each of which is incorporated herein by reference. Further provided are compositions and formulations of the above comprising a pharmaceutically acceptable carrier, salt, or combination thereof.

In particular embodiments the N-glycosylated single-chain insulin analogue connecting peptide comprises the formula Gly-Z¹-Gly-Z² wherein Z¹ is Asn or another amino acid except for tyrosine, and Z² is a peptide of 2-35 amino acids. In particular embodiments, the connecting peptide comprises a kex2 recognition sequence at the C-terminal end so that when the connecting peptide is covalently linked to the A-chain peptide by a peptide bond, the peptide bond is cleaved by the kex2 protease.

Another method for producing a recombinant insulin analogue of interest from the host cell identified and isolated as taught herein includes the following modification to the nucleotide sequence encoding the fusion protein comprising the recombinant insulin analogue precursor. The method is performed as taught herein but wherein a single stop codon is placed between the nucleic acid sequence encoding the insulin analogue A-chain peptide and the nucleic acid sequence encoding the downstream polypeptides and/or proteins, e.g., the linker and SED1 or modification motif or second binding moiety. The above non-insulin analogue sequences are fused to the insulin analogue sequences comprising the A-chain and B-chain by a terminal Lys residue, this creates a protease (e.g., trypsin or LysC) cleavage site. In the host cells, translation of mRNAs encoded by the vector is performed under conditions that increase translational readthrough through the stop codon thereby producing a population of recombinant insulin analogue precursors that comprise the downstream polypeptides and/or proteins, which can be displayed on the cell surface. After the host cells that produce the recombinant insulin analogue precursor of interest has been selected and isolated, the host cells are grown under conditions that results in an increase in translational readthrough through the stop codon, e.g., in the presence of the antibiotic G418 when the host cell is a yeast. Under the second conditions, the host cells produce a recombinant insulin analogue precursor that is secreted into the medium where the optional N-terminal extension and optionally the C-peptide may be removed by protease digestion to produce a recombinant insulin analogue heterodimer. In embodiments where the C-peptide is not removed, recombinant single-chain insulin analogues are produced. In this embodiment, the nucleic acid sequence encoding the recombinant insulin analogue precursor does not need to be recloned in an embodiment that excludes the downstream polypeptides/proteins.

### I. Host Cells

The methods disclosed herein can be performed using mammalian, plant, lower eukaryote, or insect cells. In general, lower eukaryotes such as yeast are desirable for expression of proteins because they can be economically cultured and may give high yields of the proteins. Yeast particularly offers established genetics allowing for rapid transformations, tested protein localization strategies and facile gene knock-out techniques. Suitable vectors have expression control sequences, such as promoters, including 3-phosphoglycerate kinase or other glycolytic enzymes, and an origin of replication, termination sequences and the like as desired.

While the invention has been demonstrated herein using the methylotrophic yeast Pichia pastoris, other useful lower eukaryote host cells include Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Yarrowia lipolytica and Neurospora crassa. Various yeasts, such as Kluyveromyces lactis, Pichia pastoris, Pichia methanolica, and Hansenula polymorpha are particularly suitable for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale. In the case of lower eukaryotes, cells are routinely grown from between about 1.5 to 3 days under conditions that induce expression of the pre-proinsulin analogue precursor or the capture moiety. In embodiments that include a capture moiety, induction of the pre-proinsulin analogue precursor molecule expression is performed for about 1 to 2 days under conditions where expression of the capture moiety is stopped or inhibited. Afterwards, the recombinant cells are analyzed for those recombinant cells that display the insulin analogue precursor molecule of interest.

Insulin analogue precursor molecules that are glycosylated may display pharmacodynamic and/or pharmacokinetic characteristics that are modified or improved over insulin analogues that are not glycosylated. Therefore, the protein display system disclosed herein may be used with host cells that are capable of producing glycoproteins that have particular N-glycosylation or O-glycosylation patterns to identify and select host cells that express glycosylated insulin analogues that maintain binding to the IR and/or have reduced binding to the IGF-1 receptor.

Therefore, in particular aspects, the nucleic acid molecule encoding the pre-proinsulin analogue precursor will be mutated or modified to encode at least one consensus N-linked glycosylation site motif (Asn-Xaa-Ser or Thr, wherein Xaa is any amino acid except for Pro). When this nucleic acid molecule is expressed in a host cell that is competent for N-linked glycosylation, an N-linked glycosylated insulin analogue precursor is displayed. It may be desirable that the host cell be capable of producing and displaying N-glycosylated insulin analogue precursors wherein a particular N-glycan structure or glycoform predominates. A particular predominant N-glycan species may confer differentiated functional characteristics to the N-glycosylated insulin analogue such that the clinical profile is altered or improved. For example, particular N-glycan structures might result in differences in biological activity at the receptor level (i.e., increase and/or decrease binding at the IGF-1 receptor, IR-A, IR-B) or N-linked glycosylation might influence alternative routes of clearance that result in glucose-responsive properties or differences in tissue distribution (e.g., targeting the liver) that result in a greater therapeutic index.

Yeast are particularly attractive host cells since they can be genetically modified so that they can express glycoproteins in which the N-glycosylation pattern is mammalian-like or human-like or humanized or where a particular N-glycan species is predominant. This has been achieved by eliminating selected endogenous glycosylation enzymes and/or supplying exogenous enzymes as described by Gerngross et al., U.S. Pat. No. 7,449,308, the disclosure of which is incorporated herein by reference, and general methods for reducing O-glycosylation in yeast have been described in International Application No. WO2007061631.

Thus, in particular aspects of the invention, the host cell is yeast, for example, a methylotrophic yeast such as Pichia pastoris or Ogataea minuta and mutants thereof and genetically engineered variants thereof. In this manner, glycoprotein compositions can be produced in which a specific desired glycoform is predominant in the composition. If desired, additional genetic engineering of the glycosylation can be performed, such that the glycoprotein can be produced with or without core fucosylation. Use of lower eukaryotic host cells such as yeast are further advantageous in that these cells are able to produce relatively homogenous compositions of glycoprotein, such that the predominant glycoform of the glycoprotein may be present as greater than thirty mole percent of the glycoprotein in the composition. In particular aspects, the predominant glycoform may be present in greater than forty mole percent, fifty mole percent, sixty mole percent, seventy mole percent and, most preferably, greater than eighty mole percent of the glycoprotein present in the composition. Such can be achieved by eliminating selected endogenous glycosylation enzymes and/or supplying exogenous enzymes as described by Gerngross et al., U.S. Pat. No. 7,029,872 and U.S. Pat. No. 7,449,308, the disclosures of which are incorporated herein by reference. For example, a host cell can be selected or engineered to be depleted in α1,6-mannosyl transferase activities, which would otherwise add mannose residues onto the N-glycan on a glycoprotein. For example, in yeast such an α1,6-mannosyl transferase activity is encoded by the OCH1 gene and deletion or disruption of the OCH1 inhibits the production of high mannose or hypermannosylated N-glycans in yeast such as Pichia pastoris or Saccharomyces cerevisiae. (See for example, Gerngross et al. in U.S. Pat. No. 7,029,872; Contreras et al. in U.S. Pat. No. 6,803,225; and Chiba et al. in EP1211310B1 the disclosures of which are incorporated herein by reference).

In one embodiment, the host cell further includes an α1,2-mannosidase catalytic domain fused to a cellular targeting signal peptide not normally associated with the catalytic domain and selected to target the α-1,2-mannosidase activity to the ER or Golgi apparatus of the host cell. Passage of a recombinant glycoprotein through the ER or Golgi apparatus of the host cell produces a recombinant glycoprotein comprising a Man₅GlcNAc₂ glycoform, for example, a recombinant glycoprotein composition comprising predominantly a Man₅GlcNAc₂ glycoform.

For example, U.S. Pat. No. 7,029,872, U.S. Pat. No. 7,449,308, and U.S. Published Patent Application No. 2005/0170452, the disclosures of which are all incorporated herein by reference, disclose lower eukaryote host cells capable of producing a glycoprotein comprising a Man₅GlcNAc₂ glycoform.

In a further embodiment, the immediately preceding host cell further includes an N-acetylglucosaminyltransferase I (GlcNAc transferase I or GnT I) catalytic domain fused to a cellular targeting signal peptide not normally associated with the catalytic domain and selected to target GlcNAc transferase I activity to the ER or Golgi apparatus of the host cell. Passage of the recombinant glycoprotein through the ER or Golgi apparatus of the host cell produces a recombinant glycoprotein comprising a GlcNAcMan₅GlcNAc₂ glycoform, for example a recombinant glycoprotein composition comprising predominantly a GlcNAcMan₅GlcNAc₂ glycoform. U.S. Pat. No. 7,029,872, U.S. Pat. No. 7,449,308, and U.S. Published Patent Application No. 2005/0170452, the disclosures of which are all incorporated herein by reference, disclose lower eukaryote host cells capable of producing a glycoprotein comprising a GlcNAcMan₅GlcNAc₂ glycoform. The glycoprotein produced in the above cells can be treated in vitro with a hexaminidase to produce a recombinant glycoprotein comprising a Man₅GlcNAc₂ glycoform.

In a further embodiment, the immediately preceding host cell further includes a mannosidase II catalytic domain fused to a cellular targeting signal peptide not normally associated with the catalytic domain and selected to target mannosidase II activity to the ER or Golgi apparatus of the host cell. Passage of the recombinant glycoprotein through the ER or Golgi apparatus of the host cell produces a recombinant glycoprotein comprising a GlcNAcMan₃GlcNAc₂ glycoform, for example a recombinant glycoprotein composition comprising predominantly a GlcNAcMan₃GlcNAc₂ glycoform. U.S. Pat. No. 7,029,872 and U.S. Pat. No. 7,625,756, the disclosures of which are all incorporated herein by reference, discloses lower eukaryote host cells that express mannosidase II enzymes and are capable of producing glycoproteins having predominantly a GlcNAcMan₃GlcNAc₂ glycoform. The glycoprotein produced in the above cells can be treated in vitro with a hexosaminidase that removes the terminal GlcNAc residue to produce a recombinant glycoprotein comprising a Man₃GlcNAc₂ glycoform or the hexosaminidase can be co-expressed with the glycoprotein in the host cell to produce a recombinant glycoprotein comprising a Man₃GlcNAc₂ glycoform. In a further embodiment, the immediately preceding host cell further includes N-acetylglucosaminyltransferase II (GlcNAc transferase II or GnT II) catalytic domain fused to a cellular targeting signal peptide not normally associated with the catalytic domain and selected to target GlcNAc transferase II activity to the ER or Golgi apparatus of the host cell. Passage of the recombinant glycoprotein through the ER or Golgi apparatus of the host cell produces a recombinant glycoprotein comprising a GlcNAc₂Man₃GlcNAc₂ glycoform, for example a recombinant glycoprotein composition comprising predominantly a GlcNAc₂Man₃GlcNAc₂ glycoform. U.S. Pat. Nos. 7,029,872 and 7,449,308 and U.S. Published Patent Application No. 2005/0170452, the disclosures of which are all incorporated herein by reference, disclose lower eukaryote host cells capable of producing a glycoprotein comprising a GlcNAc₂Man₃GlcNAc₂ glycoform. The glycoprotein produced in the above cells can be treated in vitro with a hexosaminidase that removes the terminal GlcNAc residues to produce a recombinant glycoprotein comprising a Man₃GlcNAc₂ glycoform or the hexosaminidase can be co-expressed with the glycoprotein in the host cell to produce a recombinant glycoprotein comprising a Man₃GlcNAc₂ glycoform.

In a further embodiment, the immediately preceding host cell further includes a galactosyltransferase catalytic domain fused to a cellular targeting signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising a GalGlcNAc₂Man₃GlcNAc₂ or Gal₂GlcNAc₂Man₃GlcNAc₂ glycoform, or mixture thereof for example a recombinant glycoprotein composition comprising predominantly a GalGlcNAc₂Man₃GlcNAc₂ glycoform or Gal₂GlcNAc₂Man₃GlcNAc₂ glycoform or mixture thereof. U.S. Pat. No. 7,029,872 and U.S. Published Patent Application No. 2006/0040353, the disclosures of which are incorporated herein by reference, discloses lower eukaryote host cells capable of producing a glycoprotein comprising a Gal₂GlcNAc₂Man₃GlcNAc₂ glycoform. The glycoprotein produced in the above cells can be treated in vitro with a galactosidase to produce a recombinant glycoprotein comprising a GlcNAc₂Man₃GlcNAc₂ glycoform, for example a recombinant glycoprotein composition comprising predominantly a GlcNAc₂Man₃GlcNAc₂ glycoform or the galactosidase can be co-expressed with the glycoprotein in the host cell to produce a recombinant glycoprotein comprising the GlcNAc₂Man₃GlcNAc₂ glycoform, for example a recombinant glycoprotein composition comprising predominantly a GlcNAc₂Man₃GlcNAc₂ glycoform.

In a further embodiment, the immediately preceding host cell further includes a sialyltransferase catalytic domain fused to a cellular targeting signal peptide not normally associated with the catalytic domain and selected to target sialyltransferase activity to the ER or Golgi apparatus of the host cell. Passage of the recombinant glycoprotein through the ER or Golgi apparatus of the host cell produces a recombinant glycoprotein comprising predominantly a Sia₂Gal₂GlcNAc₂Man₃GlcNAc₂ glycoform or SiaGal₂GlcNAc₂Man₃GlcNAc₂ glycoform or mixture thereof. For lower eukaryote host cells such as yeast and filamentous fungi, it is useful that the host cell further include a means for providing CMP-sialic acid for transfer to the N-glycan. U.S. Published Patent Application No. 2005/0260729, the disclosure of which is incorporated herein by reference, discloses a method for genetically engineering lower eukaryotes to have a CMP-sialic acid synthesis pathway and U.S. Published Patent Application No. 2006/0286637, the disclosure of which is incorporated herein by reference, discloses a method for genetically engineering lower eukaryotes to produce sialylated glycoproteins. The glycoprotein produced in the above cells can be treated in vitro with a neuraminidase to produce a recombinant glycoprotein comprising predominantly a Gal₂GlcNAc₂Man₃GlcNAc₂ glycoform or GalGlcNAc₂Man₃GlcNAc₂ glycoform or mixture thereof or the neuraminidase can be co-expressed with the glycoprotein in the host cell to produce a recombinant glycoprotein comprising predominantly a Gal₂GlcNAc₂Man₃GlcNAc₂ glycoform or GalGlcNAc₂ Man₃GlcNAc₂ glycoform or mixture thereof.

In a further aspect, the above host cell capable of making glycoproteins having a Man₅GlcNAc₂ glycoform can further include a mannosidase III catalytic domain fused to a cellular targeting signal peptide not normally associated with the catalytic domain and selected to target the mannosidase III activity to the ER or Golgi apparatus of the host cell. Passage of the recombinant glycoprotein through the ER or Golgi apparatus of the host cell produces a recombinant glycoprotein comprising a Man₃GlcNAc₂ glycoform, for example a recombinant glycoprotein composition comprising predominantly a Man₃GlcNAc₂ glycoform. U.S. Pat. No. 7,625,756, the disclosures of which are all incorporated herein by reference, discloses the use of lower eukaryote host cells that express mannosidase III enzymes and are capable of producing glycoproteins having predominantly a Man₃GlcNAc₂ glycoform.

Any one of the preceding host cells can further include one or more GlcNAc transferase selected from the group consisting of GnT III, GnT IV, GnT V, GnT VI, and GnT IX to produce glycoproteins having bisected (GnT III) and/or multiantennary (GnT IV, V, VI, and IX) N-glycan structures such as disclosed in U.S. Pat. No. 7,598,055 and U.S. Published Patent Application No. 2007/0037248, the disclosures of which are all incorporated herein by reference.

In further embodiments, the host cell that produces glycoproteins that have predominantly GlcNAcMan₅GlcNAc₂ N-glycans further includes a galactosyltransferase catalytic domain fused to a cellular targeting signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising predominantly the GalGlcNAcMan₅GlcNAc₂ glycoform.

In a further embodiment, the immediately preceding host cell that produced glycoproteins that have predominantly the GalGlcNAcMan₅GlcNAc₂ N-glycans further includes a sialyltransferase catalytic domain fused to a cellular targeting signal peptide not normally associated with the catalytic domain and selected to target sialytransferase activity to the ER or Golgi apparatus of the host cell. Passage of the recombinant glycoprotein through the ER or Golgi apparatus of the host cell produces a recombinant glycoprotein comprising a SiaGalGlcNAcMan₅GlcNAc₂ glycoform.

In general yeast and filamentous fungi are not able to make glycoproteins that have N-glycans that include fucose. Therefore, the N-glycans disclosed herein will lack fucose unless the host cell is specifically modified to include a pathway for synthesizing GDP-fucose and a fucosyltransferase. Therefore, in particular aspects where it is desirable to have glycoproteins in which the N-glycan includes fucose, any one of the aforementioned host cells is further modified to include a fucosyltransferase and a pathway for producing fucose and transporting fucose into the ER or Golgi. Examples of methods for modifying Pichia pastoris to render it capable of producing glycoproteins in which one or more of the N-glycans thereon are fucosylated are disclosed in Published International Application No. WO 2008112092, the disclosure of which is incorporated herein by reference. In particular aspects of the invention, the Pichia pastoris host cell is further modified to include a fucosylation pathway comprising a GDP-mannose-4,6-dehydratase, GDP-keto-deoxy-mannose-epimerase/GDP-keto-deoxy-galactose-reductase, GDP-fucose transporter, and a fucosyltransferase. In particular aspects, the fucosyltransferase is selected from the group consisting of α1,2-fucosyltransferase, α-1,3-fucosyltransferase, α-1,4-fucosyltransferase, and α-1,6-fucosyltransferase.

Various of the preceding host cells further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because lower eukaryote host cells such as yeast and filamentous fungi lack the above transporters, it is preferable that lower eukaryote host cells such as yeast and filamentous fungi be genetically engineered to include the above transporters.

Host cells further include Pichia pastoris that are genetically engineered to eliminate glycoproteins having phosphomannose residues by deleting or disrupting one or both of the phosphomannosyltransferase genes PNO1 and MNN4B (See for example, U.S. Pat. Nos. 7,198,921 and 7,259,007; the disclosures of which are all incorporated herein by reference), which in further aspects can also include deleting or disrupting the MNN4A gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. The host cells can further include any one of the aforementioned host cells modified to produce particular N-glycan structures.

Host cells further include lower eukaryote cells (e.g., yeast such as Pichia pastoris) that are genetically modified to control O-glycosylation of the glycoprotein by deleting or disrupting one or more of the protein O-mannosyltransferase (Dol-P-Man:Protein (Ser/Thr) Mannosyl Transferase genes) (PMTs) (See U.S. Pat. No. 5,714,377; the disclosure of which is incorporated herein by reference) or grown in the presence of Pmtp inhibitors and/or an alpha-mannosidase as disclosed in Published International Application No. WO 2007061631, the disclosure of which is incorporated herein by reference, or both. Disruption includes disrupting the open reading frame encoding the Pmtp or disrupting expression of the open reading frame or abrogating translation of RNAs encoding one or more of the Pmtps using interfering RNA, antisense RNA, or the like. The host cells can further include any one of the aforementioned host cells modified to produce particular N-glycan structures.

Pmtp inhibitors include but are not limited to a benzylidene thiazolidinediones. Examples of benzylidene thiazolidinediones that can be used are 5-[[3,4-bis(phenylmethoxy)phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic Acid.

In particular embodiments, the function or expression of at least one endogenous PMT gene is reduced, disrupted, or deleted. For example, in particular embodiments the function or expression of at least one endogenous PMT gene selected from the group consisting of the PMT1, PMT2, PMT3, and PMT4 genes is reduced, disrupted, or deleted; or the host cells are cultivated in the presence of one or more PMT inhibitors. In further embodiments, the host cells include one or more PMT gene deletions or disruptions and the host cells are cultivated in the presence of one or more Pmtp inhibitors. In particular aspects of these embodiments, the host cells also express a secreted α-1,2-mannosidase.

PMT deletions or disruptions and/or Pmtp inhibitors control O-glycosylation by reducing O-glycosylation occupancy; that is by reducing the total number of O-glycosylation sites on the glycoprotein that are glycosylated. The further addition of an α-1,2-mannosidase that is secreted by the cell controls O-glycosylation by reducing the mannose chain length of the O-glycans that are on the glycoprotein. Thus, combining PMT deletions or disruptions and/or Pmtp inhibitors with expression of a secreted α-1,2-mannosidase controls O-glycosylation by reducing occupancy and chain length. In particular circumstances, the particular combination of PMT deletions or disruptions, Pmtp inhibitors, and α-1,2-mannosidase is determined empirically as particular heterologous glycoproteins (antibodies, for example) may be expressed and transported through the Golgi apparatus with different degrees of efficiency and thus may require a particular combination of PMT deletions or disruptions, Pmtp inhibitors, and α-1,2-mannosidase. In another aspect, genes encoding one or more endogenous mannosyltransferase enzymes are deleted. The deletion(s) can be in combination with providing the secreted α-1,2-mannosidase and/or PMT inhibitors or can be in lieu of providing the secreted α-1,2-mannosidase and/or PMT inhibitors.

Thus, the control of O-glycosylation can be useful for producing particular glycoproteins in the host cells disclosed herein in better total yield or in yield of properly assembled glycoprotein. The reduction or elimination of O-glycosylation appears to have a beneficial effect on the assembly and transport of glycoproteins such as whole antibodies as they traverse the secretory pathway and are transported to the cell surface. Thus, in cells in which O-glycosylation is controlled, the yield of properly assembled glycoproteins such as antibody fragments is increased over the yield obtained in host cells in which O-glycosylation is not controlled.

To reduce or eliminate the likelihood of N-glycans and O-glycans with β-linked mannose residues, which are resistant to α-mannosidases, the recombinant glycoengineered Pichia pastoris host cells are genetically engineered to eliminate glycoproteins having α-mannosidase-resistant N-glycans by deleting or disrupting one or more of the β-mannosyltransferase genes (e.g., BMT1, BMT2, BMT3, and BMT4)(See, U.S. Pat. No. 7,465,577, U.S. Pat. No. 7,713,719, and Published International Application No. WO2011046855, each of which is incorporated herein by reference). The deletion or disruption of BMT2 and one or more of BMT1, BMT3, and BMT4 also reduces or eliminates detectable cross reactivity to antibodies against host cell protein.

In particular embodiments, the host cells do not display Alg3p protein activity or have a deletion or disruption of expression from the ALG3 gene (e.g., deletion or disruption of the open reading frame encoding the Alg3p to render the host cell alg3Δ) as described in Published U.S. Application No. 20050170452 or US20100227363, which are incorporated herein by reference. Alg3p is Man₅GlcNAc₂-PP-dolichyl alpha-1,3 mannosyltransferase that transferase a mannose residue to the mannose residue of the alpha-1,6 arm of lipid-linked Man₅GlcNAc₂ (FIG. 16, GS 1.3) in an alpha-1,3 linkage to produce lipid-linked Man₆GlcNAc₂ (FIG. 16, GS 1.4), a precursor for the synthesis of lipid-linked Glc₃Man₉GlcNAc₂, which is then transferred by an oligosaccharyltransferase to an asparagine residue of a glycoprotein followed by removal of the glucose (Glc) residues. In host cells that lack Alg3p protein activity, the lipid-linked Man₅GlcNAc₂ oligosaccharide may be transferred by an oligosaccharyltransferase to an aspargine residue of a glycoprotein. In such host cells that further include an α1,2-mannosidase, the Man₅GlcNAc₂ oligosaccharide attached to the glycoprotein is trimmed to a tri-mannose (paucimannose) Man₃GlcNAc₂ structure (FIG. 16, GS 2.1). The Man₅GlcNAc₂ (GS 1.3) structure is distinguishable from the Man₅GlcNAc₂ (GS 2.0) shown in FIG. 16, and which is produced in host cells that express the Man₅GlcNAc₂-PP-dolichyl alpha-1,3 mannosyltransferase (Alg3p).

Therefore, provided is a method for producing an N-glycosylated insulin or insulin analogue and compositions of the same in a lower eukaryote host cell, comprising a deletion or disruption ALG3 gene (alg3Δ) and includes a nucleic acid molecule encoding an insulin or insulin analogue having at least one N-glycosylation site; and culturing the host cell under conditions for expressing the insulin or insulin analogue to produce the N-glycosylated insulin or insulin analogue having predominantly a Man₅GlcNAc₂ (GS 1.3) structure. In further embodiments, the host cell further expresses an endomannosidase activity (e.g., a full-length endomannosidase or a chimeric endomannosidase comprising an endomannosidase catalytic domain fused to a cellular targeting signal peptide not normally associated with the catalytic domain and selected to target the endomannosidase activity to the ER or Golgi apparatus of the host cell. See for example, U.S. Pat. No. 7,332,299) and/or glucosidase II activity (a full-length glucosidase II or a chimeric glucosidase II comprising a glucosidase H catalytic domain fused to a cellular targeting signal peptide not normally associated with the catalytic domain and selected to target the glucosidase II activity to the ER or Golgi apparatus of the host cell. See for example, U.S. Pat. No. 6,803,225). In particular aspects, the host cell further includes a deletion or disruption of the ALG6 (α-1,3-glucosylatransferase) gene (alg6Δ), which has been shown to increase N-glycan occupancy of glycoproteins in alg3Δhost cells (See for example, De Pourcq et al., PloSOne 2012; 7(6):e39976. Epub 2012 Jun 29, which discloses genetically engineering Yarrowia lipolytica to produce glycoproteins that have Man₅GlcNAc₂ (GS 1.3) or paucimannose N-glycan structures). The nucleic acid sequence encoding the Pichia pastoris ALG6 is disclosed in EMBL database, accession number CCCA38426. In further aspects, the host cell further includes a deletion or disruption of the OCH1 gene (och1Δ).

Further provided is a method for producing an N-glycosylated insulin or insulin analogue and compositions of the same in a lower eukaryote host cell, comprising a deletion or disruption of the ALG3 gene (alg3Δ) and includes a nucleic acid molecule encoding a chimeric α-1,2-mannosidase comprising an α1,2-mannosidase catalytic domain fused to a cellular targeting signal peptide not normally associated with the catalytic domain and selected to target the α-1,2-mannosidase activity to the ER or Golgi apparatus of the host cell to overexpress the chimeric α-1,2-mannosidase and a nucleic acid molecule encoding the insulin or insulin analogue having at least one N-glycosylation site; and culturing the host cell under conditions for expressing the insulin or insulin analogue to produce the N-glycosylated insulin or insulin analogue having predominantly a Man₃GlcNAc₂ structure. In further embodiments, the host cell further expresses or overexpresses an endomannosidase activity (e.g., a full-length endomannosidase or a chimeric endomannosidase comprising an endomannosidase catalytic domain fused to a cellular targeting signal peptide not normally associated with the catalytic domain and selected to target the endomannosidase activity to the ER or Golgi apparatus of the host cell) and/or a glucosidase II activity (a full-length glucosidase II or a chimeric glucosidease II comprising a glucosidase II catalytic domain fused to a cellular targeting signal peptide not normally associated with the catalytic domain and selected to target the glucosidase II activity to the ER or Golgi apparatus of the host cell). In particular aspects, the host cell further includes a deletion or disruption of the ALG6 gene (alg6Δ). In further aspects, the host cell further includes a deletion or disruption of the OCH1 gene (och1Δ) Example 14 shows the construction of an alg3ΔPichia pastoris host cell that overexpresses a chimeric α-1,2-mannosidase and a full-length endomannosidase. The host cell was shown in Example 15 to produce insulin analogues that have paucimannose N-glycans. Similar host cells may be constructed in other yeast or filamentous fungi.

Yield of glycoprotein can in some situations be improved by overexpressing nucleic acid molecules encoding mammalian or human chaperone proteins or replacing the genes encoding one or more endogenous chaperone proteins with nucleic acid molecules encoding one or more mammalian or human chaperone proteins. In addition, the expression of mammalian or human chaperone proteins in the host cell also appears to control O-glycosylation in the cell. Thus, further included are the host cells herein wherein the function of at least one endogenous gene encoding a chaperone protein has been reduced or eliminated, and a vector encoding at least one mammalian or human homolog of the chaperone protein is expressed in the host cell. Also included are host cells in which the endogenous host cell chaperones and the mammalian or human chaperone proteins are expressed. In further aspects, the lower eukaryotic host cell is a yeast or filamentous fungi host cell. Examples of the use of chaperones of host cells in which human chaperone proteins are introduced to improve the yield and reduce or control O-glycosylation of recombinant proteins has been disclosed in Published International Application No. WO2009105357 and WO2010019487 (the disclosures of which are incorporated herein by reference). Like above, further included are lower eukaryotic host cells wherein, in addition to replacing the genes encoding one or more of the endogenous chaperone proteins with nucleic acid molecules encoding one or more mammalian or human chaperone proteins or overexpressing one or more mammalian or human chaperone proteins as described above, the function or expression of at least one endogenous gene encoding a protein O-mannosyltransferase (PMT) protein is reduced, disrupted, or deleted. In particular embodiments, the function of at least one endogenous PMT gene selected from the group consisting of the PMT1, PMT2, PMT3, and PMT4 genes is reduced, disrupted, or deleted.

Therefore, the methods disclose herein can use any host cell that has been genetically modified to produce glycoproteins wherein the predominant N-glycan is selected from the group consisting of complex N-glycans, hybrid N-glycans, and high mannose N-glycans wherein complex N-glycans are selected from the group consisting of Man₃GlcNAc₂, GlcNAc₍₁₋₄₎Man₃GlcNAc₂, Gal₍₁₋₄₎GlcNAc₍₁₋₄₎Man₃GlcNAc₂, and Sia₍₁₋₄₎Gal₍₁₋₄₎Man₃GlcNAc₂; hybrid N-glycans are selected from the group consisting of GlcNAcMan₅GlcNAc₂, GalGlcNAcMan₅GlcNAc₂, and SiaGalGlcNAcMan₅GlcNAc₂; and high Mannose N-glycans are selected from the group consisting of Man₅GlcNAc₂, Man₆GlcNAc₂, Man₇GlcNAc₂, Man₈GlcNAc₂, and Man₉GlcNAc₂.

To increase the N-glycosylation site occupancy on a glycoprotein produced in a recombinant host cell, a nucleic acid molecule encoding a heterologous single-subunit oligosaccharyltransferase, which is capable of functionally suppressing a lethal mutation of one or more essential subunits comprising the endogenous host cell hetero-oligomeric oligosaccharyltransferase (OTase) complex, is overexpressed in the recombinant host cell either before or simultaneously with the expression of the glycoprotein in the host cell. The Leishmania major STT3A protein, Leishmania major STT3B protein, and Leishmania major STT3D protein, are single-subunit oligosaccharyltransferases that have been shown to suppress the lethal phenotype of a deletion of the STT3 locus in Saccharomyces cerevisiae (Naseb et al., Molec. Biol. Cell 19: 3758-3768 (2008)). Naseb et al. (ibid.) further showed that the Leishmania major STT3D protein could suppress the lethal phenotype of a deletion of the WBP1, OST1, SWP1, or OST2 loci. Hese et al. (Glycobiology 19: 160-171 (2009)) teaches that the Leishmania major STT3A (STT3-1), STT3B (STT3-2), and STT3D (STT3-4) proteins can functionally complement deletions of the OST2, SWP1, and WBP1 loci. As shown in PCT/US2011/25878 (Published International Application No. WO2011106389, which is incorporated herein by reference), the Leishmania major STT3D (LmSTT3D) protein is a heterologous single-subunit oligosaccharyltransferases that is capable of suppressing a lethal phenotype of a Δstt3 mutation and at least one lethal phenotype of a Δwbp1, Δost1, Δswp1, and Δost2 mutation that is shown in the examples herein to be capable of enhancing the N-glycosylation site occupancy of heterologous glycoproteins, for example antibodies, produced by the host cell.

Therefore, in a further aspect of the methods herein, provided are yeast or filamentous fungus host cells genetically engineered to be capable of producing glycoproteins with mammalian- or human-like complex or hybrid N-glycans wherein the host cell further includes a nucleic acid molecule encoding a heterologous single-subunit oligosaccharyltransferase (OTase) complex.

In general, in the above methods and host cells, the single-subunit oligosaccharyltransferase is capable of functionally suppressing the lethal phenotype of a mutation of at least one essential protein of the OTase complex. In further aspects, the essential protein of the OTase complex is encoded by the STT3 locus, WBP1 locus, OST1 locus, SWP1 locus, or OST2 locus, or homologue thereof. In further aspects, the for example single-subunit oligosaccharyltransferase is the Leishmania major STT3D protein.

For genetically engineering yeast, selectable markers can be used to construct the recombinant host cells include drug resistance markers and genetic functions which allow the yeast host cell to synthesize essential cellular nutrients, e.g. amino acids. Drug resistance markers that are commonly used in yeast include chloramphenicol, kanamycin, methotrexate, G418 (geneticin), Zeocin, and the like. Genetic functions that allow the yeast host cell to synthesize essential cellular nutrients are used with available yeast strains having auxotrophic mutations in the corresponding genomic function. Common yeast selectable markers provide genetic functions for synthesizing leucine (LEU2), tryptophan (TRP1 and TRP2), proline (PRO1), uracil (URA3, URA5, URA6), histidine (HIS3), lysine (LYS2), adenine (ADE1 or ADE2), and the like. Other yeast selectable markers include the ARR3 gene from S. cerevisiae, which confers arsenite resistance to yeast cells that are grown in the presence of arsenite (Bobrowicz et al., Yeast, 13:819-828 (1997); Wysocki et al., J. Biol. Chem. 272:30061-30066 (1997)). A number of suitable integration sites include those enumerated in U.S. Pat. No. 7,479,389 (the disclosure of which is incorporated herein by reference) and include homologs to loci known for Saccharomyces cerevisiae and other yeast or fungi. Methods for integrating vectors into yeast are well known (See for example, U.S. Pat. No. 7,479,389, U.S. Pat. No. 7,514,253, U.S. Published Application No. 2009012400, and WO2009/085135; the disclosures of which are all incorporated herein by reference). Examples of insertion sites include, but are not limited to, Pichia ADE genes; Pichia TRP (including TRP1 through TRP2) genes; Pichia MCA genes; Pichia CYM genes; Pichia PEP genes; Pichia PRB genes; and Pichia LEU genes. The Pichia ADE1 and ARG4 genes have been described in Lin Cereghino et al., Gene 263:159-169 (2001) and U.S. Pat. No. 4,818,700 (the disclosure of which is incorporated herein by reference), the HIS3 and TRP1 genes have been described in Cosano et al., Yeast 14:861-867 (1998), HIS4 has been described in GenBank Accession No. X56180.

The transformation of the yeast cells is well known in the art and may for instance be effected by protoplast formation followed by transformation in a manner known per se. The medium used to cultivate the cells may be any conventional medium suitable for growing yeast organisms.

The methods disclosed herein can be adapted for use in mammalian, plant, bacteria, and insect cells. Examples of animal cells include, but are not limited to, SC-I cells, LLC-MK cells, CV-I cells, CHO cells, COS cells, murine cells, human cells, HeLa cells, 293 cells, VERO cells, MDBK cells, MDCK cells, MDOK cells, CRFK cells, RAF cells, TCMK cells, LLC-PK cells, PK15 cells, WI-38 cells, MRC-5 cells, T-FLY cells, BHK cells, SP2/0, NSO cells, carrot cells, and derivatives thereof. Insect cells include cells of Drosophila melanogaster origin. These cells can be genetically engineered to render the cells capable of making glycoproteins that have particular or predominantly particular N-glycans. For example, U.S. Pat. No. 6,949,372 discloses methods for making glycoproteins in insect cells that are sialylated. Yamane-Ohnuki et al. Biotechnol. Bioeng. 87: 614-622 (2004), Kanda et al., Biotechnol. Bioeng. 94: 680-688 (2006), Kanda et al., Glycobiol. 17: 104-118 (2006), and U.S. Pub. Application Nos. 2005/0216958 and 2007/0020260 (the disclosures of which are incorporated herein by reference) disclose mammalian cells that are capable of producing glycoproteins in which the N-glycans thereon lack fucose or have reduced fucose. U.S. Published Patent Application No. 2005/0074843 (the disclosure of which is incorporated herein by reference) discloses making antibodies in mammalian cells that have bisected N-glycans.

The regulatable promoters selected for regulating expression of the expression cassettes in mammalian, insect, or plant cells should be selected for functionality in the cell-type chosen. Examples of suitable regulatable promoters include but are not limited to the tetracycline-regulatable promoters (See for example, Berens & Hillen, Eur. J. Biochem. 270: 3109-3121 (2003)), RU 486-inducible promoters, ecdysone-inducible promoters, and kanamycin-regulatable systems. These promoters can replace the promoters exemplified in the expression cassettes described in the examples. The capture moiety can be fused to a cell surface anchoring protein suitable for use in the cell-type chosen. Cell surface anchoring proteins including GPI proteins are well known for mammalian, insect, and plant cells. GPI-anchored fusion proteins has been described by Kennard et al., Methods Biotechnol. Vo. 8: Animal Cell Biotechnology (Ed. Jenkins. Human Press, Inc., Totowa, N.J.) pp. 187-200 (1999). The genome targeting sequences for integrating the expression cassettes into the host cell genome for making stable recombinants can replace the genome targeting and integration sequences exemplified in the examples. Transfection methods for making stable and transiently transfected mammalian, insect, and plant host cells are well known in the art. Once the transfected host cells have been constructed as disclosed herein, the cells can be screened for expression of the recombinant proinsulin analogue precursor molecules of interest and selected as disclosed herein.

Therefore, in a further aspect of the above, provided is a method for displaying a recombinant insulin analogue precursor in a mammalian, plant, or insect host cell, comprising providing a mammalian or insect host cell that includes a nucleic acid molecule encoding a heterologous single-subunit oligosaccharyltransferase (e.g., Leishmania major STT3 protein) and a nucleic acid molecule encoding the fusion protein comprising pre-proinsulin analogue precursor; and culturing the host cell under conditions for displaying recombinant proinsulin analogue precursor molecules on the surface of the cell. In further aspects, the host cell is genetically engineered to produce glycoproteins with human-like N-glycans or N-glycans not normally endogenous to the host cell.

In a further aspect of the above, provided is a method for producing a heterologous glycoprotein wherein the N-glycosylation site occupancy of the heterologous glycoprotein is greater than 83% in a mammalian or insect host cell, comprising providing a mammalian or insect host cell that includes a nucleic acid molecule encoding a heterologous single-subunit oligosaccharyltransferase (e.g., Leishmania major STT3 protein) and a nucleic acid molecule encoding the heterologous glycoprotein; and culturing the host cell under conditions for expressing the heterologous glycoprotein to produce the heterologous glycoprotein wherein the N-glycosylation site occupancy of the heterologous glycoprotein is greater than 83%. In further aspects, the host cell is genetically engineered to produce glycoproteins with human-like N-glycans or N-glycans not normally endogenous to the host cell.

In a further embodiment of the above methods, the endogenous host cell genes encoding the proteins comprising the oligosaccharyltransferase (OTase) complex are expressed.

In particular embodiments of the above methods, the N-glycosylation site occupancy is at least 94%. In further still embodiments, the N-glycosylation site occupancy is at least 99%.

Further provided is a mammalian or insect host cell, comprising a first nucleic acid molecule encoding a heterologous single-subunit oligosaccharyltransferase (e.g., the Leishmania major STT3D protein); and a second nucleic acid molecule encoding a heterologous glycoprotein; and wherein the endogenous host cell genes encoding the proteins comprising the endogenous host cell oligosaccharyltransferase (OTase) complex are expressed.

Bacterial cells that may be used in the methods disclosed herein include cells modified for phage display, including phage display for N-linked glycoproteins. For example, Mazor et al., FEBS Journal 277: 2291-2303 (2010); Mazor et al., Nature Biotechnol. 25: 563-565 (2007); and Mazor et al., Nature protocols 11: 1766-1777 (2008) disclose methods for selecting recombinant bacterial cells that express full-length IgG molecules using periplasmic display and subsequence fluorescence-activated cell sorting (FACS) screening. In the disclosed methods, the IgG molecules, while aglycosylated, are folded structures in E. coli that are fully functional when displayed on the cell surface. Proinsulin analogue precursors may also be folded into a conformation that is similar to the conformation of native insulin and such would be expected to bind to the IR and/or IGF-1 receptor. Therefore, constructing recombinant bacteria that express ligands or proinsulin precursor molecules following the methods disclosed in the above references may be used to identify and isolate recombinant cells that express ligands or proinsulin analogue precursors that have a desired affinity and/or avidity for the IR and/or IGF-1 receptor. çelik et al., Protein Science 19: 2006-2013 (2010) teaches a filamentous display system in E. coli cells for N-linked glycoproteins. The methods disclosed therein may be used to display ligands or proinsulin analogue precursor molecules to identify and isolate recombinant cells that express ligands or proinsulin analogue precursors that have a desired affinity and/or avidity for the IR and/or IGF-1 receptor.

Therefore, the present invention provides a method for detecting and isolating recombinant cells that express a ligand for the insulin receptor (IR) or insulin growth factor 1 (IGF-1) receptor, comprising (a) constructing recombinant cells wherein each recombinant cell transiently or stably expresses a fusion protein comprising a polypeptide, wherein the fusion protein is secreted and capable of being displayed on the surface of the recombinant cell, by transforming host cells with nucleic acid molecules encoding the fusion protein; (b) detecting recombinant cells that display on the cell surface thereof a fusion protein comprising a polypeptide capable of binding the IR or IGF-1 receptor by contacting the recombinant cells produced in (a) with the IR or IGF-1 receptor; and (c) isolating the recombinant cells that display the fusion protein detected in step (b) to provide the recombinant cells that express the ligand for the IR or IGF-1 receptor.

In a further aspect, the present invention provides a method for detecting recombinant cells that express a ligand for the insulin receptor (IR) or insulin growth factor 1 (IGF-1) receptor; comprising (a) constructing a library of recombinant cells wherein each cell transiently or stably expresses a secreted fusion protein comprising a polypeptide by transfecting host cells with a plurality nucleic acid molecules encoding the fusion protein, wherein each recombinant cell in the library expresses a different fusion protein; and (b) contacting the library of recombinant cells produced in (a) with the IR or IGF-1 receptor to detect the recombinant cells in the library that express the ligand for the insulin receptor (IR) or insulin growth factor 1 (IGF-1) receptor.

In a further aspect, the present invention provides a method for detecting and isolating recombinant cells that express a ligand for the insulin receptor (IR) or insulin growth factor 1 (IGF-1) receptor, comprising (a) constructing recombinant cells wherein each recombinant cell transiently or stably expresses a fusion protein comprising a polypeptide fused to a cell surface anchoring protein or cell surface binding portion thereof, wherein the fusion protein is secreted and capable of being displayed on the surface of the recombinant cell, by transfecting cells with nucleic acid molecules encoding the fusion protein; (b) detecting recombinant cells that display on the cell surface thereof a fusion protein that comprises a polypeptide capable of binding the IR or IGF-1 receptor by contacting the recombinant cells produced in (a) with the IR or IGF-1 receptor; and (c) isolating the recombinant cells that display the fusion protein detected in step (b) to provide the recombinant cells that express the ligand for the insulin IR or IGF-1 receptor.

In a particular aspect, the polypeptide is fused to a cell surface anchoring moiety or protein or cell surface binding portion thereof, which in a further aspect may be selected from the group consisting of α-agglutinin, Cwp1p, Cwp2p, Gas1p, Yap3p, Flo1p, Crh2p, Pir1p, Pir4p, Sed1p, Tip1p, Hpwp1p, Als3p, and Rbt5p, and which in a particular aspect may be Sed1p.

In a particular aspect, the recombinant cells in (a) are constructed by transfecting cells with first nucleic acid molecules encoding a cell surface anchoring protein or cell surface binding portion thereof fused to a first binding moiety and second nucleic acid molecules encoding fusion proteins comprising a polypeptide fused to a second binding moiety that is specific for the first binding moiety.

In a further aspect, the first binding moiety is a first peptide and the second binding moiety is a second peptide wherein the first and second peptides are capable of a specific pairwise interaction, which in a further aspect, the first and second peptides are coiled-coil peptides that are capable of the specific pairwise interaction.

In a further aspect, the polypeptide is fused to a modification motif that is coupled to a first binding partner when the fusion proteins are expressed and which binds to a second binding partner displayed on the surface of the recombinant cells. In a further aspect, the first binding partner is biotin and the second binding partner is an avidin-like protein.

In further aspects, the recombinant cells are mutagenized to produce a library of recombinant cells expressing a variegated population of polypeptides. In a further aspect, the recombinant cells in (a) are produced by transforming or transfecting cells with a plurality of nucleic acid molecules in which the majority of the nucleic acid molecules comprise at least one mutation in the nucleotide sequence encoding the polypeptide to produce a library of recombinant cells wherein each recombinant cell in the library produces a single species of polypeptide. In a further aspect, the recombinant cells display on the cell surface thereof a plurality of different fusion proteins, wherein each fusion protein is encoded on a different nucleic acid molecule in a different recombinant cell. In particular aspects, the different fusion proteins are sequence variants of each other.

In particular aspects, the polypeptide comprising the fusion protein is an insulin or insulin analogue precursor molecule. In a particular aspect, the insulin or insulin analogue precursor molecule is displayed on the cell surface in a single-chain structure having a structure characteristic of native insulin. In a particular aspect, the insulin or insulin analogue precursor molecule is displayed on the cell surface as a split proinsulin molecule having a structure characteristic of native insulin.

In the above aspects, the host cell is a bacterial, mammalian, insect, yeast, filamentous fungus, or plant host cell. In a particular aspect, the host cell is Pichia pastoris.

In particular aspects of the above, the detecting and isolating uses FACS cell sorting.

The following examples are intended to promote a further understanding of the present invention.

### Example 1

Construction of YGLY8292, which was used to exemplify the practice of the invention is illustrated schematically in FIG. 1A-1B and described below.

The strain YGLY8292 was constructed from wild-type Pichia pastoris strain NRRL-Y 11430 using methods described earlier (See for example, U.S. Pat. No. 7,449,308; U.S. Pat. No. 7,479,389; U.S. Published Application No. 20090124000; Published PCT Application No. WO2009085135; Nett and Gerngross, Yeast 20:1279 (2003); Choi et al., Proc. Natl. Acad. Sci. USA 100:5022 (2003); Hamilton et al., Science 301:1244 (2003)). All plasmids were made in a pUC19 plasmid using standard molecular biology procedures. For nucleotide sequences that were optimized for expression in P. pastoris, the native nucleotide sequences were analyzed by the GENEOPTIMIZER software (GeneArt, Regensburg, Germany) and the results used to generate nucleotide sequences in which the codons were optimized for P. pastoris expression. Yeast strains were transformed by electroporation (using standard techniques as recommended by the manufacturer of the electroporator BioRad).

Plasmid pGLY6 (FIG. 3) is an integration vector that targets the URA5 locus. It contains a nucleic acid molecule comprising the S. cerevisiae invertase gene or transcription unit (ScSUC2; SEQ ID NO:1) flanked on one side by a nucleic acid molecule comprising a nucleotide sequence from the 5′ region of the P. pastoris URA5 gene (SEQ ID NO:2) and on the other side by a nucleic acid molecule comprising the nucleotide sequence from the 3′ region of the P. pastoris URA5 gene (SEQ ID NO:3). Plasmid pGLY6 was linearized and the linearized plasmid transformed into wild-type strain NRRL-Y 11430 to produce a number of strains in which the ScSUC2 gene was inserted into the URA5 locus by double-crossover homologous recombination. Strain YGLY1-3 was selected from the strains produced and is auxotrophic for uracil.

Plasmid pGLY40 (FIG. 4) is an integration vector that targets the OCH1 locus and contains a nucleic acid molecule comprising the P. pastoris URA5 gene or transcription unit (SEQ ID NO:4) flanked by nucleic acid molecules comprising lacZ repeats (SEQ ID NO:5) which in turn is flanked on one side by a nucleic acid molecule comprising a nucleotide sequence from the 5′ region of the OCH1 gene (SEQ ID NO:6) and on the other side by a nucleic acid molecule comprising a nucleotide sequence from the 3′ region of the OCH1 gene (SEQ ID NO:7). Plasmid pGLY40 was linearized with SfiI and the linearized plasmid transformed into strain YGLY1-3 to produce a number of strains in which the URA5 gene flanked by the lacZ repeats has been inserted into the OCH1 locus by double-crossover homologous recombination. Strain YGLY2-3 was selected from the strains produced and is prototrophic for URA5. Strain YGLY2-3 was counterselected in the presence of 5-fluoroorotic acid (5-FOA) to produce a number of strains in which the URA5 gene has been lost and only the lacZ repeats remain in the OCH1 locus. This renders the strain auxotrophic for uracil. Strain YGLY4-3 was selected.

Plasmid pGLY43a (FIG. 5) is an integration vector that targets the BMT2 locus and contains a nucleic acid molecule comprising the K. lactic UDP-N-acetylglucosamine (UDP-GlcNAc) transporter gene or transcription unit (KlMNN2-2, SEQ ID NO:8) adjacent to a nucleic acid molecule comprising the P. pastoris URA5 gene or transcription unit flanked by nucleic acid molecules comprising lacZ repeats. The adjacent genes are flanked on one side by a nucleic acid molecule comprising a nucleotide sequence from the 5′ region of the BMT2 gene (SEQ ID NO: 9) and on the other side by a nucleic acid molecule comprising a nucleotide sequence from the 3′ region of the BMT2 gene (SEQ ID NO:10). Plasmid pGLY43a was linearized with SfiI and the linearized plasmid transformed into strain YGLY4-3 to produce to produce a number of strains in which the KlMNN2-2 gene and URA5 gene flanked by the lacZ repeats has been inserted into the BMT2 locus by double-crossover homologous recombination. The BMT2 gene has been disclosed in Mille et al., J. Biol. Chem. 283: 9724-9736 (2008) and U.S. Pat. No. 7,465,557. Strain YGLY6-3 was selected from the strains produced and is prototrophic for uracil. Strain YGLY6-3 was counterselected in the presence of 5-FOA to produce strains in which the URA5 gene has been lost and only the lacZ repeats remain. This renders the strain auxotrophic for uracil. Strain YGLY8-3 was selected.

Plasmid pGLY48 (FIG. 6) is an integration vector that targets the MNN4L1 locus and contains an expression cassette comprising a nucleic acid molecule encoding the mouse homologue of the UDP-GlcNAc transporter (SEQ ID NO:11) open reading frame (ORF) operably linked at the 5′ end to a nucleic acid molecule comprising the P. pastoris GAPDH promoter (SEQ ID NO:12) and at the 3′ end to a nucleic acid molecule comprising the S. cerevisiae CYC termination sequences (SEQ ID NO:13) adjacent to a nucleic acid molecule comprising the P. pastoris URA5 gene flanked by lacZ repeats and in which the expression cassettes together are flanked on one side by a nucleic acid molecule comprising a nucleotide sequence from the 5′ region of the P. pastoris MNN4L1 gene (SEQ ID NO:14) and on the other side by a nucleic acid molecule comprising a nucleotide sequence from the 3′ region of the MNN4L1 gene (SEQ ID NO:15). Plasmid pGLY48 was linearized with SfiI and the linearized plasmid transformed into strain YGLY8-3 to produce a number of strains in which the expression cassette encoding the mouse UDP-GlcNAc transporter and the URA5 gene have been inserted into the MNN4L1 locus by double-crossover homologous recombination. The MNN4L1 gene (also referred to as MNN4B) has been disclosed in U.S. Pat. No. 7,259,007. Strain YGLY10-3 was selected from the strains produced and then counterselected in the presence of 5-FOA to produce a number of strains in which the URA5 gene has been lost and only the lacZ repeats remain. Strain YGLY12-3 was selected.

Plasmid pGLY45 (FIG. 7) is an integration vector that targets the PNO1/MNN4 loci and contains a nucleic acid molecule comprising the P. pastoris URA5 gene or transcription unit flanked by nucleic acid molecules comprising lacZ repeats which in turn is flanked on one side by a nucleic acid molecule comprising a nucleotide sequence from the 5′ region of the PNO1 gene (SEQ ID NO:16) and on the other side by a nucleic acid molecule comprising a nucleotide sequence from the 3′ region of the MNN4 gene (SEQ ID NO:17). Plasmid pGLY45 was linearized with SfiI and the linearized plasmid transformed into strain YGLY12-3 to produce a number of strains in which the URA5 gene flanked by the lacZ repeats has been inserted into the PNO1/MNN4 loci by double-crossover homologous recombination. The PNO1 gene has been disclosed in U.S. Pat. No. 7,198,921 and the MNN4 gene (also referred to as MNN4B) has been disclosed in U.S. Pat. No. 7,259,007. Strain YGLY14-3 was selected from the strains produced and then counterselected in the presence of 5-FOA to produce a number of strains in which the URA5 gene has been lost and only the lacZ repeats remain. Strain YGLY16-3 was selected.

Plasmid pGLY3419 (FIG. 8) is an integration vector that contains an expression cassette comprising the P. pastoris URA5 gene flanked by lacZ repeats flanked on one side with the 5′ nucleotide sequence of the P. pastoris BMT1 gene (SEQ ID NO:18) and on the other side with the 3′ nucleotide sequence of the P. pastoris BMT1 gene (SEQ ID NO:19). Plasmid pGLY3419 was linearized and the linearized plasmid transformed into strain YGLY16-3 to produce a number of strains in which the URA5 expression cassette has been inserted into the BMT1 locus by double-crossover homologous recombination. The strain YGLY6697 was selected from the strains produced and is prototrophic for uracil. The strains was then counterselected in the presence of 5-FOA to produce a number of strains now auxotrophic for uridine. Strain YGLY6719 was selected.

Plasmid pGLY3411 (FIG. 9) is an integration vector that contains the expression cassette comprising the P. pastoris URA5 gene flanked by lacZ repeats flanked on one side with the 5′ nucleotide sequence of the P. pastoris BMT4 gene (SEQ ID NO:20) and on the other side with the 3′ nucleotide sequence of the P. pastoris BMT4 gene (SEQ ID NO:21). Plasmid pGLY3411 was linearized and the linearized plasmid transformed into YGLY6719 to produce a number of strains in which the URA5 expression cassette has been inserted into the BMT4 locus by double-crossover homologous recombination. Strain YGLY6743 was selected from the strains produced and is prototrophic for uracil. The strain was then counterselected in the presence of 5-FOA to produce a number of strains now auxotrophic for uridine. Strain YGLY6773 was selected.

Plasmid pGLY3421 (FIG. 10) is an integration vector that contains an expression cassette comprising the P. pastoris URA5 gene flanked by lacZ repeats flanked on one side with the 5′ nucleotide sequence of the P. pastoris BMT3 gene (SEQ ID NO:22) and on the other side with the 3′ nucleotide sequence of the P. pastoris BMT3 gene (SEQ ID NO:23). Plasmid pGLY3419 was linearized and the linearized plasmid transformed into strain YGLY6773 to produce a number of strains in which the URA5 expression cassette has been inserted into the BMT1 locus by double-crossover homologous recombination. The strain YGLY7754 was selected from the strains produced and is prototrophic for uracil. The strain was then counterselected in the presence of 5-FOA to produce a number of strains now auxotrophic for uridine. Strain YGLY8252 was selected.

Plasmid pGLY1162 (FIG. 11) is a KINKO integration vector that targets the PRO1 locus without disrupting expression of the locus and contains expression cassettes encoding the T. reesei α-1,2-mannosidase catalytic domain fused at the N-terminus to S. cerevisiae αMATpre signal peptide (aMATTrMan) to target the chimeric protein to the secretory pathway and secretion from the cell. The expression cassette encoding the aMATTrMan comprises a nucleic acid molecule encoding the T. reesei catalytic domain (SEQ ID NO:24) fused at the 5′ end to a nucleic acid molecule encoding the a Saccharomyces cerevisiae alpha-mating factor signal peptide (αMATpre signal peptide) (SEQ ID NO:25 encoding SEQ ID NO:26), which is operably linked at the 5′ end to a nucleic acid molecule comprising the P. pastoris AOX1 promoter (SEQ ID NO:27) and at the 3′ end to a nucleic acid molecule comprising the S. cerevisiae CYC transcription termination sequence (SEQ ID NO:13). The cassette is flanked on one side by a nucleic acid molecule comprising a nucleotide sequence from the 5′ region and complete ORF of the PRO1 gene (SEQ ID NO:28) followed by a P. pastoris ALG3 termination sequence (SEQ ID NO:29) and on the other side by a nucleic acid molecule comprising a nucleotide sequence from the 3′ region of the PRO1 gene (SEQ ID NO:30). Plasmid pGLY1162 was linearized and the linearized plasmid transformed into strain YGLY8252 to produce a number of strains in which the URA5 expression cassette has been inserted into the PRO1 locus by double-crossover homologous recombination. The strain YGLY8292 was selected from the strains produced and is prototrophic for uracil.

### Example 2

Genetically engineered Pichia pastoris strains YGLY24426; YGLY26073; YGLY26075; and YGLY26087 express and display on the surface thereof a recombinant insulin analogue precursor. The strains comprise a nucleic acid molecule integrated into the host cell genome that encodes a fusion protein comprising a pre-proinsulin precursor molecule fused at the C-terminus to the GPI protein SED1. These strains were constructed to demonstrate operation of the protein display system for identifying and sorting host cells that produce a recombinant insulin analogue precursor displayed on the surface of the host cell.

These expression vectors have been designed for protein expression in Pichia pastoris; however, the nucleic acid molecules encoding fusion protein can be incorporated into expression vectors designed for protein expression in other host cells capable of producing N-glycosylated glycoproteins, for example, mammalian cells and fungal, plant, insect, or bacterial cells, including host cells genetically modified to produce glycoproteins having human-like N-glycans.

The expression vectors disclosed below encode a pre-proinsulin analogue precursor molecule comprising a substitution of the proline residue at position 28 of the B-chain with an asparagine residue to produce an N-glycosylation site having the tri-amino acid sequence Asn Xaa (Ser/Thr) wherein Xaa is any amino acid except Pro fused to the N-terminus of a polypeptide comprising a truncated SED1 GPI protein. During expression of the vector encoding the pre-proinsulin analogue precursor in the yeast host cell, the pre-proinsulin analogue precursor is transported to the secretory pathway where the signal peptide is removed and in the case where the host cell is competent for N-glycosylation, the molecule is processed into an N-glycosylated proinsulin analogue precursor that is folded into a structure held together by disulfide bonds that has the same configuration as that for native human insulin. The N-glycosylated proinsulin analogue precursor is then transported through the secretory pathway where the N-glycans on the N-glycosylated proinsulin analogue precursor are modified. The N-glycosylated proinsulin analogue precursor is then directed to vesicles where the propetide is removed to form an N-glycosylated insulin analogue precursor molecule that then exits the host cell and attached to the cell surface via the SED1.

Plasmid pGLY10958 (FIG. 2A) provides a nucleic acid molecule (SEQ ID NO:46) encoding fusion protein I (SEQ ID NO:47) comprising a pre-proinsulin analogue precursor having a P28N mutation fused at the C-terminus to the N-terminus of a truncated Saccharomyces cerevisiae SED1 protein. The fusion protein comprises from the N-terminus to the C-terminus the S. cerevisiae alpha-mating factor signal sequence and propeptide (Saccharomyces cerevisiae αMATprepro signal peptide; SEQ ID NO:35 encoded by SEQ ID NO:59) joined to an N-terminal 10×His peptide spacer (SEQ ID NO:36) joined to the insulin B-chain having the P28N mutation (SEQ ID NO:37) joined to a C-peptide consisting of the amino acid sequence AAK joined to the insulin A-chain (SEQ ID NO:38) joined to a c-myc peptide (SEQ ID NO:40) joined to a 3×G4S linker peptide (SEQ ID NO:41) joined to an N-terminal truncated S. cerevisiae SED1 protein (SEQ ID NO:43) encoded by SEQ ID NO:42. The insulin analogue precursor-truncated SED1 fusion protein IA that is displayed on the cell surface is shown by (SEQ ID NO:48).

Plasmid pGLY11677 (FIG. 2B) encodes fusion protein II, which is similar to fusion protein I except that the C-peptide consists of the IGF-1 C-peptide (SEQ ID NO:44). The nucleotide sequence of SEQ ID NO:49 encodes fusion protein II which has the amino acid sequence shown in SEQ ID NO:50. The insulin analogue precursor-truncated SED1 protein fusion IIA that is displayed on the cell surface is shown by SEQ ID NO:51.

Plasmid pGLY11678 (FIG. 2C) encodes fusion protein III, which is similar to fusion protein II except that the C-peptide consists of the IGF-1 C-peptide wherein the tyrosine residue at position 2 of the peptide is replaced with an alanine residue to reduce binding to the IGF-1 receptor as taught in U.S. Published Application No. US20080057004 (SEQ ID NO:45). The nucleotide sequence of SEQ ID NO:52 encodes fusion protein II which has the amino acid sequence shown in SEQ ID NO:53. The insulin analogue precursor-truncated SED1 fusion protein IIIA that is displayed on the cell surface is shown by (SEQ ID NO:54). The nucleic acid molecule encoding the above fusion proteins are each operably linked at the 5′ end to the P. pastoris AOX1 promoter (SEQ ID NO:27) and at the 3′ end to a nucleic acid molecule comprising the P. pastoris AOX1 transcription termination sequence (SEQ ID NO:31). For selecting transformants, the plasmid comprises an expression cassette encoding the Zeocin ORF in which the nucleic acid molecule encoding the ORF (SEQ ID NO:32) is operably linked at the 5′ end to a nucleic acid molecule having the S. cerevisiae TEF promoter sequence (SEQ ID NO:33) and at the 3′ end to a nucleic acid molecule having the S. cerevisiae CYC transcription termination sequence (SEQ ID NO:13). The plasmid further includes a nucleic acid molecule for targeting the TRP2 locus (SEQ ID NO:34) for integration. The plasmids are roll-in plasmids that insert multiple copies of the plasmid into the target locus. FIG. 2D shows schematically the general structure of the encoded fusion protein and shows how it is displayed on the cell surface.

Transformations of the appropriate strains disclosed herein with Insulin Analogues display plasmids pGLY10958; pGLY11677; and pGLY11678; were performed essentially as follows. Appropriate Pichia pastoris strains were grown in 50 mL YPD media (yeast extract (1%), soytone (2%), and dextrose (2%)) overnight to an OD of about 0.2 to 6. After incubation on ice for 30 minutes, cells were pelleted by centrifugation at 2500-3000 rpm for five minutes. Media was removed and the cells washed three times with ice cold sterile 1 M sorbitol before resuspension in 0.5 mL ice cold sterile 1 M sorbitol. Ten μL linearized DNA (5-20 μg) and 100 μL cell suspension were combined in an electroporation cuvette and incubated for five minutes on ice. Electroporation was in a Bio-Rad GenePulser Xcell following the preset Pichia pastoris protocol (2 kV, 25 μF, 200Ω), immediately followed by the addition of 1 mL YPDS recovery media (YPD media plus 1 M sorbitol). The transformed cells were allowed to recover for four hours to overnight at room temperature (24° C.) before plating the cells on selective media.

Strains YGLY24426, YGLY 26083, and YGLY26085 were generated by transforming pGLY10958, pGLY11677, and pGLY11678, respectively into strain YGLY8292 described in Example 2. Strains YGLY24426, YGLY 26083, and YGLY26085 were selected from the resulting clones.

### Example 3

The pGLY10958, pGLY11677, and pGLY11678 encoding the insulin analogues were linearized with Spa and the linearized plasmids were transformed into Pichia pastoris strain YGLY8292 to provide host cells displaying the insulin analogue precursor molecules on the cell surface. Transformations were performed essentially as described in Example 1.

The genomic integration of pGLY10958 at the TRP2 locus was confirmed by cPCR using the primers, c/o-ScSED1-FW (5′-TCCAGAAAGTGATAACGGTACTTCTACTGC-3′; SEQ ID NO:55) and c/o-ScSED1-RV (5′-AATGTAGTTGGTTCGGTAACTGTGTAAGTTTT-3′; SEQ ID NO:56). The PCR conditions were one cycle of 94° C. for 30 seconds, 30 cycles of 94° C. for 30 seconds, 55° C. for 30 seconds, and 72° C. for one minute; followed by one cycle of 72° C. for 2 minutes.

Protein expression for the transformed yeast strains was carried out at in shake flasks at 24° C. with buffered glycerol-complex medium (BMGY) consisting of 1% yeast extract, 2% peptone, 100 mM potassium phosphate buffer pH 6.0, 1.34% yeast nitrogen base, 4×10⁻⁵% biotin, and 2% glycerol. The induction medium for protein expression was buffered methanol-complex medium (BMMY) consisting of 2% methanol instead of glycerol in BMGY. Cells were typically harvested after two days methanol induction, centrifuged at 2,000 rpm for five minutes, and washed with ice-cold PBS (phosphate-buffered saline).

Table 2 lists antibodies and reagents used for detecting display of the recombinant insulin analogue precursor molecules on the cell surface.

TABLE 2
Reagents used for Insulin Surface Display Detection

| | | Vender & Cat. |
|---|---|---|
| Reagents | Description | Number |
| Anti-His tag antibody | Mouse monoclonal anti-His tag antibody | Abcam, ab72579 |
| | (clone AD1.1.10), Allophycocyanin (APC)- | |
| | conjugate | |
| Anti-Myc tag antibody | Mouse monoclonal anti-Myc tag antibody | Cell Signaling, |
| | (clone 9B11), Alexa Fluor 488 conjugate | 2279 |
| Anti-human insulin | Mouse monoclonal anti-human insulin | Abcam, |
| antibody | antibody (clone D3E7), Biotin-conjugate | ab20756 |
| Streptavidin-Alexa 488 | Streptavidin, Alexa Fluor 488 conjugate | Invitrogen, |
| | | S-11223 |
| Recombinant human | Recombinant Human Insulin R/CD220, | R&D Systems, |
| insulin receptor | His28-to-Arg750 (α subunit) & Ser751-to- | 1544-IR/CF |
| (Insulin R) | Lys944 with a C-terminal 10x His | GeneBank |
| | tag (β subunit) produced in Murine myeloma | Accession No. |
| | NS0 cell line. | NP_001073285 |
| Anti-insulin receptor | Goat polyclonal anti-human insulin | R&D Systems, |
| antibody | R/CD220, Allophycocyanin (APC)-conjugate | FAB1544A |
| Recombinant human | Recombinant Human IGF-1 receptor, | R&D Systems, |
| IGF-1 receptor (IGF- | produced in Murine myeloma NS0 cell line. | 391-GR |
| IR) | | GenBank |
| | | Accession No. |
| | | P08069 |
| Anti-IGF-IR antibody | Goat polyclonal to anti-human IGF-1R | Abcam, |
| | antibody | Ab10729 |
| Donkey anti-goat IgG | Donkey anti-goat IgG (H + L) antibody, Alexa | Invitrogen A21447 |
| (H + L)-Alexa 647 | 647 | |

Typically 1×10⁶ of transformed yeast cells (0.1 OD₆₀₀) were resuspended in 50 μL PBS (phosphate-buffered saline) to which one μL of anti-His, anti-cMyc or anti-insulin monoclonal antibody was added. Cells were incubated on ice for 30 minutes and washed twice with ice-cold PBS. When appropriate, 0.5 μL streptavidin-conjugated fluorephore was then added and incubated for five minutes. Cells were washed twice with ice-cold PBS and suspended in 200 μL of ice-cold PBS for flow cytometry analysis.

To detect insulin receptor binding to the proinsulin analogue on the cell surface, 1×10⁶ yeast cells (0.1 OD₆₀₀) were resuspended in 50 μL PBS (phosphate-buffered saline) to which 0.25 μg of soluble insulin receptor (in 0.25 μg/μL concentration) was added and incubated on ice for 30 minutes. Cells were washed once with ice-cold PBS and then one μL of goat anti-human insulin receptor-antibody (allophycocyanin conjugate) was added to the cell suspension and incubate the cells on ice for 15 minutes. Cells were washed twice with ice-cold PBS and suspended in 200 μL of ice-cold PBS for flow cytometry analysis.

To detect insulin-like Growth Factor 1 Receptor (IGF-1R) binding to insulin analogues displayed on the cell wall of Pichia pastoris strains, 1×10⁷ yeast cells (1 OD₆₀₀) were resuspended in 100 μL PBS (phosphate-buffered saline) to which 0.25 μg of soluble IGF-1R receptor (in 0.25 μg/μLμL concentration) was added and incubated on ice for 30 minutes. Cells were washed once with ice-cold PBS and then one μL of goat anti-human IGF-1 Receptor-antibody was added to 100 μL of cell suspension. Cells were incubated on ice for 15 minutes and subsequently washed twice with ice-cold. To detect the Anti-IGF-1R-IGF1R complex on the yeasts, one μL of donkey anti-goat antibody (allophycocyanin conjugate) was incubated in 100 μL cell suspension for 15 minutes on ice and washed twice in ice-cold PBS. Cells were resuspended in 200 μL PBS for flow cytometric analysis.

Flow Cytometry Analysis was performed with an FACSAria II cell sorter with three lasers (405 nm, 488 nm and 633 nm, Becton Dickinson, San Jose, Calif.) equipped with Diva v6.1 software was applied to flow cytometry analysis. Doublet discrimination gates were routinely used to ensure a population of single cells for analysis. For insulin detection with antibody, a blue laser (488 nm) was used for excitation and an optical filter of 530/30 nm was used to collect emission. For insulin receptor binding, a red laser (633 nm) was used for excitation and an optical filter of 660/20 nm was used to collect emission. The data was electronically recorded and processed with Diva v6.1 as histogram plots to generate the fluorescent profiles as shown in FIGS. 12, 13, and 14.

FIG. 12 depicts the flow cytometric analysis of display of recombinant insulin analogue precursor IA on yeast strain YGLY24426 detected using an anti-His antibody conjugated to APC. The green histogram on the left represents the background auto-fluorescence of empty parental strain YGLY8292. The red histogram on the right represents the cells that display the recombinant insulin analogue precursor. The entire cell population is bound to the anti-His antibodies indicating that the insulin analogue precursor is expressed and displayed on the yeast surface.

FIG. 13 depicts the flow cytometric analysis of display of insulin analogue precursor-truncated SED1 fusion protein IA on yeast strain YGLY24426 detected using an anti-cMyc antibody conjugated to fluorephore ALEXA488. The green histogram on the left represents the background auto-fluorescence of empty parental strain YGLY8292. The red histogram on the right represents the cells that display the recombinant insulin analogue precursor. The figure shows that the entire cell population is bound to the anti-cMyc antibodies indicating that the recombinant insulin analogue precursor is expressed and displayed on the yeast surface.

FIG. 14 depicts the flow cytometric analysis of insulin analogue expression on yeast detected using anti-insulin antibody; soluble IR and detection complex, and IGF-1 receptor and detection complex. Empty parental strain YGLY8292 is a negative control. All strains except strain YGLY8292 exhibited positive signals when incubated with anti-insulin antibody and soluble IR. Only strain YGLY26083, which displays a recombinant insulin analogue precursor with the native IGF-1 C-peptide, exhibited strong binding to IGF-1 receptor while strain YGLY26085, which displays a recombinant insulin analogue precursor having an IGF-1 C-peptide mutated to reduce binding to the IGF-1 receptor, exhibited low but above background binding to the IGF-1 receptor. Strains YGLY8292 and YGLY24426 did not appear to bind to soluble IGF-1 receptor. Insulin analogues comprising the IGF-1 C-peptide or modified IGF-1 C-peptide have been shown in the art to be active at the insulin receptor. The results here show that insulin analogue precursor molecules containing the IGF-1 or modified IGF-1 C-peptide can also bind the IR when the molecule is attached to the cell surface. The results shown here further showed that the insulin precursor analogue comprising the connecting tripeptide AAK was also capable of binding the IR.

FIG. 15 depicts the flow cytometric analysis of IGF-1R competing with IR binding to the recombinant insulin analogue precursor displayed on strain YGLY26083. Strain YGLY26083 was induced 24 hours in BMMY media. Afterward, cells were and rinsed and suspended in PBS. The cell density was adjusted to one OD₆₀₀. Then, 50 μL of cell suspension was incubated with mixture of IR and IGF-1 receptor in 1.5 mL tubes as follows:

| | 1 | 2 | 3 | 4 | 5 | 6 |
|---|---|---|---|---|---|---|
| IGF-1R | 10 μL | 10 μL | 10 μL | 10 μL | 10 μL | 0 |
| IR | 0 | 0.01 μL | 0.1 μL | 1 μL | 10 μL | 10 μL |

The final concentration with 10 μL of IGF-1 receptor or with 10 μL of IR was about 400 nM. After incubation at room temperature for 30 minutes, cells were rinsed with ice-cols PBS once and suspended the cells in 200 μL of ice-cold PBS. Samples were divided into two series of tubes: A and B, each containing 100 μL cell suspensions.

For A series: Add 1 μL of goat anti-human IGF-1R and incubate on ice for 15 minutes. Wash cells twice with PBS add 1 μL of donkey anti-goat Alexa 647 and incubate for on ice for 15 minutes. Afterward, wash the cells twice with ice-cold PBS and suspend the cells in 100 μL of ice-cold PBS for flow cytometry analysis.

For B series: Add 1 μL of goat anti-human insulin APC and incubate on ice for 15 minutes. Wash cells twice with PBS and then suspend the cells in 100 μL of ice-cold PBS for flow cytometry analysis.

### Example 4

This example provides a capture moiety (amino acid sequence shown in SEQ ID NO:60) comprising a truncated SED1 (SEQ ID NO:43) fused at the N-terminus to a coiled-coil peptide GR2 (SEQ ID NO:57) and a Saccharomyces cerevisiae alpha-mating factor signal peptide ((SEQ ID NO:26) and a pre-proinsulin analogue precursor molecule fused at the C-terminus to a 3×(G4S) spacer peptide (SEQ ID NO:41) fused to the N-terminus of coiled-coil peptide GR1 (SEQ ID NO:58) to produce a fusion protein has the amino acid sequence shown in SEQ ID NO:62.

Nucleic acid molecules encoding these molecules may be introduced into the appropriate Pichia pastoris host cell on an expression as described in Example 2. The capture moiety is expressed, processed in the secretory pathway to remove the signal peptide to produce a capture moiety having the sequence shown in SEQ ID NO:61, which is then secreted from the cell and becomes anchored to the cell surface. The fusion protein is processed also processed in the secretory pathway and the processed fusion protein having the amino acid sequence shown in SEQ ID NO:63 is secreted from the cell. The GR1 and GR2 coiled-coil peptides form a pairwise interaction, which results in the proinsulin analogue precursor being displayed on the cell surface.

Detection of proinsulin analogue precursor molecules that bind the IR may be performed as follows.

Typically, about 1×10⁶ of transformed yeast cells (0.1 OD₆₀₀) may be resuspended in 50 μL PBS (phosphate-buffered saline) to which one μL of anti-His, anti-cMyc or anti-insulin monoclonal antibody was added. Cells are then incubated on ice for 30 minutes and washed twice with ice-cold PBS. When appropriate, 0.5 μL streptavidin-conjugated fluorephore is then added and incubated for five minutes. Cells are washed twice with ice-cold PBS and suspended in 200 μL of ice-cold PBS for flow cytometry analysis.

To detect insulin receptor binding to the proinsulin analogue on the cell surface, about 1×10⁶ yeast cells (0.1 OD₆₀₀) may be resuspended in 50 μL PBS (phosphate-buffered saline) to which 0.25 μg of soluble insulin receptor (in 0.25 μL concentration) is added and incubated on ice for 30 minutes. Cells are washed once with ice-cold PBS and then one μL of goat anti-human insulin receptor-antibody (allophycocyanin conjugate) is added to the cell suspension and incubate the cells on ice for 15 minutes. Cells are washed twice with ice-cold PBS and suspended in 200 μL of ice-cold PBS for flow cytometry analysis.

Flow Cytometry Analysis may be performed with an FACSAria II cell sorter with three lasers (405 nm, 488 nm and 633 nm, Becton Dickinson, San Jose, Calif.) equipped with Diva v6.1 software was applied to flow cytometry analysis. Doublet discrimination gates are routinely used to ensure a population of single cells for analysis. For insulin detection with antibody, a blue laser (488 nm) may be used for excitation and an optical filter of 530/30 nm is used to collect emission. For insulin receptor binding, a red laser (633 nm) may be used for excitation and an optical filter of 660/20 nm is used to collect emission. The data may be electronically recorded and processed with Diva v6.1 as histogram plots to generate the fluorescent profiles.

### Example 5

This example shows the display of an insulin heterodimer on the surface of the host cell and host cells that the display a functional insulin heterodimer can be sorted from host cells that do not display a functional insulin heterodimer based on whether the displayed insulin is capable of binding the insulin receptor or the IGF-1 receptor.

Plasmid pGLY11680 (FIG. 20) provides a nucleic acid molecule encoding a fusion protein (SEQ ID NO:64; FIG. 17A) comprising a pre-proinsulin precursor fused at the C-terminus to the N-terminus of a truncated Saccharomyces cerevisiae SED1 protein. The fusion protein comprises from the N-terminus to the C-terminus the S. cerevisiae alpha-mating factor signal sequence and propeptide (Saccharomyces cerevisiae αMATprepro signal peptide; SEQ ID NO:35 encoded by SEQ ID NO:59) joined to the N-terminus of a native human proinsulin in which the insulin B-chain (SEQ ID NO:39) is joined to the insulin A-chain (SEQ ID NO:38) by the native human insulin C-peptide (SEQ ID NO:65) joined to a c-myc peptide (SEQ ID NO:40) joined to a GGGGSAS linker peptide (SEQ ID NO:66) joined to an N-terminal truncated S. cerevisiae SED1 protein (SEQ ID NO:43). The signal sequence and pro-peptide is linked to the N-terminus of the B-chain peptide by a kex2 protease cleavage site. In addition, the junction between the C-peptide and the A-chain peptide is also a kex2 protease cleavage site. The C-terminus of the proinsulin C-peptide contains the motif that is a substrate for Pichia pastoris Kex2 protease. The consensus motif for the kex2 cleavage site is LXKR (SEQ ID NO:68). As represented by the schematic diagram shown in FIG. 18, during passage of the fusion protein through the secretory pathway of the host cell, the kex2 cleavage sites are cleaved resulting in an split proinsulin heterodimer molecule in which the C-peptide is covalently linked to the C-terminus of the B-chain (SEQ ID NO:69) and the C-terminus of the A-chain is covalently linked to the truncated SED1 protein (SEQ ID NO:70) and the A-chain and B-chain are covalently linked by disulfide bonds between A7 and B7 and A20 and B19.

Plasmid pGLY10569 (FIG. 21) provides a nucleic acid encoding a fusion protein comprising a pre-proinsulin precursor. The fusion protein comprises from the N-terminus to the C-terminus the S. cerevisiae alpha-mating factor signal sequence and propeptide (Saccharomyces cerevisiae αMATprepro signal peptide; SEQ ID NO:35 encoded by SEQ ID NO:59) joined to the N-terminus of a native human proinsulin in which the insulin B-chain (SEQ ID NO:39) is joined to the insulin A-chain (SEQ ID NO:38) by the native human insulin C-peptide (SEQ ID NO:65). The proinsulin is secreted.

The nucleic acid sequences for pGLY11680 and pGLY10569 are shown in SEQ ID NO:71 and SEQ ID NO:72, respectively.

The nucleic acid molecule encoding the above fusion proteins are each operably linked at the 5′ end to the P. pastoris AOX1 promoter (SEQ ID NO:27) and at the 3′ end to a nucleic acid molecule comprising the P. pastoris AOX1 transcription termination sequence (SEQ ID NO:31). For selecting transformants, the plasmid comprises an expression cassette encoding the Zeocin ORF in which the nucleic acid molecule encoding the ORF (SEQ ID NO:32) is operably linked at the 5′ end to a nucleic acid molecule having the S. cerevisiae TEF promoter sequence (SEQ ID NO:33) and at the 3′ end to a nucleic acid molecule having the S. cerevisiae CYC transcription termination sequence (SEQ ID NO:13). Plasmid pGLY11680 targets the AOX1 promoter in the host cell for integration whereas the pGLY10569 plasmid further includes a nucleic acid molecule for targeting the TRP2 locus (SEQ ID NO:34) for integration. The plasmids are roll-in plasmids that insert multiple copies of the plasmid into the target locus.

Plasmid pGLY11680, encoding the human proinsulin-Sed1p fusion protein was linearized with PmeI and the linearized plasmid was transformed into Pichia pastoris wild-type strain NRRL-Y11431 to provide host wild-type cells displaying the human split proinsulin molecule on the cell surface. Transformations were performed essentially as described in Example 1.

Protein expression for the transformed yeast strains was carried out at in shake flasks at 24° C. with buffered glycerol-complex medium (BMGY) consisting of 1% yeast extract, 2% peptone, 100 mM potassium phosphate buffer pH 6.0, 1.34% yeast nitrogen base, 4×10-5% biotin, and 2% glycerol. The induction medium for protein expression was buffered methanol-complex medium (BMMY) consisting of 2% methanol instead of glycerol in BMGY. Cells were typically harvested after two days methanol induction, centrifuged at 2,000 rpm for five minutes, and washed with ice-cold PBS (phosphate-buffered saline). The expressed insulin is processed into a split proinsulin molecule tethered to the surface of the host cell via the SED1. FIG. 17A shows in the lower portion the split proinsulin tethered to the cell surface. The S. cerevisiae alpha-mating factor propeptide is removed from the N-terminus of the molecule as the molecule is transported to the molecule to the cell surface.

To detect insulin receptor binding to the split proinsulin on the cell surface, 1×10⁶ yeast cells (0.1 OD600) were resuspended in 50 μL PBS (phosphate-buffered saline) to which 0.25 μg of soluble biotin labeled insulin receptor (in 0.25 μg/μL concentration) was added and incubated on ice for 30 minutes. Cells were washed once with ice-cold PBS and then one μL of streptavidin (allophycocyanin conjugate) was added to the cell suspension and the cells incubated on ice for 15 minutes. Cells were washed twice with ice-cold PBS and suspended in 200 μL of ice-cold PBS for flow cytometry analysis. Myc detection was carried out simultaneously as described earlier. The results shown in FIG. 17B indicate that the split proinsulin fusion protein is displayed on the cell surface and can bind the insulin receptor.

Plasmid pGLY10569 encoding freely secreted proinsulin was linearized using SpeI and transformed into strain NRRL-Y11430 as described earlier. Insulin was purified using reverse phase chromatography and purified protein was submitted to LC-MS analysis to confirm protein identity. As shown in FIG. 19, LC-MS detected a two chain split proinsulin peptide. No single chain insulin was identified. The results demonstrate that under the same growing conditions used to produce the human proinsulin-Sed1p fusion protein, the kex2 site between the C-peptide and A-chain peptide was cleaved to produce a heterodimer molecule. Thus, the human proinsulin-Sed1p fusion protein displayed on the cell surface is expected to be a split proinsulin heterodimer.

TABLE 3
BRIEF DESCRIPTION OF THE SEQUENCES

| SEQ | | |
|---|---|---|
| ID | | |
| NO: | Description | Sequence |
| 1 | S. cerevisiae | AGGCCTCGCAACAACCTATAATTGAGTTAAGTGCCTTT |
| | invertase gene | CCAAGCTAAAAAGTTTGAGGTTATAGGGGCTTAGCAT |
| | (ScSUC2) ORF | CCACACGTCACAATCTCGGGTATCGAGTATAGTATGT |
| | underlined | AGAATTACGGCAGGAGGTTTCCCAATGAACAAAGGAC |
| | | AGGGGCACGGTGAGCTGTCGAAGGTATCCATTTTATC |
| | | ATGTTTCGTTTGTACAAGCACGACATACTAAGACATTT |
| | | ACCGTATGGGAGTTGTTGTCCTAGCGTAGTTCTCGCTC |
| | | CCCCAGCAAAGCTCAAAAAAGTACGTCATTTAGAATA |
| | | GTTTGTGAGCAAATTACCAGTCGGTATGCTACGTTAG |
| | | AAAGGCCCACAGTATTCTTCTACCAAAGGCGTGCCTTT |
| | | GTTGAACTCGATCCATTATGAGGGCTTCCATTATTCCC |
| | | CGCATTTTTATTACTCTGAACAGGAATAAAAAGAAAA |
| | | AACCCAGTTTAGGAAATTATCCGGGGGCGAAGAAATA |
| | | CGCGTAGCGTTAATCGACCCCACGTCCAGGGTTTTTCC |
| | | ATGGAGGTTTCTGGAAAAACTGACGAGGAATGTGATT |
| | | ATAAATCCCTTTATGTGATGTCTAAGACTTTTAAGGTA |
| | | CGCCCGATGTTTGCCTATTACCATCATAGAGACGTTTC |
| | | TTTTCGAGGAATGCTTAAACGACTTTGTTTGACAAAAA |
| | | TGTTGCCTAAGGGCTCTATAGTAAACCATTTGGAAGA |
| | | AAGATTTGACGACTTTTTTTTTTTGGATTTCGATCCTAT |
| | | AATCCTTCCTCCTGAAAAGAAACATATAAATAGATAT |
| | | GTATTATTCTTCAAAACATTCTCTTGTTCTTGTGCTTTT |
| | | TTTTTACCATATATCTTACTTTTTTTTTTCTCTCAGAGA |
| | | AACAAGCAAAACAAAAAGCTTTTCTTTTCACTAACGT |
| | | ATATGATGCTTTTGCAAGCTTTCCTTTTCCTTTTGGCTG |
| | | GTTTTGCAGCCAAAATATCTGCATCAATGACAAACGA |
| | | AACTAGCGATAGACCTTTGGTCCACTTCACACCCAAC |
| | | AAGGGCTGGATGAATGACCCAAATGGGTTGTGGTACG |
| | | ATGAAAAAGATGCCAAATGGCATCTGTACTTTCAATA |
| | | CAACCCAAATGACACCGTATGGGGTACGCCATTGTTT |
| | | TGGGGCCATGCTACTTCCGATGATTTGACTAATTGGGA |
| | | AGATCAACCCATTGCTATCGCTCCCAAGCGTAACGAT |
| | | TCAGGTGCTTTCTCTGGCTCCATGGTGGTTGATTACAA |
| | | CAACACGAGTGGGTTTTTCAATGATACTATTGATCCAA |
| | | GACAAAGATGCGTTGCGATTTGGACTTATAACACTCC |
| | | TGAAAGTGAAGAGCAATACATTAGCTATTCTCTTGAT |
| | | GGTGGTTACACTTTTACTGAATACCAAAAGAACCCTG |
| | | TTTTAGCTGCCAACTCCACTCAATTCAGAGATCCAAAG |
| | | GTGTTCTGGTATGAACCTTCTCAAAAATGGATTATGAC |
| | | GGCTGCCAAATCACAAGACTACAAAATTGAAATTTAC |
| | | TCCTCTGATGACTTGAAGTCCTGGAAGCTAGAATCTGC |
| | | ATTTGCCAATGAAGGTTTCTTAGGCTACCAATACGAAT |
| | | GTCCAGGTTTGATTGAAGTCCCAACTGAGCAAGATCC |
| | | TTCCAAATCTTATTGGGTCATGTTTATTTCTATCAACC |
| | | CAGGTGCACCTGCTGGCGGTTCCTTCAACCAATATTTT |
| | | GTTGGATCCTTCAATGGTACTCATTTTGAAGCGTTTGA |
| | | CAATCAATCTAGAGTGGTAGATTTTGGTAAGGACTAC |
| | | TATGCCTTGCAAACTTTCTTCAACACTGACCCAACCTA |
| | | CGGTTCAGCATTAGGTATTGCCTGGGCTTCAAACTGG |
| | | GAGTACAGTGCCTTTGTCCCAACTAACCCATGGAGAT |
| | | CATCCATGTCTTTGGTCCGCAAGTTTTCTTTGAACACT |
| | | GAATATCAAGCTAATCCAGAGACTGAATTGATCAATT |
| | | TGAAAGCCGAACCAATATTGAACATTAGTAATGCTGG |
| | | TCCCTGGTCTCGTTTTGCTACTAACACAACTCTAACTA |
| | | AGGCCAATTCTTACAATGTCGATTTGAGCAACTCGACT |
| | | GGTACCCTAGAGTTTGAGTTGGTTTACGCTGTTAACAC |
| | | CACACAAACCATATCCAAATCCGTCTTTGCCGACTTAT |
| | | CACTTTGGTTCAAGGGTTTAGAAGATCCTGAAGAATA |
| | | TTTGAGAATGGGTTTTGAAGTCAGTGCTTCTTCCTTCT |
| | | TTTTGGACCGTGGTAACTCTAAGGTCAAGTTTGTCAAG |
| | | GAGAACCCATATTTCACAAACAGAATGTCTGTCAACA |
| | | ACCAACCATTCAAGTCTGAGAACGACCTAAGTTACTA |
| | | TAAAGTGTACGGCCTACTGGATCAAAACATCTTGGAA |
| | | TTGTACTTCAACGATGGAGATGTGGTTTCTACAAATAC |
| | | CTACTTCATGACCACCGGTAACGCTCTAGGATCTGTGA |
| | | ACATGACCACTGGTGTCGATAATTTGTTCTACATTGAC |
| | | AAGTTCCAAGTAAGGGAAGTAAAATAGAGGTTATAA |
| | | AACTTATTGTCTTTTTTATTTTTTTCAAAAGCCATTCTA |
| | | AAGGGCTTTAGCTAACGAGTGACGAATGTAAAACTTT |
| | | ATGATTTCAAAGAATACCTCCAAACCATTGAAAATGT |
| | | ATTTTTATTTTTATTTTCTCCCGACCCCAGTTACCTGGA |
| | | ATTTGTTCTTTATGTACTTTATATAAGTATAATTCTCTT |
| | | AAAAATTTTTACTACTTTGCAATAGACATCATTTTTTC |
| | | ACGTAATAAACCCACAATCGTAATGTAGTTGCCTTAC |
| | | ACTACTAGGATGGACCTTTTTGCCTTTATCTGTTTTGTT |
| | | ACTGACACAATGAAACCGGGTAAAGTATTAGTTATGT |
| | | GAAAATTTAAAAGCATTAAGTAGAAGTATACCATATT |
| | | GTAAAAAAAAAAAGCGTTGTCTTCTACGTAAAAGTGT |
| | | TCTCAAAAAGAAGTAGTGAGGGAAATGGATACCAAGC |
| | | TATCTGTAACAGGAGCTAAAAAATCTCAGGGAAAAGC |
| | | TTCTGGTTTGGGAAACGGTCGAC |
| 2 | Sequence of the | ATCGGCCTTTGTTGATGCAAGTTTTACGTGGATCATGG |
| | 5′-Region used | ACTAAGGAGTTTTATTTGGACCAAGTTCATCGTCCTAG |
| | for knock out of | ACATTACGGAAAGGGTTCTGCTCCTCTTTTTGGAAACT |
| | PpURA5: | TTTTGGAACCTCTGAGTATGACAGCTTGGTGGATTGTA |
| | | CCCATGGTATGGCTTCCTGTGAATTTCTATTTTTTCTAC |
| | | ATTGGATTCACCAATCAAAACAAATTAGTCGCCATGG |
| | | CTTTTTGGCTTTTGGGTCTATTTGTTTGGACCTTCTTGG |
| | | AATATGCTTTGCATAGATTTTTGTTCCACTTGGACTAC |
| | | TATCTTCCAGAGAATCAAATTGCATTTACCATTCATTT |
| | | CTTATTGCATGGGATACACCACTATTTACCAATGGATA |
| | | AATACAGATTGGTGATGCCACCTACACTTTTCATTGTA |
| | | CTTTGCTACCCAATCAAGACGCTCGTCTTTTCTGTTCT |
| | | ACCATATTACATGGCTTGTTCTGGATTTGCAGGTGGAT |
| | | TCCTGGGCTATATCATGTATGATGTCACTCATTACGTT |
| | | CTGCATCACTCCAAGCTGCCTCGTTATTTCCAAGAGTT |
| | | GAAGAAATATCATTTGGAACATCACTACAAGAATTAC |
| | | GAGTTAGGCTTTGGTGTCACTTCCAAATTCTGGGACAA |
| | | AGTCTTTGGGACTTATCTGGGTCCAGACGATGTGTATC |
| | | AAAAGACAAATTAGAGTATTTATAAAGTTATGTAAGC |
| | | AAATAGGGGCTAATAGGGAAAGAAAAATTTTGGTTCT |
| | | TTATCAGAGCTGGCTCGCGCGCAGTGTTTTTCGTGCTC |
| | | CTTTGTAATAGTCATTTTTGACTACTGTTCAGATTGAA |
| | | ATCACATTGAAGATGTCACTCGAGGGGTACCAAAAAA |
| | | GGTTTTTGGATGCTGCAGTGGCTTCGC |
| 3 | Sequence of the | GGTCTTTTCAACAAAGCTCCATTAGTGAGTCAGCTGGC |
| | 3′-Region used | TGAATCTTATGCACAGGCCATCATTAACAGCAACCTG |
| | for knock out of | GAGATAGACGTTGTATTTGGACCAGCTTATAAAGGTA |
| | PpURA5: | TTCCTTTGGCTGCTATTACCGTGTTGAAGTTGTACGAG |
| | | CTCGGCGGCAAAAAATACGAAAATGTCGGATATGCGT |
| | | TCAATAGAAAAGAAAAGAAAGACCACGGAGAAGGTG |
| | | GAAGCATCGTTGGAGAAAGTCTAAAGAATAAAAGAGT |
| | | ACTGATTATCGATGATGTGATGACTGCAGGTACTGCT |
| | | ATCAACGAAGCATTTGCTATAATTGGAGCTGAAGGTG |
| | | GGAGAGTTGAAGGTAGTATTATTGCCCTAGATAGAAT |
| | | GGAGACTACAGGAGATGACTCAAATACCAGTGCTACC |
| | | CAGGCTGTTAGTCAGAGATATGGTACCCCTGTCTTGA |
| | | GTATAGTGACATTGGACCATATTGTGGCCCATTTGGGC |
| | | GAAACTTTCACAGCAGACGAGAAATCTCAAATGGAAA |
| | | CGTATAGAAAAAAGTATTTGCCCAAATAAGTATGAAT |
| | | CTGCTTCGAATGAATGAATTAATCCAATTATCTTCTCA |
| | | CCATTATTTTCTTCTGTTTCGGAGCTTTGGGCACGGCG |
| | | GCGGGTGGTGCGGGCTCAGGTTCCCTTTCATAAACAG |
| | | ATTTAGTACTTGGATGCTTAATAGTGAATGGCGAATGC |
| | | AAAGGAACAATTTCGTTCATCTTTAACCCTTTCACTCG |
| | | GGGTACACGTTCTGGAATGTACCCGCCCTGTTGCAACT |
| | | CAGGTGGACCGGGCAATTCTTGAACTTTCTGTAACGTT |
| | | GTTGGATGTTCAACCAGAAATTGTCCTACCAACTGTAT |
| | | TAGTTTCCTTTTGGTCTTATATTGTTCATCGAGATACTT |
| | | CCCACTCTCCTTGATAGCCACTCTCACTCTTCCTGGAT |
| | | TACCAAAATCTTGAGGATGAGTCTTTTCAGGCTCCAG |
| | | GATGCAAGGTATATCCAAGTACCTGCAAGCATCTAAT |
| | | ATTGTCTTTGCCAGGGGGTTCTCCACACCATACTCCTT |
| | | TTGGCGCATGC |
| | Sequence of the | TCTAGAGGGACTTATCTGGGTCCAGACGATGTGTATC |
| | PpURA5 | AAAAGACAAATTAGAGTATTTATAAAGTTATGTAAGC |
| | auxotrophic | AAATAGGGGCTAATAGGGAAAGAAAAATTTTGGTTCT |
| | marker: | TTATCAGAGCTGGCTCGCGCGCAGTGTTTTTCGTGCTC |
| | | CTTTGTAATAGTCATTTTTGACTACTGTTCAGATTGAA |
| | | ATCACATTGAAGATGTCACTGGAGGGGTACCAAAAAA |
| | | GGTTTTTGGATGCTGCAGTGGCTTCGCAGGCCTTGAAG |
| | | TTTGGAACTTTCACCTTGAAAAGTGGAAGACAGTCTC |
| | | CATACTTCTTTAACATGGGTCTTTTCAACAAAGCTCCA |
| | | TTAGTGAGTCAGCTGGCTGAATCTTATGCTCAGGCCAT |
| | | CATTAACAGCAACCTGGAGATAGACGTTGTATTTGGA |
| | | CCAGCTTATAAAGGTATTCCTTTGGCTGCTATTACCGT |
| | | GTTGAAGTTGTACGAGCTGGGCGGCAAAAAATACGAA |
| | | AATGTCGGATATGCGTTCAATAGAAAAGAAAAGAAAG |
| | | ACCACGGAGAAGGTGGAAGCATCGTTGGAGAAAGTCT |
| | | AAAGAATAAAAGAGTACTGATTATCGATGATGTGATG |
| | | ACTGCAGGTACTGCTATCAACGAAGCATTTGCTATAA |
| | | TTGGAGCTGAAGGTGGGAGAGTTGAAGGTTGTATTAT |
| | | TGCCCTAGATAGAATGGAGACTACAGGAGATGACTCA |
| | | AATACCAGTGCTACCCAGGCTGTTAGTCAGAGATATG |
| | | GTACCCCTGTCTTGAGTATAGTGACATTGGACCATATT |
| | | GTGGCCCATTTGGGCGAAACTTTCACAGCAGACGAGA |
| | | AATCTCAAATGGAAACGTATAGAAAAAAGTATTTGCC |
| | | CAAATAAGTATGAATCTGCTTCGAATGAATGAATTAA |
| | | TCCAATTATCTTCTCACCATTATTTTCTTCTGTTTCGGA |
| | | GCTTTGGGCACGGCGGCGGATCC |
| 5 | Sequence of the | CCTGCACTGGATGGTGGCGCTGGATGGTAAGCCGCTG |
| | part of the Ec | GCAAGCGGTGAAGTGCCTCTGGATGTCGCTCCACAAG |
| | lacZ gene that | GTAAACAGTTGATTGAACTGCCTGAACTACCGCAGCC |
| | was used to | GGAGAGCGCCGGGCAACTCTGGCTCACAGTACGCGTA |
| | construct the | GTGCAACCGAACGCGACCGCATGGTCAGAAGCCGGGC |
| | PpURA5 blaster | ACATCAGCGCCTGGCAGCAGTGGCGTCTGGCGGAAAA |
| | (recyclable | CCTCAGTGTGACGCTCCCCGCCGCGTCCCACGCCATCC |
| | auxotrophic | CGCATCTGACCACCAGCGAAATGGATTTTTGCATCGA |
| | marker) | GCTGGGTAATAAGCGTTGGCAATTTAACCGCCAGTCA |
| | | GGCTTTCTTTCACAGATGTGGATTGGCGATAAAAAAC |
| | | AACTGCTGACGCCGCTGCGCGATCAGTTCACCCGTGC |
| | | ACCGCTGGATAACGACATTGGCGTAAGTGAAGCGACC |
| | | CGCATTGACCCTAACGCCTGGGTCGAACGCTGGAAGG |
| | | CGGCGGGCCATTACCAGGCCGAAGCAGCGTTGTTGCA |
| | | GTGCACGGCAGATACACTTGCTGATGCGGTGCTGATT |
| | | ACGACCGCTCACGCGTGGCAGCATCAGGGGAAAACCT |
| | | TATTTATCAGCCGGAAAACCTACCGGATTGATGGTAG |
| | | TGGTCAAATGGCGATTACCGTTGATGTTGAAGTGGCG |
| | | AGCGATACACCGCATCCGGCGCGGATTGGCCTGAACT |
| | | GCCAG |
| 6 | Sequence of the | AAAACCTTTTTTCCTATTCAAACACAAGGCATTGCTTC |
| | 5′-Region used | AACACGTGTGCGTATCCTTAACACAGATACTCCATACT |
| | for knock out of | TCTAATAATGTGATAGACGAATACAAAGATGTTCACT |
| | PpOCH1: | CTGTGTTGTGTCTACAAGCATTTCTTATTCTGATTGGG |
| | | GATATTCTAGTTACAGCACTAAACAACTGGCGATACA |
| | | AACTTAAATTAAATAATCCGAATCTAGAAAATGAACT |
| | | TTTGGATGGTCCGCCTGTTGGTTGGATAAATCAATACC |
| | | GATTAAATGGATTCTATTCCAATGAGAGAGTAATCCA |
| | | AGACACTCTGATGTCAATAATCATTTGCTTGCAACAAC |
| | | AAACCCGTCATCTAATCAAAGGGTTTGATGAGGCTTA |
| | | CCTTCAATTGCAGATAAACTCATTGCTGTCCACTGCTG |
| | | TATTATGTGAGAATATGGGTGATGAATCTGGTCTTCTC |
| | | CACTCAGCTAACATGGCTGTTTGGGCAAAGGTGGTAC |
| | | AATTATACGGAGATCAGGCAATAGTGAAATTGTTGAA |
| | | TATGGCTACTGGACGATGCTTCAAGGATGTACGTCTA |
| | | GTAGGAGCCGTGGGAAGATTGCTGGCAGAACCAGTTG |
| | | GCACGTCGCAACAATCCCCAAGAAATGAAATAAGTGA |
| | | AAACGTAACGTCAAAGACAGCAATGGAGTCAATATTG |
| | | ATAACACCACTGGCAGAGCGGTTCGTACGTCGTTTTG |
| | | GAGCCGATATGAGGCTCAGCGTGCTAACAGCACGATT |
| | | GACAAGAAGACTCTCGAGTGACAGTAGGTTGAGTAAA |
| | | GTATTCGCTTAGATTCCCAACCTTCGTTTTATTCTTTCG |
| | | TAGACAAAGAAGCTGCATGCGAACATAGGGACAACTT |
| | | TTATAAATCCAATTGTCAAACCAACGTAAAACCCTCT |
| | | GGCACCATTTTCAACATATATTTGTGAAGCAGTACGC |
| | | AATATCGATAAATACTCACCGTTGTTTGTAACAGCCCC |
| | | AACTTGCATACGCCTTCTAATGACCTCAAATGGATAA |
| | | GCCGCAGCTTGTGCTAACATACCAGCAGCACCGCCCG |
| | | CGGTCAGCTGCGCCCACACATATAAAGGCAATCTACG |
| | | ATCATGGGAGGAATTAGTTTTGACCGTCAGGTCTTCA |
| | | AGAGTTTTGAACTCTTCTTCTTGAACTGTGTAACCTTT |
| | | TAAATGACGGGATCTAAATACGTCATGGATGAGATCA |
| | | TGTGTGTAAAAACTGACTCCAGCATATGGAATCATTC |
| | | CAAAGATTGTAGGAGCGAACCCACGATAAAAGTTTCC |
| | | CAACCTTGCCAAAGTGTCTAATGCTGTGACTTGAAATC |
| | | TGGGTTCCTCGTTGAAGACCCTGCGTACTATGCCCAAA |
| | | AACTTTCCTCCACGAGCCCTATTAACTTCTCTATGAGT |
| | | TTCAAATGCCAAACGGACACGGATTAGGTCCAATGGG |
| | | TAAGTGAAAAACACAGAGCAAACCCCAGCTAATGAG |
| | | CCGGCCAGTAACCGTCTTGGAGCTGTTTCATAAGAGT |
| | | CATTAGGGATCAATAACGTTCTAATCTGTTCATAACAT |
| | | ACAAATTTTATGGCTGCATAGGGAAAAATTCTCAACA |
| | | GGGTAGCCGAATGACCCTGATATAGACCTGCGACACC |
| | | ATCATACCCATAGATCTGCCTGACAGCCTTAAAGAGC |
| | | CCGCTAAAAGACCCGGAAAACCGAGAGAACTCTGGAT |
| | | TAGCAGTCTGAAAAAGAATCTTCACTCTGTCTAGTGG |
| | | AGCAATTAATGTCTTAGCGGCACTTCCTGCTACTCCGC |
| | | CAGCTACTCCTGAATAGATCACATACTGCAAAGACTG |
| | | CTTGTCGATGACCTTGGGGTTATTTAGCTTCAAGGGCA |
| | | ATTTTTGGGACATTTTGGACACAGGAGACTCAGAAAC |
| | | AGACACAGAGCGTTCTGAGTCCTGGTGCTCCTGACGT |
| | | AGGCCTAGAACAGGAATTATTGGCTTTATTTGTTTGTC |
| | | CATTTCATAGGCTTGGGGTAATAGATAGATGACAGAG |
| | | AAATAGAGAAGACCTAATATTTTTTGTTCATGGCAAAT |
| | | CGCGGGTTCGCGGTCGGGTCACACACGGAGAAGTAAT |
| | | GAGAAGAGCTGGTAATCTGGGGTAAAAGGGTTCAAAA |
| | | GAAGGTCGCCTGGTAGGGATGCAATACAAGGTTGTCT |
| | | TGGAGTTTACATTGACCAGATGATTTGGCTTTTTCTCT |
| | | GTTCAATTCACATTTTTCAGCGAGAATCGGATTGACGG |
| | | AGAAATGGCGGGGTGTGGGGTGGATAGATGGCAGAA |
| | | ATGCTCGCAATCACCGCGAAAGAAAGACTTTATGGAA |
| | | TAGAACTACTGGGTGGTGTAAGGATTACATAGCTAGT |
| | | CCAATGGAGTCCGTTGGAAAGGTAAGAAGAAGCTAAA |
| | | ACCGGCTAAGTAACTAGGGAAGAATGATCAGACTTTG |
| | | ATTTGATGAGGTCTGAAAATACTCTGCTGCTTTTTCAG |
| | | TTGCTTTTTCCCTGCAACCTATCATTTTCCTTTTCATAA |
| | | GCCTGCCTTTTCTGTTTTCACTTATATGAGTTCCGCCG |
| | | AGACTTCCCCAAATTCTCTCCTGGAACATTCTCTATCG |
| | | CTCTCCTTCCAAGTTGCGCCCCCTGGCACTGCCTAGTA |
| | | ATATTACCACGCGACTTATATTCAGTTCCACAATTTCC |
| | | AGTGTTCGTAGCAAATATCATCAGCCATGGCGAAGGC |
| | | AGATGGCAGTTTGCTCTACTATAATCCTCACAATCCAC |
| | | CCAGAAGGTATTACTTCTACATGGCTATATTCGCCGTT |
| | | TCTGTCATTTGCGTTTTGTACGGACCCTCACAACAATT |
| | | ATCATCTCCAAAAATAGACTATGATCCATTGACGCTCC |
| | | GATCACTTGATTTGAAGACTTTGGAAGCTCCTTCACAG |
| | | TTGAGTCCAGGCACCGTAGAAGATAATCTTCG |
| 7 | Sequence of the | AAAGCTAGAGTAAAATAGATATAGCGAGATTAGAGA |
| | 3′-Region used | ATGAATACCTTCTTCTAAGCGATCGTCCGTCATCATAG |
| | for knock out of | AATATCATGGACTGTATAGTTTTTTTTTTGTACATATA |
| | PpOCH1: | ATGATTAAACGGTCATCCAACATCTCGTTGACAGATCT |
| | | CTCAGTACGCGAAATCCCTGACTATCAAAGCAAGAAC |
| | | CGATGAAGAAAAAAACAACAGTAACCCAAACACCAC |
| | | AACAAACACTTTATCTTCTCCCCCCCAACACCAATCAT |
| | | CAAAGAGATGTCGGAACCAAACACCAAGAAGCAAAA |
| | | ACTAACCCCATATAAAAACATCCTGGTAGATAATGCT |
| | | GGTAACCCGCTCTCCTTCCATATTCTGGGCTACTTCAC |
| | | GAAGTCTGACCGGTCTCAGTTGATCAACATGATCCTC |
| | | GAAATGGGTGGCAAGATCGTTCCAGACCTGCCTCCTC |
| | | TGGTAGATGGAGTGTTGTTTTTGACAGGGGATTACAA |
| | | GTCTATTGATGAAGATACCCTAAAGCAACTGGGGGAC |
| | | GTTCCAATATACAGAGACTCCTTCATCTACCAGTGTTT |
| | | TGTGCACAAGACATCTCTTCCCATTGACACTTTCCGAA |
| | | TTGACAAGAACGTCGACTTGGCTCAAGATTTGATCAA |
| | | TAGGGCCCTTCAAGAGTCTGTGGATCATGTCACTTCTG |
| | | CCAGCACAGCTGCAGCTGCTGCTGTTGTTGTCGCTACC |
| | | AACGGCCTGTCTTCTAAACCAGACGCTCGTACTAGCA |
| | | AAATACAGTTCACTCCCGAAGAAGATCGTTTTATTCTT |
| | | GACTTTGTTAGGAGAAATCCTAAACGAAGAAACACAC |
| | | ATCAACTGTACACTGAGCTCGCTCAGCACATGAAAAA |
| | | CCATACGAATCATTCTATCCGCCACAGATTTCGTCGTA |
| | | ATCTTTCCGCTCAACTTGATTGGGTTTATGATATCGAT |
| | | CCATTGACCAACCAACCTCGAAAAGATGAAAACGGGA |
| | | ACTACATCAAGGTACAAGGCCTTCCA |
| 8 | K. lactis UDP- | AAACGTAACGCCTGGCACTCTATTTTCTCAAACTTCTG |
| | GlcNAc | GGACGGAAGAGCTAAATATTGTGTTGCTTGAACAAAC |
| | transporter gene | CCAAAAAAACAAAAAAATGAACAAACTAAAACTACA |
| | (KIMNN2-2) | CCTAAATAAACCGTGTGTAAAACGTAGTACCATATTA |
| | ORF underlined | CTAGAAAAGATCACAAGTGTATCACACATGTGCATCT |
| | | CATATTACATCTTTTATCCAATCCATTCTCTCTATCCCG |
| | | TCTGTTCCTGTCAGATTCTTTTTCCATAAAAAGAAGAA |
| | | GACCCCGAATCTCACCGGTACAATGCAAAACTGCTGA |
| | | AAAAAAAAGAAAGTTCACTGGATACGGGAACAGTGC |
| | | CAGTAGGCTTCACCACATGGACAAAACAATTGACGAT |
| | | AAAATAAGCAGGTGAGCTTCTTTTTCAAGTCACGATC |
| | | CCTTTATGTCTCAGAAACAATATATACAAGCTAAACC |
| | | CTTTTGAACCAGTTCTCTCTTCATAGTTATGTTCACAT |
| | | AAATTGCGGGAACAAGACTCCGCTGGCTGTCAGGTAC |
| | | ACGTTGTAACGTTTTCGTCCGCCCAATTATTAGCACAA |
| | | CATTGGCAAAAAGAAAAACTGCTCGTTTTCTCTACAG |
| | | GTAAATTACAATTTTTTTCAGTAATTTTCGCTGAAAAA |
| | | TTTAAAGGGCAGGAAAAAAAGACGATCTCGACTTTGC |
| | | ATAGATGCAAGAACTGTGGTCAAAACTTGAAATAGTA |
| | | ATTTTGCTGTGCGTGAACTAATAAATATATATATATAT |
| | | ATATATATATATTTGTGTATTTTGTATATGTAATTGTGC |
| | | ACGTCTTGGCTATTGGATATAAGATTTTCGCGGGTTGA |
| | | TGACATAGAGCGTGTACTACTGTAATAGTTGTATATTC |
| | | AAAAGCTGCTGCGTGGAGAAAGACTAAAATAGATAA |
| | | AAAGCACACATTTTGACTTCGGTACCGTCAACTTAGTG |
| | | GGACAGTCTTTTATATTTGGTGTAAGCTCATTTCTGGT |
| | | ACTATTCGAAACAGAACAGTGTTTTCTGTATTACCGTC |
| | | CAATCGTTTGTCATGAGTTTTGTATTGATTTTGTCGTT |
| | | AGTGTTCGGAGGATGTTGTTCCAATGTGATTAGTTTCG |
| | | AGCACATGGTGCAAGGCAGCAATATAAATTTGGGAAA |
| | | TATTGTTACATTCACTCAATTCGTGTCTGTGACGCTAA |
| | | TTCAGTTGCCCAATGCTTTGGACTTCTCTCACTTTCCGT |
| | | TTAGGTTGCGACCTAGACACATTCCTCTTAAGATCCAT |
| | | ATGTTAGCTGTGTTTTTGTTCTTTACCAGTTCAGTCGCC |
| | | AATAACAGTGTGTTTAAATTTGACATTTCCGTTCCGAT |
| | | TCATATTATCATTAGATTTTCAGGTACCACTTTGACGA |
| | | TGATAATAGGTTGGGCTGTTTGTAATAAGAGGTACTCC |
| | | AAACTTCAGGTGCAATCTGCCATCATTATGACGCTTGG |
| | | TGCGATTGTCGCATCATTATACCGTGACAAAGAATTTT |
| | | CAATGGACAGTTTAAAGTTGAATACGGATTCAGTGGG |
| | | TATGACCCAAAAATCTATGTTTGGTATCTTTGTTGTGC |
| | | TAGTGGCCACTGCCTTGATGTCATTGTTGTCGTTGCTC |
| | | AACGAATGGACGTATAACAAGTACGGGAAACATTGGA |
| | | AAGAAACTTTGTTCTATTCGCATTTCTTGGCTCTACCG |
| | | TTGTTTATGTTGGGGTACACAAGGCTCAGAGACGAAT |
| | | TCAGAGACCTCTTAATTTCCTCAGACTCAATGGATATT |
| | | CCTATTGTTAAATTACCAATTGCTACGAAACTTTTCAT |
| | | GCTAATAGCAAATAACGTGACCCAGTTCATTTGTATC |
| | | AAAGGTGTTAACATGCTAGCTAGTAACACGGATGCTT |
| | | TGACACTTTCTGTCGTGCTTCTAGTGCGTAAATTTGTT |
| | | AGTCTTTTACTCAGTGTCTACATCTACAAGAACGTCCT |
| | | ATCCGTGACTGCATACCTAGGGACCATCACCGTGTTCC |
| | | TGGGAGCTGGTTTGTATTCATATGGTTCGGTCAAAACT |
| | | GCACTGCCTCGCTGAAACAATCCACGTCTGTATGATA |
| | | CTCGTTTCAGAATTTTTTTGATTTTCTGCCGGATATGGT |
| | | TTCTCATCTTTACAATCGCATTCTTAATTATACCAGAA |
| | | CGTAATTCAATGATCCCAGTGACTCGTAACTCTTATAT |
| | | GTCAATTTAAGC |
| 9 | Sequence of the | GGCCGAGCGGGCCTAGATTTTCACTACAAATTTCAAA |
| | 5′-Region used | ACTACGCGGATTTATTGTCTCAGAGAGCAATTTGGCAT |
| | for knock out of | TTCTGAGCGTAGCAGGAGGCTTCATAAGATTGTATAG |
| | PpBMT2: | GACCGTACCAACAAATTGCCGAGGCACAACACGGTAT |
| | | GCTGTGCACTTATGTGGCTACTTCCCTACAACGGAATG |
| | | AAACCTTCCTCTTTCCGCTTAAACGAGAAAGTGTGTCG |
| | | CAATTGAATGCAGGTGCCTGTGCGCCTTGGTGTATTGT |
| | | TTTTGAGGGCCCAATTTATCAGGCGCCTTTTTTCTTGG |
| | | TTGTTTTCCCTTAGCCTCAAGCAAGGTTGGTCTATTTC |
| | | ATCTCCGCTTCTATACCGTGCCTGATACTGTTGGATGA |
| | | GAACACGACTCAACTTCCTGCTGCTCTGTATTGCCAGT |
| | | GTTTTGTCTGTGATTTGGATCGGAGTCCTCCTTACTTG |
| | | GAATGATAATAATCTTGGCGGAATCTCCCTAAACGGA |
| | | GGCAAGGATTCTGCCTATGATGATCTGCTATCATTGGG |
| | | AAGCTTCAACGACATGGAGGTCGACTCCTATGTCACC |
| | | AACATCTACGACAATGCTCCAGTGCTAGGATGTACGG |
| | | ATTTGTCTTATCATGGATTGTTGAAAGTCACCCCAAAG |
| | | CATGACTTAGCTTGCGATTTGGAGTTCATAAGAGCTCA |
| | | GATTTTGGACATTGACGTTTACTCCGCCATAAAAGACT |
| | | TAGAAGATAAAGCCTTGACTGTAAAACAAAAGGTTGA |
| | | AAAACACTGGTTTACGTTTTATGGTAGTTCAGTCTTTC |
| | | TGCCCGAACACGATGTGCATTACCTGGTTAGACGAGT |
| | | CATCTTTTCGGCTGAAGGAAAGGCGAACTCTCCAGTA |
| | | ACATC |
| 10 | Sequence of the | CCATATGATGGGTGTTTGCTCACTCGTATGGATCAAAA |
| | 3′-Region used | TTCCATGGTTTCTTCTGTACAACTTGTACACTTATTTGG |
| | for knock out of | ACTTTTCTAACGGTTTTTCTGGTGATTTGAGAAGTCCT |
| | PpBMT2: | TATTTTGGTGTTCGCAGCTTATCCGTGATTGAACCATC |
| | | AGAAATACTGCAGCTCGTTATCTAGTTTCAGAATGTGT |
| | | TGTAGAATACAATCAATTCTGAGTCTAGTTTGGGTGGG |
| | | TCTTGGCGACGGGACCGTTATATGCATCTATGCAGTGT |
| | | TAAGGTACATAGAATGAAAATGTAGGGGTTAATCGAA |
| | | AGCATCGTTAATTTCAGTAGAACGTAGTTCTATTCCCT |
| | | ACCCAAATAATTTGCCAAGAATGCTTCGTATCCACAT |
| | | ACGCAGTGGACGTAGCAAATTTCACTTTGGACTGTGA |
| | | CCTCAAGTCGTTATCTTCTACTTGGACATTGATGGTCA |
| | | TTACGTAATCCACAAAGAATTGGATAGCCTCTCGTTTT |
| | | ATCTAGTGCACAGCCTAATAGCACTTAAGTAAGAGCA |
| | | ATGGACAAATTTGCATAGACATTGAGCTAGATACGTA |
| | | ACTCAGATCTTGTTCACTCATGGTGTACTCGAAGTACT |
| | | GCTGGAACCGTTACCTCTTATCATTTCGCTACTGGCTC |
| | | GTGAAACTACTGGATGAAAAAAAAAAAAGAGCTGAA |
| | | AGCGAGATCATCCCATTTTGTCATCATACAAATTCACG |
| | | CTTGCAGTTTTGCTTCGTTAACAAGACAAGATGTCTTT |
| | | ATCAAAGACCCGTTTTTTCTTCTTGAAGAATACTTCCC |
| | | TGTTGAGCACATGCAAACCATATTTATCTCAGATTTCA |
| | | CTCAACTTGGGTGCTTCCAAGAGAAGTAAAATTCTTCC |
| | | CACTGCATCAACTTCCAAGAAACCCGTAGACCAGTTT |
| | | CTCTTCAGCCAAAAGAAGTTGCTCGCCGATCACCGCG |
| | | GTAACAGAGGAGTCAGAAGGTTTCACACCCTTCCATC |
| | | CCGATTTCAAAGTCAAAGTGCTGCGTTGAACCAAGGT |
| | | TTTCAGGTTGCCAAAGCCCAGTCTGCAAAAACTAGTT |
| | | CCAAATGGCCTATTAATTCCCATAAAAGTGTTGGCTAC |
| | | GTATGTATCGGTACCTCCATTCTGGTATTTGCTATTGT |
| | | TGTCGTTGGTGGGTTGACTAGACTGACCGAATCCGGT |
| | | CTTTCCATAACGGAGTGGAAACCTATCACTGGTTCGGT |
| | | TCCCCCACTGACTGAGGAAGACTGGAAGTTGGAATTT |
| | | GAAAAATACAAACAAAGCCCTGAGTTTCAGGAACTAA |
| | | ATTCTCACATAACATTGGAAGAGTTCAAGTTTATATTT |
| | | TCCATGGAATGGGGACATAGATTGTTGGGAAGGGTCA |
| | | TCGGCCTGTCGTTTGTTCTTCCCACGTTTTACTTCATTG |
| | | CCCGTCGAAAGTGTTCCAAAGATGTTGCATTGAAACT |
| | | GCTTGCAATATGCTCTATGATAGGATTCCAAGGTTTCA |
| | | TCGGCTGGTGGATGGTGTATTCCGGATTGGACAAACA |
| | | GCAATTGGCTGAACGTAACTCCAAACCAACTGTGTCT |
| | | CCATATCGCTTAACTACCCATCTTGGAACTGCATTTGT |
| | | TATTTACTGTTACATGATTTACACAGGGCTTCAAGTTT |
| | | TGAAGAACTATAAGATCATGAAACAGCCTGAAGCGTA |
| | | TGTTCAAATTTTCAAGCAAATTGCGTCTCCAAAATTGA |
| | | AAACTTTCAAGAGACTCTCTTCAGTTCTATTAGGCCTG |
| | | GTG |
| 11 | DNA encodes | ATGTCTGCCAACCTAAAATATCTTTCCTTGGGAATTTT |
| | MmSLC35A3 | GGTGTTTCAGACTACCAGTCTGGTTCTAACGATGCGGT |
| | UDP-GlcNAc | ATTCTAGGACTTTAAAAGAGGAGGGGCCTCGTTATCT |
| | transporter | GTCTTCTACAGCAGTGGTTGTGGCTGAATTTTTGAAGA |
| | | TAATGGCCTGCATCTTTTTAGTCTACAAAGACAGTAAG |
| | | TGTAGTGTGAGAGCACTGAATAGAGTACTGCATGATG |
| | | AAATTCTTAATAAGCCCATGGAAACCCTGAAGCTCGC |
| | | TATCCCGTCAGGGATATATACTCTTCAGAACAACTTAC |
| | | TCTATGTGGCACTGTCAAACCTAGATGCAGCCACTTAC |
| | | CAGGTTACATATCAGTTGAAAATACTTACAACAGCAT |
| | | TATTTTCTGTGTCTATGCTTGGTAAAAAATTAGGTGTG |
| | | TACCAGTGGCTCTCCCTAGTAATTCTGATGGCAGGAGT |
| | | TGCTTTTGTACAGTGGCCTTCAGATTCTCAAGAGCTGA |
| | | ACTCTAAGGACCTTTCAACAGGCTCACAGTTTGTAGG |
| | | CCTCATGGCAGTTCTCACAGCCTGTTTTTCAAGTGGCT |
| | | TTGCTGGAGTTTATTTTGAGAAAATCTTAAAAGAAAC |
| | | AAAACAGTCAGTATGGATAAGGAACATTCAACTTGGT |
| | | TTCTTTGGAAGTATATTTGGATTAATGGGTGTATACGT |
| | | TTATGATGGAGAATTGGTCTCAAAGAATGGATTTTTTC |
| | | AGGGATATAATCAACTGACGTGGATAGTTGTTGCTCT |
| | | GCAGGCACTTGGAGGCCTTGTAATAGCTGCTGTCATC |
| | | AAATATGCAGATAACATTTTAAAAGGATTTGCGACCT |
| | | CCTTATCCATAATATTGTCAACAATAATATCTTATTTT |
| | | TGGTTGCAAGATTTTGTGCCAACCAGTGTCTTTTTCCT |
| | | TGGAGCCATCCTTGTAATAGCAGCTACTTTCTTGTATG |
| | | GTTACGATCCCAAACCTGCAGGAAATCCCACTAAAGC |
| | | ATAG |
| 12 | PpGAPDH | TTTTTGTAGAAATGTCTTGGTGTCCTCGTCCAATCAGG |
| | promoter | TAGCCATCTCTGAAATATCTGGCTCCGTTGCAACTCCG |
| | | AACGACCTGCTGGCAACGTAAAATTCTCCGGGGTAAA |
| | | ACTTAAATGTGGAGTAATGGAACCAGAAACGTCTCTT |
| | | CCCTTCTCTCTCCTTCCACCGCCCGTTACCGTCCCTAG |
| | | GAAATTTTACTCTGCTGGAGAGCTTCTTCTACGGCCCC |
| | | CTTGCAGCAATGCTCTTCCCAGCATTACGTTGCGGGTA |
| | | AAACGGAGGTCGTGTACCCGACCTAGCAGCCCAGGGA |
| | | TGGAAAAGTCCCGGCCGTCGCTGGCAATAATAGCGGG |
| | | CGGACGCATGTCATGAGATTATTGGAAACCACCAGAA |
| | | TCGAATATAAAAGGCGAACACCTTTCCCAATTTTGGTT |
| | | TCTCCTGACCCAAAGACTTTAAATTTAATTTATTTGTC |
| | | CCTATTTCAATCAATTGAACAACTATCAAAACACA |
| 13 | ScCYC TT | ACAGGCCCCTTTTCCTTTGTCGATATCATGTAATTAGT |
| | | TATGTCACGCTTACATTCACGCCCTCCTCCCACATCCG |
| | | CTCTAACCGAAAAGGAAGGAGTTAGACAACCTGAAGT |
| | | CTAGGTCCCTATTTATTTTTTTTAATAGTTATGTTAGTA |
| | | TTAAGAACGTTATTTATATTTCAAATTTTTCTTTTTTTT |
| | | CTGTACAAACGCGTGTACGCATGTAACATTATACTGA |
| | | AAACCTTGCTTGAGAAGGTTTTGGGACGCTCGAAGGC |
| | | TTTAATTTGCAAGCTGCCGGCTCTTAAG |
| 14 | Sequence of the | GATCTGGCCATTGTGAAACTTGACACTAAAGACAAAA |
| | 5′-Region used | CTCTTAGAGTTTCCAATCACTTAGGAGACGATGTTTCC |
| | for knock out of | TACAACGAGTACGATCCCTCATTGATCATGAGCAATTT |
| | PpMNN4L1: | GTATGTGAAAAAAGTCATCGACCTTGACACCTTGGAT |
| | | AAAAGGGCTGGAGGAGGTGGAACCACCTGTGCAGGC |
| | | GGTCTGAAAGTGTTCAAGTACGGATCTACTACCAAAT |
| | | ATACATCTGGTAACCTGAACGGCGTCAGGTTAGTATA |
| | | CTGGAACGAAGGAAAGTTGCAAAGCTCCAAATTTGTG |
| | | GTTCGATCCTCTAATTACTCTCAAAAGCTTGGAGGAA |
| | | ACAGCAACGCCGAATCAATTGACAACAATGGTGTGGG |
| | | TTTTGCCTCAGCTGGAGACTCAGGCGCATGGATTCTTT |
| | | CCAAGCTACAAGATGTTAGGGAGTACCAGTCATTCAC |
| | | TGAAAAGCTAGGTGAAGCTACGATGAGCATTTTCGAT |
| | | TTCCACGGTCTTAAACAGGAGACTTCTACTACAGGGC |
| | | TTGGGGTAGTTGGTATGATTCATTCTTACGACGGTGAG |
| | | TTCAAACAGTTTGGTTTGTTCACTCCAATGACATCTAT |
| | | TCTACAAAGACTTCAACGAGTGACCAATGTAGAATGG |
| | | TGTGTAGCGGGTTGCGAAGATGGGGATGTGGACACTG |
| | | AAGGAGAACACGAATTGAGTGATTTGGAACAACTGCA |
| | | TATGCATAGTGATTCCGACTAGTCAGGCAAGAGAGAG |
| | | CCCTCAAATTTACCTCTCTGCCCCTCCTCACTCCTTTTG |
| | | GTACGCATAATTGCAGTATAAAGAACTTGCTGCCAGC |
| | | CAGTAATCTTATTTCATACGCAGTTCTATATAGCACAT |
| | | AATCTTGCTTGTATGTATGAAATTTACCGCGTTTTAGT |
| | | TGAAATTGTTTATGITGTGTGCCTTGCATGAAATCTCT |
| | | CGTTAGCCCTATCCTTACATTTAACTGGTCTCAAAACC |
| | | TCTACCAATTCCATTGCTGTACAACAATATGAGGCGG |
| | | CATTACTGTAGGGTTGGAAAAAAATTGTCATTCCAGC |
| | | TAGAGATCACACGACTTCATCACGCTTATTGCTCCTCA |
| | | TTGCTAAATCATTTACTCTTGACTTCGACCCAGAAAAG |
| | | TTCGCC |
| 15 | Sequence of the | GCATGTCAAACTTGAACACAACGACTAGATAGTTGTT |
| | 3′-Region used | TTTTCTATATAAAACGAAACGTTATCATCTTTAATAAT |
| | for knock out of | CATTGAGGTTTACCCTTATAGTTCCGTATTTTCGTTTCC |
| | PpMNN4L1: | AAACTTAGTAATCTTTTGGAAATATCATCAAAGCTGGT |
| | | GCCAATCTTCTTGTTTGAAGTTTCAAACTGCTCCACCA |
| | | AGCTACTTAGAGACTGTTCTAGGTCTGAAGCAACTTC |
| | | GAACACAGAGACAGCTGCCGCCGATTGTTCTTTTTTGT |
| | | GTTTTTCTTCTGGAAGAGGGGCATCATCTTGTATGTCC |
| | | AATGCCCGTATCCTTTCTGAGTTGTCCGACACATTGTC |
| | | CTTCGAAGAGTTTCCTGACATTGGGCTTCTTCTATCCG |
| | | TGTATTAATTTTGGGTTAAGTTCCTCGTTTGCATAGCA |
| | | GTGGATACCTCGATTTTTTTGGCTCCTATTTACCTGAC |
| | | ATAATATTCTACTATAATCCAACTTGGACGCGTCATCT |
| | | ATGATAACTAGGCTCTCCTTTGTTCAAAGGGGACGTCT |
| | | TCATAATCCACTGGCACGAAGTAAGTCTGCAACGAGG |
| | | CGGCTTTTGCAACAGAACGATAGTGTCGTTTCGTACTT |
| | | GGACTATGCTAAACAAAAGGATCTGTCAAACATTTCA |
| | | ACCGTGTTTCAAGGCACTCTTTACGAATTATCGACCAA |
| | | GACCTTCCTAGACGAACATTTCAACATATCCAGGCTA |
| | | CTGCTTCAAGGTGGTGCAAATGATAAAGGTATAGATA |
| | | TTAGATGTGTTTGGGACCTAAAACAGTTCTTGCCTGAA |
| | | GATTCCCTTGAGCAACAGGCTTCAATAGCCAAGTTAG |
| | | AGAAGCAGTACCAAATCGGTAACAAAAGGGGGAAGC |
| | | ATATAAAACCTTTACTATTGCGACAAAATCCATCCTTG |
| | | AAAGTAAAGCTGTTTGTTCAATGTAAAGCATACGAAA |
| | | CGAAGGAGGTAGATCCTAAGATGGTTAGAGAACTTAA |
| | | CGGGACATACTCCAGCTGCATCCCATATTACGATCGCT |
| | | GGAAGACTTTTTTCATGTACGTATCGCCCACCAACCTT |
| | | TCAAAGCAAGCTAGGTATGATTTTGACAGTTCTCACA |
| | | ATCCATTGGTTTTCATGCAACTTGAAAAAACCCAACTC |
| | | AAACTTCATGGGGATCCATACAATGTAAATCATTACG |
| | | AGAGGGCGAGGTTGAAAAGTTTCCATTGCAATCACGT |
| | | CGCATCATGGCTACTGAAAGGCCTTAAC |
| 16 | Sequence of the | TCATTCTATATGTTCAAGAAAAGGGTAGTGAAAGGAA |
| | 5′-Region used | AGAAAAGGCATATAGGCGAGGGAGAGTTAGCTAGCA |
| | for knock out of | TACAAGATAATGAAGGATCAATAGCGGTAGTTAAAGT |
| | PpPNO1 and | GCACAAGAAAAGAGCACCTGTTGAGGCTGATGATAAA |
| | PpMNN4: | GCTCCAATTACATTGCCACAGAGAAACACAGTAACAG |
| | | AAATAGGAGGGGATGCACCACGAGAAGAGCATTCAG |
| | | TGAACAACTTTGCCAAATTCATAACCCCAAGCGCTAA |
| | | TAAGCCAATGTCAAAGTCGGCTACTAACATTAATAGT |
| | | ACAACAACTATCGATTTTCAACCAGATGTTTGCAAGG |
| | | ACTACAAACAGACAGGTTACTGCGGATATGGTGACAC |
| | | TTGTAAGTTTTTGCACCTGAGGGATGATTTCAAACAGG |
| | | GATGGAAATTAGATAGGGAGTGGGAAAATGTCCAAA |
| | | AGAAGAAGCATAATACTCTCAAAGGGGTTAAGGAGAT |
| | | CCAAATGTTTAATGAAGATGAGCTCAAAGATATCCCG |
| | | TTTAAATGCATTATATGCAAAGGAGATTACAAATCAC |
| | | CCGTGAAAACTTCTTGCAATCATTATTTTTGCGAACAA |
| | | TGTTTCCTGCAACGGTCAAGAAGAAAACCAAATTGTA |
| | | TTATATGTGGCAGAGACACTTTAGGAGTTGCTTTACCA |
| | | GCAAAGAAGTTGTCCCAATTTCTGGCTAAGATACATA |
| | | ATAATGAAAGTAATAAAGTTTAGTAATTGCATTGCGTT |
| | | GACTATTGATTGCATTGATGTCGTGTGATACTTTCACC |
| | | GAAAAAAAACACGAAGCGCAATAGGAGCGGTTGCAT |
| | | ATTAGTCCCCAAAGCTATTTAATTGTGCCTGAAACTGT |
| | | TTTTTAAGCTCATCAAGCATAATTGTATGCATTGCGAC |
| | | GTAACCAACGTTTAGGCGCAGTTTAATCATAGCCCAC |
| | | TGCTAAGCC |
| 17 | Sequence of the | CGGAGGAATGCAAATAATAATCTCCTTAATTACCCAC |
| | 3′-Region used | TGATAAGCTCAAGAGACGCGGTTTGAAAACGATATAA |
| | for knock out of | TGAATCATTTGGATTTTATAATAAACCCTGACAGTTTT |
| | PpPNO1 and | TCCACTGTATTGTTTTAACACTCATTGGAAGCTGTATT |
| | PpMNN4: | GATTCTAAGAAGCTAGAAATCAATACGGCCATACAAA |
| | | AGATGACATTGAATAAGCACCGGCTTTTTTGATTAGC |
| | | ATATACCTTAAAGCATGCATTCATGGCTACATAGTTGT |
| | | TAAAGGGCTTCTTCCATTATCAGTATAATGAATTACAT |
| | | AATCATGCACTTATATTTGCCCATCTCTGTTCTCTCACT |
| | | CTTGCCTGGGTATATTCTATGAAATTGCGTATAGCGTG |
| | | TCTCCAGTTGAACCCCAAGCTTGGCGAGTTTGAAGAG |
| | | AATGCTAACCTTGCGTATTCCTTGCTTCAGGAAACATT |
| | | CAAGGAGAAACAGGTCAAGAAGCCAAACATTTTGATC |
| | | CTTCCCGAGTTAGCATTGACTGGCTACAATTTTCAAAG |
| | | CCAGCAGCGGATAGAGCCTTTTTTGGAGGAAACAACC |
| | | AAGGGAGCTAGTACCCAATGGGCTCAAAAAGTATCCA |
| | | AGACGTGGGATTGCTTTACTTTAATAGGATACCCAGA |
| | | AAAAAGTTTAGAGAGCCCTCCCCGTATTTACAACAGT |
| | | GCGGTACTTGTATCGCCTCAGGGAAAAGTAATGAACA |
| | | ACTACAGAAAGTCCTTCTTGTATGAAGCTGATGAACA |
| | | TTGGGGATGTTCGGAATCTTCTGATGGGTTTCAAACAG |
| | | TAGATTTATTAATTGAAGGAAAGACTGTAAAGACATC |
| | | ATTTGGAATTTGCATGGATTTGAATCCTTATAAATTTG |
| | | AAGCTCCATTCACAGACTTCGAGTTCAGTGGCCATTGC |
| | | TTGAAAACCGGTACAAGACTCATTTTGTGCCCAATGG |
| | | CCTGGTTGTCCCCTCTATCGCCTTCCATTAAAAAGGAT |
| | | CTTAGTGATATAGAGAAAAGCAGACTTCAAAAGTTCT |
| | | ACCTTGAAAAAATAGATACCCCGGAATTTGACGTTAA |
| | | TTACGAATTGAAAAAAGATGAAGTATTGCCCACCCGT |
| | | ATGAATGAAACGTTGGAAACAATTGACTTTGAGCCTT |
| | | CAAAACCGGACTACTCTAATATAAATTATTGGATACT |
| | | AAGGTTTTTTCCCTTTCTGACTCATGTCTATAAACGAG |
| | | ATGTGCTCAAAGAGAATGCAGTTGCAGTCTTATGCAA |
| | | CCGAGTTGGCATTGAGAGTGATGTCTTGTACGGAGGA |
| | | TCAACCACGATTCTAAACTTCAATGGTAAGTTAGCATC |
| | | GACACAAGAGGAGCTGGAGTTGTACGGGCAGACTAAT |
| | | AGTCTCAACCCCAGTGTGGAAGTATTGGGGGCCCTTG |
| | | GCATGGGTCAACAGGGAATTCTAGTACGAGACATTGA |
| | | ATTAACATAATATACAATATACAATAAACACAAATAA |
| | | AGAATACAAGCCTGACAAAAATTCACAAATTATTGCC |
| | | TAGACTTGTCGTTATCAGCAGCGACCTTTTTCCAATGC |
| | | TCAATTTCACGATATGCCTTTTCTAGCTCTGCTTTAAG |
| | | CTTCTCATTGGAATTGGCTAACTCGTTGACTGCTTGGT |
| | | CAGTGATGAGTTTCTCCAAGGTCCATTTCTCGATGTTG |
| | | TTGTTTTCGTTTTCCTTTAATCTCTTGATATAATCAACA |
| | | GCCTTCTTTAATATCTGAGCCTTGTTCGAGTCCCCTGT |
| | | TGGCAACAGAGCGGCCAGTTCCTTTATTCCGTGGTTTA |
| | | TATTTTCTCTTCTACGCCTTTCTACTTCTTTGTGATTCT |
| | | CTTTACGCATCTTATGCCATTCTTCAGAACCAGTGGCT |
| | | GGCTTAACCGAATAGCCAGAGCCTGAAGAAGCCGCAC |
| | | TAGAAGAAGCAGTGGCATTGTTGACTATGG |
| 18 | Sequence of the | CATATGGTGAGAGCCGTTCTGCACAACTAGATGTTTTC |
| | 5′-Region used | GAGCTTCGCATTGTTTCCTGCAGCTCGACTATTGAATT |
| | for knock out of | AAGATTTCCGGATATCTCCAATCTCACAAAAACTTATG |
| | BMT1 | TTGACCACGTGCTTTCCTGAGGCGAGGTGTTTTATATG |
| | | CAAGCTGCCAAAAATGGAAAACGAATGGCCATTTTTC |
| | | GCCCAGGCAAATTATTCGATTACTGCTGTCATAAAGA |
| | | CAGTGTTGCAAGGCTCACATTTTTTTTTAGGATCCGAG |
| | | ATAAAGTGAATACAGGACAGCTTATCTCTATATCTTGT |
| | | ACCATTCGTGAATCTTAAGAGTTCGGTTAGGGGGACT |
| | | CTAGTTGAGGGTTGGCACTCACGTATGGCTGGGCGCA |
| | | GAAATAAAATTCAGGCGCAGCAGCACTTATCGATG |
| 19 | Sequence of the | GAATTCACAGTTATAAATAAAAACAAAAACTCAAAAA |
| | 3′-Region used | GTTTGGGCTCCACAAAATAACTTAATTTAAATTTTTGT |
| | for knock out of | CTAATAAATGAATGTAATTCCAAGATTATGTGATGCA |
| | BMT1 | AGCACAGTATGCTTCAGCCCTATGCAGCTACTAATGTC |
| | | AATCTCGCCTGCGAGCGGGCCTAGATTTTCACTACAA |
| | | ATTTCAAAACTACGCGGATTTATTGTCTCAGAGAGCA |
| | | ATTTGGCATTTCTGAGCGTAGCAGGAGGCTTCATAAG |
| | | ATTGTATAGGACCGTACCAACAAATTGCCGAGGCACA |
| | | ACACGGTATGCTGTGCACTTATGTGGCTACTTCCCTAC |
| | | AACGGAATGAAACCTTCCTCTTTCCGCTTAAACGAGA |
| | | AAGTGTGTCGCAATTGAATGCAGGTGCCTGTGCGCCT |
| | | TGGTGTATTGTTTTTGAGGGCCCAATTTATCAGGCGCC |
| | | TTTTTTCTTGGTTGTTTTCCCTTAGCCTCAAGCAAGGTT |
| | | GGTCTATTTCATCTCCGCTTCTATACCGTGCCTGATAC |
| | | TGTTGGATGAGAACACGACTCAACTTCCTGCTGCTCTG |
| | | TATTGCCAGTGTTTTGTCTGTGATTTGGATCGGAGTCC |
| | | TCCTTACTTGGAATGATAATAATCTTGGCGGAATCTCC |
| | | CTAAACGGAGGCAAGGATTCTGCCTATGATGATCTGC |
| | | TATCATTGGGAAGCTT |
| 20 | Sequence of the | AAGCTTGTTCACCGTTGGGACTTTTCCGTGGACAATGT |
| | 5′-Region used | TGACTACTCCAGGAGGGATTCCAGCTTTCTCTACTAGC |
| | for knock out of | TCAGCAATAATCAATGCAGCCCCAGGCGCCCGTTCTG |
| | BMT4 | ATGGCTTGATGACCGTTGTATTGCCTGTCACTATAGCC |
| | | AGGGGTAGGGTCCATAAAGGAATCATAGCAGGGAAA |
| | | TTAAAAGGGCATATTGATGCAATCACTCCCAATGGCT |
| | | CTCTTGCCATTGAAGTCTCCATATCAGCACTAACTTCC |
| | | AAGAAGGACCCCTTCAAGTCTGACGTGATAGAGCACG |
| | | CTTGCTCTGCCACCTGTAGTCCTCTCAAAACGTCACCT |
| | | TGTGCATCAGCAAAGACTTTACCTTGCTCCAATACTAT |
| | | GACGGAGGCAATTCTGTCAAAATTCTCTCTCAGCAATT |
| | | CAACCAACTTGAAAGCAAATTGCTGTCTCTTGATGAT |
| | | GGAGACTTTTTTCCAAGATTGAAATGCAATGTGGGAC |
| | | GACTCAATTGCTTCTTCCAGCTCCTCTTCGGTTGATTG |
| | | AGGAACTTTTGAAACCACAAAATTGGTCGTTGGGTCA |
| | | TGTACATCAAACCATTCTGTAGATTTAGATTCGACGAA |
| | | AGCGTTGTTGATGAAGGAAAAGGTTGGATACGGTTTG |
| | | TCGGTCTCTTTGGTATGGCCGGTGGGGTATGCAATTGC |
| | | AGTAGAAGATAATTGGACAGCCATTGTTGAAGGTAGA |
| | | GAAAAGGTCAGGGAACTTGGGGGTTATTTATACCATT |
| | | TTACCCCACAAATAACAACTGAAAAGTACCCATTCCA |
| | | TAGTGAGAGGTAACCGACGGAAAAAGACGGGCCCAT |
| | | GTTCTGGGACCAATAGAACTGTGTAATCCATTGGGAC |
| | | TAATCAACAGACGATTGGCAATATAATGAAATAGTTC |
| | | GTTGAAAAGCCACGTCAGCTGTCTTTTCATTAACTTTG |
| | | GTCGGACACAACATTTTCTACTGTTGTATCTGTCCTAC |
| | | TTTGCTTATCATCTGCCACAGGGCAAGTGGATTTCCTT |
| | | CTCGCGCGGCTGGGTGAAAACGGTTAACGTGAA |
| 21 | Sequence of the | GCCTTGGGGGACTTCAAGTCTTTGCTAGAAACTAGAT |
| | 3′-Region used | GAGGTCAGGCCCTCTTATGGTTGTGTCCCAATTGGGCA |
| | for knock out of | ATTTCACTCACCTAAAAAGCATGACAATTATTTAGCG |
| | BMT4 | AAATAGGTAGTATATTTTCCCTCATCTCCCAAGCAGTT |
| | | TCGTTTTTGCATCCATATCTCTCAAATGAGCAGCTACG |
| | | ACTCATTAGAACCAGAGTCAAGTAGGGGTGAGCTCAG |
| | | TCATCAGCCTTCGTTTCTAAAACGATTGAGTTCTTTTG |
| | | TTGCTACAGGAAGCGCCCTAGGGAACTTTCGCACTTT |
| | | GGAAATAGATTTTGATGACCAAGAGCGGGAGTTGATA |
| | | TTAGAGAGGCTGTCCAAAGTACATGGGATCAGGCCGG |
| | | CCAAATTGATTGGTGTGACTAAACCATTGTGTACTTGG |
| | | ACACTCTATTACAAAAGCGAAGATGATTTGAAGTATT |
| | | ACAAGTCCCGAAGTGTTAGAGGATTCTATCGAGCCCA |
| | | GAATGAAATCATCAACCGTTATCAGCAGATTGATAAA |
| | | CTCTTGGAAAGCGGTATCCCATTTTCATTATTGAAGAA |
| | | CTACGATAATGAAGATGTGAGAGACGGCGACCCTCTG |
| | | AACGTAGACGAAGAAACAAATCTACTTTTGGGGTACA |
| | | ATAGAGAAAGTGAATCAAGGGAGGTATTTGTGGCCAT |
| | | AATACTCAACTCTATCATTAATG |
| 22 | Sequence of the | GATATCTCCCTGGGGACAATATGTGTTGCAACTGTTCG |
| | 5′-Region used | TTGTTGGTGCCCCAGTCCCCCAACCGGTACTAATCGGT |
| | for knock out of | CTATGTTCCCGTAACTCATATTCGGTTAGAACTAGAAC |
| | BMT3 | AATAAGTGCATCATTGTTCAACATTGTGGTTCAATTGT |
| | | CGAACATTGCTGGTGCTTATATCTACAGGGAAGACGA |
| | | TAAGCCTTTGTACAAGAGAGGTAACAGACAGTTAATT |
| | | GGTATTTCTTTGGGAGTCGTTGCCCTCTACGTTGTCTC |
| | | CAAGACATACTACATTCTGAGAAACAGATGGAAGACT |
| | | CAAAAATGGGAGAAGCTTAGTGAAGAAGAGAAAGTT |
| | | GCCTACTTGGACAGAGCTGAGAAGGAGAACCTGGGTT |
| | | CTAAGAGGCTGGACTTTTTGTTCGAGAGTTAAACTGC |
| | | ATAATTTTTTCTAAGTAAATTTCATAGTTATGAAATTT |
| | | CTGCAGCTTAGTGTTTACTGCATCGTTTACTGCATCAC |
| | | CCTGTAAATAATGTGAGCTTTTTTCCTTCCATTGCTTG |
| | | GTATCTTCCTTGCTGCTGTTT |
| 23 | Sequence of the | ACAAAACAGTCATGTACAGAACTAACGCCTTTAAGAT |
| | 3′-Region used | GCAGACCACTGAAAAGAATTGGGTCCCATTTTTCTTG |
| | for knock out of | AAAGACGACCAGGAATCTGTCCATTTTGTTTACTCGTT |
| | BMT3 | CAATCCTCTGAGAGTACTCAACTGCAGTCTTGATAAC |
| | | GGTGCATGTGATGTTCTATTTGAGTTACCACATGATTT |
| | | TGGCATGTCTTCCGAGCTACGTGGTGCCACTCCTATGC |
| | | TCAATCTTCCTCAGGCAATCCCGATGGCAGACGACAA |
| | | AGAAATTTGGGTTTCATTCCCAAGAACGAGAATATCA |
| | | GATTGCGGGTGTTCTGAAACAATGTACAGGCCAATGT |
| | | TAATGCTTTTTGTTAGAGAAGGAACAAACTTTTTTGCT |
| | | GAGC |
| 24 | DNA encodes Tr | CGCGCCGGATCTCCCAACCCTACGAGGGCGGCAGCAG |
| | ManI catalytic | TCAAGGCCGCATTCCAGACGTCGTGGAACGCTTACCA |
| | domain | CCATTTTGCCTTTCCCCATGACGACCTCCACCCGGTCA |
| | | GCAACAGCTTTGATGATGAGAGAAACGGCTGGGGCTC |
| | | GTCGGCAATCGATGGCTTGGACACGGCTATCCTCATG |
| | | GGGGATGCCGACATTGTGAACACGATCCTTCAGTATG |
| | | TACCGCAGATCAACTTCACCACGACTGCGGTTGCCAA |
| | | CCAAGGCATCTCCGTGTTCGAGACCAACATTCGGTAC |
| | | CTCGGTGGCCTGCTTTCTGCCTATGACCTGTTGCGAGG |
| | | TCCTTTCAGCTCCTTGGCGACAAACCAGACCCTGGTAA |
| | | ACAGCCTTCTGAGGCAGGCTCAAACACTGGCCAACGG |
| | | CCTCAAGGTTGCGTTCACCACTCCCAGCGGTGTCCCGG |
| | | ACCCTACCGTCTTCTTCAACCCTACTGTCCGGAGAAGT |
| | | GGTGCATCTAGCAACAACGTCGCTGAAATTGGAAGCC |
| | | TGGTGCTCGAGTGGACACGGTTGAGCGACCTGACGGG |
| | | AAACCCGCAGTATGCCCAGCTTGCGCAGAAGGGCGAG |
| | | TCGTATCTCCTGAATCCAAAGGGAAGCCCGGAGGCAT |
| | | GGCCTGGCCTGATTGGAACGTTTGTCAGCACGAGCAA |
| | | CGGTACCTTTCAGGATAGCAGCGGCAGCTGGTCCGGC |
| | | CTCATGGACAGCTTCTACGAGTACCTGATCAAGATGT |
| | | ACCTGTACGACCCGGTTGCGTTTGCACACTACAAGGA |
| | | TCGCTGGGTCCTTGCTGCCGACTCGACCATTGCGCATC |
| | | TCGCCTCTCACCCGTCGACGCGCAAGGACTTGACCTTT |
| | | TTGTCTTCGTACAACGGACAGTCTACGTCGCCAAACTC |
| | | AGGACATTTGGCCAGTTTTGCCGGTGGCAACTTCATCT |
| | | TGGGAGGCATTCTCCTGAACGAGCAAAAGTACATTGA |
| | | CTTTGGAATCAAGCTTGCCAGCTCGTACTTTGCCACGT |
| | | ACAACCAGACGGCTTCTGGAATCGGCCCCGAAGGCTT |
| | | CGCGTGGGTGGACAGCGTGACGGGCGCCGGCGGCTCG |
| | | CCGCCCTCGTCCCAGTCCGGGTTCTACTCGTCGGCAGG |
| | | ATTCTGGGTGACGGCACCGTATTACATCCTGCGGCCG |
| | | GAGACGCTGGAGAGCTTGTACTACGCATACCGCGTCA |
| | | CGGGCGACTCCAAGTGGCAGGACCTGGCGTGGGAAGC |
| | | GTTCAGTGCCATTGAGGACGCATGCCGCGCCGGCAGC |
| | | GCGTACTCGTCCATCAACGACGTGACGCAGGCCAACG |
| | | GCGGOGGTGCCTCTGACGATATGGAGAGCTTCTGGTT |
| | | TGCCGAGGCGCTCAAGTATGCGTACCTGATCTTTGCG |
| | | GAGGAGTCGGATGTGCAGGTGCAGGCCAACGGCGGG |
| | | AACAAATTTGTCTTTAACACGGAGGCGCACCCCTTTA |
| | | GCATCCGTTCATCATCACGACGGGGCGGCCACCTTGC |
| | | TTAA |
| 25 | Saccharomyces | ATGAGATTCCCATCCATCTTCACTGCTGTTTTGTTCGC |
| | cerevisiae | TGCTTCTTCTGCTTTGGCT |
| | mating factor | |
| | pre-signal | |
| | peptide (DNA) | |
| 26 | Saccharomyces | MRFPSIFTAVLFAASSALA |
| | cerevisiae | |
| | mating factor | |
| | pre-signal | |
| | peptide (protein) | |
| 27 | Pp AOX1 | AACATCCAAAGACGAAAGGTTGAATGAAACCTTTTTG |
| | promoter | CCATCCGACATCCACAGGTCCATTCTCACACATAAGT |
| | | GCCAAACGCAACAGGAGGGGATACACTAGCAGCAGA |
| | | CCGTTGCAAACGCAGGACCTCCACTCCTCTTCTCCTCA |
| | | ACACCCACTTTTGCCATCGAAAAACCAGCCCAGTTATT |
| | | GGGCTTGATTGGAGCTCGCTCATTCCAATTCCTTCTAT |
| | | TAGGCTACTAACACCATGACTTTATTAGCCTGTCTATC |
| | | CTGGCCCCCCTGGCGAGGTTCATGTTTGTTTATTTCCG |
| | | AATGCAACAAGCTCCGCATTACACCCGAACATCACTC |
| | | CAGATGAGGGCTTTCTGAGTGTGGGGTCAAATAGTTT |
| | | CATGTTCCCCAAATGGCCCAAAACTGACAGTTTAAAC |
| | | GCTGTCTTGGAACCTAATATGACAAAAGCGTGATCTC |
| | | ATCCAAGATGAACTAAGTTTGGTTCGTTGAAATGCTA |
| | | ACGGCCAGTTGGTCAAAAAGAAACTTCCAAAAGTCGG |
| | | CATACCGTTTGTCTTGTTTGGTATTGATTGACGAATGC |
| | | TCAAAAATAATCTCATTAATGCTTAGCGCAGTCTCTCT |
| | | ATCGCTTCTGAACCCCGGTGCACCTGTGCCGAAACGC |
| | | AAATGGGGAAACACCCGCTTTTTGGATGATTATGCAT |
| | | TGTCTCCACATTGTATGCTTCCAAGATTCTGGTGGGAA |
| | | TACTGCTGATAGCCTAACGTTCATGATCAAAATTTAAC |
| | | TGTTCTAACCCCTACTTGACAGCAATATATAAACAGA |
| | | AGGAAGCTGCCCTGTCTTAAACCTTTTTTTTTATCATC |
| | | ATTATTAGCTTACTTTCATAATTGCGACTGGTTCCAAT |
| | | TGACAAGCTTTTGATTTTAACGACTTTTAACGACAACT |
| | | TGAGAAGATCAAAAAACAACTAATTATTCGAAACG |
| 28 | PpPRO1 5′ | GAGCTCGGCCGGAAGGGCCATCGAATTGTCATCGTCT |
| | region and ORF | CCTCAGGTGCCATCGCTGTGGGCATGAAGAGAGTCAA |
| | | CATGAAGCGGAAACCAAAAAAGTTACAGCAAGTGCA |
| | | GGCATTGGCTGCTATAGGACAAGGCCGTTTGATAGGA |
| | | CTTTGGGACGACCTTTTCCGTCAGTTGAATCAGCCTAT |
| | | TGCGCAGATTTTACTGACTAGAACGGATTTGGTCGATT |
| | | ACACCCAGTTTAAGAACGCTGAAAATACATTGGAACA |
| | | GCTTATTAAAATGGGTATTATTCCTATTGTCAATGAGA |
| | | ATGACACCCTATCCATTCAAGAAATCAAATTTGGTGA |
| | | CAATGACACCTTATCCGCCATAACAGCTGGTATGTGTC |
| | | ATGCAGACTACCTGTTTTTGGTGACTGATGTGGACTGT |
| | | CTTTACACGGATAACCCTCGTACGAATCCGGACGCTG |
| | | AGCCAATCGTGTTAGTTAGAAATATGAGGAATCTAAA |
| | | CGTCAATACCGAAAGTGGAGGTTCCGCCGTAGGAACA |
| | | GGAGGAATGACAACTAAATTGATCGCAGCTGATTTGG |
| | | GTGTATCTGCAGGTGTTACAACGATTATTTGCAAAAGT |
| | | GAACATCCCGAGCAGATTTTGGACATTGTAGAGTACA |
| | | GTATCCGTGCTGATAGAGTCGAAAATGAGGCTAAATA |
| | | TCTGGTCATCAACGAAGAGGAAACTGTGGAACAATTT |
| | | CAAGAGATCAATCGGTCAGAACTGAGGGAGTTGAACA |
| | | AGCTGGACATTCCTTTGCATACACGTTTCGTTGGCCAC |
| | | AGTTTTAATGCTGTTAATAACAAAGAGTTTTGGTTACT |
| | | CCATGGACTAAAGGCCAACGGAGCCATTATCATTGAT |
| | | CCAGGTTGTTATAAGGCTATCACTAGAAAAAACAAAG |
| | | CTGGTATTCTTCCAGCTGGAATTATTTCCGTAGAGGGT |
| | | AATTTCCATGAATACGAGTGTGTTGATGTTAAGGTAG |
| | | GACTAAGAGATCCAGATGACCCACATTCACTAGACCC |
| | | CAATGAAGAACTTTACGTCGTTGGCCGTGCCCGTTGTA |
| | | ATTACCCCAGCAATCAAATCAACAAAATTAAGGGTCT |
| | | ACAAAGCTCGCAGATCGAGCAGGTTCTAGGTTACGCT |
| | | GACGGTGAGTATGTTGTTCACAGGGACAACTTGGCTT |
| | | TCCCAGTATTTGCCGATCCAGAACTGTTGGATGTTGTT |
| | | GAGAGTACCCTGTCTGAACAGGAGAGAGAATCCAAAC |
| | | CAAATAAATAG |
| 29 | PpALG3 TT | ATTTACAATTAGTAATATTAAGGTGGTAAAAACATTC |
| | | GTAGAATTGAAATGAATTAATATAGTATGACAATGGT |
| | | TCATGTCTATAAATCTCCGGCTTCGGTACCTTCTCCCC |
| | | AATTGAATACATTGTCAAAATGAATGGTTGAACTATT |
| | | AGGTTCGCCAGTTTCGTTATTAAGAAAACTGTTAAAAT |
| | | CAAATTCCATATCATCGGTTCCAGTGGGAGGACCAGT |
| | | TCCATCGCCAAAATCCTGTAAGAATCCATTGTCAGAA |
| | | CCTGTAAAGTCAGTTTGAGATGAAATTTTTCCGGTCTT |
| | | TGTTGACTTGGAAGCTTCGTTAAGGTTAGGTGAAACA |
| | | GTTTGATCAACCAGCGGCTCCCGTTTTCGTCGCTTAGT |
| | | AG |
| 30 | PpPRO1 3′ | AATTTCACATATGCTGCTTGATTATGTAATTATACCTT |
| | region | GCGTTCGATGGCATCGATTTCCTCTTCTGTCAATCGCG |
| | | CATCGCATTAAAAGTATACTTTTTTTTTTTTCCTATAGT |
| | | ACTATTCGCCTTATTATAAACTTTGCTAGTATGAGTTC |
| | | TACCCCCAAGAAAGAGCCTGATTTGACTCCTAAGAAG |
| | | AGTCAGCCTCCAAAGAATAGTCTCGGTGGGGGTAAAG |
| | | GCTTTAGTGAGGAGGGTTTCTCCCAAGGGGACTTCAG |
| | | CGCTAAGCATATACTAAATCGTCGCCCTAACACCGAA |
| | | GGCTCTTCTGTGGCTTCGAACGTCATCAGTTCGTCATC |
| | | ATTGCAAAGGTTACCATCCTCTGGATCTGGAAGCGTT |
| | | GCTGTGGGAAGTGTGTTGGGATCTTCGCCATTAACTCT |
| | | TTCTGGAGGGTTCCACGGGCTTGATCCAACCAAGAAT |
| | | AAAATAGACGTTCCAAAGTCGAAACAGTCAAGGAGA |
| | | CAAAGTGTTCTTTCTGACATGATTTCCACTTCTCATGC |
| | | AGCTAGAAATGATCACTCAGAGCAGCAGTTACAAACT |
| | | GGACAACAATCAGAACAAAAAGAAGAAGATGGTAGT |
| | | CGATCTTCTTTTTCTGTTTCTTCCCCCGCAAGAGATATC |
| | | CGGCACCCAGATGTACTGAAAACTGTCGAGAAACATC |
| | | TTGCCAATGACAGCGAGATCGACTCATCTTTACAACTT |
| | | CAAGGTGGAGATGTCACTAGAGGCATTTATCAATGGG |
| | | TAACTGGAGAAAGTAGTCAAAAAGATAACCCGCCTTT |
| | | GAAACGAGCAAATAGTTTTAATGATTTTTCTTCTGTGC |
| | | ATGGTGACGAGGTAGGCAAGGCAGATGCTGACCACG |
| | | ATCGTGAAAGCGTATTCGACGAGGATGATATCTCCAT |
| | | TGATGATATCAAAGTTCCGGGAGGGATGCGTCGAAGT |
| | | TTTTTATTACAAAAGCATAGAGACCAACAACTTTCTGG |
| | | ACTGAATAAAACGGCTCACCAACCAAAACAACTTACT |
| | | AAACCTAATTTCTTCACGAACAACTTTATAGAGTTTTT |
| | | GGCATTGTATGGGCATTTTGCAGGTGAAGATTTGGAG |
| | | GAAGACGAAGATGAAGATTTAGACAGTGGTTCCGAAT |
| | | CAGTCGCAGTCAGTGATAGTGAGGGAGAATTCAGTGA |
| | | GGCTGACAACAATTTGTTGTATGATGAAGAGTCTCTCC |
| | | TATTAGCACCTAGTACCTCCAACTATGCGAGATCAAG |
| | | AATAGGAAGTATTCGTACTCCTACTTATGGATCTTTCA |
| | | GTTCAAATGTTGGTTCTTCGTCTATTCATCAGCAGTTA |
| | | ATGAAAAGTCAAATCCCGAAGCTGAAGAAACGTGGA |
| | | CAGCACAAGCATAAAACACAATCAAAAATACGCTCGA |
| | | AGAAGCAAACTACCACCGTAAAAGCAGTGTTGCTGCT |
| | | ATTAAAgGCcTTCAT |
| 31 | PpAOX1 TT | TCAAGAGGATGTCAGAATGCCATTTGCCTGAGAGATG |
| | | CAGGCTTCATTTTGATACTTTTTTATTTGTAACCTATAT |
| | | AGTATAGGATTTTTTTTGTCATTTTGTTTCTTCTCGTAC |
| | | GAGCTTGCTCCTGATCAGCCTATCTCGCAGCTGATGAA |
| | | TATCTTGTGGTAGGGGTTTGGGAAAATCATTCGAGTTT |
| | | GATGTTTTTCTTGGTATTTCCCACTCCTCTTCAGAGTAC |
| | | AGAAGATTAAGTGAGACGTTCGTTTGTGCA |
| 32 | Sequence of the | ATGGCCAAGTTGACCAGTGCCGTTCCGGTGCTCACCG |
| | Sh ble ORF | CGCGCGACGTCGCCGGAGCGGTCGAGTTCTGGACCGA |
| | (Zeocin | CCGGCTCGGGTTCTCCCGGGACTTCGTGGAGGACGAC |
| | resistance | TTCGCCGGTGTGGTCCGGGACGACGTGACCCTGTTCAT |
| | marker): | CAGCGCGGTCCAGGACCAGGTGGTGCCGGACAACACC |
| | | CTGGCCTGGGTGTGGGTGCGCGGCCTGGACGAGCTGT |
| | | ACGCCGAGTGGTCGGAGGTCGTGTCCACGAACTTCCG |
| | | GGACGCCTCCGGGCCGGCCATGACCGAGATCGGCGAG |
| | | CAGCCGTGGOGGCGGGAGTTCGCCCTGCGCGACCCGG |
| | | CCGGCAACTGCGTGCACTTCGTGGCCGAGGAGCAGGA |
| | | CTGA |
| 33 | S cTEF1 | GATCCCCCACACACCATAGCTTCAAAATGTTTCTACTC |
| | promoter | CTTTTTTACTCTTCCAGATTTTCTCGGACTCCGCGCATC |
| | | GCCGTACCACTTCAAAACACCCAAGCACAGCATACTA |
| | | AATTTCCCCTCTTTCTTCCTCTAGGGTGTCGTTAATTAC |
| | | CCGTACTAAAGGTTTGGAAAAGAAAAAAGAGACCGC |
| | | CTCGTTTCTTTTTCTTCGTCGAAAAAGGCAATAAAAAT |
| | | TTTTATCACGTTTCTTTTTCTTGAAAATTTTTTTTTTTG |
| | | ATTTTTTTCTCTTTCGATGACCTCCCATTGATATTTAAG |
| | | TTAATAAACGGTCTTCAATTTCTCAAGTTTCAGTTTCA |
| | | TTTTTCTTGTTCTATTACAACTTTTTTTACTTCTTGCTC |
| | | ATTAGAAAGAAAGCATAGCAATCTAATCTAAGTTTTA |
| | | ATTACAAA |
| 34 | PpTRP2 Region | ATGAGTGTAAGTGATAGTCATCTTGCAACAGATTATTT |
| | | TGGAACGCAACTAACAAAGCAGATACACCCTTCAGCA |
| | | GAATCCTTTCTGGATATTGTGAAGAATGATCGCCAAA |
| | | GTCACAGTCCTGAGACAGTTCCTAATCTTTACCCCATT |
| | | TACAAGTTCATCCAATCAGACTTCTTAACGCCTCATCT |
| | | GGCTTATATCAAGCTTACCAACAGTTCAGAAACTCCC |
| | | AGTCCAAGTTTCTTGCTTGAAAGTGCGAAGAATGGTG |
| | | ACACCGTTGACAGGTACACCTTTATGGGACATTCCCCC |
| | | AGAAAAATAATCAAGACTGGGCCTTTAGAGGGTGCTG |
| | | AAGTTGACCCCTTGGTGCTTCTGGAAAAAGAACTGAA |
| | | GGGCACCAGACAAGCGCAACTTCCTGGTATTCCTCGT |
| | | CTAAGTGGTGGTGCCATAGGATACATCTCGTACGATT |
| | | GTATTAAGTACTTTGAACCAAAAACTGAAAGAAAACT |
| | | GAAAGATGTTTTGCAACTTCCGGAAGCAGCTTTGATG |
| | | TTGTTCGACACGATCGTGGCTTTTGACAATGTTTATCA |
| | | AAGATTCCAGGTAATTGGAAACGTTTCTCTATCCGTTG |
| | | ATGACTCGGACGAAGCTATTCTTGAGAAATATTATAA |
| | | GACAAGAGAAGAAGTGGAAAAGATCAGTAAAGTGGT |
| | | ATTTGACAATAAAACTGTTCCCTACTATGAACAGAAA |
| | | GATATTATTCAAGGCCAAACGTTCACCTCTAATATTGG |
| | | TCAGGAAGGGTATGAAAACCATGTTCGCAAGCTGAAA |
| | | GAACATATTCTGAAAGGAGACATCTTCCAAGCTGTTC |
| | | CCTCTCAAAGGGTAGCCAGGCCGACCTCATTGCACCC |
| | | TTTCAACATCTATCGTCATTTGAGAACTGTCAATCCTT |
| | | CTCCATACATGTTCTATATTGACTATCTAGACTTCCAA |
| | | GTTGTTGGTGCTTCACCTGAATTACTAGTTAAATCCGA |
| | | CAACAACAACAAAATCATCACACATCCTATTGCTGGA |
| | | ACTCTTCCCAGAGGTAAAACTATCGAAGAGGACGACA |
| | | ATTATGCTAAGCAATTGAAGTCGTCTTTGAAAGACAG |
| | | GGCCGAGCACGTCATGCTGGTAGATTTGGCCAGAAAT |
| | | GATATTAACCGTGTGTGTGAGCCCACCAGTACCACGG |
| | | TTGATCGTTTATTGACTGTGGAGAGATTTTCTCATGTG |
| | | ATGCATCTTGTGTCAGAAGTCAGTGGAACATTGAGAC |
| | | CAAACAAGACTCGCTTCGATGCTTTCAGATCCATTTTC |
| | | CCAGCAGGAACCGTCTCCGGTGCTCCGAAGGTAAGAG |
| | | CAATGCAACTCATAGGAGAATTGGAAGGAGAAAAGA |
| | | GAGGTGTTTATGCGGGGGCCGTAGGACACTGGTCGTA |
| | | CGATGGAAAATCGATGGACACATGTATTGCCTTAAGA |
| | | ACAATGGTCGTCAAGGACGGTGTCGCTTACCTTCAAG |
| | | CCGGAGGTGGAATTGTCTACGATTCTGACCCCTATGA |
| | | CGAGTACATCGAAACCATGAACAAAATGAGATCCAAC |
| | | AATAACACCATCTTGGAGGCTGAGAAAATCTGGACCG |
| | | ATAGGTTGGCCAGAGACGAGAATCAAAGTGAATCCGA |
| | | AGAAAACGATCAATGA |
| 35 | Sc alpha mating | MRFPSIFTAVLFAASSALAAPVNTTTEDETAQIPAEAVIG |
| | factor signal | YSDLEGDFDVAVLPFSNSTNNGLLFINTTIASIAAKEEGVS |
| | sequence and | LEKR |
| | pro-peptide | |
| 36 | Sequence of the | EEGHHHHHHHHHHEPK |
| | N-terminal 10X | |
| | His peptide | |
| | spacer | |
| 37 | Insulin P28N B | FVNQHLCGSHLVEALYLVCGERGFFYTNKT |
| | chain | |
| 38 | Insulin A chain | GIVEQCCTSICSLYQLENYCN |
| 39 | Insulin B chain | FVNQHLCGSHLVEALYLVCGERGFFYTPKT |
| 40 | cMyc peptide | EQKLISEEDL |
| 41 | 3xG4S spacer or | GGGGSGGGGSGGGGS |
| | linker peptide | |
| 42 | Sequence of the | CAATTTTCTAATTCTACATCAGCATCTTCAACAGACGT |
| | truncated | AACTTCCAGTTCTTCAATATCAACTTCCAGTGGTTCCG |
| | ScSED1 | TCACTATCACATCTTCAGAAGCTCCAGAAAGTGATAA |
| | | CGGTACTTCTACTGCAGCCCCTACAGAAACCTCAACT |
| | | GAAGCCCCAACCACTGCTATTCCTACTAATGGTACATC |
| | | TACCGAAGCACCAACAACCGCCATACCTACAAACGGT |
| | | ACTTCTACAGAAGCACCAACTGATACTACAACCGAAG |
| | | CTCCAACTACAGCATTGCCTACAAATGGTACTTCTACT |
| | | GAAGCCCCAACTGACACCACTACAGAAGCTCCAACCA |
| | | CTGGTTTGCCTACAAACGGTACAACCTCAGCTTTTCCA |
| | | CCTACTACATCCTTACCACCTAGTAATACCACTACAAC |
| | | CCCACCTTATAACCCATCTACTGATTATACTACAGACT |
| | | ACACAGTTGTAACTGAATATACCACTTACTGTCCAGA |
| | | ACCTACAACCTTCACTACAAATGGTAAAACATACACC |
| | | GTTACTGAACCAACCACTTTAACAATAACCGATTGTCC |
| | | ATGCACAATCGAAAAGCCTACAACCACTTCTACAACC |
| | | GAATACACAGTCGTTACTGAATACACTACATACTGTC |
| | | CAGAACCTACCACTTTCACAACCAATGGTAAAACTTA |
| | | CACAGTTACCGAACCAACTACATTGACTATTACAGAC |
| | | TGTCCTTGCACTATAGAAAAGTCAGAAGCTCCAGAAT |
| | | CCAGTGTACCTGTCACAGAATCCAAAGGTACTACTAC |
| | | AAAGGAAACTGGTGTTACCACTAAACAAACAACCGCA |
| | | AATCCATCTTTAACAGTCTCAACTGTAGTCCCTGTTTC |
| | | TTCATCCGCCAGTTCTCATTCAGTTGTAATTAATTCCA |
| | | ACGGTGCTAATGTTGTCGTTCCAGGTGCTTTGGGTTTG |
| | | GCAGGTGTTGCTATGTTGTTTTTG |
| 43 | Truncated SED1 | QFSNSTSASSTDVTSSSSISTSSGSVTITSSEAPESDNGTST |
| | | AAPTETSTEAPTTAIPTNGTSTEAPTTAIPTNGTSTEAPTD |
| | | TTTEAPTTALPTNGTSTEAPTDTTTEAPTTGLPTNGTTSA |
| | | FPPTTSLPPSNTTTTPPYNPSTDYTTDYTVVTEYTTYCPEP |
| | | TTFTTNGKTYTVTEPTTLTITDCPCTIEKPTTTSTTEYTVV |
| | | TEYTTYCPEPTTFTTNGKTYTVTEPTTLTITDCPCTIEKSE |
| | | APESSVPVTESKGTTTKETGVTTKQTTANPSLTVSTVVPV |
| | | SSSASSHSVVINSNGANVVVPGALGLAGVAMLFL |
| 44 | IGF-1 C-peptide | GYGSSSRRAPQT |
| 45 | IGF-1 (Y2A) C- | GAGSSSRRAPQT |
| | peptide | |
| 46 | DNA encoding | ATGAGATTTCCAAGTATTTTTACCGCCGTCTTATTTGC |
| | fusion protein I | TGCCTCCTCCGCTTTAGCCGCCCCAGTCAACACCACCA |
| | | CCGAAGATGAAACAGCTCAAATCCCAGCTGAAGCAGT |
| | | TATTGGTTATTCAGATTTGGAGGGTGACTTTGACGTCG |
| | | CAGTTTTGCCTTTCTCAAATTCCACTAACAACGGTTTG |
| | | TTGTTTATTAACACTACAATAGCCAGTATCGCTGCAAA |
| | | AGAAGAAGGTGTTTCTTTGGAAAAGAGAGAAGAAGGT |
| | | CATCACCACCATCATCACCATCACCATCACGAACCAA |
| | | AATTCGTAAATCAACATTTGTGTGGTTCTCACTTAGTT |
| | | GAAGCTTTGTATTTGGTATGCGGTGAAAGAGGTTTCTT |
| | | TTATACCAACAAAACTGCCGCTAAGGGTATCGTTGAA |
| | | CAATGTTGCACTTCCATATGTAGTTTGTACCAATTGGA |
| | | AAACTACTGCAACTCTCATGGTTCAGAACAAAAGTTG |
| | | ATCTCAGAAGAAGATTTGTTGGAAGGTGGTGGTGGTT |
| | | CCGGTGGTGGTGGTTCTGGTGGTGGTGGTTCTGTTGAT |
| | | CAATTTTCTAATTCTACATCAGCATCTTCAACAGACGT |
| | | AACTTCCAGTTCTTCAATATCAACTTCCAGTGGTTCCG |
| | | TCACTATCACATCTTCAGAAGCTCCAGAAAGTGATAA |
| | | CGGTACTTCTACTGCAGCCCCTACAGAAACCTCAACT |
| | | GAAGCCCCAACCACTGCTATTCCTACTAATGGTACATC |
| | | TACCGAAGCACCAACAACCGCCATACCTACAAACGGT |
| | | ACTTCTACAGAAGCACCAACTGATACTACAACCGAAG |
| | | CTCCAACTACAGCATTGCCTACAAATGGTACTTCTACT |
| | | GAAGCCCCAACTGACACCACTACAGAAGCTCCAACCA |
| | | CTGGTTTGCCTACAAACGGTACAACCTCAGCTTTTCCA |
| | | CCTACTACATCCTTACCACCTAGTAATACCACTACAAC |
| | | CCCACCTTATAACCCATCTACTGATTATACTACAGACT |
| | | ACACAGTTGTAACTGAATATACCACTTACTGTCCAGA |
| | | ACCTACAACCTTCACTACAAATGGTAAAACATACACC |
| | | GTTACTGAACCAACCACTTTAACAATAACCGATTGTCC |
| | | ATGCACAATCGAAAAGCCTACAACCACTTCTACAACC |
| | | GAATACACAGTCGTTACTGAATACACTACATACTGTC |
| | | CAGAACCTACCACTTTCACAACCAATGGTAAAACTTA |
| | | CACAGTTACCGAACCAACTACATTGACTATTACAGAC |
| | | TGTCCTTGCACTATAGAAAAGTCAGAAGCTCCAGAAT |
| | | CCAGTGTACCTGTCACAGAATCCAAAGGTACTACTAC |
| | | AAAGGAAACTGGTGTTACCACTAAACAAACAACCGCA |
| | | AATCCATCTTTAACAGTCTCAACTGTAGTCCCTGTTTC |
| | | TTCATCCGCCAGTTCTCATTCAGTTGTAATTAATTCCA |
| | | ACGGTGCTAATGTTGTCGTTCCAGGTGCTTTGGGTTTG |
| | | GCAGGTGTTGCTATGTTGTTTTTG |
| 47 | Fusion protein I | MRFPSIFTAVLFAASSALAAPVNTTTEDETAQIPAEAVIGYS |
| | | DLEGDFDVAVLPFSNSTNNGLLFINTTIASIAAKEEGVSLEK |
| | | REEGHHHHHHHHHHEPKFVNQHLCGSHLVEALYLVCGERGFF |
| | | YTNKTAAKGIVEQCCTSICSLYQLENYCNSHGSEQKLISEED |
| | | LLEGGGGSGGGGSGGGGSVDQFSNSTSASSTDVTSSSSISTS |
| | | SGSVTITSSEAPESDNGTSTAAPTETSTEAPTTAIPTNGTST |
| | | EAPTTAIPTNGTSTEAPTDTTTEAPTTALPTNGTSTEAPTDT |
| | | TTEAPTTGLPTNGTTSAFPPTTSLPPSNTTTTPPYNPSTDYT |
| | | TDYTVVTEYTTYCPEPTTFTTNGKTYTVTEPTTLTITDCPCT |
| | | IEKPTTTSTTEYTVVTEYTTYCPEPTTFTTNGKTYTVTEPTT |
| | | LTITDCPCTIEKSEAPESSVPVTESKGTTTKETGVTTKQTTA |
| | | NPSLTVSTVVPVSSSASSHSVVINSNIGANVVVPGALGLAG |
| | | VAMLFL |
| 48 | Fusion protein | EEGHHHHHHHHHHEPKFVNQHLCGSHLVEALYLVCGERGFFY |
| | IA | TNKTAAKGIVEQCCTSICSLYQLENYCNSHGSEQKLISEEDL |
| | | LEGGGGSGGGGSGGGGSVDQFSNSTSASSTDVTSSSSISTSS |
| | | GSVTITSSEAPESDNGTSTAAPTETSTEAPTTAIPTNGTSTE |
| | | APTTAIPTNGTSTEAPTDTTTEAPTTALPTNGTSTEAPTDTT |
| | | TEAPTTGLPTNGTTSAFPPTTSLPPSNTTTTPPYNPSTDYTT |
| | | DYTVVTEYTTYCPEPTTFTTNGKTYTVTEPTTLTITDCPCTI |
| | | EKPTTTSTTEYTVVTEYTTYCPEPTTFTTNGKTYTVTEPTTL |
| | | TITDCPCTIEKSEAPESSVPVTESKGTTTKETGVTTKQTTAN |
| | | PSLTVSTVVPVSSSASSHSVVINSNGANVVVPGALGLAGV |
| | | AMLFL |
| 49 | DNA encoding | ATGAGATTTCCAAGTATTTTTACCGCCGTCTTATTTGC |
| | fusion protein | TGCCTCCTCCGCTTTAGCCGCCCCAGTCAACACCACCA |
| | II | CCGAAGATGAAACAGCTCAAATCCCAGCTGAAGCAGT |
| | | TATTGGTTATTCAGATTTGGAGGGTGACTTTGACGTCG |
| | | CAGTTTTGCCTTTCTCAAATTCCACTAACAACGGTTTG |
| | | TTGTTTATTAACACTACAATAGCCAGTATCGCTGCAAA |
| | | AGAAGAAGGTGTTTCTTTGGAAAAGAGAGAAGAAGGT |
| | | CATCACCACCATCATCACCATCACCATCACGAACCAA |
| | | AATTCGTAAATCAACATTTGTGTGGTTCTCACTTAGTT |
| | | GAAGCTTTGTATTTGGTATGCGGTGAAAGAGGTTTCTT |
| | | TTATACCAACAAAACTGGTTATGGATCTTCCTCAAGA |
| | | AGAGCCCCACAAACCGGTATCGTTGAACAATGTTGCA |
| | | CTTCCATATGTAGTTTGTACCAATTGGAAAACTACTGC |
| | | AACTCTCATGGTTCAGAACAAAAGTTGATCTCAGAAG |
| | | AAGATTTGTTGGAAGGTGGTGGTGGTTCCGGTGGTGG |
| | | TGGTTCTGGTGGTGGTGGTTCTGTTGATCAATTTTCTA |
| | | ATTCTACATCAGCATCTTCAACAGACGTAACTTCCAGT |
| | | TCTTCAATATCAACTTCCAGTGGTTCCGTCACTATCAC |
| | | ATCTTCAGAAGCTCCAGAAAGTGATAACGGTACTTCT |
| | | ACTGCAGCCCCTACAGAAACCTCAACTGAAGCCCCAA |
| | | CCACTGCTATTCCTACTAATGGTACATCTACCGAAGCA |
| | | CCAACAACCGCCATACCTACAAACGGTACTTCTACAG |
| | | AAGCACCAACTGATACTACAACCGAAGCTCCAACTAC |
| | | AGCATTGCCTACAAATGGTACTTCTACTGAAGCCCCA |
| | | ACTGACACCACTACAGAAGCTCCAACCACTGGTTTGC |
| | | CTACAAACGGTACAACCTCAGCTTTTCCACCTACTACA |
| | | TCCTTACCACCTAGTAATACCACTACAACCCCACCTTA |
| | | TAACCCATCTACTGATTATACTACAGACTACACAGTTG |
| | | TAACTGAATATACCACTTACTGTCCAGAACCTACAAC |
| | | CTTCACTACAAATGGTAAAACATACACCGTTACTGAA |
| | | CCAACCACTTTAACAATAACCGATTGTCCATGCACAA |
| | | TCGAAAAGCCTACAACCACTTCTACAACCGAATACAC |
| | | AGTCGTTACTGAATACACTACATACTGTCCAGAACCT |
| | | ACCACTTTCACAACCAATGGTAAAACTTACACAGTTA |
| | | CCGAACCAACTACATTGACTATTACAGACTGTCCTTGC |
| | | ACTATAGAAAAGTCAGAAGCTCCAGAATCCAGTGTAC |
| | | CTGTCACAGAATCCAAAGGTACTACTACAAAGGAAAC |
| | | TGGTGTTACCACTAAACAAACAACCGCAAATCCATCT |
| | | TTAACAGTCTCAACTGTAGTCCCTGTTTCTTCATCCGC |
| | | CAGTTCTCATTCAGTTGTAATTAATTCCAACGGTGCTA |
| | | ATGTTGTCGTTCCAGGTGCTTTGGGTTTGGCAGGTGTT |
| | | GCTATGTTGTTTTTG |
| 50 | Fusion protein | MRFPSIFTAVLFAASSALAAPVNTTTEDETAQIPAEAVIG |
| | II | YSDLEGDFDVAVLPFSNSTNNGLLFINTTIASIAAKEEGVS |
| | | LEKREEGHHHHHHHHHHEPKFVNQHLCGSHLVEALYLV |
| | | CGERGFFYTNKTGYGSSSRRAPQTGIVEQCCTSICSLYQL |
| | | ENYCNSHGSEQKLISEEDLLEGGGGSGGGGSGGGGSVDQ |
| | | FSNSTSASSTDVTSSSSISTSSGSVTITSSEAPESDNGTSTA |
| | | APTETSTEAPTTAIPTNGTSTEAPTTAIPTNGTSTEAPTDT |
| | | TTEAPTTALPTNGTSTEAPTDTTTEAPTTGLPTNGTTSAFP |
| | | PTTSLPPSNTTTTPPYNPSTDYTTDYTVVTEYTTYCPEPTT |
| | | FTTNGKTYTVTEPTTLTITDCPCTIEKPTTTSTTEYTVVTE |
| | | YTTYCPEPTTFTTNGKTYTVTEPTTLTITDCPCTIEKSEAP |
| | | ESSVPVTESKGTTTKETGVTTKQTTANPSLTVSTVVPVSS |
| | | SASSHSVVINSNGANVVVPGALGLAGVAMLFL |
| 51 | Fusion protein | EEGHHHHHHHHHHEPKFVNQHLCGSHLVEALYLVCGER |
| | IIA | GFFYTNKTGYGSSSRRAPQTGIVEQCCTSICSLYQLENYC |
| | | NSHGSEQKLISEEDLLEGGGGSGGGGSGGGGSVDQFSNS |
| | | TSASSTDVTSSSSISTSSGSVTITSSEAPESDNGTSTAAPTE |
| | | TSTEAPTTAIPTNGTSTEAPTTAIPTNGTSTEAPTDTTTEA |
| | | PTTALPTNGTSTEAPTDTTTEAPTTGLPTNGTTSAFPPTTS |
| | | LPPSNTTTTPPYNPSTDYTTDYTVVTEYTTYCPEPTTFTT |
| | | NGKTYTVTEPTTLTITDCPCTIEKPTTTSTTEYTVVTEYTT |
| | | YCPEPTTFTTNGKTYTVTEPTTLTITDCPCTIEKSEAPESS |
| | | VPVTESKGTTTKETGVTTKQTTANPSLTVSTVVPVSSSAS |
| | | SHSVVINSNGANVVVPGALGLAGVAMLFL |
| 52 | DNA encoding | ATGAGATTTCCAAGTATTTTTACCGCCGTCTTATTTGC |
| | fusion protein | TGCCTCCTCCGCTTTAGCCGCCCCAGTCAACACCACCA |
| | III | CCGAAGATGAAACAGCTCAAATCCCAGCTGAAGCAGT |
| | | TATTGGTTATTCAGATTTGGAGGGTGACTTTGACGTCG |
| | | CAGTTTTGCCTTTCTCAAATTCCACTAACAACGGTTTG |
| | | TTGTTTATTAACACTACAATAGCCAGTATCGCTGCAAA |
| | | AGAAGAAGGTGTTTCTTTGGAAAAGAGAGAAGAAGGT |
| | | CATCACCACCATCATCACCATCACCATCACGAACCAA |
| | | AATTCGTAAATCAACATTTGTGTGGTTCTCACTTAGTT |
| | | GAAGCTTTGTATTTGGTATGCGGTGAAAGAGGTTTCTT |
| | | TTATACCAACAAAACTGGTGCTGGATCTTCCTCAAGA |
| | | AGAGCCCCACAAACCGGTATCGTTGAACAATGTTGCA |
| | | CTTCCATATGTAGTTTGTACCAATTGGAAAACTACTGC |
| | | AACTCTCATGGTTCAGAACAAAAGTTGATCTCAGAAG |
| | | AAGATTTGTTGGAAGGTGGTGGTGGTTCCGGTGGTGG |
| | | TGGTTCTGGTGGTGGTGGTTCTGTTGATCAATTTTCTA |
| | | ATTCTACATCAGCATCTTCAACAGACGTAACTTCCAGT |
| | | TCTTCAATATCAACTTCCAGTGGTTCCGTCACTATCAC |
| | | ATCTTCAGAAGCTCCAGAAAGTGATAACGGTACTTCT |
| | | ACTGCAGCCCCTACAGAAACCTCAACTGAAGCCCCAA |
| | | CCACTGCTATTCCTACTAATGGTACATCTACCGAAGCA |
| | | CCAACAACCGCCATACCTACAAACGGTACTTCTACAG |
| | | AAGCACCAACTGATACTACAACCGAAGCTCCAACTAC |
| | | AGCATTGCCTACAAATGGTACTTCTACTGAAGCCCCA |
| | | ACTGACACCACTACAGAAGCTCCAACCACTGGTTTGC |
| | | CTACAAACGGTACAACCTCAGCTTTTCCACCTACTACA |
| | | TCCTTACCACCTAGTAATACCACTACAACCCCACCTTA |
| | | TAACCCATCTACTGATTATACTACAGACTACACAGTTG |
| | | TAACTGAATATACCACTTACTGTCCAGAACCTACAAC |
| | | CTTCACTACAAATGGTAAAACATACACCGTTACTGAA |
| | | CCAACCACTTTAACAATAACCGATTGTCCATGCACAA |
| | | TCGAAAAGCCTACAACCACTTCTACAACCGAATACAC |
| | | AGTCGTTACTGAATACACTACATACTGTCCAGAACCT |
| | | ACCACTTTCACAACCAATGGTAAAACTTACACAGTTA |
| | | CCGAACCAACTACATTGACTATTACAGACTGTCCTTGC |
| | | ACTATAGAAAAGTCAGAAGCTCCAGAATCCAGTGTAC |
| | | CTGTCACAGAATCCAAAGGTACTACTACAAAGGAAAC |
| | | TGGTGTTACCACTAAACAAACAACCGCAAATCCATCT |
| | | TTAACAGTCTCAACTGTAGTCCCTGTTTCTTCATCCGC |
| | | CAGTTCTCATTCAGTTGTAATTAATTCCAACGGTGCTA |
| | | ATGTTGTCGTTCCAGGTGCTTTGGGTTTGGCAGGTGTT |
| | | GCTATGTTGTTTTTG |
| 53 | Fusion protein | MRFPSIFTAVLFAASSALAAPVNTTTEDETAQIPAEAVIGYS |
| | III | DLEGDFDVAVLPFSNSTNNGLLFINTTIASIAAKEEGVSLEK |
| | | REEGHHHHHHHHHHEPKFVNQHLCGSHLVEALYLVCGERGFF |
| | | YTNKTGAGSSSRRAPQTGIVEQCCTSICSLYQLENYCNSHGS |
| | | EQKLISEEDLLEGGGGSGGGGSGGGGSVDQFSNSTSASSTDV |
| | | TSSSSISTSSGSVTITSSEAPESDNGTSTAAPTETSTEAPTT |
| | | AIPTNGTSTEAPTTAIPTNGTSTEAPTDTTTEAPTTALPTNG |
| | | TSTEAPTDTTTEAPTTGLPTNGTTSAFPPTTSLPPSNTTTTP |
| | | PYNPSTDYTTDYTVVTEYTTYCPEPTTFTTNGKTYTVTEPTT |
| | | LTITDCPCTIEKPTTTSTTEYTVVTEYTTYCPEPTTFTTNGK |
| | | TYTVTEPTTLTITDCPCTIEKSEAPESSVPVTESKGTTTKET |
| | | GVTTKQTTANPSLTVSTVVPVSSSASSHSVVINSNGANVVVP |
| | | GALGLAGVAMLFL |
| 54 | Fusion protein | EEGHHHHHHHHHHEPKFVNQHLCGSHLVEALYLVCGER |
| | IIIA | GFFYTNKTGAGSSSRRAPQTGIVEQCCTSICSLYQLENYC |
| | | NSHGSEQKLISEEDLLEGGGGSGGGGSGGGGSVDQFSNS |
| | | TSASSTDVTSSSSISTSSGSVTITSSEAPESDNGTSTAAPTE |
| | | TSTEAPTTAIPTNGTSTEAPTTAIPTNGTSTEAPTDTTTEA |
| | | PTTALPTNGTSTEAPTDTTTEAPTTGLPTNGTTSAFPPTTS |
| | | LPPSNTTTTPPYNPSTDYTTDYTVVTEYTTYCPEPTTFTT |
| | | NGKTYTVTEPTTLTITDCPCTIEKPTTTSTTEYTVVTEYTT |
| | | YCPEPTTFTTNGKTYTVTEPTTLTITDCPCTIEKSEAPESS |
| | | VPVTESKGTTTKETGVTTKQTTANPSLTVSTVVPVSSSAS |
| | | SHSVVINSNGANVVVPGALGLAGVAMLFL |
| 55 | PCR primer c/o- | TCCAGAAAGTGATAACGGTACTTCTACTGC |
| | ScSED1-FW | |
| 56 | PCR primer c/o- | AATGTAGTTGGTTCGGTAACTGTGTAAGTTTT |
| | S cSED1-RV | |
| 57 | Human GR2 | TSRLEGLQSENHRLRMKITELDKDLEEVTMQLQDVGGC |
| | coiled coil | |
| | peptide sequence | |
| 58 | Human GR1 | EEKSRLLEKENRELEKIIAEKEERVSELRHQLQSVGGC |
| | coiled coil | |
| | peptide sequence | |
| 59 | DNA encodes Sc | ATGAGATTTCCTTCAATTTTTACTGCAGTTTTATTCGC |
| | alpha mating | AGCATCCTCCGCATTAGCTGCTCCAGTCAACACTACA |
| | factor signal and | ACAGAAGATGAAACGGCACAAATTCCGGCTGAAGCTG |
| | pro-peptide | TCATCGGTTACTCAGATTTAGAAGGGGATTTCGATGTT |
| | | GCTGTTTTGCCATTTTCCAACAGCACAAATAACGGGTT |
| | | ATTGTTTATAAATACTACTATTGCCAGCATTGCTGCTA |
| | | AAGAAGAAGGGGTATCTCTCGAGAAAAGG |
| 60 | SED 1 Fusion | MRFPSIFTAVLFAASSALATSRLEGLQSENHRLRMKITE |
| | with signal seq, | LDKDLEEVTMQLQDVGGCEQKLISEEDLVDQFSNSTSA |
| | GR2, and cMyc | SSTDVTSSSSISTSSGSVTITSSEAPESDNGTSTAAPTETST |
| | | EAPTTAIPTNGTSTEAPTTAIPTNGTSTEAPTDTTTEAPTT |
| | | ALPTNGTSTEAPTDTTTEAPTTGLPTNGTTSAFPPTTSLPP |
| | | SNTTTTPPYNPSTDYTTDYTVVTEYTTYCPEPTTFTTNGK |
| | | TYTVTEPTTLTITDCPCTIEKPTTTSTTEYTVVTEYTTYCP |
| | | EPTTFTTNGKTYTVTEPTTLTITDCPCTIEKSEAPESSVPV |
| | | TESKGTTTKETGVTTKQTTANPSLTVSTVVPVSSSASSHS |
| | | VVINSNGANVVVPGALGLAGVAMLFL |
| 61 | SED 1 Fusion | TSRLEGLQSENHRLRMKITELDKDLEEVTMQLQDVG |
| | with GR2 and c- | GCEQKLISEEDLVDQFSNSTSASSTDVTSSSSISTSSGSVTI |
| | Myc | TSSEAPESDNGTSTAAPTETSTEAPTTAIPTNGTSTEAPTT |
| | | AIPTNGTSTEAPTDTTTEAPTTALPTNGTSTEAPTDTTTEA |
| | | PTTGLPTNGTTSAFPPTTSLPPSNTTTTPPYNPSTDYTTDY |
| | | TVVTEYTTYCPEPTTFTTNGKTYTVTEPTTLTITDCPCTIE |
| | | KPTTTSTTEYTVVTEYTTYCPEPTTFTTNGKTYTVTEPTT |
| | | LTITDCPCTIEKSEAPESSVPVTESKOTTTKETGVTTKQTT |
| | | ANPSLTVSTVVPVSSSASSHSVVINSNGANVVVPGALGL |
| | | AGVAMLFL |
| 62 | Pre-proinsulin | MRFPSIFTAVLFAASSALAAPVNTTTEDETAQIPAEAVIGYS |
| | analogue | DLEGDFDVAVLPFSNSTNNGLLFINTTIASIAAKEEGVSLEK |
| | precursor GR1 | EEGHHHHHHHHHHEPKFVNQHLCGSHLVEALYLVCGERGFFY |
| | fusion with | TNKTAAKGIVEQCCTSICSLYQLENYCNSHGSEQKLISEEDL |
| | cMyc | LEGGGGSGGGGSGGGGSEEKSRLLEKENRELEKIIAEKEERV |
| | | SELRHQLQSVGGC |
| 63 | Insulin analogue | EEGHHHHHHHHHHEPKFVNQHLCGSHLVEALYLVCGERGFFY |
| | precursor GR1 | TNKTAAKGIVEQCCTSICSLYQLENYCNSHGSEQKLISEEDL |
| | fusion | LEGGGGSGGGGSGGGGSEEKSRLLEKENFtELEKIIAEKEERV |
| | | SELRHQLQSVGGC |
| 64 | pre-proinsulin | MRFPSIFTAVLFAASSALAAPVNTTTEDETAQIPAEAVIG |
| | precursor fused | YSDLEGDFDVAVLPFSNSTNNGLLFINTTIASIAAKEEGVS |
| | at the C- | LEKRFVNQHLCGSHLVEALYLVCGERGFFYTPKTRREAE |
| | terminus to the | DLQVGQVELGGGPGAGSLQPLALEGSLQKRGIVEQCCTS |
| | N-terminus of a | ICSLYQLENYCNSHGSEQKLISEEDLGGGGSASVDQFSNS |
| | truncated | TSASSTDVTSSSSISTSSGSVTITSSEAPESDNGTSTAAPTE |
| | Saccharomyces | TSTEAPTTAIPTNGTSTEAPTTAIPTNGTSTEAPTDTTTEA |
| | cerevisiae SED1 | PTTALPTNGTSTEAPTDTTTEAPTTGLPTNGTTSAFPPTTS |
| | protein | LPPSNTTTTPPYNPSTDYTTDYTVVTEYTTYCPEPTTFTT |
| | | NGKTYTVTEPTTLTITDCPCTIEKPTTTSTTEYTVVTEYTT |
| | | YCPEPTTFTTNGKTYTVTEPTTLTITDCPCTIEKSEAPESS |
| | | VPVTESKGTTTKETGVTTKQTTANPSLTVSTVVPVSSSAS |
| | | SHSVVINSNGANVVVPGALGLAGVAMLFL |
| 65 | Human insulin | RREAEDLQVGQVELGGGPGAGSLQPLALEGSLQKR |
| | C-peptide | |
| 66 | Spacer or linker | GGGGSAS |
| | peptide | |
| 67 | Kex2 cleavage | LQKR |
| | site | |
| 68 | Kex2 consensus | LXKR |
| | cleavage site | |
| 69 | B-chain | FVNQHLCGSHLVEALYLVCGERGFFYTPKTRREAEDLQV |
| | peptide/C- | GQVELGGGPGAGSLQPLALEGSLQKR |
| | peptide fusion | |
| 70 | A-chain | GIVEQCCTSICSLYQLENYCNSHGSEQKLISEEDLGGGGS |
| | peptide/sed1p | ASVDQFSNSTSASSTDVTSSSSISTSSGSVTITSSEAPESDN |
| | fusion | GTSTAAPTETSTEAPTTAIPTNGTSTEAPTTAIPTNGTSTE |
| | | APTDTTTEAPTTALPTNGTSTEAPTDTTTEAPTTGLPTNG |
| | | TTSAFPPTTSLPPSNTTTTPPYNPSTDYTTDYTVVTEYTTY |
| | | CPEPTTFTTNGKTYTVTEPTTLTITDCPCTIEKPTTTSTTEY |
| | | TVVTEYTTYCPEPTTFTTNGKTYTVTEPTTLTITDCPCTIE |
| | | KSEAPESSVPVTESKGTTTKETGVTTKQTTANPSLTVSTV |
| | | VPVSSSASSHSVVINSNGANVVVPGALGLAGVAMLFL |

1. A method for detecting and isolating recombinant cells that express a ligand for the insulin receptor (IR) or insulin growth factor 1 (IGF-1) receptor, comprising:
(a) constructing recombinant cells wherein each recombinant cell transiently or stably expresses a fusion protein comprising a polypeptide, wherein the fusion protein is secreted and capable of being displayed on the surface of the recombinant cell, by transforming host cells with nucleic acid molecules encoding the fusion protein;
(b) detecting recombinant cells that display on the cell surface thereof a fusion protein comprising a polypeptide capable of binding the IR or IGF-1 receptor by contacting the recombinant cells produced in (a) with the IR or IGF-1 receptor; and
(c) isolating the recombinant cells that display the fusion protein detected in step (b) to provide the recombinant cells that express the ligand for the IR or IGF-1 receptor.

2. The method of claim 1, wherein the polypeptide is fused to a cell surface anchoring moiety or protein or cell surface binding portion thereof.

3. The method of claim 2, wherein the cell surface anchoring protein is Sed1p.

4. The method of claim 1, wherein in the recombinant cells in (a) are constructed by transfecting cells with first nucleic acid molecules encoding a cell surface anchoring protein or cell surface binding portion thereof fused to a first binding moiety and second nucleic acid molecules encoding fusion proteins comprising a polypeptide fused to a second binding moiety that is specific for the first binding moiety.

5. The method of claim 4, wherein the first binding moiety is a first peptide and the second binding moiety is a second peptide wherein the first and second peptides are capable of a specific pairwise interaction.

6. The method of claim 5, wherein the first and second peptides are coiled-coil peptides that are capable of the specific pairwise interaction.

7-9. (canceled)

10. The method of claim 1, wherein the recombinant cells in (a) are produced by transforming or transfecting cells with a plurality of nucleic acid molecules in which the majority of the nucleic acid molecules comprise at least one mutation in the nucleotide sequence encoding the polypeptide to produce a library of recombinant cells wherein each recombinant cell in the library produces a single species of polypeptide.

11. The method of claim 1, wherein the recombinant cells display on the cell surface thereof a plurality of different fusion proteins, wherein each fusion protein is encoded on a different nucleic acid molecule in a different recombinant cell.

12. (canceled)

13. The method of claim 1, wherein the polypeptide comprising the fusion protein is an insulin or insulin analogue precursor molecule.

14. The method of claim 13, wherein the insulin or insulin analogue precursor molecule is displayed on the cell surface in a single-chain structure having a structure characteristic of native insulin.

15. The method of claim 13, wherein the insulin or insulin analogue precursor molecule is displayed on the cell surface as a split proinsulin molecule having a structure characteristic of native insulin.

16. The method of claim 1, wherein the host cell is a bacterial, mammalian, insect, yeast, filamentous fungus, or plant host cell.

17. The method of claim 1, wherein the host cell is Pichia pastoris.

18. A method for detecting recombinant cells that express a ligand for the insulin receptor (IR) or insulin growth factor 1 (IGF-1) receptor; comprising
(a) constructing a library of recombinant cells wherein each cell transiently or stably expresses a secreted fusion protein comprising a polypeptide by transfecting host cells with a plurality nucleic acid molecules encoding the fusion protein, wherein each recombinant cell in the library expresses a different fusion protein; and
(b) contacting the library of recombinant cells produced in (a) with the IR or IGF-1 receptor to detect the recombinant cells in the library that express the ligand for the insulin receptor (IR) or insulin growth factor 1 (IGF-1) receptor.

19. The method of claim 18, wherein the polypeptide is fused to a cell surface anchoring protein or cell surface binding portion thereof.

20. The method of claim 19, wherein the cell surface anchoring protein is Sed1p.

21. The method of claim 18, wherein in the recombinant cells in (a) are constructed by transfecting cells with first nucleic acid molecules encoding a cell surface anchoring protein or cell surface binding portion thereof fused to a first binding moiety and second nucleic acid molecules encoding fusion proteins comprising a polypeptide fused to a second binding moiety that is specific for the first binding moiety.

22. The method of claim 21, wherein the first binding moiety is a first peptide and the second binding moiety is a second peptide wherein the first and second peptides are capable of a specific pairwise interaction.

23. The method of claim 18, wherein the polypeptide is fused to a modification motif that is coupled to a first binding partner when the fusion proteins are expressed and which binds to a second binding partner displayed on the surface of the recombinant cells.

24. (canceled)

25. A method for detecting and isolating recombinant cells that express a ligand for the insulin receptor (IR) or insulin growth factor 1 (IGF-1) receptor, comprising:
(a) constructing recombinant cells wherein each recombinant cell transiently or stably expresses a fusion protein comprising a polypeptide fused to a cell surface anchoring protein or cell surface binding portion thereof, wherein the fusion protein is secreted and capable of being displayed on the surface of the recombinant cell, by transfecting cells with nucleic acid molecules encoding the fusion protein;
(b) detecting recombinant cells that display on the cell surface thereof a fusion protein that comprises a polypeptide capable of binding the IR or IGF-1 receptor by contacting the recombinant cells produced in (a) with the IR or IGF-1 receptor; and
(c) isolating the recombinant cells that display the fusion protein detected in step (b) to provide the recombinant cells that express the ligand for the insulin IR or IGF-1 receptor.

26-31. (canceled)


SEQUENCE LISTING (72 sequences)

SEQ ID NO: 1 - DNA, 3029 bp, Sacchromyces cerevisiea
aggcctcgcaacaacctataattgagttaagtgcctttccaagctaaaaagtttgaggttataggggcttagcatccacacgtcacaatctcgggtatcgagtatagtatgtagaattacggcaggaggtttcccaatgaacaaaggacaggggcacggtgagctgtcgaaggtatccattttatcatgtttcgtttgtacaagcacgacatactaagacatttaccgtatgggagttgttgtcctagcgtagttctcgctcccccagcaaagctcaaaaaagtacgtcatttagaatagtttgtgagcaaattaccagtcggtatgctacgttagaaaggcccacagtattcttctaccaaaggcgtgcctttgttgaactcgatccattatgagggcttccattattccccgcatttttattactctgaacaggaataaaaagaaaaaacccagtttaggaaattatccgggggcgaagaaatacgcgtagcgttaatcgaccccacgtccagggtttttccatggaggtttctggaaaaactgacgaggaatgtgattataaatccctttatgtgatgtctaagacttttaaggtacgcccgatgtttgcctattaccatcatagagacgtttcttttcgaggaatgcttaaacgactttgtttgacaaaaatgttgcctaagggctctatagtaaaccatttggaagaaagatttgacgactttttttttttggatttcgatcctataatccttcctcctgaaaagaaacatataaatagatatgtattattcttcaaaacattctcttgttcttgtgctttttttttaccatatatcttacttttttttttctctcagagaaacaagcaaaacaaaaagcttttcttttcactaacgtatatgatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagaggttataaaacttattgtcttttttatttttttcaaaagccattctaaagggctttagctaacgagtgacgaatgtaaaactttatgatttcaaagaatacctccaaaccattgaaaatgtatttttatttttattttctcccgaccccagttacctggaatttgttctttatgtactttatataagtataattctcttaaaaatttttactactttgcaatagacatcattttttcacgtaataaacccacaatcgtaatgtagttgccttacactactaggatggacctttttgcctttatctgttttgttactgacacaatgaaaccgggtaaagtattagttatgtgaaaatttaaaagcattaagtagaagtataccatattgtaaaaaaaaaaagcgttgtcttctacgtaaaagtgttctcaaaaagaagtagtgagggaaatggataccaagctatctgtaacaggagctaaaaaatctcagggaaaagcttctggtttgggaaacggtcgac

SEQ ID NO: 2 - DNA, 898 bp, Artificial Sequence
  Sequence of the 5′-Region used for knock out of PpURA5
atcggcctttgttgatgcaagttttacgtggatcatggactaaggagttttatttggaccaagttcatcgtcctagacattacggaaagggttctgctcctctttttggaaactttttggaacctctgagtatgacagcttggtggattgtacccatggtatggcttcctgtgaatttctattttttctacattggattcaccaatcaaaacaaattagtcgccatggctttttggcttttgggtctatttgtttggaccttcttggaatatgctttgcatagatttttgttccacttggactactatcttccagagaatcaaattgcatttaccattcatttcttattgcatgggatacaccactatttaccaatggataaatacagattggtgatgccacctacacttttcattgtactttgctacccaatcaagacgctcgtcttttctgttctaccatattacatggcttgttctggatttgcaggtggattcctgggctatatcatgtatgatgtcactcattacgttctgcatcactccaagctgcctcgttatttccaagagttgaagaaatatcatttggaacatcactacaagaattacgagttaggctttggtgtcacttccaaattctgggacaaagtctttgggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactcgaggggtaccaaaaaaggtttttggatgctgcagtggcttcgc

SEQ ID NO: 3 - DNA, 1060 bp, Artificial Sequence
  Sequence of the 3′-Region used for knock out of PpURA5
ggtcttttcaacaaagctccattagtgagtcagctggctgaatcttatgcacaggccatcattaacagcaacctggagatagacgttgtatttggaccagcttataaaggtattcctttggctgctattaccgtgttgaagttgtacgagctcggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggtagtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcgggtggtgcgggctcaggttccctttcataaacagatttagtacttggatgcttaatagtgaatggcgaatgcaaaggaacaatttcgttcatctttaaccctttcactcggggtacacgttctggaatgtacccgccctgttgcaactcaggtggaccgggcaattcttgaactttctgtaacgttgttggatgttcaaccagaaattgtcctaccaactgtattagtttccttttggtcttatattgttcatcgagatacttcccactctccttgatagccactctcactcttcctggattaccaaaatcttgaggatgagtcttttcaggctccaggatgcaaggtatatccaagtacctgcaagcatctaatattgtctttgccagggggttctccacaccatactccttttggcgcatgc

SEQ ID NO: 4 - DNA, 957 bp, Artificial Sequence
  PpURA5 auxotrophic marker
tctagagggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactggaggggtaccaaaaaaggtttttggatgctgcagtggcttcgcaggccttgaagtttggaactttcaccttgaaaagtggaagacagtctccatacttctttaacatgggtcttttcaacaaagctccattagtgagtcagctggctgaatcttatgctcaggccatcattaacagcaacctggagatagacgttgtatttggaccagcttataaaggtattcctttggctgctattaccgtgttgaagttgtacgagctgggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggttgtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcggatcc

SEQ ID NO: 5 - DNA, 709 bp, Artificial Sequence
  Sequence of the part of the Ec lacZ gene that was used to construct the PpURA5 blaster (recyclable auxotrophic marker)
cctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccag

SEQ ID NO: 6 - DNA, 2875 bp, Artificial Sequence
  Sequence of the 5′-Region used for knock out of PpOCH1
aaaaccttttttcctattcaaacacaaggcattgcttcaacacgtgtgcgtatccttaacacagatactccatacttctaataatgtgatagacgaatacaaagatgttcactctgtgttgtgtctacaagcatttcttattctgattggggatattctagttacagcactaaacaactggcgatacaaacttaaattaaataatccgaatctagaaaatgaacttttggatggtccgcctgttggttggataaatcaataccgattaaatggattctattccaatgagagagtaatccaagacactctgatgtcaataatcatttgcttgcaacaacaaacccgtcatctaatcaaagggtttgatgaggcttaccttcaattgcagataaactcattgctgtccactgctgtattatgtgagaatatgggtgatgaatctggtcttctccactcagctaacatggctgtttgggcaaaggtggtacaattatacggagatcaggcaatagtgaaattgttgaatatggctactggacgatgcttcaaggatgtacgtctagtaggagccgtgggaagattgctggcagaaccagttggcacgtcgcaacaatccccaagaaatgaaataagtgaaaacgtaacgtcaaagacagcaatggagtcaatattgataacaccactggcagagcggttcgtacgtcgttttggagccgatatgaggctcagcgtgctaacagcacgattgacaagaagactctcgagtgacagtaggttgagtaaagtattcgcttagattcccaaccttcgttttattctttcgtagacaaagaagctgcatgcgaacatagggacaacttttataaatccaattgtcaaaccaacgtaaaaccctctggcaccattttcaacatatatttgtgaagcagtacgcaatatcgataaatactcaccgttgtttgtaacagccccaacttgcatacgccttctaatgacctcaaatggataagccgcagcttgtgctaacataccagcagcaccgcccgcggtcagctgcgcccacacatataaaggcaatctacgatcatgggaggaattagttttgaccgtcaggtcttcaagagttttgaactcttcttcttgaactgtgtaaccttttaaatgacgggatctaaatacgtcatggatgagatcatgtgtgtaaaaactgactccagcatatggaatcattccaaagattgtaggagcgaacccacgataaaagtttcccaaccttgccaaagtgtctaatgctgtgacttgaaatctgggttcctcgttgaagaccctgcgtactatgcccaaaaactttcctccacgagccctattaacttctctatgagtttcaaatgccaaacggacacggattaggtccaatgggtaagtgaaaaacacagagcaaaccccagctaatgagccggccagtaaccgtcttggagctgtttcataagagtcattagggatcaataacgttctaatctgttcataacatacaaattttatggctgcatagggaaaaattctcaacagggtagccgaatgaccctgatatagacctgcgacaccatcatacccatagatctgcctgacagccttaaagagcccgctaaaagacccggaaaaccgagagaactctggattagcagtctgaaaaagaatcttcactctgtctagtggagcaattaatgtcttagcggcacttcctgctactccgccagctactcctgaatagatcacatactgcaaagactgcttgtcgatgaccttggggttatttagcttcaagggcaatttttgggacattttggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagccatggcgaaggcagatggcagtttgctctactataatcctcacaatccacccagaaggtattacttctacatggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcg

SEQ ID NO: 7 - DNA, 997 bp, Artificial Sequence
  Sequence of the 3′-Region used for knock out of PpOCH1
aaagctagagtaaaatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaaccaaacaccaagaagcaaaaactaaccccatataaaaacatcctggtagataatgctggtaacccgctctccttccatattctgggctacttcacgaagtctgaccggtctcagttgatcaacatgatcctcgaaatgggtggcaagatcgttccagacctgcctcctctggtagatggagtgttgtttttgacaggggattacaagtctattgatgaagataccctaaagcaactgggggacgttccaatatacagagactccttcatctaccagtgttttgtgcacaagacatctcttcccattgacactttccgaattgacaagaacgtcgacttggctcaagatttgatcaatagggcccttcaagagtctgtggatcatgtcacttctgccagcacagctgcagctgctgctgttgttgtcgctaccaacggcctgtcttctaaaccagacgctcgtactagcaaaatacagttcactcccgaagaagatcgttttattcttgactttgttaggagaaatcctaaacgaagaaacacacatcaactgtacactgagctcgctcagcacatgaaaaaccatacgaatcattctatccgccacagatttcgtcgtaatctttccgctcaacttgattgggtttatgatatcgatccattgaccaaccaacctcgaaaagatgaaaacgggaactacatcaaggtacaaggccttcca

SEQ ID NO: 8 - DNA, 2159 bp, Artificial Sequence
  K. lactis UDP-GlcNAc transporter gene (KIMNN2-2)
aaacgtaacgcctggcactctattttctcaaacttctgggacggaagagctaaatattgtgttgcttgaacaaacccaaaaaaacaaaaaaatgaacaaactaaaactacacctaaataaaccgtgtgtaaaacgtagtaccatattactagaaaagatcacaagtgtatcacacatgtgcatctcatattacatcttttatccaatccattctctctatcccgtctgttcctgtcagattctttttccataaaaagaagaagaccccgaatctcaccggtacaatgcaaaactgctgaaaaaaaaagaaagttcactggatacgggaacagtgccagtaggcttcaccacatggacaaaacaattgacgataaaataagcaggtgagcttctttttcaagtcacgatccctttatgtctcagaaacaatatatacaagctaaacccttttgaaccagttctctcttcatagttatgttcacataaattgcgggaacaagactccgctggctgtcaggtacacgttgtaacgttttcgtccgcccaattattagcacaacattggcaaaaagaaaaactgctcgttttctctacaggtaaattacaatttttttcagtaattttcgctgaaaaatttaaagggcaggaaaaaaagacgatctcgactttgcatagatgcaagaactgtggtcaaaacttgaaatagtaattttgctgtgcgtgaactaataaatatatatatatatatatatatatatttgtgtattttgtatatgtaattgtgcacgtcttggctattggatataagattttcgcgggttgatgacatagagcgtgtactactgtaatagttgtatattcaaaagctgctgcgtggagaaagactaaaatagataaaaagcacacattttgacttcggtaccgtcaacttagtgggacagtcttttatatttggtgtaagctcatttctggtactattcgaaacagaacagtgttttctgtattaccgtccaatcgtttgtcatgagttttgtattgattttgtcgttagtgttcggaggatgttgttccaatgtgattagtttcgagcacatggtgcaaggcagcaatataaatttgggaaatattgttacattcactcaattcgtgtctgtgacgctaattcagttgcccaatgctttggacttctctcactttccgtttaggttgcgacctagacacattcctcttaagatccatatgttagctgtgtttttgttctttaccagttcagtcgccaataacagtgtgtttaaatttgacatttccgttccgattcatattatcattagattttcaggtaccactttgacgatgataataggttgggctgtttgtaataagaggtactccaaacttcaggtgcaatctgccatcattatgacgcttggtgcgattgtcgcatcattataccgtgacaaagaattttcaatggacagtttaaagttgaatacggattcagtgggtatgacccaaaaatctatgtttggtatctttgttgtgctagtggccactgccttgatgtcattgttgtcgttgctcaacgaatggacgtataacaagtacgggaaacattggaaagaaactttgttctattcgcatttcttggctctaccgttgtttatgttggggtacacaaggctcagagacgaattcagagacctcttaatttcctcagactcaatggatattcctattgttaaattaccaattgctacgaaacttttcatgctaatagcaaataacgtgacccagttcatttgtatcaaaggtgttaacatgctagctagtaacacggatgctttgacactttctgtcgtgcttctagtgcgtaaatttgttagtcttttactcagtgtctacatctacaagaacgtcctatccgtgactgcatacctagggaccatcaccgtgttcctgggagctggtttgtattcatatggttcggtcaaaactgcactgcctcgctgaaacaatccacgtctgtatgatactcgtttcagaatttttttgattttctgccggatatggtttctcatctttacaatcgcattcttaattataccagaacgtaattcaatgatcccagtgactcgtaactcttatatgtcaatttaagc

SEQ ID NO: 9 - DNA, 870 bp, Artificial Sequence
  Sequence of the 5′-Region used for knock out of PpBMT2
ggccgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagcttcaacgacatggaggtcgactcctatgtcaccaacatctacgacaatgctccagtgctaggatgtacggatttgtcttatcatggattgttgaaagtcaccccaaagcatgacttagcttgcgatttggagttcataagagctcagattttggacattgacgtttactccgccataaaagacttagaagataaagccttgactgtaaaacaaaaggttgaaaaacactggtttacgttttatggtagttcagtctttctgcccgaacacgatgtgcattacctggttagacgagtcatcttttcggctgaaggaaaggcgaactctccagtaacatc

SEQ ID NO: 10 - DNA, 1733 bp, Artificial Sequence
  Sequence of the 3′-Region used for knock out of PpBMT2
ccatatgatgggtgtttgctcactcgtatggatcaaaattccatggtttcttctgtacaacttgtacacttatttggacttttctaacggtttttctggtgatttgagaagtccttattttggtgttcgcagcttatccgtgattgaaccatcagaaatactgcagctcgttatctagtttcagaatgtgttgtagaatacaatcaattctgagtctagtttgggtgggtcttggcgacgggaccgttatatgcatctatgcagtgttaaggtacatagaatgaaaatgtaggggttaatcgaaagcatcgttaatttcagtagaacgtagttctattccctacccaaataatttgccaagaatgcttcgtatccacatacgcagtggacgtagcaaatttcactttggactgtgacctcaagtcgttatcttctacttggacattgatggtcattacgtaatccacaaagaattggatagcctctcgttttatctagtgcacagcctaatagcacttaagtaagagcaatggacaaatttgcatagacattgagctagatacgtaactcagatcttgttcactcatggtgtactcgaagtactgctggaaccgttacctcttatcatttcgctactggctcgtgaaactactggatgaaaaaaaaaaaagagctgaaagcgagatcatcccattttgtcatcatacaaattcacgcttgcagttttgcttcgttaacaagacaagatgtctttatcaaagacccgttttttcttcttgaagaatacttccctgttgagcacatgcaaaccatatttatctcagatttcactcaacttgggtgcttccaagagaagtaaaattcttcccactgcatcaacttccaagaaacccgtagaccagtttctcttcagccaaaagaagttgctcgccgatcaccgcggtaacagaggagtcagaaggtttcacacccttccatcccgatttcaaagtcaaagtgctgcgttgaaccaaggttttcaggttgccaaagcccagtctgcaaaaactagttccaaatggcctattaattcccataaaagtgttggctacgtatgtatcggtacctccattctggtatttgctattgttgtcgttggtgggttgactagactgaccgaatccggtctttccataacggagtggaaacctatcactggttcggttcccccactgactgaggaagactggaagttggaatttgaaaaatacaaacaaagccctgagtttcaggaactaaattctcacataacattggaagagttcaagtttatattttccatggaatggggacatagattgttgggaagggtcatcggcctgtcgtttgttcttcccacgttttacttcattgcccgtcgaaagtgttccaaagatgttgcattgaaactgcttgcaatatgctctatgataggattccaaggtttcatcggctggtggatggtgtattccggattggacaaacagcaattggctgaacgtaactccaaaccaactgtgtctccatatcgcttaactacccatcttggaactgcatttgttatttactgttacatgatttacacagggcttcaagttttgaagaactataagatcatgaaacagcctgaagcgtatgttcaaattttcaagcaaattgcgtctccaaaattgaaaactttcaagagactctcttcagttctattaggcctggtg

SEQ ID NO: 11 - DNA, 981 bp, Artificial Sequence
  DNA encodes MmSLC35A3 UDP-GlcNAc transporter
atgtctgccaacctaaaatatctttccttgggaattttggtgtttcagactaccagtctggttctaacgatgcggtattctaggactttaaaagaggaggggcctcgttatctgtcttctacagcagtggttgtggctgaatttttgaagataatggcctgcatctttttagtctacaaagacagtaagtgtagtgtgagagcactgaatagagtactgcatgatgaaattcttaataagcccatggaaaccctgaagctcgctatcccgtcagggatatatactcttcagaacaacttactctatgtggcactgtcaaacctagatgcagccacttaccaggttacatatcagttgaaaatacttacaacagcattattttctgtgtctatgcttggtaaaaaattaggtgtgtaccagtggctctccctagtaattctgatggcaggagttgcttttgtacagtggccttcagattctcaagagctgaactctaaggacctttcaacaggctcacagtttgtaggcctcatggcagttctcacagcctgtttttcaagtggctttgctggagtttattttgagaaaatcttaaaagaaacaaaacagtcagtatggataaggaacattcaacttggtttctttggaagtatatttggattaatgggtgtatacgtttatgatggagaattggtctcaaagaatggattttttcagggatataatcaactgacgtggatagttgttgctctgcaggcacttggaggccttgtaatagctgctgtcatcaaatatgcagataacattttaaaaggatttgcgacctccttatccataatattgtcaacaataatatcttatttttggttgcaagattttgtgccaaccagtgtctttttccttggagccatccttgtaatagcagctactttcttgtatggttacgatcccaaacctgcaggaaatcccactaaagcatag

SEQ ID NO: 12 - DNA, 486 bp, Artificial Sequence
  PpGAPDH promoter
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 13 - DNA, 293 bp, Artificial Sequence
  ScCYC TT
acaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgccggctcttaag

SEQ ID NO: 14 - DNA, 1128 bp, Artificial Sequence
  Sequence of the 5′-Region used for knock out of PpMNN4L1
gatctggccattgtgaaacttgacactaaagacaaaactcttagagtttccaatcacttaggagacgatgtttcctacaacgagtacgatccctcattgatcatgagcaatttgtatgtgaaaaaagtcatcgaccttgacaccttggataaaagggctggaggaggtggaaccacctgtgcaggcggtctgaaagtgttcaagtacggatctactaccaaatatacatctggtaacctgaacggcgtcaggttagtatactggaacgaaggaaagttgcaaagctccaaatttgtggttcgatcctctaattactctcaaaagcttggaggaaacagcaacgccgaatcaattgacaacaatggtgtgggttttgcctcagctggagactcaggcgcatggattctttccaagctacaagatgttagggagtaccagtcattcactgaaaagctaggtgaagctacgatgagcattttcgatttccacggtcttaaacaggagacttctactacagggcttggggtagttggtatgattcattcttacgacggtgagttcaaacagtttggtttgttcactccaatgacatctattctacaaagacttcaacgagtgaccaatgtagaatggtgtgtagcgggttgcgaagatggggatgtggacactgaaggagaacacgaattgagtgatttggaacaactgcatatgcatagtgattccgactagtcaggcaagagagagccctcaaatttacctctctgcccctcctcactccttttggtacgcataattgcagtataaagaacttgctgccagccagtaatcttatttcatacgcagttctatatagcacataatcttgcttgtatgtatgaaatttaccgcgttttagttgaaattgtttatgttgtgtgccttgcatgaaatctctcgttagccctatccttacatttaactggtctcaaaacctctaccaattccattgctgtacaacaatatgaggcggcattactgtagggttggaaaaaaattgtcattccagctagagatcacacgacttcatcacgcttattgctcctcattgctaaatcatttactcttgacttcgacccagaaaagttcgcc

SEQ ID NO: 15 - DNA, 1231 bp, Artificial Sequence
  Sequence of the 3′-Region used for knock out of PpMNN4L1
gcatgtcaaacttgaacacaacgactagatagttgttttttctatataaaacgaaacgttatcatctttaataatcattgaggtttacccttatagttccgtattttcgtttccaaacttagtaatcttttggaaatatcatcaaagctggtgccaatcttcttgtttgaagtttcaaactgctccaccaagctacttagagactgttctaggtctgaagcaacttcgaacacagagacagctgccgccgattgttcttttttgtgtttttcttctggaagaggggcatcatcttgtatgtccaatgcccgtatcctttctgagttgtccgacacattgtccttcgaagagtttcctgacattgggcttcttctatccgtgtattaattttgggttaagttcctcgtttgcatagcagtggatacctcgatttttttggctcctatttacctgacataatattctactataatccaacttggacgcgtcatctatgataactaggctctcctttgttcaaaggggacgtcttcataatccactggcacgaagtaagtctgcaacgaggcggcttttgcaacagaacgatagtgtcgtttcgtacttggactatgctaaacaaaaggatctgtcaaacatttcaaccgtgtttcaaggcactctttacgaattatcgaccaagaccttcctagacgaacatttcaacatatccaggctactgcttcaaggtggtgcaaatgataaaggtatagatattagatgtgtttgggacctaaaacagttcttgcctgaagattcccttgagcaacaggcttcaatagccaagttagagaagcagtaccaaatcggtaacaaaagggggaagcatataaaacctttactattgcgacaaaatccatccttgaaagtaaagctgtttgttcaatgtaaagcatacgaaacgaaggaggtagatcctaagatggttagagaacttaacgggacatactccagctgcatcccatattacgatcgctggaagacttttttcatgtacgtatcgcccaccaacctttcaaagcaagctaggtatgattttgacagttctcacaatccattggttttcatgcaacttgaaaaaacccaactcaaacttcatggggatccatacaatgtaaatcattacgagagggcgaggttgaaaagtttccattgcaatcacgtcgcatcatggctactgaaaggccttaac

SEQ ID NO: 16 - DNA, 937 bp, Artificial Sequence
  Sequence of the 5′-Region used for knock out of PpPNO1 and PpMNN4
tcattctatatgttcaagaaaagggtagtgaaaggaaagaaaaggcatataggcgagggagagttagctagcatacaagataatgaaggatcaatagcggtagttaaagtgcacaagaaaagagcacctgttgaggctgatgataaagctccaattacattgccacagagaaacacagtaacagaaataggaggggatgcaccacgagaagagcattcagtgaacaactttgccaaattcataaccccaagcgctaataagccaatgtcaaagtcggctactaacattaatagtacaacaactatcgattttcaaccagatgtttgcaaggactacaaacagacaggttactgcggatatggtgacacttgtaagtttttgcacctgagggatgatttcaaacagggatggaaattagatagggagtgggaaaatgtccaaaagaagaagcataatactctcaaaggggttaaggagatccaaatgtttaatgaagatgagctcaaagatatcccgtttaaatgcattatatgcaaaggagattacaaatcacccgtgaaaacttcttgcaatcattatttttgcgaacaatgtttcctgcaacggtcaagaagaaaaccaaattgtattatatgtggcagagacactttaggagttgctttaccagcaaagaagttgtcccaatttctggctaagatacataataatgaaagtaataaagtttagtaattgcattgcgttgactattgattgcattgatgtcgtgtgatactttcaccgaaaaaaaacacgaagcgcaataggagcggttgcatattagtccccaaagctatttaattgtgcctgaaactgttttttaagctcatcaagcataattgtatgcattgcgacgtaaccaacgtttaggcgcagtttaatcatagcccactgctaagcc

SEQ ID NO: 17 - DNA, 1906 bp, Artificial Sequence
  Sequence of the 3′-Region used for knock out of PpPNO1 and PpMNN4
cggaggaatgcaaataataatctccttaattacccactgataagctcaagagacgcggtttgaaaacgatataatgaatcatttggattttataataaaccctgacagtttttccactgtattgttttaacactcattggaagctgtattgattctaagaagctagaaatcaatacggccatacaaaagatgacattgaataagcaccggcttttttgattagcatataccttaaagcatgcattcatggctacatagttgttaaagggcttcttccattatcagtataatgaattacataatcatgcacttatatttgcccatctctgttctctcactcttgcctgggtatattctatgaaattgcgtatagcgtgtctccagttgaaccccaagcttggcgagtttgaagagaatgctaaccttgcgtattccttgcttcaggaaacattcaaggagaaacaggtcaagaagccaaacattttgatccttcccgagttagcattgactggctacaattttcaaagccagcagcggatagagccttttttggaggaaacaaccaagggagctagtacccaatgggctcaaaaagtatccaagacgtgggattgctttactttaataggatacccagaaaaaagtttagagagccctccccgtatttacaacagtgcggtacttgtatcgcctcagggaaaagtaatgaacaactacagaaagtccttcttgtatgaagctgatgaacattggggatgttcggaatcttctgatgggtttcaaacagtagatttattaattgaaggaaagactgtaaagacatcatttggaatttgcatggatttgaatccttataaatttgaagctccattcacagacttcgagttcagtggccattgcttgaaaaccggtacaagactcattttgtgcccaatggcctggttgtcccctctatcgccttccattaaaaaggatcttagtgatatagagaaaagcagacttcaaaagttctaccttgaaaaaatagataccccggaatttgacgttaattacgaattgaaaaaagatgaagtattgcccacccgtatgaatgaaacgttggaaacaattgactttgagccttcaaaaccggactactctaatataaattattggatactaaggttttttccctttctgactcatgtctataaacgagatgtgctcaaagagaatgcagttgcagtcttatgcaaccgagttggcattgagagtgatgtcttgtacggaggatcaaccacgattctaaacttcaatggtaagttagcatcgacacaagaggagctggagttgtacgggcagactaatagtctcaaccccagtgtggaagtattgggggcccttggcatgggtcaacagggaattctagtacgagacattgaattaacataatatacaatatacaataaacacaaataaagaatacaagcctgacaaaaattcacaaattattgcctagacttgtcgttatcagcagcgacctttttccaatgctcaatttcacgatatgccttttctagctctgctttaagcttctcattggaattggctaactcgttgactgcttggtcagtgatgagtttctccaaggtccatttctcgatgttgttgttttcgttttcctttaatctcttgatataatcaacagccttctttaatatctgagccttgttcgagtcccctgttggcaacagagcggccagttcctttattccgtggtttatattttctcttctacgcctttctacttctttgtgattctctttacgcatcttatgccattcttcagaaccagtggctggcttaaccgaatagccagagcctgaagaagccgcactagaagaagcagtggcattgttgactatgg

SEQ ID NO: 18 - DNA, 411 bp, Artificial Sequence
  Sequence of the 5′-Region used for knock out of BMT1
catatggtgagagccgttctgcacaactagatgttttcgagcttcgcattgtttcctgcagctcgactattgaattaagatttccggatatctccaatctcacaaaaacttatgttgaccacgtgctttcctgaggcgaggtgttttatatgcaagctgccaaaaatggaaaacgaatggccatttttcgcccaggcaaattattcgattactgctgtcataaagacagtgttgcaaggctcacatttttttttaggatccgagataaagtgaatacaggacagcttatctctatatcttgtaccattcgtgaatcttaagagttcggttagggggactctagttgagggttggcactcacgtatggctgggcgcagaaataaaattcaggcgcagcagcacttatcgatg

SEQ ID NO: 19 - DNA, 692 bp, Artificial Sequence
  Sequence of the 3′-Region used for knock out of BMT1
gaattcacagttataaataaaaacaaaaactcaaaaagtttgggctccacaaaataacttaatttaaatttttgtctaataaatgaatgtaattccaagattatgtgatgcaagcacagtatgcttcagccctatgcagctactaatgtcaatctcgcctgcgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagctt

SEQ ID NO: 20 - DNA, 1043 bp, Artificial Sequence
  Sequence of the 5′-Region used for knock out of BMT4
aagcttgttcaccgttgggacttttccgtggacaatgttgactactccaggagggattccagctttctctactagctcagcaataatcaatgcagccccaggcgcccgttctgatggcttgatgaccgttgtattgcctgtcactatagccaggggtagggtccataaaggaatcatagcagggaaattaaaagggcatattgatgcaatcactcccaatggctctcttgccattgaagtctccatatcagcactaacttccaagaaggaccccttcaagtctgacgtgatagagcacgcttgctctgccacctgtagtcctctcaaaacgtcaccttgtgcatcagcaaagactttaccttgctccaatactatgacggaggcaattctgtcaaaattctctctcagcaattcaaccaacttgaaagcaaattgctgtctcttgatgatggagacttttttccaagattgaaatgcaatgtgggacgactcaattgcttcttccagctcctcttcggttgattgaggaacttttgaaaccacaaaattggtcgttgggtcatgtacatcaaaccattctgtagatttagattcgacgaaagcgttgttgatgaaggaaaaggttggatacggtttgtcggtctctttggtatggccggtggggtatgcaattgcagtagaagataattggacagccattgttgaaggtagagaaaaggtcagggaacttgggggttatttataccattttaccccacaaataacaactgaaaagtacccattccatagtgagaggtaaccgacggaaaaagacgggcccatgttctgggaccaatagaactgtgtaatccattgggactaatcaacagacgattggcaatataatgaaatagttcgttgaaaagccacgtcagctgtcttttcattaactttggtcggacacaacattttctactgttgtatctgtcctactttgcttatcatctgccacagggcaagtggatttccttctcgcgcggctgggtgaaaacggttaacgtgaa

SEQ ID NO: 21 - DNA, 695 bp, Artificial Sequence
  Sequence of the 3′-Region used for knock out of BMT4
gccttgggggacttcaagtctttgctagaaactagatgaggtcaggccctcttatggttgtgtcccaattgggcaatttcactcacctaaaaagcatgacaattatttagcgaaataggtagtatattttccctcatctcccaagcagtttcgtttttgcatccatatctctcaaatgagcagctacgactcattagaaccagagtcaagtaggggtgagctcagtcatcagccttcgtttctaaaacgattgagttcttttgttgctacaggaagcgccctagggaactttcgcactttggaaatagattttgatgaccaagagcgggagttgatattagagaggctgtccaaagtacatgggatcaggccggccaaattgattggtgtgactaaaccattgtgtacttggacactctattacaaaagcgaagatgatttgaagtattacaagtcccgaagtgttagaggattctatcgagcccagaatgaaatcatcaaccgttatcagcagattgataaactcttggaaagcggtatcccattttcattattgaagaactacgataatgaagatgtgagagacggcgaccctctgaacgtagacgaagaaacaaatctacttttggggtacaatagagaaagtgaatcaagggaggtatttgtggccataatactcaactctatcattaatg

SEQ ID NO: 22 - DNA, 546 bp, Artificial Sequence
  Sequence of the 5′-Region used for knock out of BMT3
gatatctccctggggacaatatgtgttgcaactgttcgttgttggtgccccagtcccccaaccggtactaatcggtctatgttcccgtaactcatattcggttagaactagaacaataagtgcatcattgttcaacattgtggttcaattgtcgaacattgctggtgcttatatctacagggaagacgataagcctttgtacaagagaggtaacagacagttaattggtatttctttgggagtcgttgccctctacgttgtctccaagacatactacattctgagaaacagatggaagactcaaaaatgggagaagcttagtgaagaagagaaagttgcctacttggacagagctgagaaggagaacctgggttctaagaggctggactttttgttcgagagttaaactgcataattttttctaagtaaatttcatagttatgaaatttctgcagcttagtgtttactgcatcgtttactgcatcaccctgtaaataatgtgagcttttttccttccattgcttggtatcttccttgctgctgttt

SEQ ID NO: 23 - DNA, 378 bp, Artificial Sequence
  Sequence of the 3′-Region used for knock out of BMT3
acaaaacagtcatgtacagaactaacgcctttaagatgcagaccactgaaaagaattgggtcccatttttcttgaaagacgaccaggaatctgtccattttgtttactcgttcaatcctctgagagtactcaactgcagtcttgataacggtgcatgtgatgttctatttgagttaccacatgattttggcatgtcttccgagctacgtggtgccactcctatgctcaatcttcctcaggcaatcccgatggcagacgacaaagaaatttgggtttcattcccaagaacgagaatatcagattgcgggtgttctgaaacaatgtacaggccaatgttaatgctttttgttagagaaggaacaaacttttttgctgagc

SEQ ID NO: 24 - DNA, 1494 bp, Artificial Sequence
  DNA encodes Tr ManI catalytic domain
cgcgccggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgcttaa

SEQ ID NO: 25 - DNA, 57 bp, Artificial Sequence
  DNA encodes Saccharomyces cerevisiae mating factor pre-signal peptide
atgagattcccatccatcttcactgctgttttgttcgctgcttcttctgctttggct

SEQ ID NO: 26 - protein, 19 aa, Artificial Sequence
  Saccharomyces cerevisiae mating factor pre-signal peptide
MRFPSIFTAVLFAASSALA

SEQ ID NO: 27 - DNA, 934 bp, Artificial Sequence
  Pp AOX1 promoter
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 28 - DNA, 1242 bp, Artificial Sequence
  PpPRO1 5′ region and ORF
gagctcggccggaagggccatcgaattgtcatcgtctcctcaggtgccatcgctgtgggcatgaagagagtcaacatgaagcggaaaccaaaaaagttacagcaagtgcaggcattggctgctataggacaaggccgtttgataggactttgggacgaccttttccgtcagttgaatcagcctattgcgcagattttactgactagaacggatttggtcgattacacccagtttaagaacgctgaaaatacattggaacagcttattaaaatgggtattattcctattgtcaatgagaatgacaccctatccattcaagaaatcaaatttggtgacaatgacaccttatccgccataacagctggtatgtgtcatgcagactacctgtttttggtgactgatgtggactgtctttacacggataaccctcgtacgaatccggacgctgagccaatcgtgttagttagaaatatgaggaatctaaacgtcaataccgaaagtggaggttccgccgtaggaacaggaggaatgacaactaaattgatcgcagctgatttgggtgtatctgcaggtgttacaacgattatttgcaaaagtgaacatcccgagcagattttggacattgtagagtacagtatccgtgctgatagagtcgaaaatgaggctaaatatctggtcatcaacgaagaggaaactgtggaacaatttcaagagatcaatcggtcagaactgagggagttgaacaagctggacattcctttgcatacacgtttcgttggccacagttttaatgctgttaataacaaagagttttggttactccatggactaaaggccaacggagccattatcattgatccaggttgttataaggctatcactagaaaaaacaaagctggtattcttccagctggaattatttccgtagagggtaatttccatgaatacgagtgtgttgatgttaaggtaggactaagagatccagatgacccacattcactagaccccaatgaagaactttacgtcgttggccgtgcccgttgtaattaccccagcaatcaaatcaacaaaattaagggtctacaaagctcgcagatcgagcaggttctaggttacgctgacggtgagtatgttgttcacagggacaacttggctttcccagtatttgccgatccagaactgttggatgttgttgagagtaccctgtctgaacaggagagagaatccaaaccaaataaatag

SEQ ID NO: 29 - DNA, 376 bp, Artificial Sequence
  PpALG3 TT
atttacaattagtaatattaaggtggtaaaaacattcgtagaattgaaatgaattaatatagtatgacaatggttcatgtctataaatctccggcttcggtaccttctccccaattgaatacattgtcaaaatgaatggttgaactattaggttcgccagtttcgttattaagaaaactgttaaaatcaaattccatatcatcggttccagtgggaggaccagttccatcgccaaaatcctgtaagaatccattgtcagaacctgtaaagtcagtttgagatgaaatttttccggtctttgttgacttggaagcttcgttaaggttaggtgaaacagtttgatcaaccagcggctcccgttttcgtcgcttagtag

SEQ ID NO: 30 - DNA, 1434 bp, Artificial Sequence
  PpPRO1 3′ region
aatttcacatatgctgcttgattatgtaattataccttgcgttcgatggcatcgatttcctcttctgtcaatcgcgcatcgcattaaaagtatacttttttttttttcctatagtactattcgccttattataaactttgctagtatgagttctacccccaagaaagagcctgatttgactcctaagaagagtcagcctccaaagaatagtctcggtgggggtaaaggctttagtgaggagggtttctcccaaggggacttcagcgctaagcatatactaaatcgtcgccctaacaccgaaggctcttctgtggcttcgaacgtcatcagttcgtcatcattgcaaaggttaccatcctctggatctggaagcgttgctgtgggaagtgtgttgggatcttcgccattaactctttctggagggttccacgggcttgatccaaccaagaataaaatagacgttccaaagtcgaaacagtcaaggagacaaagtgttctttctgacatgatttccacttctcatgcagctagaaatgatcactcagagcagcagttacaaactggacaacaatcagaacaaaaagaagaagatggtagtcgatcttctttttctgtttcttcccccgcaagagatatccggcacccagatgtactgaaaactgtcgagaaacatcttgccaatgacagcgagatcgactcatctttacaacttcaaggtggagatgtcactagaggcatttatcaatgggtaactggagaaagtagtcaaaaagataacccgcctttgaaacgagcaaatagttttaatgatttttcttctgtgcatggtgacgaggtaggcaaggcagatgctgaccacgatcgtgaaagcgtattcgacgaggatgatatctccattgatgatatcaaagttccgggagggatgcgtcgaagttttttattacaaaagcatagagaccaacaactttctggactgaataaaacggctcaccaaccaaaacaacttactaaacctaatttcttcacgaacaactttatagagtttttggcattgtatgggcattttgcaggtgaagatttggaggaagacgaagatgaagatttagacagtggttccgaatcagtcgcagtcagtgatagtgagggagaattcagtgaggctgacaacaatttgttgtatgatgaagagtctctcctattagcacctagtacctccaactatgcgagatcaagaataggaagtattcgtactcctacttatggatctttcagttcaaatgttggttcttcgtctattcatcagcagttaatgaaaagtcaaatcccgaagctgaagaaacgtggacagcacaagcataaaacacaatcaaaaatacgctcgaagaagcaaactaccaccgtaaaagcagtgttgctgctattaaaggccttcat

SEQ ID NO: 31 - DNA, 260 bp, Artificial Sequence
  PpAOX1 TT
tcaagaggatgtcagaatgccatttgcctgagagatgcaggcttcattttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 32 - DNA, 375 bp, Artificial Sequence
  Encodes Sh ble ORF (Zeocin resistance marker)
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 33 - DNA, 427 bp, Artificial Sequence
  ScTEF1 promoter
gatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 34 - DNA, 1617 bp, Artificial Sequence
  PpTRP2 Region
atgagtgtaagtgatagtcatcttgcaacagattattttggaacgcaactaacaaagcagatacacccttcagcagaatcctttctggatattgtgaagaatgatcgccaaagtcacagtcctgagacagttcctaatctttaccccatttacaagttcatccaatcagacttcttaacgcctcatctggcttatatcaagcttaccaacagttcagaaactcccagtccaagtttcttgcttgaaagtgcgaagaatggtgacaccgttgacaggtacacctttatgggacattcccccagaaaaataatcaagactgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggaaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatga

SEQ ID NO: 35 - protein, 85 aa, Artificial Sequence
  Sc alpha mating factor signal sequence and pro-peptide
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 36 - protein, 16 aa, Artificial Sequence
  Sequence of the N-terminal 10X His peptide spacer
EEGHHHHHHHHHHEPK

SEQ ID NO: 37 - protein, 30 aa, Artificial Sequence
  Insulin P28N B chain
FVNQHLCGSHLVEALYLVCGERGFFYTNKT

SEQ ID NO: 38 - protein, 21 aa, Homo sapiens
GIVEQCCTSICSLYQLENYCN

SEQ ID NO: 39 - protein, 30 aa, Homo sapiens
FVNQHLCGSHLVEALYLVCGERGFFYTPKT

SEQ ID NO: 40 - protein, 10 aa, Artificial Sequence
  cMyc peptide
EQKLISEEDL

SEQ ID NO: 41 - protein, 15 aa, Artificial Sequence
  3xG4S spacer or linker peptide
GGGGSGGGGSGGGGS

SEQ ID NO: 42 - DNA, 960 bp, Artificial Sequence
  Encodes truncated ScSED1
caattttctaattctacatcagcatcttcaacagacgtaacttccagttcttcaatatcaacttccagtggttccgtcactatcacatcttcagaagctccagaaagtgataacggtacttctactgcagcccctacagaaacctcaactgaagccccaaccactgctattcctactaatggtacatctaccgaagcaccaacaaccgccatacctacaaacggtacttctacagaagcaccaactgatactacaaccgaagctccaactacagcattgcctacaaatggtacttctactgaagccccaactgacaccactacagaagctccaaccactggtttgcctacaaacggtacaacctcagcttttccacctactacatccttaccacctagtaataccactacaaccccaccttataacccatctactgattatactacagactacacagttgtaactgaatataccacttactgtccagaacctacaaccttcactacaaatggtaaaacatacaccgttactgaaccaaccactttaacaataaccgattgtccatgcacaatcgaaaagcctacaaccacttctacaaccgaatacacagtcgttactgaatacactacatactgtccagaacctaccactttcacaaccaatggtaaaacttacacagttaccgaaccaactacattgactattacagactgtccttgcactatagaaaagtcagaagctccagaatccagtgtacctgtcacagaatccaaaggtactactacaaaggaaactggtgttaccactaaacaaacaaccgcaaatccatctttaacagtctcaactgtagtccctgtttcttcatccgccagttctcattcagttgtaattaattccaacggtgctaatgttgtcgttccaggtgctttgggtttggcaggtgttgctatgttgtttttg

SEQ ID NO: 43 - protein, 320 aa, Artificial Sequence
  Truncated ScSED1
QFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 44 - protein, 12 aa, Homo sapiens
GYGSSSRRAPQT

SEQ ID NO: 45 - protein, 12 aa, Artificial Sequence
  IGF-1 (Y2A) C-peptide
GAGSSSRRAPQT

SEQ ID NO: 46 - DNA, 1524 bp, Artificial Sequence
  DNA encoding fusion protein I
atgagatttccaagtatttttaccgccgtcttatttgctgcctcctccgctttagccgccccagtcaacaccaccaccgaagatgaaacagctcaaatcccagctgaagcagttattggttattcagatttggagggtgactttgacgtcgcagttttgcctttctcaaattccactaacaacggtttgttgtttattaacactacaatagccagtatcgctgcaaaagaagaaggtgtttctttggaaaagagagaagaaggtcatcaccaccatcatcaccatcaccatcacgaaccaaaattcgtaaatcaacatttgtgtggttctcacttagttgaagctttgtatttggtatgcggtgaaagaggtttcttttataccaacaaaactgccgctaagggtatcgttgaacaatgttgcacttccatatgtagtttgtaccaattggaaaactactgcaactctcatggttcagaacaaaagttgatctcagaagaagatttgttggaaggtggtggtggttccggtggtggtggttctggtggtggtggttctgttgatcaattttctaattctacatcagcatcttcaacagacgtaacttccagttcttcaatatcaacttccagtggttccgtcactatcacatcttcagaagctccagaaagtgataacggtacttctactgcagcccctacagaaacctcaactgaagccccaaccactgctattcctactaatggtacatctaccgaagcaccaacaaccgccatacctacaaacggtacttctacagaagcaccaactgatactacaaccgaagctccaactacagcattgcctacaaatggtacttctactgaagccccaactgacaccactacagaagctccaaccactggtttgcctacaaacggtacaacctcagcttttccacctactacatccttaccacctagtaataccactacaaccccaccttataacccatctactgattatactacagactacacagttgtaactgaatataccacttactgtccagaacctacaaccttcactacaaatggtaaaacatacaccgttactgaaccaaccactttaacaataaccgattgtccatgcacaatcgaaaagcctacaaccacttctacaaccgaatacacagtcgttactgaatacactacatactgtccagaacctaccactttcacaaccaatggtaaaacttacacagttaccgaaccaactacattgactattacagactgtccttgcactatagaaaagtcagaagctccagaatccagtgtacctgtcacagaatccaaaggtactactacaaaggaaactggtgttaccactaaacaaacaaccgcaaatccatctttaacagtctcaactgtagtccctgtttcttcatccgccagttctcattcagttgtaattaattccaacggtgctaatgttgtcgttccaggtgctttgggtttggcaggtgttgctatgttgtttttg

SEQ ID NO: 47 - protein, 508 aa, Artificial Sequence
  Fusion protein I
MRFPSIFTAVLFAASSALAAPVNTTTEDETAQIPAEAVIGYSDLEGDFDVAVLPFSNSTNNGLLFINTTIASIAAKEEGVSLEKREEGHHHHHHHHHHEPKFVNQHLCGSHLVEALYLVCGERGFFYTNKTAAKGIVEQCCTSICSLYQLENYCNSHGSEQKLISEEDLLEGGGGSGGGGSGGGGSVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 48 - protein, 423 aa, Artificial Sequence
  Fusion protein IA
EEGHHHHHHHHHHEPKFVNQHLCGSHLVEALYLVCGERGFFYTNKTAAKGIVEQCCTSICSLYQLENYCNSHGSEQKLISEEDLLEGGGGSGGGGSGGGGSVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 49 - DNA, 1551 bp, Artificial Sequence
  DNA encoding fusion protein II
atgagatttccaagtatttttaccgccgtcttatttgctgcctcctccgctttagccgccccagtcaacaccaccaccgaagatgaaacagctcaaatcccagctgaagcagttattggttattcagatttggagggtgactttgacgtcgcagttttgcctttctcaaattccactaacaacggtttgttgtttattaacactacaatagccagtatcgctgcaaaagaagaaggtgtttctttggaaaagagagaagaaggtcatcaccaccatcatcaccatcaccatcacgaaccaaaattcgtaaatcaacatttgtgtggttctcacttagttgaagctttgtatttggtatgcggtgaaagaggtttcttttataccaacaaaactggttatggatcttcctcaagaagagccccacaaaccggtatcgttgaacaatgttgcacttccatatgtagtttgtaccaattggaaaactactgcaactctcatggttcagaacaaaagttgatctcagaagaagatttgttggaaggtggtggtggttccggtggtggtggttctggtggtggtggttctgttgatcaattttctaattctacatcagcatcttcaacagacgtaacttccagttcttcaatatcaacttccagtggttccgtcactatcacatcttcagaagctccagaaagtgataacggtacttctactgcagcccctacagaaacctcaactgaagccccaaccactgctattcctactaatggtacatctaccgaagcaccaacaaccgccatacctacaaacggtacttctacagaagcaccaactgatactacaaccgaagctccaactacagcattgcctacaaatggtacttctactgaagccccaactgacaccactacagaagctccaaccactggtttgcctacaaacggtacaacctcagcttttccacctactacatccttaccacctagtaataccactacaaccccaccttataacccatctactgattatactacagactacacagttgtaactgaatataccacttactgtccagaacctacaaccttcactacaaatggtaaaacatacaccgttactgaaccaaccactttaacaataaccgattgtccatgcacaatcgaaaagcctacaaccacttctacaaccgaatacacagtcgttactgaatacactacatactgtccagaacctaccactttcacaaccaatggtaaaacttacacagttaccgaaccaactacattgactattacagactgtccttgcactatagaaaagtcagaagctccagaatccagtgtacctgtcacagaatccaaaggtactactacaaaggaaactggtgttaccactaaacaaacaaccgcaaatccatctttaacagtctcaactgtagtccctgtttcttcatccgccagttctcattcagttgtaattaattccaacggtgctaatgttgtcgttccaggtgctttgggtttggcaggtgttgctatgttgtttttg

SEQ ID NO: 50 - protein, 517 aa, Artificial Sequence
  Fusion protein II
MRFPSIFTAVLFAASSALAAPVNTTTEDETAQIPAEAVIGYSDLEGDFDVAVLPFSNSTNNGLLFINTTIASIAAKEEGVSLEKREEGHHHHHHHHHHEPKFVNQHLCGSHLVEALYLVCGERGFFYTNKTGYGSSSRRAPQTGIVEQCCTSICSLYQLENYCNSHGSEQKLISEEDLLEGGGGSGGGGSGGGGSVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 51 - protein, 432 aa, Artificial Sequence
  Fusion protein IIA
EEGHHHHHHHHHHEPKFVNQHLCGSHLVEALYLVCGERGFFYTNKTGYGSSSRRAPQTGIVEQCCTSICSLYQLENYCNSHGSEQKLISEEDLLEGGGGSGGGGSGGGGSVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 52 - DNA, 1551 bp, Artificial Sequence
  DNA encoding fusion protein III
atgagatttccaagtatttttaccgccgtcttatttgctgcctcctccgctttagccgccccagtcaacaccaccaccgaagatgaaacagctcaaatcccagctgaagcagttattggttattcagatttggagggtgactttgacgtcgcagttttgcctttctcaaattccactaacaacggtttgttgtttattaacactacaatagccagtatcgctgcaaaagaagaaggtgtttctttggaaaagagagaagaaggtcatcaccaccatcatcaccatcaccatcacgaaccaaaattcgtaaatcaacatttgtgtggttctcacttagttgaagctttgtatttggtatgcggtgaaagaggtttcttttataccaacaaaactggtgctggatcttcctcaagaagagccccacaaaccggtatcgttgaacaatgttgcacttccatatgtagtttgtaccaattggaaaactactgcaactctcatggttcagaacaaaagttgatctcagaagaagatttgttggaaggtggtggtggttccggtggtggtggttctggtggtggtggttctgttgatcaattttctaattctacatcagcatcttcaacagacgtaacttccagttcttcaatatcaacttccagtggttccgtcactatcacatcttcagaagctccagaaagtgataacggtacttctactgcagcccctacagaaacctcaactgaagccccaaccactgctattcctactaatggtacatctaccgaagcaccaacaaccgccatacctacaaacggtacttctacagaagcaccaactgatactacaaccgaagctccaactacagcattgcctacaaatggtacttctactgaagccccaactgacaccactacagaagctccaaccactggtttgcctacaaacggtacaacctcagcttttccacctactacatccttaccacctagtaataccactacaaccccaccttataacccatctactgattatactacagactacacagttgtaactgaatataccacttactgtccagaacctacaaccttcactacaaatggtaaaacatacaccgttactgaaccaaccactttaacaataaccgattgtccatgcacaatcgaaaagcctacaaccacttctacaaccgaatacacagtcgttactgaatacactacatactgtccagaacctaccactttcacaaccaatggtaaaacttacacagttaccgaaccaactacattgactattacagactgtccttgcactatagaaaagtcagaagctccagaatccagtgtacctgtcacagaatccaaaggtactactacaaaggaaactggtgttaccactaaacaaacaaccgcaaatccatctttaacagtctcaactgtagtccctgtttcttcatccgccagttctcattcagttgtaattaattccaacggtgctaatgttgtcgttccaggtgctttgggtttggcaggtgttgctatgttgtttttg

SEQ ID NO: 53 - protein, 517 aa, Artificial Sequence
  Fusion protein III
MRFPSIFTAVLFAASSALAAPVNTTTEDETAQIPAEAVIGYSDLEGDFDVAVLPFSNSTNNGLLFINTTIASIAAKEEGVSLEKREEGHHHHHHHHHHEPKFVNQHLCGSHLVEALYLVCGERGFFYTNKTGAGSSSRRAPQTGIVEQCCTSICSLYQLENYCNSHGSEQKLISEEDLLEGGGGSGGGGSGGGGSVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 54 - protein, 432 aa, Artificial Sequence
  Fusion protein IIIA
EEGHHHHHHHHHHEPKFVNQHLCGSHLVEALYLVCGERGFFYTNKTGAGSSSRRAPQTGIVEQCCTSICSLYQLENYCNSHGSEQKLISEEDLLEGGGGSGGGGSGGGGSVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 55 - DNA, 30 bp, Artificial Sequence
  PCR primer c/o-ScSED1-FW
tccagaaagtgataacggtacttctactgc

SEQ ID NO: 56 - DNA, 32 bp, Artificial Sequence
  PCR primer c/o-ScSED1-RV
aatgtagttggttcggtaactgtgtaagtttt

SEQ ID NO: 57 - protein, 38 aa, Artificial Sequence
  Human GR2 coiled coil peptide sequence
TSRLEGLQSENHRLRMKITELDKDLEEVTMQLQDVGGC

SEQ ID NO: 58 - protein, 38 aa, Artificial Sequence
  Human GR1 coiled coil peptide sequence
EEKSRLLEKENRELEKIIAEKEERVSELRHQLQSVGGC

SEQ ID NO: 59 - DNA, 255 bp, Artificial Sequence
  Encodes Sc alpha mating factor signal sequence and pro-peptide
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagg

SEQ ID NO: 60 - protein, 389 aa, Artificial Sequence
  SED 1 Fusion with signal seq, GR2, and cMyc
MRFPSIFTAVLFAASSALATSRLEGLQSENHRLRMKITELDKDLEEVTMQLQDVGGCEQKLISEEDLVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 61 - protein, 370 aa, Artificial Sequence
  SED 1 Fusion with signal seq, GR1, and cMyc
TSRLEGLQSENHRLRMKITELDKDLEEVTMQLQDVGGCEQKLISEEDLVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 62 - protein, 223 aa, Artificial Sequence
  Pre-proinsulin analogue precursor GR1 fusion with cMyc
MRFPSIFTAVLFAASSALAAPVNTTTEDETAQIPAEAVIGYSDLEGDFDVAVLPFSNSTNNGLLFINTTIASIAAKEEGVSLEKEEGHHHHHHHHHHEPKFVNQHLCGSHLVEALYLVCGERGFFYTNKTAAKGIVEQCCTSICSLYQLENYCNSHGSEQKLISEEDLLEGGGGSGGGGSGGGGSEEKSRLLEKENRELEKIIAEKEERVSELRHQLQSVGGC

SEQ ID NO: 63 - protein, 139 aa, Artificial Sequence
  Insulin analogue precursor GR1 fusion
EEGHHHHHHHHHHEPKFVNQHLCGSHLVEALYLVCGERGFFYTNKTAAKGIVEQCCTSICSLYQLENYCNSHGSEQKLISEEDLLEGGGGSGGGGSGGGGSEEKSRLLEKENRELEKIIAEKEERVSELRHQLQSVGGC

SEQ ID NO: 64 - protein, 514 aa, Artificial Sequence
  Pre-proinsulin precursor fused at the C-terminus to the N-terminus of a truncated Saccharomyces cerevisiae SED1 protein
MRFPSIFTAVLFAASSALAAPVNTTTEDETAQIPAEAVIGYSDLEGDFDVAVLPFSNSTNNGLLFINTTIASIAAKEEGVSLEKRFVNQHLCGSHLVEALYLVCGERGFFYTPKTRREAEDLQVGQVELGGGPGAGSLQPLALEGSLQKRGIVEQCCTSICSLYQLENYCNSHGSEQKLISEEDLGGGGSASVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 65 - protein, 35 aa, Artificial Sequence
  Human insulin C-peptide
RREAEDLQVGQVELGGGPGAGSLQPLALEGSLQKR

SEQ ID NO: 66 - protein, 7 aa, Artificial Sequence
  Spacer or linker peptide
GGGGSAS

SEQ ID NO: 67 - protein, 4 aa, Artificial Sequence
  Kex2 cleavage site
LQKR

SEQ ID NO: 68 - protein, 4 aa, Artificial Sequence
  Kex2 consensus cleavage site
LXKR

SEQ ID NO: 69 - protein, 65 aa, Artificial Sequence
  B-chain peptide/C-peptide fusion
FVNQHLCGSHLVEALYLVCGERGFFYTPKTRREAEDLQVGQVELGGGPGAGSLQPLALEGSLQKR

SEQ ID NO: 70 - protein, 364 aa, Artificial Sequence
  A-chain peptide/sed1p fusion
GIVEQCCTSICSLYQLENYCNSHGSEQKLISEEDLGGGGSASVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 71 - DNA, 6549 bp, Artificial Sequence
  Sequence of plasmid pGLY11680
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaggtttgttaaccaacatttgtgtggatcccaccttgttgaagcattgtaccttgtctgcggagagagaggtttcttttacactccaaagacaagaagagaagctgaggatttgcaagttggtcaggtcgaacttggtggaggtccaggagctggttcattgcaacctttggcccttgaaggaagtttgcaaaagagaggtattgtcgagcagtgttgcacttctatctgttccttgtaccagcttgagaactattgcaattctcatggttcagaacaaaagttgatctcagaagaagatttgggtggaggcggttctgctagcgtcgaccaattctctaactctacttccgcttcctctactgacgttacttcctcctcctctatttctacttcctccggttccgttactattacttcctctgaggctccagaatctgacaacggtacttctactgctgctccaactgaaacttctactgaggctcctactactgctattccaactaacggaacttccacagaggctccaacaacagctatccctacaaacggtacatccactgaagctcctactgacactactacagaagctccaactactgctttgcctactaatggtacatcaacagaggctcctacagatacaacaactgaagctccaacaactggattgccaacaaacggtactacttctgctttcccaccaactacttccttgccaccatccaacactactactactccaccatacaacccatccactgactacactactgactacacagttgttactgagtacactacttactgtccagagccaactactttcacaacaaacggaaagacttacactgttactgagcctactactttgactatcactgactgtccatgtactatcgagaagccaactactacttccactacagagtatactgttgttacagaatacacaacatattgtcctgagccaacaacattcactactaatggaaaaacatacacagttacagaaccaactacattgacaattacagattgtccttgtacaattgagaagtccgaggctcctgaatcttctgttccagttactgaatccaagggtactactactaaagaaactggtgttactactaagcagactactgctaacccatccttgactgtttccactgttgttccagtttcttcctctgcttcttcccactccgttgttatcaactccaacggtgctaacgttgttgttcctggtgctttgggattggctggtgttgctatgttgttcttgtaatagggccggccatttaaattcaagaggatgtcagaatgccatttgcctgagagatgcaggcttcattttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgcagcggccgcttacgcgccgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaattaattaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggcctgccatgaccgagatcggcgagcagccgtgggggcgggagttcgccctgcgcgacccggccggcaactgcgtgcacttcgtggccgaggagcaggactgattaattaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgcggcctaaggcgcgccaggccataatggcctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 72 - DNA, 7334 bp, Artificial Sequence
  Sequence of plasmid pGLY10569
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaggtttgttaaccaacatttgtgtggatcccaccttgttgaagcattgtaccttgtctgcggagagagaggtttcttttacactccaaagacaagaagagaagctgaggatttgcaagttggtcaggtcgaacttggtggaggtccaggagctggttcattgcaacctttggcccttgaaggaagtttgcaaaagagaggtattgtcgagcagtgttgcacttctatctgttccttgtaccagcttgagaactattgcaattaatagggccggccatttaaattcaagaggatgtcagaatgccatttgcctgagagatgcaggcttcattttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgcagcggccgcttacgcgccgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaattaattaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggcctgccatgaccgagatcggcgagcagccgtgggggcgggagttcgccctgcgcgacccggccggcaactgcgtgcacttcgtggccgaggagcaggactgattaattaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgcggcctaaggcgcgccaggccataatggccaaacggtttctcaattactatatactactaaccatttacctgtagcgtatttcttttccctcttcgcgaaagctcaagggcatcttcttgactcatgaaaaatatctggatttcttctgacagatcatcacccttgagcccaactctctagcctatgagtgtaagtgatagtcatcttgcaacagattattttggaacgcaactaacaaagcagatacacccttcagcagaatcctttctggatattgtgaagaatgatcgccaaagtcacagtcctgagacagttcctaatctttaccccatttacaagttcatccaatcagacttcttaacgcctcatctggcttatatcaagcttaccaacagttcagaaactcccagtccaagtttcttgcttgaaagtgcgaagaatggtgacaccgttgacaggtacacctttatgggacattcccccagaaaaataatcaagactgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggaaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatgaacggaggacgtaagtaggaatttatggtttggccataatggcctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc